(19) United States
(12) Patent Application Publication
(10) Pub. No.: US 2024/0052429 A1
(43) Pub. Date: Feb. 15, 2024

(54) LIVER CANCER DETECTION KIT OR DEVICE, AND DETECTION METHOD

(71) Applicants: TORAY INDUSTRIES, INC., Tokyo (JP); NATIONAL CANCER CENTER, Tokyo (JP)

(72) Inventors: Satoshi KONDOU, Kamakura-shi (JP); Hitoshi NOBUMASA, Kamakura-shi (JP); Satoko KOZONO, Kamakura-shi (JP); Hiroko SUDO, Kamakura-shi (JP); Junpei KAWAUCHI, Kamakura-shi (JP); Atsushi OCHIAI, Kashiwa-shi (JP); Motohiro KOJIMA, Kashiwa-shi (JP)

(73) Assignees: TORAY INDUSTRIES, INC., Tokyo (JP); NATIONAL CANCER CENTER, Tokyo (JP)

(21) Appl. No.: 18/491,272

(22) Filed: Oct. 20, 2023

Related U.S. Application Data
(62) Division of application No. 17/970,824, filed on Oct. 21, 2022, now Pat. No. 11,827,941.
Application No. 17/970,824 is a division of application No. 16/785,233, filed on Feb. 7, 2020, now Pat. No. 11,512,355.
Application No. 16/785,233 is a division of application No. 15/319,585, filed on Dec. 16, 2016, now Pat. No. 10,590,487.

(30) Foreign Application Priority Data
Jun. 18, 2014 (JP) 2014-124880

(51) Int. Cl. C12Q 1/6886; G01N 33/574; G01N 37/00; G01N 33/53; C12N 15/09; C12Q 1/04; C12M 1/34

(52) U.S. Cl. CPC C12Q 1/6886; G01N 33/574; G01N 37/00; G01N 33/53; C12N 15/09; C12Q 1/04; C12M 1/34; C12Q 2600/118; C12Q 2600/158; C12Q 2600/178

(57) ABSTRACT

It is intended to provide a kit or device for the detection of liver cancer and a method for detecting liver cancer. The present invention relates to a kit or device for the detection of liver cancer, comprising a nucleic acid capable of specifically binding to miRNA in a sample of a subject, and a method for detecting liver cancer, comprising measuring the miRNA in vitro.

### CROSS-REFERENCE TO RELATED APPLICATIONS

This application is a Divisional of copending application No. 17,970,824 filed Oct. 21, 2022, which is a Divisional of copending application Ser. No. 16/785,233, filed on Feb. 7, 2020, now U.S. Pat. No. 11,512,355, which is a Divisional of application Ser. No. 15/319,585, filed on Dec. 16, 2016, now U.S. Pat. No. 10,590,487, which is the National Phase under 35 U.S.C. § 371 of International Application No. PCT/JP2015/067552, filed on Jun. 18, 2015, which claims the benefit under 35 U.S.C. § 119(a) to Patent Application No. 2014-124880, filed in Japan on Jun. 18, 2014, all of which are hereby expressly incorporated by reference into the present application.

### Reference to Electronic Sequence Listing

This application contains a Sequence Listing which has been submitted electronically in .XML format and is hereby incorporated by reference in its entirety. Said .XML copy, created on Oct. 6, 2022, is named “PH-6238-PCT-US-DIV1-DIV1.xml” and is 688,621 bytes in size. The sequence listing contained in this .XML file is part of the specification and is hereby incorporated by reference herein in its entirety.

### TECHNICAL FIELD

The present invention relates to a kit or a device for the detection of liver cancer, comprising a nucleic acid capable of specifically binding to a particular miRNA, which is used for examining the presence or absence of liver cancer in a subject, and a method for detecting liver cancer, comprising measuring an expression level of the miRNA using the nucleic acid.

### BACKGROUND ART

The liver is the largest organ in the body and is positioned in the upper right portion of the abdomen. Its main roles are the metabolism of nutrients and the detoxication and elimination of harmful substances. According to the 2011 statistics of cancer types in Japan disclosed by the Center for Cancer Control and Information Services, National Cancer Center, the number of individuals affected by liver cancer is 47,271 people. Namely, it is estimated that one out of every 35 Japanese individuals experience liver cancer. The number of individuals affected by liver cancer among other cancer types takes the 6th in place. Also, men are nearly twice as likely as women to develop liver cancer. The number of liver cancer deaths in men and women together climbed to 30,690 people and takes the 4th in place. An estimate of the number of American individuals affected by liver cancer in 2014 climbs to 33,190 people, among which approximately 23,000 people will die (Non-Patent Literature 1).

In general, primary liver cancer often refers to hepatocellular carcinoma which accounts for approximately 80% of primary liver cancer cases. However, there are other subtypes of primary liver cancer such as intrahepatic bile duct carcinoma which accounts for 10 to 20% of all primary liver cancer cases, and biliary cystadenocarcinoma which is a rarer cancer type.

The stages of liver cancer progression are specified separately for hepatocellular carcinoma and intrahepatic bile duct carcinoma in Non-Patent Literature 2. Herein, particularly, the hepatocellular carcinoma is classified into stage I (T1/N0/M0), stage II (T2/N0/M0), stage IIIA (T3a/N0/M0), stage IIB (T3b/N0/M0), stage IIIC (T4/N0/M0), stage IVA (N1/M0), and stage IVB (M1) according to the degrees of tumor spread (T0 to T4), lymph node metastasis (N0 and N1), and distant metastasis (M0 and M1).

The 5-year relative survival rate of liver cancer differs depending on the stages of progression. According to Non-Patent Literature 1, the 5-year relative survival rate of liver cancer is reportedly 28% for tumors localized within liver (stage 1, stage 2 and some cases of stage 3), 7% for tumors found to have metastasized to a surrounding area of liver (stage IIIC and stage IVA), and 2% for tumors found to have metastasized distantly (stage IVB). Thus, the detection and treatment of liver cancer at an early stage before metastasis makes a significant contribution to improvement in the survival rate.

The treatment of liver cancer is performed mainly by 3 procedures: surgical therapy mainly involving resection and/or liver transplantation; local therapy which involves injecting a drug through centesis or performing cauterization to kill cancer; and hepatic arterial embolization. These procedures are used in combination with drug therapy or radiotherapy. Particularly, early liver cancer which is found not to metastasize to a blood vessel or an adjacent site is often cured by the partial resection of the liver (Non-Patent Literature 1). On the other hand, even if cancer is localized, liver transplantation is desirable for the cases where such resection is impossible on the ground that the tumors have a large size or are placed in proximity to a blood vessel, for example. If metastasis is found, systemic drug therapy or radiotherapy is performed (Non-Patent Literature 1).

As described in Non-Patent Literature 1, primary tests of liver cancer are inspection and palpation as well as imaging tests such as ultrasonography, CT scan, MRI scan, and angiography. For example, AFP (alpha fetoprotein) and PIVKA-II are known as tumor markers for the detection of liver cancer. The tests using these tumor markers are often performed in combination with ultrasonography. When there are findings that suspect liver cancer by these primary tests, pathological examination which involves inserting a needle into a lesion and collecting cells or tissues, which are then examined under a microscope is carried out as a secondary test.

Meanwhile, it is known that the most important leading cause of liver cancer is prolonged infection with hepatitis B or C virus. Therefore, subjects suspected of having liver cancer may be subjected to a hepatitis virus test in addition to the primary tests described above.

As shown in Patent Literatures 1 to 5, there are reports, albeit at a research stage, on methods for detecting liver cancer using the expression levels of microRNAs (miRNAs) in biological samples including blood and hepatic tissues.

Patent Literature 1 discloses a method for detecting leukemia, breast cancer, and liver cancer using miRNAs: hsa-miR-92a-3p, hsa-miR-92b-3p, hsa-miR-92a-2-5p, and hsa-miR-92b-5p in tissues as markers.

Patent Literature 2 has reported a method for diagnosing various cancers using, as markers, miRNAs such as hsa-miR-23a-3p, hsa-miR-23b-3p, hsa-miR-24-3p, hsa-miR-557, hsa-miR-564, hsa-miR-614, hsa-miR-150-3p, and hsa-miR-486-3p contained in vesicles circulating in body fluids.

Patent Literature 3 discloses a method for detecting various diseases including liver cancer using miRNAs such as hsa-miR-23b-3p, hsa-miR-30c-1-3p, hsa-miR-125a-3p, and hsa-miR-486-3p in tissues or body fluids as markers.

Patent Literature 4 discloses a method for detecting various pathological conditions including liver cancer using, as markers, miRNAs such as hsa-miR-16-5p, hsa-miR-92a-3p, hsa-miR-663a, hsa-miR-1913, and hsa-miR-625-3p, or proteins contained in vesicles circulating in body fluids.

Patent Literature 5 discloses that hsa-miR-187-5p, hsa-miR-92a-3p, hsa-miR-16-5p, and hsa-miR-30c-1-3p in plasma are markers for colorectal cancer, liver cancer, and lung cancer.

### CITATION LIST

### Patent Literature

Patent Literature 1: International Publication No. WO 2010/123043
Patent Literature 2: U.S. Patent Application Publication No. 2011/003704
Patent Literature 3: International Publication No. WO 2010/054386
Patent Literature 4: International Publication No. WO 2012/174282
Patent Literature 5: International Publication No. WO 2011/076142

### Non-Patent Literature

Non-Patent Literature 1: American Cancer Society, “Liver Cancer”, 2013, p. 5 to 8, 14 to 15, 17 to 23, and 27 to 41
Non-Patent Literature 2: Sobin, L. et al., “TNM Classification of Malignant Tumours, the 7th edition”, 2010, p. 104-107
Non-Patent Literature 3: NCCN Guidelines, “Hepatobiliary Cancers, the 2nd edition”, 2014, MS-4
Non-Patent Literature 4: Zhang, B and Yang, B., 1999, Journal of Medical Screening, Vol. 6 (2), p. 108-110
Non-Patent Literature 5: Takahashi, A. et al., 2008, World Journal of Gastroenterology, Vol. 14 (1), p. 129-31

### SUMMARY OF INVENTION

### Problem to be Solved by Invention

An object of the present invention is to find a novel tumor marker for liver cancer and to provide a method that can effectively detect liver cancer using a nucleic acid capable of specifically binding to the marker.

Liver cancer progresses without particular symptoms and is therefore difficult to detect early. Since the most part of the liver is housed in the right rib, liver cancer is difficult to detect by palpation. An effective method for liver cancer screening has not yet been established for ordinary people lacking a risk of liver cancer such as hepatitis virus infection or liver cirrhosis (Non-Patent Literature 1). Ultrasonography is a widely prevalent method for liver cancer screening because this method places less burden on patients and is convenient. Nonetheless, liver cancer may be difficult to detect depending on its site of occurrence by ultrasonography. In addition, examination results of ultrasonography largely depend on the skill of technicians. Therefore, it is considered to be desirable that ultrasonography should be used in combination with a tumor marker (Non-Patent Literature 3). Although AFP is known as a tumor marker for the detection of liver cancer, liver cancer found to have an elevated level of AFP is already at an advanced stage and is impossible to resect or has metastasized to an area outside the liver in many cases (Non-Patent Literature 1). It has been reported that some liver cancers do not produce AFP. Meanwhile, AFP is known to also elevate in cancers other than liver cancer, for example, testicular cancer or ovary cancer, and further to elevate in non-cancer liver diseases, for example, sustained hepatitis virus infection, and is therefore regarded as a low specific marker (Non-Patent Literature 1). For example, false diagnosis of other cancers as liver cancer wastes appropriate therapeutic opportunity or places unnecessary economical and physical burdens on patients due to the application of wrong medicine. According to results of large-scale screening research targeting hepatitis B-infected people and prolonged hepatitis patients (Non-Patent Literature 4), the AFP test has liver cancer detection sensitivity as low as 69% and thus has insufficient examination performance for use as a liver cancer screening test. Furthermore, CT scan or MRI scan can detect liver cancer with high performance, but is not suitable as a widely prevalent primary test because these tests require a specific apparatus and high examination cost.

As described below, there are reports, albeit at a research stage, on the determination of liver cancer using the expression levels of microRNAs (miRNAs) in biological samples including blood, none of which, however, have yet been brought into practical use.

Patent Literature 1 discloses a method for detecting leukemia, breast cancer, and liver cancer using miRNAs hsa-miR-92a-3p, hsa-miR-92b-3p, hsa-miR-92a-2-5p, and hsa-miR-92b-5p in blood cells or tissues as markers. This detection method, however, inevitably requires tissue resection by surgical operation for obtaining samples, and this step places a heavy physical burden on patients. Therefore, this method is not favorable as an examination method. In addition, Patent Literature 1 does not describe specific detection performance such as accuracy, sensitivity, or specificity for determining liver cancer as to this detection method, which is thus industrially less practical.

Patent Literature 2 has reported a method for diagnosing various cancers using, as markers, miRNAs such as hsa-miR-23a-3p, hsa-miR-23b-3p, hsa-miR-24-3p, hsa-miR-557, hsa-miR-564, hsa-miR-614, hsa-miR-150-3p, and hsa-miR-486-3p contained in vesicles circulating in body fluids. Patent Literature 2, however, neither describes a specific method for diagnosing liver cancer by use of this detection method nor describes detection performance such as accuracy, sensitivity, or specificity for determining liver cancer. Therefore, this detection method is industrially less practical.

Patent Literature 3 discloses a method for detecting various diseases including liver cancer using miRNAs such as hsa-miR-23b-3p, hsa-miR-30c-1-3p, hsa-miR-125a-3p, and hsa-miR-486-3p in tissues or body fluids as markers. This detection method, however, is based on results of experiments using mouse models, and the detection of liver cancer in humans is unknown about the method. In addition, Patent Literature 3 does not describe detection performance such as accuracy, sensitivity, or specificity for determining liver cancer. Therefore, this detection method is industrially less practical.

Patent Literature 4 discloses a method for detecting various pathological conditions including liver cancer using, as markers, miRNAs such as hsa-miR-16-5p, hsa-miR-92a-3p, hsa-miR-663a, hsa-miR-1913, and hsa-miR-625-3p, or proteins contained in vesicles circulating in body fluids. Patent Literature 4, however, neither describes a specific method for diagnosing liver cancer by use of this detection method nor validated these miRNA markers in an independent sample group. Therefore, this detection method is less reliable.

Patent Literature 5 discloses that hsa-miR-187-5p, hsa-miR-92a-3p, hsa-miR-16-5p, and hsa-miR-30c-1-3p in plasma are markers for colorectal cancer, liver cancer, and lung cancer. These markers, however, are markers for discriminating a group of colorectal cancers from a group of liver cancers, lung cancers, and healthy subjects and is not a marker for detecting liver cancer.

As mentioned above, the existing tumor markers exhibit low performance in the detection of liver cancer, and neither performance nor detection methods are specifically shown as to the markers at a research stage. Therefore, use of these markers might lead to carrying out needless extra examination due to the false detection of healthy subjects as being liver cancer patients, or might waste therapeutic opportunity because of overlooking liver cancer patients. In addition, the measurement of several dozens to several hundreds of miRNAs increases examination cost and is therefore difficult to use in large-scale screening such as medical checkup. Furthermore, the collection of liver tissues for measuring the tumor markers is highly invasive to patients and is not favorable. Hence, there is a demand for a highly accurate liver cancer marker that is detectable from blood, which can be collected with limited invasiveness, and is capable of correctly determining a liver cancer patient as a liver cancer patient and a healthy subject as a healthy subject. Particularly, the early detection and treatment of liver cancer can improve the survival rates. In addition, such liver cancer is often cured by the partial resection of the liver. Therefore, a highly sensitive liver cancer marker capable of detecting liver cancer even at an early stage of progression is desired.

### Means for Solution of Problem

The present inventors have conducted diligent studies to attain the object and consequently completed the present invention by finding multiple genes usable as markers for the detection of liver cancer from blood, which can be collected with limited invasiveness, and finding that liver cancer can be significantly detected by using nucleic acid(s) capable of specifically binding to any of these markers.

### SUMMARY OF INVENTION

Specifically, the present invention has the following features:

(1) A kit for the detection of liver cancer, comprising nucleic acid(s) capable of specifically binding to at least one or more polynucleotide(s) selected from the group consisting of liver cancer markers: miR-1343-3p, miR-6726-5p, miR-6515-3p, miR-4651, miR-4257, miR-3188, miR-6131, miR-6766-3p, miR-7641, miR-1249, miR-3679-3p, miR-6787-5p, miR-4454, miR-3135b, miR-6765-3p, miR-7975, miR-204-3p, miR-7977, miR-7110-5p, miR-6717-5p, miR-6870-5p, miR-663b, miR-6875-5p, miR-8072, miR-6816-5p, miR-4281, miR-6729-5p, miR-8069, miR-4706, miR-7108-5p, miR-4433b-3p, miR-6893-5p, miR-6857-5p, miR-1227-5p, miR-6741-5p, miR-451a, miR-8063, miR-3622a-5p, miR-615-5p, miR-128-1-5p, miR-6825-5p, miR-1260b, miR-4433-3p, miR-4665-5p, miR-7845-5p, miR-1908-5p, miR-6840-3p, miR-6765-5p, miR-296-5p, miR-3675-3p, miR-6781-5p, miR-423-5p, miR-3663-3p, miR-6784-5p, miR-6749-5p, miR-1231, miR-4746-3p, miR-6780b-5p, miR-4758-5p, miR-3679-5p, miR-3184-5p, miR-6125, miR-6721-5p, miR-6791-5p, miR-3185, miR-1260a, miR-3197, miR-6845-5p, miR-6887-5p, miR-6738-5p, miR-6872-3p, miR-4497, miR-1229-5p, miR-6820-5p, miR-6777-5p, miR-3917, miR-5787, miR-4286, miR-6877-5p, miR-1225-3p, miR-6088, miR-6800-5p, miR-1246, miR-4467, miR-4419b, miR-1914-3p, miR-4632-5p, miR-1915-5p, miR-3940-5p, miR-1185-2-3p, miR-6746-5p, miR-5001-5p, miR-1228-5p, miR-5572, miR-4327, miR-4638-5p, miR-6799-5p, miR-6861-5p, miR-6727-5p, miR-4513, miR-6805-3p, miR-6808-5p, miR-4449, miR-1199-5p, miR-1275, miR-4792, miR-4443, miR-6891-5p, miR-6826-5p, miR-6807-5p, miR-7150, miR-4534, miR-4476, miR-4649-5p, miR-4525, miR-1915-3p, miR-4516, miR-4417, miR-642b-3p, miR-3141, miR-5100, miR-6848-5p, miR-4739, miR-4459, miR-1237-5p, miR-296-3p, miR-4665-3p, miR-6786-5p, miR-4258, miR-6510-5p, miR-1343-5p, miR-1247-3p, miR-6805-5p, miR-4492, miR-1469, miR-1268b, miR-6858-5p, miR-3937, miR-939-5p, miR-3656, miR-744-5p, miR-4687-3p, miR-4763-3p, miR-3620-5p, miR-3195, miR-6842-5p, miR-4707-5p, miR-642a-3p, miR-7113-3p, miR-4728-5p, miR-5195-3p, miR-1185-1-3p, miR-6774-5p, miR-8059, miR-3131, miR-7847-3p, miR-4463, miR-128-2-5p, miR-4508, miR-6806-5p, miR-7111-5p, miR-6782-5p, miR-4734, miR-3162-5p, miR-887-3p, miR-6752-5p, miR-6724-5p, miR-6757-5p, miR-4448, miR-671-5p, miR-3178, miR-4725-3p, miR-940, miR-6789-5p, miR-4484, miR-4634, miR-4745-5p, miR-4730, miR-6803-5p, miR-6798-5p, miR-3648, miR-4783-3p and miR-6836-3p.

(2) The kit according to (1), wherein miR-1343-3p is hsa-miR-1343-3p, miR-6726-5p is hsa-miR-6726-5p, miR-6515-3p is hsa-miR-6515-3p, miR-4651 is hsa-miR-4651, miR-4257 is hsa-miR-4257, miR-3188 is hsa-miR-3188, miR-6131 is hsa-miR-6131, miR-6766-3p is hsa-miR-6766-3p, miR-7641 is hsa-miR-7641, miR-1249 is hsa-miR-1249, miR-3679-3p is hsa-miR-3679-3p, miR-6787-5p is hsa-miR-6787-5p, miR-4454 is hsa-miR-4454, miR-3135b is hsa-miR-3135b, miR-6765-3p is hsa-miR-6765-3p, miR-7975 is hsa-miR-7975, miR-204-3p is hsa-miR-204-3p, miR-7977 is hsa-miR-7977, miR-7110-5p is hsa-miR-7110-5p, miR-6717-5p is hsa-miR-6717-5p, miR-6870-5p is hsa-miR-6870-5p, miR-663b is hsa-miR-663b, miR-6875-5p is hsa-miR-6875-5p, miR-8072 is hsa-miR-8072, miR-6816-5p is hsa-miR-6816-5p, miR-4281 is hsa-miR-4281, miR-6729-5p is hsa-miR-6729-5p, miR-8069 is hsa-miR-8069, miR-4706 is hsa-miR-4706, miR-7108-5p is hsa-miR-7108-5p, miR-4433b-3p is hsa-miR-4433b-3p, miR-6893-5p is hsa-miR-6893-5p, miR-6857-5p is hsa-miR-6857-5p, miR-1227-5p is hsa-miR-1227-5p, miR-6741-5p is hsa-miR-6741-5p, miR-451a is hsa-miR-451a, miR-8063 is hsa-miR-8063, miR-3622a-5p is hsa-miR-3622a-5p, miR-615-5p is hsa-miR-615-5p, miR-128-1-5p is hsa-miR-128-1-5p, miR-6825-5p is hsa-miR-6825-5p, miR-1260b is hsa-miR-1260b, miR-4433-3p is hsa-miR-4433-3p, miR-4665-5p is hsa-miR-4665-5p, miR-7845-5p is hsa-miR-7845-5p, miR-1908-5p is hsa-miR-1908-5p, miR-6840-3p is hsa-miR-6840-3p, miR-6765-5p is hsa-miR-6765-5p, miR-296-5p is hsa-miR-296-5p, miR-3675-3p is hsa-miR-3675-3p, miR-6781-5p is hsa-miR-6781-5p, miR-423-5p is hsa-miR-423-5p, miR-3663-3p is hsa-miR-3663-3p, miR-6784-5p is hsa-miR-6784-5p, miR-6749-5p is hsa-miR-6749-5p, miR-1231 is hsa-miR-1231, miR-4746-3p is hsa-miR-4746-3p, miR-6780b-5p is hsa-miR-6780b-5p, miR-4758-5p is hsa-miR-4758-5p, miR-3679-5p is hsa-miR-3679-5p, miR-3184-5p is hsa-miR-3184-5p, miR-6125 is hsa-miR-6125, miR-6721-5p is hsa-miR-6′721-5p, miR-6791-5p is hsa-miR-6791-5p, miR-3185 is hsa-miR-3185, miR-1260a is hsa-miR-1260a, miR-3197 is hsa-miR-3197, miR-6845-5p is hsa-miR-6845-5p, miR-6887-5p is hsa-miR-6887-5p, miR-6738-5p is hsa-miR-6738-5p, miR-6872-3p is hsa-miR-6872-3p, miR-4497 is hsa-miR-4497, miR-1229-5p is hsa-miR-1229-5p, miR-6820-5p is hsa-miR-6820-5p, miR-6777-5p is hsa-miR-6777-5p, miR-3917 is hsa-miR-3917, miR-5787 is hsa-miR-5787, miR-4286 is hsa-miR-4286, miR-6877-5p is hsa-miR-6877-5p, miR-1225-3p is hsa-miR-1225-3p, miR-6088 is hsa-miR-6088, miR-6800-5p is hsa-miR-6800-5p, miR-1246 is hsa-miR-1246, miR-4467 is hsa-miR-4467, miR-4419b is hsa-miR-4419b, miR-1914-3p is hsa-miR-1914-3p, miR-4632-5p is hsa-miR-4632-5p, miR-1915-5p is hsa-miR-1915-5p, miR-3940-5p is hsa-miR-3940-5p, miR-1185-2-3p is hsa-miR-1185-2-3p, miR-6746-5p is hsa-miR-6746-5p, miR-5001-5p is hsa-miR-5001-5p, miR-1228-5p is hsa-miR-1228-5p, miR-5572 is hsa-miR-5572, miR-4327 is hsa-miR-4327, miR-4638-5p is hsa-miR-4638-5p, miR-6799-5p is hsa-miR-6799-5p, miR-6861-5p is hsa-miR-6861-5p, miR-6727-5p is hsa-miR-6727-5p, miR-4513 is hsa-miR-4513, miR-6805-3p is hsa-miR-6805-3p, miR-6808-5p is hsa-miR-6808-5p, miR-4449 is hsa-miR-4449, miR-1199-5p is hsa-miR-1199-5p, miR-1275 is hsa-miR-1275, miR-4792 is hsa-miR-4792, miR-4443 is hsa-miR-4443, miR-6891-5p is hsa-miR-6891-5p, miR-6826-5p is hsa-miR-6826-5p, miR-6807-5p is hsa-miR-6807-5p, miR-7150 is hsa-miR-7150, miR-4534 is hsa-miR-4534, miR-4476 is hsa-miR-4476, miR-4649-5p is hsa-miR-4649-5p, miR-4525 is hsa-miR-4525, miR-1915-3p is hsa-miR-1915-3p, miR-4516 is hsa-miR-4516, miR-4417 is hsa-miR-4417, miR-642b-3p is hsa-miR-642b-3p, miR-3141 is hsa-miR-3141, miR-5100 is hsa-miR-5100, miR-6848-5p is hsa-miR-6848-5p, miR-4739 is hsa-miR-4739, miR-4459 is hsa-miR-4459, miR-1237-5p is hsa-miR-1237-5p, miR-296-3p is hsa-miR-296-3p, miR-4665-3p is hsa-miR-4665-3p, miR-6786-5p is hsa-miR-6786-5p, miR-4258 is hsa-miR-4258, miR-6510-5p is hsa-miR-6510-5p, miR-1343-5p is hsa-miR-1343-5p, miR-1247-3p is hsa-miR-1247-3p, miR-6805-5p is hsa-miR-6805-5p, miR-4492 is hsa-miR-4492, miR-1469 is hsa-miR-1469, miR-1268b is hsa-miR-1268b, miR-6858-5p is hsa-miR-6858-5p, miR-3937 is hsa-miR-3937, miR-939-5p is hsa-miR-939-5p, miR-3656 is hsa-miR-3656, miR-744-5p is hsa-miR-744-5p, miR-4687-3p is hsa-miR-4687-3p, miR-4763-3p is hsa-miR-4763-3p, miR-3620-5p is hsa-miR-3620-5p, miR-3195 is hsa-miR-3195, miR-6842-5p is hsa-miR-6842-5p, miR-4707-5p is hsa-miR-4707-5p, miR-642a-3p is hsa-miR-642a-3p, miR-7113-3p is hsa-miR-7113-3p, miR-4728-5p is hsa-miR-4728-5p, miR-5195-3p is hsa-miR-5195-3p, miR-1185-1-3p is hsa-miR-1185-1-3p, miR-6774-5p is hsa-miR-6774-5p, miR-8059 is hsa-miR-8059, miR-3131 is hsa-miR-3131, miR-7847-3p is hsa-miR-7847-3p, miR-4463 is hsa-miR-4463, miR-128-2-5p is hsa-miR-128-2-5p, miR-4508 is hsa-miR-4508, miR-6806-5p is hsa-miR-6806-5p, miR-7111-5p is hsa-miR-7111-5p, miR-6782-5p is hsa-miR-6782-5p, miR-4734 is hsa-miR-4734, miR-3162-5p is hsa-miR-3162-5p, miR-887-3p is hsa-miR-887-3p, miR-6752-5p is hsa-miR-6752-5p, miR-6724-5p is hsa-miR-6724-5p, miR-6757-5p is hsa-miR-6757-5p, miR-4448 is hsa-miR-4448, miR-671-5p is hsa-miR-671-5p, miR-3178 is hsa-miR-3178, miR-4725-3p is hsa-miR-4725-3p, miR-940 is hsa-miR-940, miR-6789-5p is hsa-miR-6789-5p, miR-4484 is hsa-miR-4484, miR-4634 is hsa-miR-4634, miR-4745-5p is hsa-miR-4745-5p, miR-4730 is hsa-miR-4730, miR-6803-5p is hsa-miR-6803-5p, miR-6798-5p is hsa-miR-6798-5p, miR-3648 is hsa-miR-3648, miR-4783-3p is hsa-miR-4783-3p, and miR-6836-3p is hsa-miR-6836-3p.

(3) The kit according to (1) or (2), wherein the nucleic acid is a polynucleotide selected from the group consisting of the following polynucleotides (a) to (e):
(a) a polynucleotide consisting of a nucleotide sequence represented by any of SEQ ID NOs: 1 to 167 and 714 to 729 or a nucleotide sequence derived from the nucleotide sequence by the replacement of u with t, a variant thereof, a derivative thereof, or a fragment thereof comprising 15 or more consecutive nucleotides,
(b) a polynucleotide comprising a nucleotide sequence represented by any of SEQ ID NOs: 1 to 167 and 714 to 729,
(c) a polynucleotide consisting of a nucleotide sequence complementary to a nucleotide sequence represented by any of SEQ ID NOs: 1 to 167 and 714 to 729 or a nucleotide sequence derived from the nucleotide sequence by the replacement of u with t, a variant thereof, a derivative thereof, or a fragment thereof comprising 15 or more consecutive nucleotides,
(d) a polynucleotide comprising a nucleotide sequence complementary to a nucleotide sequence represented by any of SEQ ID NOs: 1 to 167 and 714 to 729 or a nucleotide sequence derived from the nucleotide sequence by the replacement of u with t, and
(e) a polynucleotide hybridizing under stringent conditions to any of the polynucleotides (a) to (d).

(4) The kit according to any of (1) to (3), wherein the kit further comprises nucleic acid(s) capable of specifically binding to at least one or more polynucleotide(s) selected from the group consisting of other liver cancer markers: miR-23b-3p, miR-23a-3p, miR-625-3p, miR-1228-3p, miR-614, miR-1913, miR-92a-2-5p, miR-187-5p, miR-16-5p, miR-92b-3p, miR-150-3p, miR-564, miR-125a-3p, miR-92b-5p, miR-92a-3p and miR-663a.

(5) The kit according to (4), wherein miR-23b-3p is hsa-miR-23b-3p, miR-23a-3p is hsa-miR-23a-3p, miR-625-3p is hsa-miR-625-3p, miR-1228-3p is hsa-miR-1228-3p, miR-614 is hsa-miR-614, miR-1913 is hsa-miR-1913, miR-92a-2-5p is hsa-miR-92a-2-5p, miR-187-5p is hsa-miR-187-5p, miR-16-5p is hsa-miR-16-5p, miR-92b-3p is hsa-miR-92b-3p, miR-150-3p is hsa-miR-150-3p, miR-564 is hsa-miR-564, miR-125a-3p is hsa-miR-125a-3p, miR-92b-5p is hsa-miR-92b-5p, miR-92a-3p is hsa-miR-92a-3p, and miR-663a is hsa-miR-663a.

(6) The kit according to (4) or (5), wherein the nucleic acid is a polynucleotide selected from the group consisting of the following polynucleotides (f) to (j):
(f) a polynucleotide consisting of a nucleotide sequence represented by any of SEQ ID NOs: 168 to 183 or a nucleotide sequence derived from the nucleotide sequence by the replacement of u with t, a variant thereof, a derivative thereof, or a fragment thereof comprising 15 or more consecutive nucleotides,
(g) a polynucleotide comprising a nucleotide sequence represented by any of SEQ ID NOs: 168 to 183,
(h) a polynucleotide consisting of a nucleotide sequence complementary to a nucleotide sequence represented by any of SEQ ID NOs: 168 to 183 or a nucleotide sequence derived from the nucleotide sequence by the replacement of u with t, a variant thereof, a derivative thereof, or a fragment thereof comprising 15 or more consecutive nucleotides,
(i) a polynucleotide comprising a nucleotide sequence complementary to a nucleotide sequence represented by any of SEQ ID NOs: 168 to 183 or a nucleotide sequence derived from the nucleotide sequence by the replacement of u with t, and
(j) a polynucleotide hybridizing under stringent conditions to any of the polynucleotides (f) to (i).

(7) The kit according to any of (1) to (6), wherein the kit further comprises nucleic acid(s) capable of specifically binding to at least one or more polynucleotide(s) selected from the group consisting of other liver cancer markers: miR-4688, miR-4648, miR-6085, miR-6126, miR-6880-5p, miR-328-5p, miR-6768-5p, miR-3180, miR-6087, miR-1273g-3p, miR-1225-5p, miR-3196, miR-4695-5p, miR-6732-5p, miR-638, miR-6813-5p, miR-665, miR-486-3p, miR-4466, miR-30c-1-3p, miR-3621, miR-6743-5p, miR-4298, miR-4741, miR-3619-3p, miR-6824-5p, miR-5698, miR-371a-5p, miR-4488, miR-1233-5p, miR-4723-5p, miR-24-3p, miR-1238-5p, miR-4442, miR-3928-3p, miR-6716-5p, miR-6089, miR-6124, miR-6778-5p, miR-557 and miR-6090.

(8) The kit according to (7), wherein miR-4688 is hsa-miR-4688, miR-4648 is hsa-miR-4648, miR-6085 is hsa-miR-6085, miR-6126 is hsa-miR-6126, miR-6880-5p is hsa-miR-6880-5p, miR-328-5p is hsa-miR-328-5p, miR-6768-5p is hsa-miR-6768-5p, miR-3180 is hsa-miR-3180, miR-6087 is hsa-miR-6087, miR-1273g-3p is hsa-miR-1273g-3p, miR-1225-5p is hsa-miR-1225-5p, miR-3196 is hsa-miR-3196, miR-4695-5p is hsa-miR-4695-5p, miR-6732-5p is hsa-miR-6732-5p, miR-638 is hsa-miR-638, miR-6813-5p is hsa-miR-6813-5p, miR-665 is hsa-miR-665, miR-486-3p is hsa-miR-486-3p, miR-4466 is hsa-miR-4466, miR-30c-1-3p is hsa-miR-30c-1-3p, miR-3621 is hsa-miR-3621, miR-6743-5p is hsa-miR-6743-5p, miR-4298 is hsa-miR-4298, miR-4741 is hsa-miR-4741, miR-3619-3p is hsa-miR-3619-3p, miR-6824-5p is hsa-miR-6824-5p, miR-5698 is hsa-miR-5698, miR-371a-5p is hsa-miR-371a-5p, miR-4488 is hsa-miR-4488, miR-1233-5p is hsa-miR-1233-5p, miR-4723-5p is hsa-miR-4723-5p, miR-24-3p is hsa-miR-24-3p, miR-1238-5p is hsa-miR-1238-5p, miR-4442 is hsa-miR-4442, miR-3928-3p is hsa-miR-3928-3p, miR-6716-5p is hsa-miR-6716-5p, miR-6089 is hsa-miR-6089, miR-6124 is hsa-miR-6124, miR-6778-5p is hsa-miR-6778-5p, miR-557 is hsa-miR-557, and miR-6090 is hsa-miR-6090.

(9) The kit according to (7) or (8), wherein the nucleic acid is a polynucleotide selected from the group consisting of the following polynucleotides (k) to (o):
(k) a polynucleotide consisting of a nucleotide sequence represented by any of SEQ ID NOs: 184 to 224 or a nucleotide sequence derived from the nucleotide sequence by the replacement of u with t, a variant thereof, a derivative thereof, or a fragment thereof comprising 15 or more consecutive nucleotides,
(1) a polynucleotide comprising a nucleotide sequence represented by any of SEQ ID NOs: 184 to 224,
(m) a polynucleotide consisting of a nucleotide sequence complementary to a nucleotide sequence represented by any of SEQ ID NOs: 184 to 224 or a nucleotide sequence derived from the nucleotide sequence by the replacement of u with t, a variant thereof, a derivative thereof, or a fragment thereof comprising 15 or more consecutive nucleotides,
(n) a polynucleotide comprising a nucleotide sequence complementary to a nucleotide sequence represented by any of SEQ ID NOs: 184 to 224 or a nucleotide sequence derived from the nucleotide sequence by the replacement of u with t, and
(o) a polynucleotide hybridizing under stringent conditions to any of the polynucleotides (k) to (n).

(10) The kit according to any one of (1) to (9), wherein the kit comprises at least two or more nucleic acids capable of specifically binding to at least two or more polynucleotides, respectively, selected from all of the liver cancer markers according to (1) or (2).

(11) A device for the detection of liver cancer, comprising nucleic acid(s) capable of specifically binding to at least one or more polynucleotide(s) selected from the group consisting of liver cancer markers: miR-1343-3p, miR-6726-5p, miR-6515-3p, miR-4651, miR-4257, miR-3188, miR-6131, miR-6766-3p, miR-7641, miR-1249, miR-3679-3p, miR-6787-5p, miR-4454, miR-3135b, miR-6765-3p, miR-7975, miR-204-3p, miR-7977, miR-7110-5p, miR-6717-5p, miR-6870-5p, miR-663b, miR-6875-5p, miR-8072, miR-6816-5p, miR-4281, miR-6729-5p, miR-8069, miR-4706, miR-7108-5p, miR-4433b-3p, miR-6893-5p, miR-6857-5p, miR-1227-5p, miR-6741-5p, miR-451a, miR-8063, miR-3622a-5p, miR-615-5p, miR-128-1-5p, miR-6825-5p, miR-1260b, miR-4433-3p, miR-4665-5p, miR-7845-5p, miR-1908-5p, miR-6840-3p, miR-6765-5p, miR-296-5p, miR-3675-3p, miR-6781-5p, miR-423-5p, miR-3663-3p, miR-6784-5p, miR-6749-5p, miR-1231, miR-4746-3p, miR-6780b-5p, miR-4758-5p, miR-3679-5p, miR-3184-5p, miR-6125, miR-6721-5p, miR-6791-5p, miR-3185, miR-1260a, miR-3197, miR-6845-5p, miR-6887-5p, miR-6738-5p, miR-6872-3p, miR-4497, miR-1229-5p, miR-6820-5p, miR-6777-5p, miR-3917, miR-5787, miR-4286, miR-6877-5p, miR-1225-3p, miR-6088, miR-6800-5p, miR-1246, miR-4467, miR-4419b, miR-1914-3p, miR-4632-5p, miR-1915-5p, miR-3940-5p, miR-1185-2-3p, miR-6746-5p, miR-5001-5p, miR-1228-5p, miR-5572, miR-4327, miR-4638-5p, miR-6799-5p, miR-6861-5p, miR-6727-5p, miR-4513, miR-6805-3p, miR-6808-5p, miR-4449, miR-1199-5p, miR-1275, miR-4792, miR-4443, miR-6891-5p, miR-6826-5p, miR-6807-5p, miR-7150, miR-4534, miR-4476, miR-4649-5p, miR-4525, miR-1915-3p, miR-4516, miR-4417, miR-642b-3p, miR-3141, miR-5100, miR-6848-5p, miR-4739, miR-4459, miR-1237-5p, miR-296-3p, miR-4665-3p, miR-6786-5p, miR-4258, miR-6510-5p, miR-1343-5p, miR-1247-3p, miR-6805-5p, miR-4492, miR-1469, miR-1268b, miR-6858-5p, miR-3937, miR-939-5p, miR-3656, miR-744-5p, miR-4687-3p, miR-4763-3p, miR-3620-5p, miR-3195, miR-6842-5p, miR-4707-5p, miR-642a-3p, miR-7113-3p, miR-4728-5p, miR-5195-3p, miR-1185-1-3p, miR-6774-5p, miR-8059, miR-3131, miR-7847-3p, miR-4463, miR-128-2-5p, miR-4508, miR-6806-5p, miR-7111-5p, miR-6782-5p, miR-4734, miR-3162-5p, miR-887-3p, miR-6752-5p, miR-6724-5p, miR-6757-5p, miR-4448, miR-671-5p, miR-3178, miR-4725-3p, miR-940, miR-6789-5p, miR-4484, miR-4634, miR-4745-5p, miR-4730, miR-6803-5p, miR-6798-5p, miR-3648, miR-4783-3p and miR-6836-3p.

(12) The device according to (11), wherein miR-1343-3p is hsa-miR-1343-3p, miR-6726-5p is hsa-miR-6726-5p, miR-6515-3p is hsa-miR-6515-3p, miR-4651 is hsa-miR-4651, miR-4257 is hsa-miR-4257, miR-3188 is hsa-miR-3188, miR-6131 is hsa-miR-6131, miR-6766-3p is hsa-miR-6766-3p, miR-7641 is hsa-miR-7641, miR-1249 is hsa-miR-1249, miR-3679-3p is hsa-miR-3679-3p, miR-6787-5p is hsa-miR-6787-5p, miR-4454 is hsa-miR-4454, miR-3135b is hsa-miR-3135b, miR-6765-3p is hsa-miR-6765-3p, miR-7975 is hsa-miR-7975, miR-204-3p is hsa-miR-204-3p, miR-7977 is hsa-miR-7977, miR-7110-5p is hsa-miR-7110-5p, miR-6717-5p is hsa-miR-6717-5p, miR-6870-5p is hsa-miR-6870-5p, miR-663b is hsa-miR-663b, miR-6875-5p is hsa-miR-6875-5p, miR-8072 is hsa-miR-8072, miR-6816-5p is hsa-miR-6816-5p, miR-4281 is hsa-miR-4281, miR-6729-5p is hsa-miR-6729-5p, miR-8069 is hsa-miR-8069, miR-4706 is hsa-miR-4706, miR-7108-5p is hsa-miR-7108-5p, miR-4433b-3p is hsa-miR-4433b-3p, miR-6893-5p is hsa-miR-6893-5p, miR-6857-5p is hsa-miR-6857-5p, miR-1227-5p is hsa-miR-1227-5p, miR-6741-5p is hsa-miR-6741-5p, miR-451a is hsa-miR-451a, miR-8063 is hsa-miR-8063, miR-3622a-5p is hsa-miR-3622a-5p, miR-615-5p is hsa-miR-615-5p, miR-128-1-5p is hsa-miR-128-1-5p, miR-6825-5p is hsa-miR-6825-5p, miR-1260b is hsa-miR-1260b, miR-4433-3p is hsa-miR-4433-3p, miR-4665-5p is hsa-miR-4665-5p, miR-7845-5p is hsa-miR-7845-5p, miR-1908-5p is hsa-miR-1908-5p, miR-6840-3p is hsa-miR-6840-3p, miR-6765-5p is hsa-miR-6765-5p, miR-296-5p is hsa-miR-296-5p, miR-3675-3p is hsa-miR-3675-3p, miR-6781-5p is hsa-miR-6781-5p, miR-423-5p is hsa-miR-423-5p, miR-3663-3p is hsa-miR-3663-3p, miR-6784-5p is hsa-miR-6784-5p, miR-6749-5p is hsa-miR-6749-5p, miR-1231 is hsa-miR-1231, miR-4746-3p is hsa-miR-4746-3p, miR-6780b-5p is hsa-miR-6780b-5p, miR-4758-5p is hsa-miR-4758-5p, miR-3679-5p is hsa-miR-3679-5p, miR-3184-5p is hsa-miR-3184-5p, miR-6125 is hsa-miR-6125, miR-6721-5p is hsa-miR-6721-5p, miR-6791-5p is hsa-miR-6791-5p, miR-3185 is hsa-miR-3185, miR-1260a is hsa-miR-1260a, miR-3197 is hsa-miR-3197, miR-6845-5p is hsa-miR-6845-5p, miR-6887-5p is hsa-miR-6887-5p, miR-6738-5p is hsa-miR-6738-5p, miR-6872-3p is hsa-miR-6872-3p, miR-4497 is hsa-miR-4497, miR-1229-5p is hsa-miR-1229-5p, miR-6820-5p is hsa-miR-6820-5p, miR-6777-5p is hsa-miR-6777-5p, miR-3917 is hsa-miR-3917, miR-5787 is hsa-miR-5787, miR-4286 is hsa-miR-4286, miR-6877-5p is hsa-miR-6877-5p, miR-1225-3p is hsa-miR-1225-3p, miR-6088 is hsa-miR-6088, miR-6800-5p is hsa-miR-6800-5p, miR-1246 is hsa-miR-1246, miR-4467 is hsa-miR-4467, miR-4419b is hsa-miR-4419b, miR-1914-3p is hsa-miR-1914-3p, miR-4632-5p is hsa-miR-4632-5p, miR-1915-5p is hsa-miR-1915-5p, miR-3940-5p is hsa-miR-3940-5p, miR-1185-2-3p is hsa-miR-1185-2-3p, miR-6746-5p is hsa-miR-6746-5p, miR-5001-5p is hsa-miR-5001-5p, miR-1228-5p is hsa-miR-1228-5p, miR-5572 is hsa-miR-5572, miR-4327 is hsa-miR-4327, miR-4638-5p is hsa-miR-4638-5p, miR-6799-5p is hsa-miR-6799-5p, miR-6861-5p is hsa-miR-6861-5p, miR-6727-5p is hsa-miR-6727-5p, miR-4513 is hsa-miR-4513, miR-6805-3p is hsa-miR-6805-3p, miR-6808-5p is hsa-miR-6808-5p, miR-4449 is hsa-miR-4449, miR-1199-5p is hsa-miR-1199-5p, miR-1275 is hsa-miR-1275, miR-4792 is hsa-miR-4792, miR-4443 is hsa-miR-4443, miR-6891-5p is hsa-miR-6891-5p, miR-6826-5p is hsa-miR-6826-5p, miR-6807-5p is hsa-miR-6807-5p, miR-7150 is hsa-miR-7150, miR-4534 is hsa-miR-4534, miR-4476 is hsa-miR-4476, miR-4649-5p is hsa-miR-4649-5p, miR-4525 is hsa-miR-4525, miR-1915-3p is hsa-miR-1915-3p, miR-4516 is hsa-miR-4516, miR-4417 is hsa-miR-4417, miR-642b-3p is hsa-miR-642b-3p, miR-3141 is hsa-miR-3141, miR-5100 is hsa-miR-5100, miR-6848-5p is hsa-miR-6848-5p, miR-4739 is hsa-miR-4739, miR-4459 is hsa-miR-4459, miR-1237-5p is hsa-miR-1237-5p, miR-296-3p is hsa-miR-296-3p, miR-4665-3p is hsa-miR-4665-3p, miR-6786-5p is hsa-miR-6786-5p, miR-4258 is hsa-miR-4258, miR-6510-5p is hsa-miR-6510-5p, miR-1343-5p is hsa-miR-1343-5p, miR-1247-3p is hsa-miR-1247-3p, miR-6805-5p is hsa-miR-6805-5p, miR-4492 is hsa-miR-4492, miR-1469 is hsa-miR-1469, miR-1268b is hsa-miR-1268b, miR-6858-5p is hsa-miR-6858-5p, miR-3937 is hsa-miR-3937, miR-939-5p is hsa-miR-939-5p, miR-3656 is hsa-miR-3656, miR-744-5p is hsa-miR-744-5p, miR-4687-3p is hsa-miR-4687-3p, miR-4763-3p is hsa-miR-4763-3p, miR-3620-5p is hsa-miR-3620-5p, miR-3195 is hsa-miR-3195, miR-6842-5p is hsa-miR-6842-5p, miR-4707-5p is hsa-miR-4707-5p, miR-642a-3p is hsa-miR-642a-3p, miR-7113-3p is hsa-miR-7113-3p, miR-4728-5p is hsa-miR-4728-5p, miR-5195-3p is hsa-miR-5195-3p, miR-1185-1-3p is hsa-miR-1185-1-3p, miR-6774-5p is hsa-miR-6774-5p, miR-8059 is hsa-miR-8059, miR-3131 is hsa-miR-3131, miR-7847-3p is hsa-miR-7847-3p, miR-4463 is hsa-miR-4463, miR-128-2-5p is hsa-miR-128-2-5p, miR-4508 is hsa-miR-4508, miR-6806-5p is hsa-miR-6806-5p, miR-7111-5p is hsa-miR-7111-5p, miR-6782-5p is hsa-miR-6782-5p, miR-4734 is hsa-miR-4734, miR-3162-5p is hsa-miR-3162-5p, miR-887-3p is hsa-miR-887-3p, miR-6752-5p is hsa-miR-6752-5p, miR-6724-5p is hsa-miR-6724-5p, miR-6757-5p is hsa-miR-6757-5p, miR-4448 is hsa-miR-4448, miR-671-5p is hsa-miR-671-5p, miR-3178 is hsa-miR-3178, miR-4725-3p is hsa-miR-4725-3p, miR-940 is hsa-miR-940, miR-6789-5p is hsa-miR-6789-5p, miR-4484 is hsa-miR-4484, miR-4634 is hsa-miR-4634, miR-4745-5p is hsa-miR-4745-5p, miR-4730 is hsa-miR-4730, miR-6803-5p is hsa-miR-6803-5p, miR-6798-5p is hsa-miR-6798-5p, miR-3648 is hsa-miR-3648, miR-4783-3p is hsa-miR-4783-3p, and miR-6836-3p is hsa-miR-6836-3p.

(13) The device according to (11) or (12), wherein the nucleic acid is a polynucleotide selected from the group consisting of the following polynucleotides (a) to (e):
(a) a polynucleotide consisting of a nucleotide sequence represented by any of SEQ ID NOs: 1 to 167 and 714 to 729 or a nucleotide sequence derived from the nucleotide sequence by the replacement of u with t, a variant thereof, a derivative thereof, or a fragment thereof comprising 15 or more consecutive nucleotides,
(b) a polynucleotide comprising a nucleotide sequence represented by any of SEQ ID NOs: 1 to 167 and 714 to 729,
(c) a polynucleotide consisting of a nucleotide sequence complementary to a nucleotide sequence represented by any of SEQ ID NOs: 1 to 167 and 714 to 729 or a nucleotide sequence derived from the nucleotide sequence by the replacement of u with t, a variant thereof, a derivative thereof, or a fragment thereof comprising 15 or more consecutive nucleotides,
(d) a polynucleotide comprising a nucleotide sequence complementary to a nucleotide sequence represented by any of SEQ ID NOs: 1 to 167 and 714 to 729 or a nucleotide sequence derived from the nucleotide sequence by the replacement of u with t, and
(e) a polynucleotide hybridizing under stringent conditions to any of the polynucleotides (a) to (d).

(14) The device according to any of (11) to (13), wherein the device further comprises nucleic acid(s) capable of specifically binding to at least one or more polynucleotide(s) selected from the group consisting of other liver cancer markers: miR-23b-3p, miR-23a-3p, miR-625-3p, miR-1228-3p, miR-614, miR-1913, miR-92a-2-5p, miR-187-5p, miR-16-5p, miR-92b-3p, miR-150-3p, miR-564, miR-125a-3p, miR-92b-5p, miR-92a-3p and miR-663a.

(15) The device according to (14), wherein miR-23b-3p is hsa-miR-23b-3p, miR-23a-3p is hsa-miR-23a-3p, miR-625-3p is hsa-miR-625-3p, miR-1228-3p is hsa-miR-1228-3p, miR-614 is hsa-miR-614, miR-1913 is hsa-miR-1913, miR-92a-2-5p is hsa-miR-92a-2-5p, miR-187-5p is hsa-miR-187-5p, miR-16-5p is hsa-miR-16-5p, miR-92b-3p is hsa-miR-92b-3p, miR-150-3p is hsa-miR-150-3p, miR-564 is hsa-miR-564, miR-125a-3p is hsa-miR-125a-3p, miR-92b-5p is hsa-miR-92b-5p, miR-92a-3p is hsa-miR-92a-3p, and miR-663a is hsa-miR-663a.

(16) The device according to (14) or (15), wherein the nucleic acid is a polynucleotide selected from the group consisting of the following polynucleotides (f) to (j):
(f) a polynucleotide consisting of a nucleotide sequence represented by any of SEQ ID NOs: 168 to 183 or a nucleotide sequence derived from the nucleotide sequence by the replacement of u with t, a variant thereof, a derivative thereof, or a fragment thereof comprising 15 or more consecutive nucleotides,
(g) a polynucleotide comprising a nucleotide sequence represented by any of SEQ ID NOs: 168 to 183,
(h) a polynucleotide consisting of a nucleotide sequence complementary to a nucleotide sequence represented by any of SEQ ID NOs: 168 to 183 or a nucleotide sequence derived from the nucleotide sequence by the replacement of u with t, a variant thereof, a derivative thereof, or a fragment thereof comprising 15 or more consecutive nucleotides,
(i) a polynucleotide comprising a nucleotide sequence complementary to a nucleotide sequence represented by any of SEQ ID NOs: 168 to 183 or a nucleotide sequence derived from the nucleotide sequence by the replacement of u with t, and
(j) a polynucleotide hybridizing under stringent conditions to any of the polynucleotides (f) to (i).

(17) The device according to any of (11) to (16), wherein the device further comprises nucleic acid(s) capable of specifically binding to at least one or more polynucleotide(s) selected from the group consisting of other liver cancer markers: miR-4688, miR-4648, miR-6085, miR-6126, miR-6880-5p, miR-328-5p, miR-6768-5p, miR-3180, miR-6087, miR-1273g-3p, miR-1225-5p, miR-3196, miR-4695-5p, miR-6732-5p, miR-638, miR-6813-5p, miR-665, miR-486-3p, miR-4466, miR-30c-1-3p, miR-3621, miR-6743-5p, miR-4298, miR-4741, miR-3619-3p, miR-6824-5p, miR-5698, miR-371a-5p, miR-4488, miR-1233-5p, miR-4723-5p, miR-24-3p, miR-1238-5p, miR-4442, miR-3928-3p, miR-6716-5p, miR-6089, miR-6124, miR-6778-5p, miR-557 and miR-6090.

(18) The device according to (17), wherein miR-4688 is hsa-miR-4688, miR-4648 is hsa-miR-4648, miR-6085 is hsa-miR-6085, miR-6126 is hsa-miR-6126, miR-6880-5p is hsa-miR-6880-5p, miR-328-5p is hsa-miR-328-5p, miR-6768-5p is hsa-miR-6768-5p, miR-3180 is hsa-miR-3180, miR-6087 is hsa-miR-6087, miR-1273g-3p is hsa-miR-1273g-3p, miR-1225-5p is hsa-miR-1225-5p, miR-3196 is hsa-miR-3196, miR-4695-5p is hsa-miR-4695-5p, miR-6732-5p is hsa-miR-6732-5p, miR-638 is hsa-miR-638, miR-6813-5p is hsa-miR-6813-5p, miR-665 is hsa-miR-665, miR-486-3p is hsa-miR-486-3p, miR-4466 is hsa-miR-4466, miR-30c-1-3p is hsa-miR-30c-1-3p, miR-3621 is hsa-miR-3621, miR-6743-5p is hsa-miR-6743-5p, miR-4298 is hsa-miR-4298, miR-4741 is hsa-miR-4741, miR-3619-3p is hsa-miR-3619-3p, miR-6824-5p is hsa-miR-6824-5p, miR-5698 is hsa-miR-5698, miR-371a-5p is hsa-miR-371a-5p, miR-4488 is hsa-miR-4488, miR-1233-5p is hsa-miR-1233-5p, miR-4723-5p is hsa-miR-4723-5p, miR-24-3p is hsa-miR-24-3p, miR-1238-5p is hsa-miR-1238-5p, miR-4442 is hsa-miR-4442, miR-3928-3p is hsa-miR-3928-3p, miR-6716-5p is hsa-miR-6716-5p, miR-6089 is hsa-miR-6089, miR-6124 is hsa-miR-6124, miR-6778-5p is hsa-miR-6778-5p, miR-557 is hsa-miR-557, and miR-6090 is hsa-miR-6090.

(19) The device according to (17) or (18), wherein the nucleic acid is a polynucleotide selected from the group consisting of the following polynucleotides (k) to (o):
(k) a polynucleotide consisting of a nucleotide sequence represented by any of SEQ ID NOs: 184 to 224 or a nucleotide sequence derived from the nucleotide sequence by the replacement of u with t, a variant thereof, a derivative thereof, or a fragment thereof comprising 15 or more consecutive nucleotides,
(l) a polynucleotide comprising a nucleotide sequence represented by any of SEQ ID NOs: 184 to 224,
(m) a polynucleotide consisting of a nucleotide sequence complementary to a nucleotide sequence represented by any of SEQ ID NOs: 184 to 224 or a nucleotide sequence derived from the nucleotide sequence by the replacement of u with t, a variant thereof, a derivative thereof, or a fragment thereof comprising 15 or more consecutive nucleotides,
(n) a polynucleotide comprising a nucleotide sequence complementary to a nucleotide sequence represented by any of SEQ ID NOs: 184 to 224 or a nucleotide sequence derived from the nucleotide sequence by the replacement of u with t, and
(o) a polynucleotide hybridizing under stringent conditions to any of the polynucleotides (k) to (n).

(20) The device according to any one of (11) to (19), wherein the device is a device for measurement by a hybridization technique.

(21) The device according to (20), wherein the hybridization technique is a nucleic acid array technique.

(22) The device according to any one of (11) to (21), wherein the device comprises at least two or more nucleic acids capable of specifically binding to at least two or more polynucleotides, respectively, selected from all of the liver cancer markers according to (11) or (12).

(23) A method for detecting liver cancer, comprising measuring an expression level of a target nucleic acid in a sample of a subject using the kit according to any one of (1) to (10) or the device according to any one of (11) to (22); and evaluating in vitro whether or not the subject has liver cancer using the measured expression level and a control expression level for a healthy subject measured in the same way.

(24) The method according to (23), wherein the subject is a human.

(25) The method according to (23) or (24), wherein the sample is blood, serum, or plasma.

<Definition of Term>

The terms used herein are defined as follows.

Abbreviations or terms such as nucleotide, polynucleotide, DNA, and RNA abide by “Guidelines for the preparation of specification which contain nucleotide and/or amino acid sequences” (edited by Japan Patent Office) and common use in the art.

The term “polynucleotide” used herein refers to a nucleic acid, including any of RNA, DNA, and RNA/DNA (chimera). The DNA includes any of cDNA, genomic DNA, and synthetic DNA. The RNA includes all of total RNA, mRNA, rRNA, miRNA, siRNA, snoRNA, snRNA, non-coding RNA and synthetic RNA. The “synthetic DNA” and the “synthetic RNA” used herein refer to DNA and RNA artificially prepared using, for example, an automated nucleic acid synthesizer, on the basis of predetermined nucleotide sequences (which may be any of natural and non-natural sequences). The “non-natural sequence” used herein is intended to be used in a broad sense and includes, for example, a sequence containing substitution, deletion, insertion, and/or addition of one or more nucleotide(s) (i.e., a variant sequence) and a sequence containing one or more modified nucleotide(s) (i.e., a modified sequence), which are different from the natural sequence. As used herein, the term “polynucleotide” is used interchangeably with the term “nucleic acid.”

The term “fragment” used herein is a polynucleotide having a nucleotide sequence having a consecutive portion of a polynucleotide and desirably has a length of 15 or more nucleotides, preferably 17 or more nucleotides, more preferably 19 or more nucleotides.

The term “gene” used herein is intended to include not only RNA and double-stranded DNA but also each single-stranded DNA such as a plus strand (or a sense strand) or a complementary strand (or an antisense strand) constituting the duplex. The gene is not particularly limited by its length.

Thus, the “gene” used herein includes all of double-stranded DNA including human genomic DNA, single-stranded DNA (plus strand), single-stranded DNA having a sequence complementary to the plus strand (complementary strand) including cDNA, microRNA (miRNA), and their fragments, and transcripts, unless otherwise specified. The “gene” includes not only a “gene” represented by a particular nucleotide sequence (or SEQ ID NO) but “nucleic acids” encoding RNAs having biological functions equivalent to RNA encoded by the gene, for example, a congener (i.e., a homolog or an ortholog), a variant (e.g., a genetic polymorph), and a derivative. Specific examples of such a “nucleic acid” encoding a congener, a variant, or a derivative can include a “nucleic acid” having a nucleotide sequence hybridizing under stringent conditions described later to a complementary sequence of a nucleotide sequence represented by any of SEQ ID NOs: 1 to 765 or a nucleotide sequence derived from the nucleotide sequence by the replacement of u with t. The “gene” is not particularly limited by its functional region and can contain, for example, an expression regulatory region, a coding region, an exon, or an intron. The “gene” may be contained in a cell or may exist alone after being released into the outside of a cell. Alternatively, the “gene” may be in a state enclosed in a vesicle called exosome.

The term “exosome” used herein is a vesicle that is delimited by a lipid bilayer and secreted from a cell. The exosome is derived from a multivesicular endosome and may incorporate biomaterials such as “gene(s)” (e.g., RNA or DNA) or protein(s) when released into an extracellular environment. The exosome is known to be contained in a body fluid such as blood, serum, plasma, serum, or lymph.

The term “transcript” used herein refers to RNA synthesized from the DNA sequence of a gene as a template. RNA polymerase binds to a site called a promoter located upstream of the gene and adds ribonucleotides complementary to the nucleotide sequence of the DNA to the 3′ end to synthesize RNA. This RNA contains not only the gene itself but also the whole sequence from a transcription initiation site to the end of a polyA sequence, including an expression regulatory region, a coding region, an exon, or an intron.

The term “microRNA (miRNA)” used herein is intended to mean a 15- to 25-nucleotide non-coding RNA that is transcribed as an RNA precursor having a hairpin-like structure, cleaved by a dsRNA-cleaving enzyme which has RNase III cleavage activity, and integrated into a protein complex called RISC, and involved in the suppression of translation of mRNA, unless otherwise specified. The term “miRNA” used herein includes not only a “miRNA” represented by a particular nucleotide sequence (or SEQ ID NO) but a precursor of the “miRNA” (pre-miRNA or pri-miRNA), and miRNAs having biological functions equivalent thereto, for example, a congener (i.e., a homolog or an ortholog), a variant (e.g., a genetic polymorph), and a derivative. Such a precursor, a congener, a variant, or a derivative can be specifically identified using miRBase Release 20 (http://www.mirbase.org/), and examples thereof can include a “miRNA” having a nucleotide sequence hybridizing under stringent conditions described later to a complementary sequence of any particular nucleotide sequence represented by any of SEQ ID NOs: 1 to 765. The term “miRNA” used herein may be a gene product of a miR gene. Such a gene product includes a mature miRNA (e.g., a 15- to 25-nucleotide or 19- to 25-nucleotide non-coding RNA involved in the suppression of translation of mRNA as described above) or a miRNA precursor (e.g., pre-miRNA or pri-miRNA as described above).

The term “probe” used herein includes a polynucleotide that is used for specifically detecting RNA resulting from the expression of a gene or a polynucleotide derived from the RNA, and/or a polynucleotide complementary thereto.

The term “primer” used herein includes a polynucleotide that specifically recognizes and amplifies RNA resulting from the expression of a gene or a polynucleotide derived from the RNA, and/or a polynucleotide complementary thereto.

In this context, the complementary polynucleotide (complementary strand or reverse strand) means a polynucleotide in a complementary relationship of A:T (U) and G:C base pairs with the full-length sequence of a polynucleotide consisting of a nucleotide sequence defined by any of SEQ ID NOs: 1 to 765 or a nucleotide sequence derived from the nucleotide sequence by the replacement of u with t, or a partial sequence thereof (here, this full-length or partial sequence is referred to as a plus strand for the sake of convenience). However, such a complementary strand is not limited to a sequence completely complementary to the nucleotide sequence of the target plus strand and may have a complementary relationship to an extent that permits hybridization under stringent conditions to the target plus strand.

The term “stringent conditions” used herein refers to conditions under which a nucleic acid probe hybridizes to its target sequence to a larger extent (e.g., a measurement value equal to or larger than a mean of background measurement values+a standard deviation of the background measurement values×2) than that for other sequences. The stringent conditions are dependent on a sequence and differ depending on an environment where hybridization is performed. A target sequence that is 100% complementary to the nucleic acid probe can be identified by controlling the stringency of hybridization and/or washing conditions. Specific examples of the “stringent conditions” is mentioned later.

The term “Tm value” used herein means a temperature at which the double-stranded moiety of a polynucleotide is denatured into single strands so that the double strands and the single strands exist at a ratio of 1:1.

The term “variant” used herein means, in the case of a nucleic acid, a natural variant attributed to polymorphism, mutation, or the like; a variant containing the deletion, substitution, addition, or insertion of 1 or 2 or more nucleotides in a nucleotide sequence represented by any of SEQ ID NOs: 1 to 765 or a nucleotide sequence derived from the nucleotide sequence by the replacement of u with t, or a partial sequence thereof; a variant that exhibits percent (%) identity of approximately 90% or higher, approximately 95% or higher, approximately 97% or higher, approximately 98% or higher, approximately 99% or higher to each of these nucleotide sequences or the partial sequence thereof; or a nucleic acid hybridizing under the stringent conditions defined above to a polynucleotide or an oligonucleotide comprising each of these nucleotide sequences or the partial sequence thereof.

The term “several” used herein means an integer of approximately 10, 9, 8, 7, 6, 5, 4, 3, or 2.

The variant used herein can be prepared by use of a well-known technique such as site-directed mutagenesis or PCR-based mutagenesis.

The term “percent (%) identity” used herein can be determined with or without an introduced gap, using a protein or gene search system based on BLAST or FASTA described above (Zheng Zhang et al., 2000, J. Comput. Biol., Vol. 7, p. 203-214; Altschul, S. F. et al., 1990, Journal of Molecular Biology, Vol. 215, p. 403-410; and Pearson, W. R. et al., 1988, Proc. Natl. Acad. Sci. U.S.A, Vol. 85, p. 2444-2448).

The term “derivative” used herein is meant to include a modified nucleic acid, for example, a derivative labeled with a fluorophore or the like, a derivative containing a modified nucleotide (e.g., a nucleotide containing a group such as halogen, alkyl such as methyl, alkoxy such as methoxy, thio, or carboxymethyl, and a nucleotide that has undergone base rearrangement, double bond saturation, deamination, replacement of an oxygen molecule with a sulfur atom, etc.), PNA (peptide nucleic acid; Nielsen, P. E. et al., 1991, Science, Vol. 254, p. 1497-500), and LNA (locked nucleic acid; Obika, S. et al., 1998, Tetrahedron Lett., Vol. 39, p. 5401-5404) without any limitation.

As used herein, the “nucleic acid” capable of specifically binding to a polynucleotide selected from the group of the miRNAs described above which are the liver cancer markers is a synthesized or prepared nucleic acid and specifically includes a “nucleic acid probe” or a “primer”. The “nucleic acid” is utilized directly or indirectly for detecting the presence or absence of liver cancer in a subject, for diagnosing the presence or absence of liver cancer, the severity of liver cancer, the presence or absence of amelioration or the degree of amelioration of liver cancer, or the therapeutic sensitivity of liver cancer, or for screening for a candidate substance useful in the prevention, amelioration, or treatment of liver cancer. The “nucleic acid” includes a nucleotide, an oligonucleotide, and a polynucleotide capable of specifically recognizing and binding to a transcript represented by any of SEQ ID NOs: 1 to 765 or a synthetic cDNA nucleic acid thereof in vivo, particularly, in a sample such as a body fluid (e.g., blood or urine), in relation to the development of liver cancer. The nucleotide, the oligonucleotide, and the polynucleotide can be effectively used as probes for detecting the aforementioned gene expressed in vivo, in tissues, in cells, or the like on the basis of the properties described above, or as primers for amplifying the aforementioned gene expressed in vivo.

The term “detection” used herein is interchangeable with the term “examination”, “measurement”, “detection” or “decision support”. The term “evaluation” used herein is meant to include diagnosis or evaluation support on the basis of examination results or measurement results.

The term “subject” used herein means a mammal such as a primate including a human and a chimpanzee, a pet animal including a dog and a cat, a livestock animal including cattle, a horse, sheep, and a goat, and a rodent including a mouse and a rat. The term “healthy subject” also means such a mammal without the cancer to be detected.

The term “liver cancer” used herein means “primary liver cancer”, which develops primarily in the liver. The liver cancer includes, for example, “hepatocellular carcinoma” and “combined hepatocellular and cholangiocellular carcinoma” caused by the malignant transformation of cells of the liver.

The term “P” or “P value” used herein refers to a probability at which a more extreme statistic than that actually calculated from data under null hypothesis is observed in a statistical test. Thus, smaller “P” or “P value” is regarded as being more significant difference between subjects to be compared.

The term “sensitivity” used herein means a value of (the number of true positives)/(the number of true positives+the number of false negatives). High sensitivity allows liver cancer to be detected early, leading to the complete resection of cancer sites and reduction in the rate of recurrence.

The term “specificity” used herein means a value of (the number of true negatives)/(the number of true negatives+the number of false positives). High specificity prevents needless extra examination for healthy subjects misjudged as being liver cancer patients, leading to reduction in burden on patients and reduction in medical expense.

The term “accuracy” used herein means a value of (the number of true positives+the number of true negatives)/(the total number of cases). The accuracy indicates the ratio of samples that are correctly identified in the discriminant results to all samples, and serves as a primary index for evaluating detection performance.

As used herein, the “sample” that is subjected to determination, detection, or diagnosis refers to a tissue and a biological material in which the expression of the gene of the present invention varies as liver cancer develops, as liver cancer progresses, or as therapeutic effects on liver cancer are exerted. Specifically, the “sample” refers to a hepatic tissue, a perihepatic vascular channel, lymph node, and organ, an organ suspected of having metastasis, the skin, a body fluid such as blood, urine, saliva, sweat, or tissue exudates, serum or plasma prepared from blood, feces, hair, and the like. The “sample” further refers to a biological sample extracted therefrom, specifically, a gene such as RNA or miRNA.

The term “hsa-miR-1343-3p gene” or “hsa-miR-1343-3p” used herein includes the hsa-miR-1343-3p gene (miRBase Accession No. MIMAT0019776) described in SEQ ID NO: 1, a homolog or an ortholog of a different organism species, and the like. The hsa-miR-1343-3p gene can be obtained by a method described in Persson H et al., 2011, Cancer Res, Vol. 71, p. 78-86. Also, “hsa-mir-1343” (miRBase Accession No. MI0017320, SEQ ID NO: 225) having a hairpin-like structure is known as a precursor of “hsa-miR-1343-3p”.

The term “hsa-miR-6726-5p gene” or “hsa-miR-6726-5p” used herein includes the hsa-miR-6726-5p gene (miRBase Accession No. MIMAT0027353) described in SEQ ID NO: 2, a homolog or an ortholog of a different organism species, and the like. The hsa-miR-6726-5p gene can be obtained by a method described in Ladewig E et al., 2012, Genome Res, Vol. 22, p. 1634-1645. Also, “hsa-mir-6726” (miRBase Accession No. MI0022571, SEQ ID NO: 226) having a hairpin-like structure is known as a precursor of “hsa-miR-6726-5p”.

The term “hsa-miR-6515-3p gene” or “hsa-miR-6515-3p” used herein includes the hsa-miR-6515-3p gene (miRBase Accession No. MIMAT0025487) described in SEQ ID NO: 3, a homolog or an ortholog of a different organism species, and the like. The hsa-miR-6515-3p gene can be obtained by a method described in Joyce C E et al., 2011, Hum Mol Genet, Vol. 20, p. 4025-4040. Also, “hsa-mir-6515” (miRBase Accession No. M10022227, SEQ ID NO: 227) having a hairpin-like structure is known as a precursor of “hsa-miR-6515-3p”.

The term “hsa-miR-4651 gene” or “hsa-miR-4651” used herein includes the hsa-miR-4651 gene (miRBase Accession No. MIMAT0019715) described in SEQ ID NO: 4, a homolog or an ortholog of a different organism species, and the like. The hsa-miR-4651 gene can be obtained by a method described in Persson H et al., 2011, Cancer Res, Vol. 71, p. 78-86. Also, “hsa-mir-4651” (miRBase Accession No. M10017279, SEQ ID NO: 228) having a hairpin-like structure is known as a precursor of “hsa-miR-4651”.

The term “hsa-miR-4257 gene” or “hsa-miR-4257” used herein includes the hsa-miR-4257 gene (miRBase Accession No. MIMAT0016878) described in SEQ ID NO: 5, a homolog or an ortholog of a different organism species, and the like. The hsa-miR-4257 gene can be obtained by a method described in Goff L A et al., 2009, PLoS One, Vol. 4, e7192. Also, “hsa-mir-4257” (miRBase Accession No. MI0015856, SEQ ID NO: 229) having a hairpin-like structure is known as a precursor of “hsa-miR-4257”.

The term “hsa-miR-3188 gene” or “hsa-miR-3188” used herein includes the hsa-miR-3188 gene (miRBase Accession No. MIMAT0015070) described in SEQ ID NO: 6, a homolog or an ortholog of a different organism species, and the like. The hsa-miR-3188 gene can be obtained by a method described in Stark M S et al., 2010, PLoS One, Vol. 5, e9685. Also, “hsa-mir-3188” (miRBase Accession No. M10014232, SEQ ID NO: 230) having a hairpin-like structure is known as a precursor of “hsa-miR-3188”.

The term “hsa-miR-6131 gene” or “hsa-miR-6131” used herein includes the hsa-miR-6131 gene (miRBase Accession No. MIMAT0024615) described in SEQ ID NO: 7, a homolog or an ortholog of a different organism species, and the like. The hsa-miR-6131 gene can be obtained by a method described in Dannemann M et al., 2012, Genome Biol Evol, Vol. 4, p. 552-564. Also, “hsa-mir-6131” (miRBase Accession No. M10021276, SEQ ID NO: 231) having a hairpin-like structure is known as a precursor of “hsa-miR-6131”.

The term “hsa-miR-6766-3p gene” or “hsa-miR-6766-3p” used herein includes the hsa-miR-6766-3p gene (miRBase Accession No. MIMAT0027433) described in SEQ ID NO: 8, a homolog or an ortholog of a different organism species, and the like. The hsa-miR-6766-3p gene can be obtained by a method described in Ladewig E et al., 2012, Genome Res, Vol. 22, p. 1634-1645. Also, “hsa-mir-6766” (miRBase Accession No. M10022611, SEQ ID NO: 232) having a hairpin-like structure is known as a precursor of “hsa-miR-6766-3p”.

The term “hsa-miR-7641 gene” or “hsa-miR-7641” used herein includes the hsa-miR-7641 gene (miRBase Accession No. MIMAT0029782) described in SEQ ID NO: 9, a homolog or an ortholog of a different organism species, and the like. The hsa-miR-7641 gene can be obtained by a method described in Yoo J K et al., 2013, Arch Pharm Res, Vol. 36, p. 353-358. Also, “hsa-mir-7641-1” and “hsa-mir-7641-2” (miRBase Accession Nos. M10024975 and M10024976, SEQ ID NOs: 233 and 234) having a hairpin-like structure are known as precursors of “hsa-miR-7641”.

The term “hsa-miR-1249 gene” or “hsa-miR-1249” used herein includes the hsa-miR-1249 gene (miRBase Accession No. MIMAT0005901) described in SEQ ID NO: 10, a homolog or an ortholog of a different organism species, and the like. The hsa-miR-1249 gene can be obtained by a method described in Morin R D et al., 2008, Genome Res, Vol. 18, p. 610-621. Also, “hsa-mir-1249” (miRBase Accession No. M10006384, SEQ ID NO: 235) having a hairpin-like structure is known as a precursor of “hsa-miR-1249”.

The term “hsa-miR-3679-3p gene” or “hsa-miR-3679-3p” used herein includes the hsa-miR-3679-3p gene (miRBase Accession No. MIMAT0018105) described in SEQ ID NO: 11, a homolog or an ortholog of a different organism species, and the like. The hsa-miR-3679-3p gene can be obtained by a method described in Creighton C J et al., 2010, PLoS One, Vol. 5, e9637. Also, “hsa-mir-3679” (miRBase Accession No. M10016080, SEQ ID NO: 236) having a hairpin-like structure is known as a precursor of “hsa-miR-3679-3p”.

The term “hsa-miR-6787-5p gene” or “hsa-miR-6787-5p” used herein includes the hsa-miR-6787-5p gene (miRBase Accession No. MIMAT0027474) described in SEQ ID NO: 12, a homolog or an ortholog of a different organism species, and the like. The hsa-miR-6787-5p gene can be obtained by a method described in Ladewig E et al., 2012, Genome Res, Vol. 22, p. 1634-1645. Also, “hsa-mir-6787” (miRBase Accession No. M10022632, SEQ ID NO: 237) having a hairpin-like structure is known as a precursor of “hsa-miR-6787-5p”.

The term “hsa-miR-4454 gene” or “hsa-miR-4454” used herein includes the hsa-miR-4454 gene (miRBase Accession No. MIMAT0018976) described in SEQ ID NO: 13, a homolog or an ortholog of a different organism species, and the like. The hsa-miR-4454 gene can be obtained by a method described in Jima D D et al., 2010, Blood, Vol. 116, e118-e127. Also, “hsa-mir-4454” (miRBase Accession No. M10016800, SEQ ID NO: 238) having a hairpin-like structure is known as a precursor of “hsa-miR-4454”.

The term “hsa-miR-3135b gene” or “hsa-miR-3135b” used herein includes the hsa-miR-3135b gene (miRBase Accession No. MIMAT0018985) described in SEQ ID NO: 14, a homolog or an ortholog of a different organism species, and the like. The hsa-miR-3135b gene can be obtained by a method described in Jima D D et al., 2010, Blood, Vol. 116, e118-e127. Also, “hsa-mir-3135b” (miRBase Accession No. M10016809, SEQ ID NO: 239) having a hairpin-like structure is known as a precursor of “hsa-miR-3135b”.

The term “hsa-miR-6765-3p gene” or “hsa-miR-6765-3p” used herein includes the hsa-miR-6765-3p gene (miRBase Accession No. MIMAT0027431) described in SEQ ID NO: 15, a homolog or an ortholog of a different organism species, and the like. The hsa-miR-6765-3p p. 1634-1645. Also, “hsa-mir-6765” (miRBase Accession No. M10022610, SEQ ID NO: 240) having a hairpin-like structure is known as a precursor of “hsa-miR-6765-3p”.

The term “hsa-miR-7975 gene” or “hsa-miR-7975” used herein includes the hsa-miR-7975 gene (miRBase Accession No. MIMAT0031178) described in SEQ ID NO: 16, a homolog or an ortholog of a different organism species, and the like. The hsa-miR-7975 gene can be obtained by a method described in Velthut-Meikas A et al., 2013, Mol Endocrinol, online. Also, “hsa-mir-7975” (miRBase Accession No. M10025751, SEQ ID NO: 241) having a hairpin-like structure is known as a precursor of “hsa-miR-7975”.

The term “hsa-miR-204-3p gene” or “hsa-miR-204-3p” used herein includes the hsa-miR-204-3p gene (miRBase Accession No. MIMAT0022693) described in SEQ ID NO: 17, a homolog or an ortholog of a different organism species, and the like. The hsa-miR-204-3p gene can be obtained by a method described in Lim L P et al., 2003, Science, Vol. 299, p. 1540. Also, “hsa-mir-204” (miRBase Accession No. M10000284, SEQ ID NO: 242) having a hairpin-like structure is known as a precursor of “hsa-miR-204-3p”.

The term “hsa-miR-7977 gene” or “hsa-miR-7977” used herein includes the hsa-miR-7977 gene (miRBase Accession No. MIMAT0031180) described in SEQ ID NO: 18, a homolog or an ortholog of a different organism species, and the like. The hsa-miR-7977 gene can be obtained by a method described in Velthut-Meikas A et al., 2013, Mol Endocrinol, online. Also, “hsa-mir-7977” (miRBase Accession No. M10025753, SEQ ID NO: 243) having a hairpin-like structure is known as a precursor of “hsa-miR-7977”.

The term “hsa-miR-7110-5p gene” or “hsa-miR-7110-5p” used herein includes the hsa-miR-7110-5p gene (miRBase Accession No. MIMAT0028117) described in SEQ ID NO: 19, a homolog or an ortholog of a different organism species, and the like. The hsa-miR-7110-5p p. 1634-1645. Also, “hsa-mir-7110” (miRBase Accession No. M10022961, SEQ ID NO: 244) having a hairpin-like structure is known as a precursor of “hsa-miR-7110-5p”.

The term “hsa-miR-6717-5p gene” or “hsa-miR-6717-5p” used herein includes the hsa-miR-6717-5p gene (miRBase Accession No. MIMAT0025846) described in SEQ ID NO: 20, a homolog or an ortholog of a different organism species, and the like. The hsa-miR-6717-5p gene can be obtained by a method described in Li Y et al., 2012, Gene, Vol. 497, p. 330-335. Also, “hsa-mir-6717” (miRBase Accession No. M10022551, SEQ ID NO: 245) having a hairpin-like structure is known as a precursor of “hsa-miR-6717-5p”.

The term “hsa-miR-6870-5p gene” or “hsa-miR-6870-5p” used herein includes the hsa-miR-6870-5p gene (miRBase Accession No. MIMAT0027640) described in SEQ ID NO: 21, a homolog or an ortholog of a different organism species, and the like. The hsa-miR-6870-5p gene can be obtained by a method described in Ladewig E et al., 2012, Genome Res, Vol. 22, p. 1634-1645. Also, “hsa-mir-6870” (miRBase Accession No. M10022717, SEQ ID NO: 246) having a hairpin-like structure is known as a precursor of “hsa-miR-6870-5p”.

The term “hsa-miR-663b gene” or “hsa-miR-663b” used herein includes the hsa-miR-663b gene (miRBase Accession No. MIMAT0005867) described in SEQ ID NO: 22, a homolog or an ortholog of a different organism species, and the like. The hsa-miR-663b gene can be obtained by a method described in Takada S et al., 2008, Leukemia, Vol. 22, p. 1274-1278. Also, “hsa-mir-663b” (miRBase Accession No. M10006336, SEQ ID NO: 247) having a hairpin-like structure is known as a precursor of “hsa-miR-663b”.

The term “hsa-miR-6875-5p gene” or “hsa-miR-6875-5p” used herein includes the hsa-miR-6875-5p gene (miRBase Accession No. MIMAT0027650) described in SEQ ID NO: 23, a homolog or an ortholog of a different organism species, and the like. The hsa-miR-6875-5p p. 1634-1645. Also, “hsa-mir-6875” (miRBase Accession No. M10022722, SEQ ID NO: 248) having a hairpin-like structure is known as a precursor of “hsa-miR-6875-5p”.

The term “hsa-miR-8072 gene” or “hsa-miR-8072” used herein includes the hsa-miR-8072 gene (miRBase Accession No. MIMAT0030999) described in SEQ ID NO: 24, a homolog or an ortholog of a different organism species, and the like. The hsa-miR-8072 gene can be obtained by a method described in Wang H J et al., 2013, Shock, Vol. 39, p. 480-487. Also, “hsa-mir-8072” (miRBase Accession No. M10025908, SEQ ID NO: 249) having a hairpin-like structure is known as a precursor of “hsa-miR-8072”.

The term “hsa-miR-6816-5p gene” or “hsa-miR-6816-5p” used herein includes the hsa-miR-6816-5p gene (miRBase Accession No. MIMAT0027532) described in SEQ ID NO: 25, a homolog or an ortholog of a different organism species, and the like. The hsa-miR-6816-5p gene can be obtained by a method described in Ladewig E et al., 2012, Genome Res, Vol. 22, p. 1634-1645. Also, “hsa-mir-6816” (miRBase Accession No. M10022661, SEQ ID NO: 250) having a hairpin-like structure is known as a precursor of “hsa-miR-6816-5p”.

The term “hsa-miR-4281 gene” or “hsa-miR-4281” used herein includes the hsa-miR-4281 gene (miRBase Accession No. MIMAT0016907) described in SEQ ID NO: 26, a homolog or an ortholog of a different organism species, and the like. The hsa-miR-4281 gene can be obtained by a method described in Goff L A et al., 2009, PLoS One, Vol. 4, e7192. Also, “hsa-mir-4281” (miRBase Accession No. M10015885, SEQ ID NO: 251) having a hairpin-like structure is known as a precursor of “hsa-miR-4281”.

The term “hsa-miR-6729-5p gene” or “hsa-miR-6729-5p” used herein includes the hsa-miR-6729-5p gene (miRBase Accession No. MIMAT0027359) described in SEQ ID NO: 27, a homolog or an ortholog of a different organism species, and the like. The hsa-miR-6729-5p p. 1634-1645. Also, “hsa-mir-6729” (miRBase Accession No. M10022574, SEQ ID NO: 252) having a hairpin-like structure is known as a precursor of “hsa-miR-6729-5p”.

The term “hsa-miR-8069 gene” or “hsa-miR-8069” used herein includes the hsa-miR-8069 gene (miRBase Accession No. MIMAT0030996) described in SEQ ID NO: 28, a homolog or an ortholog of a different organism species, and the like. The hsa-miR-8069 gene can be obtained by a method described in Wang H J et al., 2013, Shock, Vol. 39, p. 480-487. Also, “hsa-mir-8069” (miRBase Accession No. M10025905, SEQ ID NO: 253) having a hairpin-like structure is known as a precursor of “hsa-miR-8069”.

The term “hsa-miR-4706 gene” or “hsa-miR-4706” used herein includes the hsa-miR-4706 gene (miRBase Accession No. MIMAT0019806) described in SEQ ID NO: 29, a homolog or an ortholog of a different organism species, and the like. The hsa-miR-4706 gene can be obtained by a method described in Persson H et al., 2011, Cancer Res, Vol. 71, p. 78-86. Also, “hsa-mir-4706” (miRBase Accession No. M10017339, SEQ ID NO: 254) having a hairpin-like structure is known as a precursor of “hsa-miR-4706”.

The term “hsa-miR-7108-5p gene” or “hsa-miR-7108-5p” used herein includes the hsa-miR-7108-5p gene (miRBase Accession No. MIMAT0028113) described in SEQ ID NO: 30, a homolog or an ortholog of a different organism species, and the like. The hsa-miR-7108-5p gene can be obtained by a method described in Ladewig E et al., 2012, Genome Res, Vol. 22, p. 1634-1645. Also, “hsa-mir-7108” (miRBase Accession No. M10022959, SEQ ID NO: 255) having a hairpin-like structure is known as a precursor of “hsa-miR-7108-5p”.

The term “hsa-miR-4433b-3p gene” or “hsa-miR-4433b-3p” used herein includes the hsa-miR-4433b-3p gene (miRBase Accession No. MIMAT0030414) described in SEQ ID NO: 31, a homolog or an ortholog of a different organism species, and the like. The hsa-miR-4433b-3p gene can be obtained by a method described in Ple H et al., 2012, PLoS One, Vol. 7, e50746. Also, “hsa-mir-4433b” (miRBase Accession No. M10025511, SEQ ID NO: 256) having a hairpin-like structure is known as a precursor of “hsa-miR-4433b-3p”.

The term “hsa-miR-6893-5p gene” or “hsa-miR-6893-5p” used herein includes the hsa-miR-6893-5p gene (miRBase Accession No. MIMAT0027686) described in SEQ ID NO: 32, a homolog or an ortholog of a different organism species, and the like. The hsa-miR-6893-5p gene can be obtained by a method described in Ladewig E et al., 2012, Genome Res, Vol. 22, p. 1634-1645. Also, “hsa-mir-6893” (miRBase Accession No. M10022740, SEQ ID NO: 257) having a hairpin-like structure is known as a precursor of “hsa-miR-6893-5p”.

The term “hsa-miR-6857-5p gene” or “hsa-miR-6857-5p” used herein includes the hsa-miR-6857-5p gene (miRBase Accession No. MIMAT0027614) described in SEQ ID NO: 33, a homolog or an ortholog of a different organism species, and the like. The hsa-miR-6857-5p gene can be obtained by a method described in Ladewig E et al., 2012, Genome Res, Vol. 22, p. 1634-1645. Also, “hsa-mir-6857” (miRBase Accession No. M10022703, SEQ ID NO: 258) having a hairpin-like structure is known as a precursor of “hsa-miR-6857-5p”.

The term “hsa-miR-1227-5p gene” or “hsa-miR-1227-5p” used herein includes the hsa-miR-1227-5p gene (miRBase Accession No. MIMAT0022941) described in SEQ ID NO: 34, a homolog or an ortholog of a different organism species, and the like. The hsa-miR-1227-5p gene can be obtained by a method described in Berezikov E et al., 2007, Mol Cell, Vol. 28, p. 328-336. Also, “hsa-mir-1227” (miRBase Accession No. M10006316, SEQ ID NO: 259) having a hairpin-like structure is known as a precursor of “hsa-miR-1227-5p”.

The term “hsa-miR-6741-5p gene” or “hsa-miR-6741-5p” used herein includes the hsa-miR-6741-5p gene (miRBase Accession No. MIMAT0027383) described in SEQ ID NO: 35, a homolog or an ortholog of a different organism species, and the like. The hsa-miR-6741-5p p. 1634-1645. Also, “hsa-mir-6741” (miRBase Accession No. M10022586, SEQ ID NO: 260) having a hairpin-like structure is known as a precursor of “hsa-miR-6741-5p”.

The term “hsa-miR-451a gene” or “hsa-miR-451a” used herein includes the hsa-miR-451a gene (miRBase Accession No. MIMAT0001631) described in SEQ ID NO: 36, a homolog or an ortholog of a different organism species, and the like. The hsa-miR-451a gene can be obtained by a method described in Altuvia Y et al., 2005, Nucleic Acids Res, Vol. 33, p. 2697-2706. Also, “hsa-mir-451a” (miRBase Accession No. M10001729, SEQ ID NO: 261) having a hairpin-like structure is known as a precursor of “hsa-miR-451a”.

The term “hsa-miR-8063 gene” or “hsa-miR-8063” used herein includes the hsa-miR-8063 gene (miRBase Accession No. MIMAT0030990) described in SEQ ID NO: 37, a homolog or an ortholog of a different organism species, and the like. The hsa-miR-8063 gene can be obtained by a method described in Wang H J et al., 2013, Shock, Vol. 39, p. 480-487. Also, “hsa-mir-8063” (miRBase Accession No. M10025899, SEQ ID NO: 262) having a hairpin-like structure is known as a precursor of “hsa-miR-8063”.

The term “hsa-miR-3622a-5p gene” or “hsa-miR-3622a-5p” used herein includes the hsa-miR-3622a-5p gene (miRBase Accession No. MIMAT0018003) described in SEQ ID NO: 38, a homolog or an ortholog of a different organism species, and the like. The hsa-miR-3622a-5p gene can be obtained by a method described in Witten D et al., 2010, BMC Biol, Vol. 8, p. 58. Also, “hsa-mir-3622a” (miRBase Accession No. M10016013, SEQ ID NO: 263) having a hairpin-like structure is known as a precursor of “hsa-miR-3622a-5p”.

The term “hsa-miR-615-5p gene” or “hsa-miR-615-5p” used herein includes the hsa-miR-615-5p gene (miRBase Accession No. MIMAT0004804) described in SEQ ID NO: 39, a homolog or an ortholog of a different organism species, and the like. The hsa-miR-615-5p gene can be obtained by a method described in Cummins J M et al., 2006, Proc Natl Acad Sci USA, Vol. 103, p. 3687-3692. Also, “hsa-mir-615” (miRBase Accession No. M10003628, SEQ ID NO: 264) having a hairpin-like structure is known as a precursor of “hsa-miR-615-5p”.

The term “hsa-miR-128-1-5p gene” or “hsa-miR-128-1-5p” used herein includes the hsa-miR-128-1-5p gene (miRBase Accession No. MIMAT0026477) described in SEQ ID NO: 40, a homolog or an ortholog of a different organism species, and the like. The hsa-miR-128-1-5p gene can be obtained by a method described in Lagos-Quintana M et al., 2002, Curr Biol, Vol. 12, p. 735-739. Also, “hsa-mir-128-1” (miRBase Accession No. M10000447, SEQ ID NO: 265) having a hairpin-like structure is known as a precursor of “hsa-miR-128-1-5p”.

The term “hsa-miR-6825-5p gene” or “hsa-miR-6825-5p” used herein includes the hsa-miR-6825-5p gene (miRBase Accession No. MIMAT0027550) described in SEQ ID NO: 41, a homolog or an ortholog of a different organism species, and the like. The hsa-miR-6825-5p gene can be obtained by a method described in Ladewig E et al., 2012, Genome Res, Vol. 22, p. 1634-1645. Also, “hsa-mir-6825” (miRBase Accession No. M10022670, SEQ ID NO: 266) having a hairpin-like structure is known as a precursor of “hsa-miR-6825-5p”.

The term “hsa-miR-1260b gene” or “hsa-miR-1260b” used herein includes the hsa-miR-1260b gene (miRBase Accession No. MIMAT0015041) described in SEQ ID NO: 42, a homolog or an ortholog of a different organism species, and the like. The hsa-miR-1260b gene can be obtained by a method described in Stark M S et al., 2010, PLoS One, Vol. 5, e9685. Also, “hsa-mir-1260b” (miRBase Accession No. M10014197, SEQ ID NO: 267) having a hairpin-like structure is known as a precursor of “hsa-miR-1260b”.

The term “hsa-miR-4433-3p gene” or “hsa-miR-4433-3p” used herein includes the hsa-miR-4433-3p gene (miRBase Accession No. MIMAT0018949) described in SEQ ID NO: 43, a homolog or an ortholog of a different organism species, and the like. The hsa-miR-4433-3p gene can be obtained by a method described in Jima D D et al., 2010, Blood, Vol. 116, e118-e127. Also, “hsa-mir-4433” (miRBase Accession No. MI0016773, SEQ ID NO: 268) having a hairpin-like structure is known as a precursor of “hsa-miR-4433-3p”.

The term “hsa-miR-4665-5p gene” or “hsa-miR-4665-5p” used herein includes the hsa-miR-4665-5p gene (miRBase Accession No. MIMAT0019739) described in SEQ ID NO: 44, a homolog or an ortholog of a different organism species, and the like. The hsa-miR-4665-5p gene can be obtained by a method described in Persson H et al., 2011, Cancer Res, Vol. 71, p. 78-86. Also, “hsa-mir-4665” (miRBase Accession No. MI0017295, SEQ ID NO: 269) having a hairpin-like structure is known as a precursor of “hsa-miR-4665-5p”.

The term “hsa-miR-7845-5p gene” or “hsa-miR-7845-5p” used herein includes the hsa-miR-7845-5p gene (miRBase Accession No. MIMAT0030420) described in SEQ ID NO: 45, a homolog or an ortholog of a different organism species, and the like. The hsa-miR-7845-5p gene can be obtained by a method described in Ple H et al., 2012, PLoS One, Vol. 7, e50746. Also, “hsa-mir-7845” (miRBase Accession No. MI0025515, SEQ ID NO: 270) having a hairpin-like structure is known as a precursor of “hsa-miR-7845-5p”.

The term “hsa-miR-1908-5p gene” or “hsa-miR-1908-5p” used herein includes the hsa-miR-1908-5p gene (miRBase Accession No. MIMAT0007881) described in SEQ ID NO: 46, a homolog or an ortholog of a different organism species, and the like. The hsa-miR-1908-5p gene can be obtained by a method described in Bar M et al., 2008, Stem Cells, Vol. 26, p. 2496-2505. Also, “hsa-mir-1908” (miRBase Accession No. MI0008329, SEQ ID NO: 271) having a hairpin-like structure is known as a precursor of “hsa-miR-1908-5p”.

The term “hsa-miR-6840-3p gene” or “hsa-miR-6840-3p” used herein includes the hsa-miR-6840-3p gene (miRBase Accession No. MIMAT0027583) described in SEQ ID NO: 47, a homolog or an ortholog of a different organism species, and the like. The hsa-miR-6840-3p p. 1634-1645. Also, “hsa-mir-6840” (miRBase Accession No. M10022686, SEQ ID NO: 272) having a hairpin-like structure is known as a precursor of “hsa-miR-6840-3p”.

The term “hsa-miR-6765-5p gene” or “hsa-miR-6765-5p” used herein includes the hsa-miR-6765-5p gene (miRBase Accession No. MIMAT0027430) described in SEQ ID NO: 48, a homolog or an ortholog of a different organism species, and the like. The hsa-miR-6765-5p gene can be obtained by a method described in Ladewig E et al., 2012, Genome Res, Vol. 22, p. 1634-1645. Also, “hsa-mir-6765” (miRBase Accession No. M10022610, SEQ ID NO: 240) having a hairpin-like structure is known as a precursor of “hsa-miR-6765-5p”.

The term “hsa-miR-296-5p gene” or “hsa-miR-296-5p” used herein includes the hsa-miR-296-5p gene (miRBase Accession No. MIMAT0000690) described in SEQ ID NO: 49, a homolog or an ortholog of a different organism species, and the like. The hsa-miR-296-5p gene can be obtained by a method described in Houbaviy H B et al., 2003, Dev Cell, Vol. 5, p. 351-358. Also, “hsa-mir-296” (miRBase Accession No. M10000747, SEQ ID NO: 273) having a hairpin-like structure is known as a precursor of “hsa-miR-296-5p”.

The term “hsa-miR-3675-3p gene” or “hsa-miR-3675-3p” used herein includes the hsa-miR-3675-3p gene (miRBase Accession No. MIMAT0018099) described in SEQ ID NO: 50, a homolog or an ortholog of a different organism species, and the like. The hsa-miR-3675-3p gene can be obtained by a method described in Vaz C et al., 2010, BMC Genomics, Vol. 11, p. 288. Also, “hsa-mir-3675” (miRBase Accession No. M10016076, SEQ ID NO: 274) having a hairpin-like structure is known as a precursor of “hsa-miR-3675-3p”.

The term “hsa-miR-6781-5p gene” or “hsa-miR-6781-5p” used herein includes the hsa-miR-6781-5p gene (miRBase Accession No. MIMAT0027462) described in SEQ ID NO: 51, a homolog or an ortholog of a different organism species, and the like. The hsa-miR-6781-5p p. 1634-1645. Also, “hsa-mir-6781” (miRBase Accession No. M10022626, SEQ ID NO: 275) having a hairpin-like structure is known as a precursor of “hsa-miR-6781-5p”.

The term “hsa-miR-423-5p gene” or “hsa-miR-423-5p” used herein includes the hsa-miR-423-5p gene (miRBase Accession No. MIMAT0004748) described in SEQ ID NO: 52, a homolog or an ortholog of a different organism species, and the like. The hsa-miR-423-5p gene can be obtained by a method described in Kasashima K et al., 2004, Biochem Biophys Res Commun, Vol. 322, p. 403-410. Also, “hsa-mir-423” (miRBase Accession No. MI0001445, SEQ ID NO: 276) having a hairpin-like structure is known as a precursor of “hsa-miR-423-5p”.

The term “hsa-miR-3663-3p gene” or “hsa-miR-3663-3p” used herein includes the hsa-miR-3663-3p gene (miRBase Accession No. MIMAT0018085) described in SEQ ID NO: 53, a homolog or an ortholog of a different organism species, and the like. The hsa-miR-3663-3p gene can be obtained by a method described in Liao J Y et al., 2010, PLoS One, Vol. 5, e10563. Also, “hsa-mir-3663” (miRBase Accession No. M10016064, SEQ ID NO: 277) having a hairpin-like structure is known as a precursor of “hsa-miR-3663-3p”.

The term “hsa-miR-6784-5p gene” or “hsa-miR-6784-5p” used herein includes the hsa-miR-6784-5p gene (miRBase Accession No. MIMAT0027468) described in SEQ ID NO: 54, a homolog or an ortholog of a different organism species, and the like. The hsa-miR-6784-5p gene can be obtained by a method described in Ladewig E et al., 2012, Genome Res, Vol. 22, p. 1634-1645. Also, “hsa-mir-6784” (miRBase Accession No. M10022629, SEQ ID NO: 278) having a hairpin-like structure is known as a precursor of “hsa-miR-6784-5p”.

The term “hsa-miR-6749-5p gene” or “hsa-miR-6749-5p” used herein includes the hsa-miR-6749-5p gene (miRBase Accession No. MIMAT0027398) described in SEQ ID NO: 55, a homolog or an ortholog of a different organism species, and the like. The hsa-miR-6749-5p p. 1634-1645. Also, “hsa-mir-6749” (miRBase Accession No. M10022594, SEQ ID NO: 279) having a hairpin-like structure is known as a precursor of “hsa-miR-6749-5p”.

The term “hsa-miR-1231 gene” or “hsa-miR-1231” used herein includes the hsa-miR-1231 gene (miRBase Accession No. MIMAT0005586) described in SEQ ID NO: 56, a homolog or an ortholog of a different organism species, and the like. The hsa-miR-1231 gene can be obtained by a method described in Berezikov E et al., 2007, Mol Cell, Vol. 28, p. 328-336. Also, “hsa-mir-1231” (miRBase Accession No. M10006321, SEQ ID NO: 280) having a hairpin-like structure is known as a precursor of “hsa-miR-1231”.

The term “hsa-miR-4746-3p gene” or “hsa-miR-4746-3p” used herein includes the hsa-miR-4746-3p gene (miRBase Accession No. MIMAT0019881) described in SEQ ID NO: 57, a homolog or an ortholog of a different organism species, and the like. The hsa-miR-4746-3p gene can be obtained by a method described in Persson H et al., 2011, Cancer Res, Vol. 71, p. 78-86. Also, “hsa-mir-4746” (miRBase Accession No. M10017385, SEQ ID NO: 281) having a hairpin-like structure is known as a precursor of “hsa-miR-4746-3p”.

The term “hsa-miR-6780b-5p gene” or “hsa-miR-6780b-5p” used herein includes the hsa-miR-6780b-5p gene (miRBase Accession No. MIMAT0027572) described in SEQ ID NO: 58, a homolog or an ortholog of a different organism species, and the like. The hsa-miR-6780b-5p gene can be obtained by a method described in Ladewig E et al., 2012, Genome Res, Vol. 22, p. 1634-1645. Also, “hsa-mir-6780b” (miRBase Accession No. M10022681, SEQ ID NO: 282) having a hairpin-like structure is known as a precursor of “hsa-miR-6780b-5p”.

The term “hsa-miR-4758-5p gene” or “hsa-miR-4758-5p” used herein includes the hsa-miR-4758-5p gene (miRBase Accession No. MIMAT0019903) described in SEQ ID NO: 59, a homolog or an ortholog of a different organism species, and the like. The hsa-miR-4758-5p 78-86. Also, “hsa-mir-4758” (miRBase Accession No. M10017399, SEQ ID NO: 283) having a hairpin-like structure is known as a precursor of “hsa-miR-4758-5p”.

The term “hsa-miR-3679-5p gene” or “hsa-miR-3679-5p” used herein includes the hsa-miR-3679-5p gene (miRBase Accession No. MIMAT0018104) described in SEQ ID NO: 60, a homolog or an ortholog of a different organism species, and the like. The hsa-miR-3679-5p gene can be obtained by a method described in Creighton C J et al., 2010, PLoS One, Vol. 5, e9637. Also, “hsa-mir-3679” (miRBase Accession No. M10016080, SEQ ID NO: 236) having a hairpin-like structure is known as a precursor of “hsa-miR-3679-5p”.

The term “hsa-miR-3184-5p gene” or “hsa-miR-3184-5p” used herein includes the hsa-miR-3184-5p gene (miRBase Accession No. MIMAT0015064) described in SEQ ID NO: 61, a homolog or an ortholog of a different organism species, and the like. The hsa-miR-3184-5p gene can be obtained by a method described in Stark M S et al., 2010, PLoS One, Vol. 5, e9685. Also, “hsa-mir-3184” (miRBase Accession No. M10014226, SEQ ID NO: 284) having a hairpin-like structure is known as a precursor of “hsa-miR-3184-5p”.

The term “hsa-miR-6125 gene” or “hsa-miR-6125” used herein includes the hsa-miR-6125 gene (miRBase Accession No. MIMAT0024598) described in SEQ ID NO: 62, a homolog or an ortholog of a different organism species, and the like. The hsa-miR-6125 gene can be obtained by a method described in Smith J L et al., 2012, J Virol, Vol. 86, p. 5278-5287. Also, “hsa-mir-6125” (miRBase Accession No. M10021259, SEQ ID NO: 285) having a hairpin-like structure is known as a precursor of “hsa-miR-6125”.

The term “hsa-miR-6721-5p gene” or “hsa-miR-6721-5p” used herein includes the hsa-miR-6721-5p gene (miRBase Accession No. MIMAT0025852) described in SEQ ID NO: 63, a homolog or an ortholog of a different organism species, and the like. The hsa-miR-6721-5p gene can be obtained by a method described in Li Y et al., 2012, Gene, Vol. 497, p. 330-335. Also, “hsa-mir-6721” (miRBase Accession No. M10022556, SEQ ID NO: 286) having a hairpin-like structure is known as a precursor of “hsa-miR-6721-5p”.

The term “hsa-miR-6791-5p gene” or “hsa-miR-6791-5p” used herein includes the hsa-miR-6791-5p gene (miRBase Accession No. MIMAT0027482) described in SEQ ID NO: 64, a homolog or an ortholog of a different organism species, and the like. The hsa-miR-6791-5p gene can be obtained by a method described in Ladewig E et al., 2012, Genome Res, Vol. 22, p. 1634-1645. Also, “hsa-mir-6791” (miRBase Accession No. M10022636, SEQ ID NO: 287) having a hairpin-like structure is known as a precursor of “hsa-miR-6791-5p”.

The term “hsa-miR-3185 gene” or “hsa-miR-3185” used herein includes the hsa-miR-3185 gene (miRBase Accession No. MIMAT0015065) described in SEQ ID NO: 65, a homolog or an ortholog of a different organism species, and the like. The hsa-miR-3185 gene can be obtained by a method described in Stark M S et al., 2010, PLoS One, Vol. 5, e9685. Also, “hsa-mir-3185” (miRBase Accession No. M10014227, SEQ ID NO: 288) having a hairpin-like structure is known as a precursor of “hsa-miR-3185”.

The term “hsa-miR-1260a gene” or “hsa-miR-1260a” used herein includes the hsa-miR-1260a gene (miRBase Accession No. MIMAT0005911) described in SEQ ID NO: 66, a homolog or an ortholog of a different organism species, and the like. The hsa-miR-1260a gene can be obtained by a method described in Morin R D et al., 2008, Genome Res, Vol. 18, p. 610-621. Also, “hsa-mir-1260a” (miRBase Accession No. M10006394, SEQ ID NO: 289) having a hairpin-like structure is known as a precursor of “hsa-miR-1260a”.

The term “hsa-miR-3197 gene” or “hsa-miR-3197” used herein includes the hsa-miR-3197 gene (miRBase Accession No. MIMAT0015082) described in SEQ ID NO: 67, a homolog or an ortholog of a different organism species, and the like. The hsa-miR-3197 gene can be obtained by a method described in Stark M S et al., 2010, PLoS One, Vol. 5, e9685. Also, “hsa-mir-3197” (miRBase Accession No. M10014245, SEQ ID NO: 290) having a hairpin-like structure is known as a precursor of “hsa-miR-3197”.

The term “hsa-miR-6845-5p gene” or “hsa-miR-6845-5p” used herein includes the hsa-miR-6845-5p gene (miRBase Accession No. MIMAT0027590) described in SEQ ID NO: 68, a homolog or an ortholog of a different organism species, and the like. The hsa-miR-6845-5p gene can be obtained by a method described in Ladewig E et al., 2012, Genome Res, Vol. 22, p. 1634-1645. Also, “hsa-mir-6845” (miRBase Accession No. M10022691, SEQ ID NO: 291) having a hairpin-like structure is known as a precursor of “hsa-miR-6845-5p”.

The term “hsa-miR-6887-5p gene” or “hsa-miR-6887-5p” used herein includes the hsa-miR-6887-5p gene (miRBase Accession No. MIMAT0027674) described in SEQ ID NO: 69, a homolog or an ortholog of a different organism species, and the like. The hsa-miR-6887-5p gene can be obtained by a method described in Ladewig E et al., 2012, Genome Res, Vol. 22, p. 1634-1645. Also, “hsa-mir-6887” (miRBase Accession No. M10022734, SEQ ID NO: 292) having a hairpin-like structure is known as a precursor of “hsa-miR-6887-5p”.

The term “hsa-miR-6738-5p gene” or “hsa-miR-6738-5p” used herein includes the hsa-miR-6738-5p gene (miRBase Accession No. MIMAT0027377) described in SEQ ID NO: 70, a homolog or an ortholog of a different organism species, and the like. The hsa-miR-6738-5p gene can be obtained by a method described in Ladewig E et al., 2012, Genome Res, Vol. 22, p. 1634-1645. Also, “hsa-mir-6738” (miRBase Accession No. M10022583, SEQ ID NO: 293) having a hairpin-like structure is known as a precursor of “hsa-miR-6738-5p”.

The term “hsa-miR-6872-3p gene” or “hsa-miR-6872-3p” used herein includes the hsa-miR-6872-3p gene (miRBase Accession No. MIMAT0027645) described in SEQ ID NO: 71, a homolog or an ortholog of a different organism species, and the like. The hsa-miR-6872-3p p. 1634-1645. Also, “hsa-mir-6872” (miRBase Accession No. M10022719, SEQ ID NO: 294) having a hairpin-like structure is known as a precursor of “hsa-miR-6872-3p”.

The term “hsa-miR-4497 gene” or “hsa-miR-4497” used herein includes the hsa-miR-4497 gene (miRBase Accession No. MIMAT0019032) described in SEQ ID NO: 72, a homolog or an ortholog of a different organism species, and the like. The hsa-miR-4497 gene can be obtained by a method described in Jima D D et al., 2010, Blood, Vol. 116, e118-e127. Also, “hsa-mir-4497” (miRBase Accession No. M10016859, SEQ ID NO: 295) having a hairpin-like structure is known as a precursor of “hsa-miR-4497”.

The term “hsa-miR-1229-5p gene” or “hsa-miR-1229-5p” used herein includes the hsa-miR-1229-5p gene (miRBase Accession No. MIMAT0022942) described in SEQ ID NO: 73, a homolog or an ortholog of a different organism species, and the like. The hsa-miR-1229-5p gene can be obtained by a method described in Berezikov E et al., 2007, Mol Cell, Vol. 28, p. 328-336. Also, “hsa-mir-1229” (miRBase Accession No. M10006319, SEQ ID NO: 296) having a hairpin-like structure is known as a precursor of “hsa-miR-1229-5p”.

The term “hsa-miR-6820-5p gene” or “hsa-miR-6820-5p” used herein includes the hsa-miR-6820-5p gene (miRBase Accession No. MIMAT0027540) described in SEQ ID NO: 74, a homolog or an ortholog of a different organism species, and the like. The hsa-miR-6820-5p gene can be obtained by a method described in Ladewig E et al., 2012, Genome Res, Vol. 22, p. 1634-1645. Also, “hsa-mir-6820” (miRBase Accession No. M10022665, SEQ ID NO: 297) having a hairpin-like structure is known as a precursor of “hsa-miR-6820-5p”.

The term “hsa-miR-6777-5p gene” or “hsa-miR-6777-5p” used herein includes the hsa-miR-6777-5p gene (miRBase Accession No. MIMAT0027454) described in SEQ ID NO: 75, a homolog or an ortholog of a different organism species, and the like. The hsa-miR-6777-5p p. 1634-1645. Also, “hsa-mir-6777” (miRBase Accession No. M10022622, SEQ ID NO: 298) having a hairpin-like structure is known as a precursor of “hsa-miR-6777-5p”.

The term “hsa-miR-3917 gene” or “hsa-miR-3917” used herein includes the hsa-miR-3917 gene (miRBase Accession No. MIMAT0018191) described in SEQ ID NO: 76, a homolog or an ortholog of a different organism species, and the like. The hsa-miR-3917 gene can be obtained by a method described in Creighton C J et al., 2010, PLoS One, Vol. 5, e9637. Also, “hsa-mir-3917” (miRBase Accession No. M10016423, SEQ ID NO: 299) having a hairpin-like structure is known as a precursor of “hsa-miR-3917”.

The term “hsa-miR-5787 gene” or “hsa-miR-5787” used herein includes the hsa-miR-5787 gene (miRBase Accession No. MIMAT0023252) described in SEQ ID NO: 77, a homolog or an ortholog of a different organism species, and the like. The hsa-miR-5787 gene can be obtained by a method described in Yoo H et al., 2011, Biochem Biophys Res Commun, Vol. 415, p. 567-572. Also, “hsa-mir-5787” (miRBase Accession No. M10019797, SEQ ID NO: 300) having a hairpin-like structure is known as a precursor of “hsa-miR-5787”.

The term “hsa-miR-4286 gene” or “hsa-miR-4286” used herein includes the hsa-miR-4286 gene (miRBase Accession No. MIMAT0016916) described in SEQ ID NO: 78, a homolog or an ortholog of a different organism species, and the like. The hsa-miR-4286 gene can be obtained by a method described in Goff L A et al., 2009, PLoS One, Vol. 4, e7192. Also, “hsa-mir-4286” (miRBase Accession No. MI0015894, SEQ ID NO: 301) having a hairpin-like structure is known as a precursor of “hsa-miR-4286”.

The term “hsa-miR-6877-5p gene” or “hsa-miR-6877-5p” used herein includes the hsa-miR-6877-5p gene (miRBase Accession No. MIMAT0027654) described in SEQ ID NO: 79, a homolog or an ortholog of a different organism species, and the like. The hsa-miR-6877-5p p. 1634-1645. Also, “hsa-mir-6877” (miRBase Accession No. M10022724, SEQ ID NO: 302) having a hairpin-like structure is known as a precursor of “hsa-miR-6877-5p”.

The term “hsa-miR-1225-3p gene” or “hsa-miR-1225-3p” used herein includes the hsa-miR-1225-3p gene (miRBase Accession No. MIMAT0005573) described in SEQ ID NO: 80, a homolog or an ortholog of a different organism species, and the like. The hsa-miR-1225-3p gene can be obtained by a method described in Berezikov E et al., 2007, Mol Cell, Vol. 28, p. 328-336. Also, “hsa-mir-1225” (miRBase Accession No. M10006311, SEQ ID NO: 303) having a hairpin-like structure is known as a precursor of “hsa-miR-1225-3p”.

The term “hsa-miR-6088 gene” or “hsa-miR-6088” used herein includes the hsa-miR-6088 gene (miRBase Accession No. MIMAT0023713) described in SEQ ID NO: 81, a homolog or an ortholog of a different organism species, and the like. The hsa-miR-6088 gene can be obtained by a method described in Yoo J K et al., 2012, Stem Cells Dev, Vol. 21, p. 2049-2057. Also, “hsa-mir-6088” (miRBase Accession No. M10020365, SEQ ID NO: 304) having a hairpin-like structure is known as a precursor of “hsa-miR-6088”.

The term “hsa-miR-6800-5p gene” or “hsa-miR-6800-5p” used herein includes the hsa-miR-6800-5p gene (miRBase Accession No. MIMAT0027500) described in SEQ ID NO: 82, a homolog or an ortholog of a different organism species, and the like. The hsa-miR-6800-5p gene can be obtained by a method described in Ladewig E et al., 2012, Genome Res, Vol. 22, p. 1634-1645. Also, “hsa-mir-6800” (miRBase Accession No. M10022645, SEQ ID NO: 305) having a hairpin-like structure is known as a precursor of “hsa-miR-6800-5p”.

The term “hsa-miR-1246 gene” or “hsa-miR-1246” used herein includes the hsa-miR-1246 gene (miRBase Accession No. MIMAT0005898) described in SEQ ID NO: 83, a homolog or an ortholog of a different organism species, and the like. The hsa-miR-1246 gene can be obtained by a method described in Morin R D et al., 2008, Genome Res, Vol. 18, p. 610-621. Also, “hsa-mir-1246” (miRBase Accession No. M10006381, SEQ ID NO: 306) having a hairpin-like structure is known as a precursor of “hsa-miR-1246”.

The term “hsa-miR-4467 gene” or “hsa-miR-4467” used herein includes the hsa-miR-4467 gene (miRBase Accession No. MIMAT0018994) described in SEQ ID NO: 84, a homolog or an ortholog of a different organism species, and the like. The hsa-miR-4467 gene can be obtained by a method described in Jima D D et al., 2010, Blood, Vol. 116, e118-e127. Also, “hsa-mir-4467” (miRBase Accession No. M10016818, SEQ ID NO: 307) having a hairpin-like structure is known as a precursor of “hsa-miR-4467”.

The term “hsa-miR-4419b gene” or “hsa-miR-4419b” used herein includes the hsa-miR-4419b gene (miRBase Accession No. MIMAT0019034) described in SEQ ID NO: 85, a homolog or an ortholog of a different organism species, and the like. The hsa-miR-4419b gene can be obtained by a method described in Jima D D et al., 2010, Blood, Vol. 116, e118-e127. Also, “hsa-mir-4419b” (miRBase Accession No. M10016861, SEQ ID NO: 308) having a hairpin-like structure is known as a precursor of “hsa-miR-4419b”.

The term “hsa-miR-1914-3p gene” or “hsa-miR-1914-3p” used herein includes the hsa-miR-1914-3p gene (miRBase Accession No. MIMAT0007890) described in SEQ ID NO: 86, a homolog or an ortholog of a different organism species, and the like. The hsa-miR-1914-3p gene can be obtained by a method described in Bar M et al., 2008, Stem Cells, Vol. 26, p. 2496-2505. Also, “hsa-mir-1914” (miRBase Accession No. M10008335, SEQ ID NO: 309) having a hairpin-like structure is known as a precursor of “hsa-miR-1914-3p”.

The term “hsa-miR-4632-5p gene” or “hsa-miR-4632-5p” used herein includes the hsa-miR-4632-5p gene (miRBase Accession No. MIMAT0022977) described in SEQ ID NO: 87, a homolog or an ortholog of a different organism species, and the like. The hsa-miR-4632-5p 78-86. Also, “hsa-mir-4632” (miRBase Accession No. M10017259, SEQ ID NO: 310) having a hairpin-like structure is known as a precursor of “hsa-miR-4632-5p”.

The term “hsa-miR-1915-5p gene” or “hsa-miR-1915-5p” used herein includes the hsa-miR-1915-5p gene (miRBase Accession No. MIMAT0007891) described in SEQ ID NO: 88, a homolog or an ortholog of a different organism species, and the like. The hsa-miR-1915-5p gene can be obtained by a method described in Bar M et al., 2008, Stem Cells, Vol. 26, p. 2496-2505. Also, “hsa-mir-1915” (miRBase Accession No. M10008336, SEQ ID NO: 311) having a hairpin-like structure is known as a precursor of “hsa-miR-1915-5p”.

The term “hsa-miR-3940-5p gene” or “hsa-miR-3940-5p” used herein includes the hsa-miR-3940-5p gene (miRBase Accession No. MIMAT0019229) described in SEQ ID NO: 89, a homolog or an ortholog of a different organism species, and the like. The hsa-miR-3940-5p gene can be obtained by a method described in Liao J Y et al., 2010, PLoS One, Vol. 5, e10563. Also, “hsa-mir-3940” (miRBase Accession No. M10016597, SEQ ID NO: 312) having a hairpin-like structure is known as a precursor of “hsa-miR-3940-5p”.

The term “hsa-miR-1185-2-3p gene” or “hsa-miR-1185-2-3p” used herein includes the hsa-miR-1185-2-3p gene (miRBase Accession No. MIMAT0022713) described in SEQ ID NO: 90, a homolog or an ortholog of a different organism species, and the like. The hsa-miR-1185-2-3p gene can be obtained by a method described in Berezikov E et al., 2006, Genome Res, Vol. 16, p. 1289-1298. Also, “hsa-mir-1185-2” (miRBase Accession No. M10003821, SEQ ID NO: 313) having a hairpin-like structure is known as a precursor of “hsa-miR-1185-2-3p”.

The term “hsa-miR-6746-5p gene” or “hsa-miR-6746-5p” used herein includes the hsa-miR-6746-5p gene (miRBase Accession No. MIMAT0027392) described in SEQ ID NO: 91, a homolog or an ortholog of a different organism species, and the like. The hsa-miR-6746-5p p. 1634-1645. Also, “hsa-mir-6746” (miRBase Accession No. M10022591, SEQ ID NO: 314) having a hairpin-like structure is known as a precursor of “hsa-miR-6746-5p”.

The term “hsa-miR-5001-5p gene” or “hsa-miR-5001-5p” used herein includes the hsa-miR-5001-5p gene (miRBase Accession No. MIMAT0021021) described in SEQ ID NO: 92, a homolog or an ortholog of a different organism species, and the like. The hsa-miR-5001-5p gene can be obtained by a method described in Hansen T B et al., 2011, RNA Biol, Vol. 8, p. 378-383. Also, “hsa-mir-5001” (miRBase Accession No. M10017867, SEQ ID NO: 315) having a hairpin-like structure is known as a precursor of “hsa-miR-5001-5p”.

The term “hsa-miR-1228-5p gene” or “hsa-miR-1228-5p” used herein includes the hsa-miR-1228-5p gene (miRBase Accession No. MIMAT0005582) described in SEQ ID NO: 93, a homolog or an ortholog of a different organism species, and the like. The hsa-miR-1228-5p gene can be obtained by a method described in Berezikov E et al., 2007, Mol Cell, Vol. 28, p. 328-336. Also, “hsa-mir-1228” (miRBase Accession No. M10006318, SEQ ID NO: 316) having a hairpin-like structure is known as a precursor of “hsa-miR-1228-5p”.

The term “hsa-miR-5572 gene” or “hsa-miR-5572” used herein includes the hsa-miR-5572 gene (miRBase Accession No. MIMAT0022260) described in SEQ ID NO: 94, a homolog or an ortholog of a different organism species, and the like. The hsa-miR-5572 gene can be obtained by a method described in Tandon Metal., 2012, Oral Dis, Vol. 18, p. 127-131. Also, “hsa-mir-5572” (miRBase Accession No. M10019117, SEQ ID NO: 317) having a hairpin-like structure is known as a precursor of “hsa-miR-5572”.

The term “hsa-miR-4327 gene” or “hsa-miR-4327” used herein includes the hsa-miR-4327 gene (miRBase Accession No. MIMAT0016889) described in SEQ ID NO: 95, a homolog or an ortholog of a different organism species, and the like. The hsa-miR-4327 gene can be obtained by a method described in Goff L A et al., 2009, PLoS One, Vol. 4, e7192. Also, “hsa-mir-4327” (miRBase Accession No. M10015867, SEQ ID NO: 318) having a hairpin-like structure is known as a precursor of “hsa-miR-4327”.

The term “hsa-miR-4638-5p gene” or “hsa-miR-4638-5p” used herein includes the hsa-miR-4638-5p gene (miRBase Accession No. MIMAT0019695) described in SEQ ID NO: 96, a homolog or an ortholog of a different organism species, and the like. The hsa-miR-4638-5p gene can be obtained by a method described in Persson H et al., 2011, Cancer Res, Vol. 71, p. 78-86. Also, “hsa-mir-4638” (miRBase Accession No. M10017265, SEQ ID NO: 319) having a hairpin-like structure is known as a precursor of “hsa-miR-4638-5p”.

The term “hsa-miR-6799-5p gene” or “hsa-miR-6799-5p” used herein includes the hsa-miR-6799-5p gene (miRBase Accession No. MIMAT0027498) described in SEQ ID NO: 97, a homolog or an ortholog of a different organism species, and the like. The hsa-miR-6799-5p gene can be obtained by a method described in Ladewig E et al., 2012, Genome Res, Vol. 22, p. 1634-1645. Also, “hsa-mir-6799” (miRBase Accession No. M10022644, SEQ ID NO: 320) having a hairpin-like structure is known as a precursor of “hsa-miR-6799-5p”.

The term “hsa-miR-6861-5p gene” or “hsa-miR-6861-5p” used herein includes the hsa-miR-6861-5p gene (miRBase Accession No. MIMAT0027623) described in SEQ ID NO: 98, a homolog or an ortholog of a different organism species, and the like. The hsa-miR-6861-5p gene can be obtained by a method described in Ladewig E et al., 2012, Genome Res, Vol. 22, p. 1634-1645. Also, “hsa-mir-6861” (miRBase Accession No. M10022708, SEQ ID NO: 321) having a hairpin-like structure is known as a precursor of “hsa-miR-6861-5p”.

The term “hsa-miR-6727-5p gene” or “hsa-miR-6727-5p” used herein includes the hsa-miR-6727-5p gene (miRBase Accession No. MIMAT0027355) described in SEQ ID NO: 99, a homolog or an ortholog of a different organism species, and the like. The hsa-miR-6727-5p p. 1634-1645. Also, “hsa-mir-6727” (miRBase Accession No. M10022572, SEQ ID NO: 322) having a hairpin-like structure is known as a precursor of “hsa-miR-6727-5p”.

The term “hsa-miR-4513 gene” or “hsa-miR-4513” used herein includes the hsa-miR-4513 gene (miRBase Accession No. MIMAT0019050) described in SEQ ID NO: 100, a homolog or an ortholog of a different organism species, and the like. The hsa-miR-4513 gene can be obtained by a method described in Jima D D et al., 2010, Blood, Vol. 116, e118-e127. Also, “hsa-mir-4513” (miRBase Accession No. M10016879, SEQ ID NO: 323) having a hairpin-like structure is known as a precursor of “hsa-miR-4513”.

The term “hsa-miR-6805-3p gene” or “hsa-miR-6805-3p” used herein includes the hsa-miR-6805-3p gene (miRBase Accession No. MIMAT0027511) described in SEQ ID NO: 101, a homolog or an ortholog of a different organism species, and the like. The hsa-miR-6805-3p gene can be obtained by a method described in Ladewig E et al., 2012, Genome Res, Vol. 22, p. 1634-1645. Also, “hsa-mir-6805” (miRBase Accession No. M10022650, SEQ ID NO: 324) having a hairpin-like structure is known as a precursor of “hsa-miR-6805-3p”.

The term “hsa-miR-6808-5p gene” or “hsa-miR-6808-5p” used herein includes the hsa-miR-6808-5p gene (miRBase Accession No. MIMAT0027516) described in SEQ ID NO: 102, a homolog or an ortholog of a different organism species, and the like. The hsa-miR-6808-5p gene can be obtained by a method described in Ladewig E et al., 2012, Genome Res, Vol. 22, p. 1634-1645. Also, “hsa-mir-6808” (miRBase Accession No. M10022653, SEQ ID NO: 325) having a hairpin-like structure is known as a precursor of “hsa-miR-6808-5p”.

The term “hsa-miR-4449 gene” or “hsa-miR-4449” used herein includes the hsa-miR-4449 gene (miRBase Accession No. MIMAT0018968) described in SEQ ID NO: 103, a homolog or an ortholog of a different organism species, and the like. The hsa-miR-4449 gene Also, “hsa-mir-4449” (miRBase Accession No. MI0016792, SEQ ID NO: 326) having a hairpin-like structure is known as a precursor of “hsa-miR-4449”.

The term “hsa-miR-1199-5p gene” or “hsa-miR-1199-5p” used herein includes the hsa-miR-1199-5p gene (miRBase Accession No. MIMAT0031119) described in SEQ ID NO: 104, a homolog or an ortholog of a different organism species, and the like. The hsa-miR-1199-5p gene can be obtained by a method described in Salvi A et al., 2013, Int J Oncol, Vol. 42, p. 391-402. Also, “hsa-mir-1199” (miRBase Accession No. MI0020340, SEQ ID NO: 327) having a hairpin-like structure is known as a precursor of “hsa-miR-1199-5p”.

The term “hsa-miR-1275 gene” or “hsa-miR-1275” used herein includes the hsa-miR-1275 gene (miRBase Accession No. MIMAT0005929) described in SEQ ID NO: 105, a homolog or an ortholog of a different organism species, and the like. The hsa-miR-1275 gene can be obtained by a method described in Morin R D et al., 2008, Genome Res, Vol. 18, p. 610-621. Also, “hsa-mir-1275” (miRBase Accession No. MI0006415, SEQ ID NO: 328) having a hairpin-like structure is known as a precursor of “hsa-miR-1275”.

The term “hsa-miR-4792 gene” or “hsa-miR-4792” used herein includes the hsa-miR-4792 gene (miRBase Accession No. MIMAT0019964) described in SEQ ID NO: 106, a homolog or an ortholog of a different organism species, and the like. The hsa-miR-4792 gene can be obtained by a method described in Persson H et al., 2011, Cancer Res, Vol. 71, p. 78-86. Also, “hsa-mir-4792” (miRBase Accession No. MI0017439, SEQ ID NO: 329) having a hairpin-like structure is known as a precursor of “hsa-miR-4792”.

The term “hsa-miR-4443 gene” or “hsa-miR-4443” used herein includes the hsa-miR-4443 gene (miRBase Accession No. MIMAT0018961) described in SEQ ID NO: 107, a homolog or an ortholog of a different organism species, and the like. The hsa-miR-4443 gene Also, “hsa-mir-4443” (miRBase Accession No. M10016786, SEQ ID NO: 330) having a hairpin-like structure is known as a precursor of “hsa-miR-4443”.

The term “hsa-miR-6891-5p gene” or “hsa-miR-6891-5p” used herein includes the hsa-miR-6891-5p gene (miRBase Accession No. MIMAT0027682) described in SEQ ID NO: 108, a homolog or an ortholog of a different organism species, and the like. The hsa-miR-6891-5p gene can be obtained by a method described in Ladewig E et al., 2012, Genome Res, Vol. 22, p. 1634-1645. Also, “hsa-mir-6891” (miRBase Accession No. M10022738, SEQ ID NO: 331) having a hairpin-like structure is known as a precursor of “hsa-miR-6891-5p”.

The term “hsa-miR-6826-5p gene” or “hsa-miR-6826-5p” used herein includes the hsa-miR-6826-5p gene (miRBase Accession No. MIMAT0027552) described in SEQ ID NO: 109, a homolog or an ortholog of a different organism species, and the like. The hsa-miR-6826-5p gene can be obtained by a method described in Ladewig E et al., 2012, Genome Res, Vol. 22, p. 1634-1645. Also, “hsa-mir-6826” (miRBase Accession No. M10022671, SEQ ID NO: 332) having a hairpin-like structure is known as a precursor of “hsa-miR-6826-5p”.

The term “hsa-miR-6807-5p gene” or “hsa-miR-6807-5p” used herein includes the hsa-miR-6807-5p gene (miRBase Accession No. MIMAT0027514) described in SEQ ID NO: 110, a homolog or an ortholog of a different organism species, and the like. The hsa-miR-6807-5p gene can be obtained by a method described in Ladewig E et al., 2012, Genome Res, Vol. 22, p. 1634-1645. Also, “hsa-mir-6807” (miRBase Accession No. M10022652, SEQ ID NO: 333) having a hairpin-like structure is known as a precursor of “hsa-miR-6807-5p”.

The term “hsa-miR-7150 gene” or “hsa-miR-7150” used herein includes the hsa-miR-7150 gene (miRBase Accession No. MIMAT0028211) described in SEQ ID NO: 111, a homolog or an ortholog of a different organism species, and the like. The hsa-miR-7150 gene can be obtained by a method described in Oulas A et al., 2009, Nucleic Acids Res, Vol. 37, p. 3276-3287. Also, “hsa-mir-7150” (miRBase Accession No. M10023610, SEQ ID NO: 334) having a hairpin-like structure is known as a precursor of “hsa-miR-7150”.

The term “hsa-miR-4534 gene” or “hsa-miR-4534” used herein includes the hsa-miR-4534 gene (miRBase Accession No. MIMAT0019073) described in SEQ ID NO: 112, a homolog or an ortholog of a different organism species, and the like. The hsa-miR-4534 gene can be obtained by a method described in Jima D D et al., 2010, Blood, Vol. 116, e118-e127. Also, “hsa-mir-4534” (miRBase Accession No. M10016901, SEQ ID NO: 335) having a hairpin-like structure is known as a precursor of “hsa-miR-4534”.

The term “hsa-miR-4476 gene” or “hsa-miR-4476” used herein includes the hsa-miR-4476 gene (miRBase Accession No. MIMAT0019003) described in SEQ ID NO: 113, a homolog or an ortholog of a different organism species, and the like. The hsa-miR-4476 gene can be obtained by a method described in Jima D D et al., 2010, Blood, Vol. 116, e118-e127. Also, “hsa-mir-4476” (miRBase Accession No. M10016828, SEQ ID NO: 336) having a hairpin-like structure is known as a precursor of “hsa-miR-4476”.

The term “hsa-miR-4649-5p gene” or “hsa-miR-4649-5p” used herein includes the hsa-miR-4649-5p gene (miRBase Accession No. MIMAT0019711) described in SEQ ID NO: 114, a homolog or an ortholog of a different organism species, and the like. The hsa-miR-4649-5p gene can be obtained by a method described in Persson H et al., 2011, Cancer Res, Vol. 71, p. 78-86. Also, “hsa-mir-4649” (miRBase Accession No. M10017276, SEQ ID NO: 337) having a hairpin-like structure is known as a precursor of “hsa-miR-4649-5p”.

The term “hsa-miR-4525 gene” or “hsa-miR-4525” used herein includes the hsa-miR-4525 gene (miRBase Accession No. MIMAT0019064) described in SEQ ID NO: 115, a homolog or an ortholog of a different organism species, and the like. The hsa-miR-4525 gene Also, “hsa-mir-4525” (miRBase Accession No. M10016892, SEQ ID NO: 338) having a hairpin-like structure is known as a precursor of “hsa-miR-4525”.

The term “hsa-miR-1915-3p gene” or “hsa-miR-1915-3p” used herein includes the hsa-miR-1915-3p gene (miRBase Accession No. MIMAT0007892) described in SEQ ID NO: 116, a homolog or an ortholog of a different organism species, and the like. The hsa-miR-1915-3p gene can be obtained by a method described in Bar M et al., 2008, Stem Cells, Vol. 26, p. 2496-2505. Also, “hsa-mir-1915” (miRBase Accession No. M10008336, SEQ ID NO: 311) having a hairpin-like structure is known as a precursor of “hsa-miR-1915-3p”.

The term “hsa-miR-4516 gene” or “hsa-miR-4516” used herein includes the hsa-miR-4516 gene (miRBase Accession No. MIMAT0019053) described in SEQ ID NO: 117, a homolog or an ortholog of a different organism species, and the like. The hsa-miR-4516 gene can be obtained by a method described in Jima D D et al., 2010, Blood, Vol. 116, e118-e127. Also, “hsa-mir-4516” (miRBase Accession No. M10016882, SEQ ID NO: 339) having a hairpin-like structure is known as a precursor of “hsa-miR-4516”.

The term “hsa-miR-4417 gene” or “hsa-miR-4417” used herein includes the hsa-miR-4417 gene (miRBase Accession No. MIMAT0018929) described in SEQ ID NO: 118, a homolog or an ortholog of a different organism species, and the like. The hsa-miR-4417 gene can be obtained by a method described in Jima D D et al., 2010, Blood, Vol. 116, e118-e127. Also, “hsa-mir-4417” (miRBase Accession No. M10016753, SEQ ID NO: 340) having a hairpin-like structure is known as a precursor of “hsa-miR-4417”.

The term “hsa-miR-642b-3p gene” or “hsa-miR-642b-3p” used herein includes the hsa-miR-642b-3p gene (miRBase Accession No. MIMAT0018444) described in SEQ ID NO: 119, a homolog or an ortholog of a different organism species, and the like. The hsa-miR-642b-3p gene can be obtained by a method described in Witten D et al., 2010, BMC Biol, Vol. 8, p. 58. Also, “hsa-mir-642b” (miRBase Accession No. M10016685, SEQ ID NO: 341) having a hairpin-like structure is known as a precursor of “hsa-miR-642b-3p”.

The term “hsa-miR-3141 gene” or “hsa-miR-3141” used herein includes the hsa-miR-3141 gene (miRBase Accession No. MIMAT0015010) described in SEQ ID NO: 120, a homolog or an ortholog of a different organism species, and the like. The hsa-miR-3141 gene can be obtained by a method described in Stark M S et al., 2010, PLoS One, Vol. 5, e9685. Also, “hsa-mir-3141” (miRBase Accession No. M10014165, SEQ ID NO: 342) having a hairpin-like structure is known as a precursor of “hsa-miR-3141”.

The term “hsa-miR-5100 gene” or “hsa-miR-5100” used herein includes the hsa-miR-5100 gene (miRBase Accession No. MIMAT0022259) described in SEQ ID NO: 121, a homolog or an ortholog of a different organism species, and the like. The hsa-miR-5100 gene can be obtained by a method described in Tandon Metal., 2012, Oral Dis, Vol. 18, p. 127-131. Also, “hsa-mir-5100” (miRBase Accession No. M10019116, SEQ ID NO: 343) having a hairpin-like structure is known as a precursor of “hsa-miR-5100”.

The term “hsa-miR-6848-5p gene” or “hsa-miR-6848-5p” used herein includes the hsa-miR-6848-5p gene (miRBase Accession No. MIMAT0027596) described in SEQ ID NO: 122, a homolog or an ortholog of a different organism species, and the like. The hsa-miR-6848-5p gene can be obtained by a method described in Ladewig E et al., 2012, Genome Res, Vol. 22, p. 1634-1645. Also, “hsa-mir-6848” (miRBase Accession No. M10022694, SEQ ID NO: 344) having a hairpin-like structure is known as a precursor of “hsa-miR-6848-5p”.

The term “hsa-miR-4739 gene” or “hsa-miR-4739” used herein includes the hsa-miR-4739 gene (miRBase Accession No. MIMAT0019868) described in SEQ ID NO: 123, a homolog or an ortholog of a different organism species, and the like. The hsa-miR-4739 gene Also, “hsa-mir-4739” (miRBase Accession No. M10017377, SEQ ID NO: 345) having a hairpin-like structure is known as a precursor of “hsa-miR-4739”.

The term “hsa-miR-4459 gene” or “hsa-miR-4459” used herein includes the hsa-miR-4459 gene (miRBase Accession No. MIMAT0018981) described in SEQ ID NO: 124, a homolog or an ortholog of a different organism species, and the like. The hsa-miR-4459 gene can be obtained by a method described in Jima D D et al., 2010, Blood, Vol. 116, e118-e127. Also, “hsa-mir-4459” (miRBase Accession No. M10016805, SEQ ID NO: 346) having a hairpin-like structure is known as a precursor of “hsa-miR-4459”.

The term “hsa-miR-1237-5p gene” or “hsa-miR-1237-5p” used herein includes the hsa-miR-1237-5p gene (miRBase Accession No. MIMAT0022946) described in SEQ ID NO: 125, a homolog or an ortholog of a different organism species, and the like. The hsa-miR-1237-5p gene can be obtained by a method described in Berezikov E et al., 2007, Mol Cell, Vol. 28, p. 328-336. Also, “hsa-mir-1237” (miRBase Accession No. M10006327, SEQ ID NO: 347) having a hairpin-like structure is known as a precursor of “hsa-miR-1237-5p”.

The term “hsa-miR-296-3p gene” or “hsa-miR-296-3p” used herein includes the hsa-miR-296-3p gene (miRBase Accession No. MIMAT0004679) described in SEQ ID NO: 126, a homolog or an ortholog of a different organism species, and the like. The hsa-miR-296-3p gene can be obtained by a method described in Houbaviy H B et al., 2003, Dev Cell, Vol. 5, p. 351-358. Also, “hsa-mir-296” (miRBase Accession No. M10000747, SEQ ID NO: 273) having a hairpin-like structure is known as a precursor of “hsa-miR-296-3p”.

The term “hsa-miR-4665-3p gene” or “hsa-miR-4665-3p” used herein includes the hsa-miR-4665-3p gene (miRBase Accession No. MIMAT0019740) described in SEQ ID NO: 127, a homolog or an ortholog of a different organism species, and the like. The hsa-miR-4665-3p 78-86. Also, “hsa-mir-4665” (miRBase Accession No. MI0017295, SEQ ID NO: 269) having a hairpin-like structure is known as a precursor of “hsa-miR-4665-3p”.

The term “hsa-miR-6786-5p gene” or “hsa-miR-6786-5p” used herein includes the hsa-miR-6786-5p gene (miRBase Accession No. MIMAT0027472) described in SEQ ID NO: 128, a homolog or an ortholog of a different organism species, and the like. The hsa-miR-6786-5p gene can be obtained by a method described in Ladewig E et al., 2012, Genome Res, Vol. 22, p. 1634-1645. Also, “hsa-mir-6786” (miRBase Accession No. MI0022631, SEQ ID NO: 348) having a hairpin-like structure is known as a precursor of “hsa-miR-6786-5p”.

The term “hsa-miR-4258 gene” or “hsa-miR-4258” used herein includes the hsa-miR-4258 gene (miRBase Accession No. MIMAT0016879) described in SEQ ID NO: 129, a homolog or an ortholog of a different organism species, and the like. The hsa-miR-4258 gene can be obtained by a method described in Goff L A et al., 2009, PLoS One, Vol. 4, e7192. Also, “hsa-mir-4258” (miRBase Accession No. MI0015857, SEQ ID NO: 349) having a hairpin-like structure is known as a precursor of “hsa-miR-4258”.

The term “hsa-miR-6510-5p gene” or “hsa-miR-6510-5p” used herein includes the hsa-miR-6510-5p gene (miRBase Accession No. MIMAT0025476) described in SEQ ID NO: 130, a homolog or an ortholog of a different organism species, and the like. The hsa-miR-6510-5p gene can be obtained by a method described in Joyce C E et al., 2011, Hum Mol Genet, Vol. 20, p. 4025-4040. Also, “hsa-mir-6510” (miRBase Accession No. MI0022222, SEQ ID NO: 350) having a hairpin-like structure is known as a precursor of “hsa-miR-6510-5p”.

The term “hsa-miR-1343-5p gene” or “hsa-miR-1343-5p” used herein includes the hsa-miR-1343-5p gene (miRBase Accession No. MIMAT0027038) described in SEQ ID NO: 131, a homolog or an ortholog of a different organism species, and the like. The hsa-miR-1343-5p 78-86. Also, “hsa-mir-1343” (miRBase Accession No. M10017320, SEQ ID NO: 225) having a hairpin-like structure is known as a precursor of “hsa-miR-1343-5p”.

The term “hsa-miR-1247-3p gene” or “hsa-miR-1247-3p” used herein includes the hsa-miR-1247-3p gene (miRBase Accession No. MIMAT0022721) described in SEQ ID NO: 132, a homolog or an ortholog of a different organism species, and the like. The hsa-miR-1247-3p gene can be obtained by a method described in Morin R D et al., 2008, Genome Res, Vol. 18, p. 610-621. Also, “hsa-mir-1247” (miRBase Accession No. M10006382, SEQ ID NO: 351) having a hairpin-like structure is known as a precursor of “hsa-miR-1247-3p”.

The term “hsa-miR-6805-5p gene” or “hsa-miR-6805-5p” used herein includes the hsa-miR-6805-5p gene (miRBase Accession No. MIMAT0027510) described in SEQ ID NO: 133, a homolog or an ortholog of a different organism species, and the like. The hsa-miR-6805-5p gene can be obtained by a method described in Ladewig E et al., 2012, Genome Res, Vol. 22, p. 1634-1645. Also, “hsa-mir-6805” (miRBase Accession No. M10022650, SEQ ID NO: 324) having a hairpin-like structure is known as a precursor of “hsa-miR-6805-5p”.

The term “hsa-miR-4492 gene” or “hsa-miR-4492” used herein includes the hsa-miR-4492 gene (miRBase Accession No. MIMAT0019027) described in SEQ ID NO: 134, a homolog or an ortholog of a different organism species, and the like. The hsa-miR-4492 gene can be obtained by a method described in Jima D D et al., 2010, Blood, Vol. 116, e118-e127. Also, “hsa-mir-4492” (miRBase Accession No. M10016854, SEQ ID NO: 352) having a hairpin-like structure is known as a precursor of “hsa-miR-4492”.

The term “hsa-miR-1469 gene” or “hsa-miR-1469” used herein includes the hsa-miR-1469 gene (miRBase Accession No. MIMAT0007347) described in SEQ ID NO: 135, a homolog or an ortholog of a different organism species, and the like. The hsa-miR-1469 gene can be obtained by a method described in Kawaji H et al., 2008, BMC Genomics, Vol. 9, p. 157. Also, “hsa-mir-1469” (miRBase Accession No. M10007074, SEQ ID NO: 353) having a hairpin-like structure is known as a precursor of “hsa-miR-1469”.

The term “hsa-miR-1268b gene” or “hsa-miR-1268b” used herein includes the hsa-miR-1268b gene (miRBase Accession No. MIMAT0018925) described in SEQ ID NO: 136, a homolog or an ortholog of a different organism species, and the like. The hsa-miR-1268b gene can be obtained by a method described in Jima D D et al., 2010, Blood, Vol. 116, e118-e127. Also, “hsa-mir-1268b” (miRBase Accession No. M10016748, SEQ ID NO: 354) having a hairpin-like structure is known as a precursor of “hsa-miR-1268b”.

The term “hsa-miR-6858-5p gene” or “hsa-miR-6858-5p” used herein includes the hsa-miR-6858-5p gene (miRBase Accession No. MIMAT0027616) described in SEQ ID NO: 137, a homolog or an ortholog of a different organism species, and the like. The hsa-miR-6858-5p gene can be obtained by a method described in Ladewig E et al., 2012, Genome Res, Vol. 22, p. 1634-1645. Also, “hsa-mir-6858” (miRBase Accession No. M10022704, SEQ ID NO: 355) having a hairpin-like structure is known as a precursor of “hsa-miR-6858-5p”.

The term “hsa-miR-3937 gene” or “hsa-miR-3937” used herein includes the hsa-miR-3937 gene (miRBase Accession No. MIMAT0018352) described in SEQ ID NO: 138, a homolog or an ortholog of a different organism species, and the like. The hsa-miR-3937 gene can be obtained by a method described in Liao J Y et al., 2010, PLoS One, Vol. 5, e10563. Also, “hsa-mir-3937” (miRBase Accession No. M10016593, SEQ ID NO: 356) having a hairpin-like structure is known as a precursor of “hsa-miR-3937”.

The term “hsa-miR-939-5p gene” or “hsa-miR-939-5p” used herein includes the hsa-miR-939-5p gene (miRBase Accession No. MIMAT0004982) described in SEQ ID NO: 139, a homolog or an ortholog of a different organism species, and the like. The hsa-miR-939-5p gene can be obtained by a method described in Lui W O et al., 2007, Cancer Res, Vol. 67, p. 6031-6043. Also, “hsa-mir-939” (miRBase Accession No. M10005761, SEQ ID NO: 357) having a hairpin-like structure is known as a precursor of “hsa-miR-939-5p”.

The term “hsa-miR-3656 gene” or “hsa-miR-3656” used herein includes the hsa-miR-3656 gene (miRBase Accession No. MIMAT0018076) described in SEQ ID NO: 140, a homolog or an ortholog of a different organism species, and the like. The hsa-miR-3656 gene can be obtained by a method described in Meiri E et al., 2010, Nucleic Acids Res, Vol. 38, p. 6234-6246. Also, “hsa-mir-3656” (miRBase Accession No. M10016056, SEQ ID NO: 358) having a hairpin-like structure is known as a precursor of “hsa-miR-3656”.

The term “hsa-miR-744-5p gene” or “hsa-miR-744-5p” used herein includes the hsa-miR-744-5p gene (miRBase Accession No. MIMAT0004945) described in SEQ ID NO: 141, a homolog or an ortholog of a different organism species, and the like. The hsa-miR-744-5p gene can be obtained by a method described in Berezikov E et al., 2006, Genome Res, Vol. 16, p. 1289-1298. Also, “hsa-mir-744” (miRBase Accession No. M10005559, SEQ ID NO: 359) having a hairpin-like structure is known as a precursor of “hsa-miR-744-5p”.

The term “hsa-miR-4687-3p gene” or “hsa-miR-4687-3p” used herein includes the hsa-miR-4687-3p gene (miRBase Accession No. MIMAT0019775) described in SEQ ID NO: 142, a homolog or an ortholog of a different organism species, and the like. The hsa-miR-4687-3p gene can be obtained by a method described in Persson H et al., 2011, Cancer Res, Vol. 71, p. 78-86. Also, “hsa-mir-4687” (miRBase Accession No. M10017319, SEQ ID NO: 360) having a hairpin-like structure is known as a precursor of “hsa-miR-4687-3p”.

The term “hsa-miR-4763-3p gene” or “hsa-miR-4763-3p” used herein includes the hsa-miR-4763-3p gene (miRBase Accession No. MIMAT0019913) described in SEQ ID NO: 143, a homolog or an ortholog of a different organism species, and the like. The hsa-miR-4763-3p 78-86. Also, “hsa-mir-4763” (miRBase Accession No. M10017404, SEQ ID NO: 361) having a hairpin-like structure is known as a precursor of “hsa-miR-4763-3p”.

The term “hsa-miR-3620-5p gene” or “hsa-miR-3620-5p” used herein includes the hsa-miR-3620-5p gene (miRBase Accession No. MIMAT0022967) described in SEQ ID NO: 144, a homolog or an ortholog of a different organism species, and the like. The hsa-miR-3620-5p gene can be obtained by a method described in Witten D et al., 2010, BMC Biol, Vol. 8, p. 58. Also, “hsa-mir-3620” (miRBase Accession No. M10016011, SEQ ID NO: 362) having a hairpin-like structure is known as a precursor of “hsa-miR-3620-5p”.

The term “hsa-miR-3195 gene” or “hsa-miR-3195” used herein includes the hsa-miR-3195 gene (miRBase Accession No. MIMAT0015079) described in SEQ ID NO: 145, a homolog or an ortholog of a different organism species, and the like. The hsa-miR-3195 gene can be obtained by a method described in Stark M S et al., 2010, PLoS One, Vol. 5, e9685. Also, “hsa-mir-3195” (miRBase Accession No. M10014240, SEQ ID NO: 363) having a hairpin-like structure is known as a precursor of “hsa-miR-3195”.

The term “hsa-miR-6842-5p gene” or “hsa-miR-6842-5p” used herein includes the hsa-miR-6842-5p gene (miRBase Accession No. MIMAT0027586) described in SEQ ID NO: 146, a homolog or an ortholog of a different organism species, and the like. The hsa-miR-6842-5p gene can be obtained by a method described in Ladewig E et al., 2012, Genome Res, Vol. 22, p. 1634-1645. Also, “hsa-mir-6842” (miRBase Accession No. M10022688, SEQ ID NO: 364) having a hairpin-like structure is known as a precursor of “hsa-miR-6842-5p”.

The term “hsa-miR-4707-5p gene” or “hsa-miR-4707-5p” used herein includes the hsa-miR-4707-5p gene (miRBase Accession No. MIMAT0019807) described in SEQ ID NO: 147, a homolog or an ortholog of a different organism species, and the like. The hsa-miR-4707-5p 78-86. Also, “hsa-mir-4707” (miRBase Accession No. MI0017340, SEQ ID NO: 365) having a hairpin-like structure is known as a precursor of “hsa-miR-4707-5p”.

The term “hsa-miR-642a-3p gene” or “hsa-miR-642a-3p” used herein includes the hsa-miR-642a-3p gene (miRBase Accession No. MIMAT0020924) described in SEQ ID NO: 148, a homolog or an ortholog of a different organism species, and the like. The hsa-miR-642a-3p gene can be obtained by a method described in Cummins J M et al., 2006, Proc Natl Acad Sci USA, Vol. 103, p. 3687-3692. Also, “hsa-mir-642a” (miRBase Accession No. MI0003657, SEQ ID NO: 366) having a hairpin-like structure is known as a precursor of “hsa-miR-642a-3p”.

The term “hsa-miR-7113-3p gene” or “hsa-miR-7113-3p” used herein includes the hsa-miR-7113-3p gene (miRBase Accession No. MIMAT0028124) described in SEQ ID NO: 149, a homolog or an ortholog of a different organism species, and the like. The hsa-miR-7113-3p gene can be obtained by a method described in Ladewig E et al., 2012, Genome Res, Vol. 22, p. 1634-1645. Also, “hsa-mir-7113” (miRBase Accession No. MI0022964, SEQ ID NO: 367) having a hairpin-like structure is known as a precursor of “hsa-miR-7113-3p”.

The term “hsa-miR-4728-5p gene” or “hsa-miR-4728-5p” used herein includes the hsa-miR-4728-5p gene (miRBase Accession No. MIMAT0019849) described in SEQ ID NO: 150, a homolog or an ortholog of a different organism species, and the like. The hsa-miR-4728-5p gene can be obtained by a method described in Persson H et al., 2011, Cancer Res, Vol. 71, p. 78-86. Also, “hsa-mir-4728” (miRBase Accession No. MI0017365, SEQ ID NO: 368) having a hairpin-like structure is known as a precursor of “hsa-miR-4728-5p”.

The term “hsa-miR-5195-3p gene” or “hsa-miR-5195-3p” used herein includes the hsa-miR-5195-3p gene (miRBase Accession No. MIMAT0021127) described in SEQ ID NO: 151, a homolog or an ortholog of a different organism species, and the like. The hsa-miR-5195-3p gene can be obtained by a method described in Schotte D et al., 2011, Leukemia, Vol. 25, p. 1389-1399. Also, “hsa-mir-5195” (miRBase Accession No. M10018174, SEQ ID NO: 369) having a hairpin-like structure is known as a precursor of “hsa-miR-5195-3p”.

The term “hsa-miR-1185-1-3p gene” or “hsa-miR-1185-1-3p” used herein includes the hsa-miR-1185-1-3p gene (miRBase Accession No. MIMAT0022838) described in SEQ ID NO: 152, a homolog or an ortholog of a different organism species, and the like. The hsa-miR-1185-1-3p gene can be obtained by a method described in Berezikov E et al., 2006, Genome Res, Vol. 16, p. 1289-1298. Also, “hsa-mir-1185-1” (miRBase Accession No. M10003844, SEQ ID NO: 370) having a hairpin-like structure is known as a precursor of “hsa-miR-1185-1-3p”.

The term “hsa-miR-6774-5p gene” or “hsa-miR-6774-5p” used herein includes the hsa-miR-6774-5p gene (miRBase Accession No. MIMAT0027448) described in SEQ ID NO: 153, a homolog or an ortholog of a different organism species, and the like. The hsa-miR-6774-5p gene can be obtained by a method described in Ladewig E et al., 2012, Genome Res, Vol. 22, p. 1634-1645. Also, “hsa-mir-6774” (miRBase Accession No. M10022619, SEQ ID NO: 371) having a hairpin-like structure is known as a precursor of “hsa-miR-6774-5p”.

The term “hsa-miR-8059 gene” or “hsa-miR-8059” used herein includes the hsa-miR-8059 gene (miRBase Accession No. MIMAT0030986) described in SEQ ID NO: 154, a homolog or an ortholog of a different organism species, and the like. The hsa-miR-8059 gene can be obtained by a method described in Wang H J et al., 2013, Shock, Vol. 39, p. 480-487. Also, “hsa-mir-8059” (miRBase Accession No. M10025895, SEQ ID NO: 372) having a hairpin-like structure is known as a precursor of “hsa-miR-8059”.

The term “hsa-miR-3131 gene” or “hsa-miR-3131” used herein includes the hsa-miR-3131 gene (miRBase Accession No. MIMAT0014996) described in SEQ ID NO: 155, a homolog or an ortholog of a different organism species, and the like. The hsa-miR-3131 gene can be obtained by a method described in Stark M S et al., 2010, PLoS One, Vol. 5, e9685. Also, “hsa-mir-3131” (miRBase Accession No. M10014151, SEQ ID NO: 373) having a hairpin-like structure is known as a precursor of “hsa-miR-3131”.

The term “hsa-miR-7847-3p gene” or “hsa-miR-7847-3p” used herein includes the hsa-miR-7847-3p gene (miRBase Accession No. MIMAT0030422) described in SEQ ID NO: 156, a homolog or an ortholog of a different organism species, and the like. The hsa-miR-7847-3p gene can be obtained by a method described in Ple H et al., 2012, PLoS One, Vol. 7, e50746. Also, “hsa-mir-7847” (miRBase Accession No. M10025517, SEQ ID NO: 374) having a hairpin-like structure is known as a precursor of “hsa-miR-7847-3p”.

The term “hsa-miR-4463 gene” or “hsa-miR-4463” used herein includes the hsa-miR-4463 gene (miRBase Accession No. MIMAT0018987) described in SEQ ID NO: 157, a homolog or an ortholog of a different organism species, and the like. The hsa-miR-4463 gene can be obtained by a method described in Jima D D et al., 2010, Blood, Vol. 116, e118-e127. Also, “hsa-mir-4463” (miRBase Accession No. M10016811, SEQ ID NO: 375) having a hairpin-like structure is known as a precursor of “hsa-miR-4463”.

The term “hsa-miR-128-2-5p gene” or “hsa-miR-128-2-5p” used herein includes the hsa-miR-128-2-5p gene (miRBase Accession No. MIMAT0031095) described in SEQ ID NO: 158, a homolog or an ortholog of a different organism species, and the like. The hsa-miR-128-2-5p gene can be obtained by a method described in Lagos-Quintana M et al., 2002, Curr Biol, Vol. 12, p. 735-739. Also, “hsa-mir-128-2” (miRBase Accession No. M10000727, SEQ ID NO: 376) having a hairpin-like structure is known as a precursor of “hsa-miR-128-2-5p”.

The term “hsa-miR-4508 gene” or “hsa-miR-4508” used herein includes the hsa-miR-4508 gene (miRBase Accession No. MIMAT0019045) described in SEQ ID NO: 159, a homolog or an ortholog of a different organism species, and the like. The hsa-miR-4508 gene Also, “hsa-mir-4508” (miRBase Accession No. M10016872, SEQ ID NO: 377) having a hairpin-like structure is known as a precursor of “hsa-miR-4508”.

The term “hsa-miR-6806-5p gene” or “hsa-miR-6806-5p” used herein includes the hsa-miR-6806-5p gene (miRBase Accession No. MIMAT0027512) described in SEQ ID NO: 160, a homolog or an ortholog of a different organism species, and the like. The hsa-miR-6806-5p gene can be obtained by a method described in Ladewig E et al., 2012, Genome Res, Vol. 22, p. 1634-1645. Also, “hsa-mir-6806” (miRBase Accession No. M10022651, SEQ ID NO: 378) having a hairpin-like structure is known as a precursor of “hsa-miR-6806-5p”.

The term “hsa-miR-7111-5p gene” or “hsa-miR-7111-5p” used herein includes the hsa-miR-7111-5p gene (miRBase Accession No. MIMAT0028119) described in SEQ ID NO: 161, a homolog or an ortholog of a different organism species, and the like. The hsa-miR-7111-5p gene can be obtained by a method described in Ladewig E et al., 2012, Genome Res, Vol. 22, p. 1634-1645. Also, “hsa-mir-7111” (miRBase Accession No. M10022962, SEQ ID NO: 379) having a hairpin-like structure is known as a precursor of “hsa-miR-7111-5p”.

The term “hsa-miR-6782-5p gene” or “hsa-miR-6782-5p” used herein includes the hsa-miR-6782-5p gene (miRBase Accession No. MIMAT0027464) described in SEQ ID NO: 162, a homolog or an ortholog of a different organism species, and the like. The hsa-miR-6782-5p gene can be obtained by a method described in Ladewig E et al., 2012, Genome Res, Vol. 22, p. 1634-1645. Also, “hsa-mir-6782” (miRBase Accession No. M10022627, SEQ ID NO: 380) having a hairpin-like structure is known as a precursor of “hsa-miR-6782-5p”.

The term “hsa-miR-4734 gene” or “hsa-miR-4734” used herein includes the hsa-miR-4734 gene (miRBase Accession No. MIMAT0019859) described in SEQ ID NO: 163, a homolog or an ortholog of a different organism species, and the like. The hsa-miR-4734 gene Also, “hsa-mir-4734” (miRBase Accession No. M10017371, SEQ ID NO: 381) having a hairpin-like structure is known as a precursor of “hsa-miR-4734”.

The term “hsa-miR-3162-5p gene” or “hsa-miR-3162-5p” used herein includes the hsa-miR-3162-5p gene (miRBase Accession No. MIMAT0015036) described in SEQ ID NO: 164, a homolog or an ortholog of a different organism species, and the like. The hsa-miR-3162-5p gene can be obtained by a method described in Stark M S et al., 2010, PLoS One, Vol. 5, e9685. Also, “hsa-mir-3162” (miRBase Accession No. M10014192, SEQ ID NO: 382) having a hairpin-like structure is known as a precursor of “hsa-miR-3162-5p”.

The term “hsa-miR-887-3p gene” or “hsa-miR-887-3p” used herein includes the hsa-miR-887-3p gene (miRBase Accession No. MIMAT0004951) described in SEQ ID NO: 165, a homolog or an ortholog of a different organism species, and the like. The hsa-miR-887-3p gene can be obtained by a method described in Berezikov E et al., 2006, Genome Res, Vol. 16, p. 1289-1298. Also, “hsa-mir-887” (miRBase Accession No. M10005562, SEQ ID NO: 383) having a hairpin-like structure is known as a precursor of “hsa-miR-887-3p”.

The term “hsa-miR-6752-5p gene” or “hsa-miR-6752-5p” used herein includes the hsa-miR-6752-5p gene (miRBase Accession No. MIMAT0027404) described in SEQ ID NO: 166, a homolog or an ortholog of a different organism species, and the like. The hsa-miR-6752-5p gene can be obtained by a method described in Ladewig E et al., 2012, Genome Res, Vol. 22, p. 1634-1645. Also, “hsa-mir-6752” (miRBase Accession No. M10022597, SEQ ID NO: 384) having a hairpin-like structure is known as a precursor of “hsa-miR-6752-5p”.

The term “hsa-miR-6724-5p gene” or “hsa-miR-6724-5p” used herein includes the hsa-miR-6724-5p gene (miRBase Accession No. MIMAT0025856) described in SEQ ID NO: 167, a homolog or an ortholog of a different organism species, and the like. The hsa-miR-6724-5p gene can be obtained by a method described in Li Y et al., 2012, Gene, Vol. 497, p. 330-335. Also, “hsa-mir-6724” (miRBase Accession No. M10022559, SEQ ID NO: 385) having a hairpin-like structure is known as a precursor of “hsa-miR-6724-5p”.

The term “hsa-miR-23b-3p gene” or “hsa-miR-23b-3p” used herein includes the hsa-miR-23b-3p gene (miRBase Accession No. MIMAT0000418) described in SEQ ID NO: 168, a homolog or an ortholog of a different organism species, and the like. The hsa-miR-23b-3p gene can be obtained by a method described in Lagos-Quintana M et al., 2002, Curr Biol, Vol. 12, p. 735-739. Also, “hsa-mir-23b” (miRBase Accession No. M10000439, SEQ ID NO: 386) having a hairpin-like structure is known as a precursor of “hsa-miR-23b-3p”.

The term “hsa-miR-23a-3p gene” or “hsa-miR-23a-3p” used herein includes the hsa-miR-23a-3p gene (miRBase Accession No. MIMAT0000078) described in SEQ ID NO: 169, a homolog or an ortholog of a different organism species, and the like. The hsa-miR-23a-3p gene can be obtained by a method described in Lagos-Quintana M et al., 2001, Science, Vol. 294, p. 853-858. Also, “hsa-mir-23a” (miRBase Accession No. M10000079, SEQ ID NO: 387) having a hairpin-like structure is known as a precursor of “hsa-miR-23a-3p”.

The term “hsa-miR-625-3p gene” or “hsa-miR-625-3p” used herein includes the hsa-miR-625-3p gene (miRBase Accession No. MIMAT0004808) described in SEQ ID NO: 170, a homolog or an ortholog of a different organism species, and the like. The hsa-miR-625-3p gene can be obtained by a method described in Cummins J M et al., 2006, Proc Natl Acad Sci U.S.A., Vol. 103, p. 3687-3692. Also, “hsa-mir-625” (miRBase Accession No. M10003639, SEQ ID NO: 388) having a hairpin-like structure is known as a precursor of “hsa-miR-625-3p”.

The term “hsa-miR-1228-3p gene” or “hsa-miR-1228-3p” used herein includes the hsa-miR-1228-3p gene (miRBase Accession No. MIMAT0005583) described in SEQ ID NO: 171, a homolog or an ortholog of a different organism species, and the like. The hsa-miR-1228-3p gene can be obtained by a method described in Berezikov E et al., 2007, Mol Cell, Vol. 28, p. 328-336. Also, “hsa-mir-1228” (miRBase Accession No. M10006318, SEQ ID NO: 316) having a hairpin-like structure is known as a precursor of “hsa-miR-1228-3p”.

The term “hsa-miR-614 gene” or “hsa-miR-614” used herein includes the hsa-miR-614 gene (miRBase Accession No. MIMAT0003282) described in SEQ ID NO: 172, a homolog or an ortholog of a different organism species, and the like. The hsa-miR-614 gene can be obtained by a method described in Cummins J M et al., 2006, Proc Natl Acad Sci USA, Vol. 103, p. 3687-3692. Also, “hsa-mir-614” (miRBase Accession No. M10003627, SEQ ID NO: 389) having a hairpin-like structure is known as a precursor of “hsa-miR-614”.

The term “hsa-miR-1913 gene” or “hsa-miR-1913” used herein includes the hsa-miR-1913 gene (miRBase Accession No. MIMAT0007888) described in SEQ ID NO: 173, a homolog or an ortholog of a different organism species, and the like. The hsa-miR-1913 gene can be obtained by a method described in Bar M et al., 2008, Stem Cells, Vol. 26, p. 2496-2505. Also, “hsa-mir-1913” (miRBase Accession No. M10008334, SEQ ID NO: 390) having a hairpin-like structure is known as a precursor of “hsa-miR-1913”.

The term “hsa-miR-92a-2-5p gene” or “hsa-miR-92a-2-5p” used herein includes the hsa-miR-92a-2-5p gene (miRBase Accession No. MIMAT0004508) described in SEQ ID NO: 174, a homolog or an ortholog of a different organism species, and the like. The hsa-miR-92a-2-5p gene can be obtained by a method described in Mourelatos Z et al., 2002, Genes Dev, Vol. 16, p. 720-728. Also, “hsa-mir-92a-2” (miRBase Accession No. M10000094, SEQ ID NO: 391) having a hairpin-like structure is known as a precursor of “hsa-miR-92a-2-5p”.

The term “hsa-miR-187-5p gene” or “hsa-miR-187-5p” used herein includes the hsa-miR-187-5p gene (miRBase Accession No. MIMAT0004561) described in SEQ ID NO: 175, a homolog or an ortholog of a different organism species, and the like. The hsa-miR-187-5p gene can be obtained by a method described in Lim L P et al., 2003, Science, Vol. 299, p. 1540. Also, “hsa-mir-187” (miRBase Accession No. MI0000274, SEQ ID NO: 392) having a hairpin-like structure is known as a precursor of “hsa-miR-187-5p”.

The term “hsa-miR-16-5p gene” or “hsa-miR-16-5p” used herein includes the hsa-miR-16-5p gene (miRBase Accession No. MIMAT0000069) described in SEQ ID NO: 176, a homolog or an ortholog of a different organism species, and the like. The hsa-miR-16-5p gene can be obtained by a method described in Lagos-Quintana M et al., 2001, Science, Vol. 294, p. 853-858. Also, “hsa-mir-16-1” and “hsa-mir-16-2” (miRBase Accession Nos. MI0000070 and MI0000115, SEQ ID NOs: 393 and 394) having a hairpin-like structure are known as precursors of “hsa-miR-16-5p”.

The term “hsa-miR-92b-3p gene” or “hsa-miR-92b-3p” used herein includes the hsa-miR-92b-3p gene (miRBase Accession No. MIMAT0003218) described in SEQ ID NO: 177, a homolog or an ortholog of a different organism species, and the like. The hsa-miR-92b-3p gene can be obtained by a method described in Cummins J M et al., 2006, Proc Natl Acad Sci USA, Vol. 103, p. 3687-3692. Also, “hsa-mir-92b” (miRBase Accession No. MI0003560, SEQ ID NO: 395) having a hairpin-like structure is known as a precursor of “hsa-miR-92b-3p”.

The term “hsa-miR-150-3p gene” or “hsa-miR-150-3p” used herein includes the hsa-miR-150-3p gene (miRBase Accession No. MIMAT0004610) described in SEQ ID NO: 178, a homolog or an ortholog of a different organism species, and the like. The hsa-miR-150-3p gene can be obtained by a method described in Lagos-Quintana M et al., 2002, Curr Biol, Vol. 12, p. 735-739. Also, “hsa-mir-150” (miRBase Accession No. MI0000479, SEQ ID NO: 396) having a hairpin-like structure is known as a precursor of “hsa-miR-150-3p”.

The term “hsa-miR-564 gene” or “hsa-miR-564” used herein includes the hsa-miR-564 gene (miRBase Accession No. MIMAT0003228) described in SEQ ID NO: 179, a homolog or an ortholog of a different organism species, and the like. The hsa-miR-564 gene can be 103, p. 3687-3692. Also, “hsa-mir-564” (miRBase Accession No. M10003570, SEQ ID NO: 397) having a hairpin-like structure is known as a precursor of “hsa-miR-564”.

The term “hsa-miR-125a-3p gene” or “hsa-miR-125a-3p” used herein includes the hsa-miR-125a-3p gene (miRBase Accession No. MIMAT0004602) described in SEQ ID NO: 180, a homolog or an ortholog of a different organism species, and the like. The hsa-miR-125a-3p gene can be obtained by a method described in Lagos-Quintana M et al., 2002, Curr Biol, Vol. 12, p. 735-739. Also, “hsa-mir-125a” (miRBase Accession No. M10000469, SEQ ID NO: 398) having a hairpin-like structure is known as a precursor of “hsa-miR-125a-3p”.

The term “hsa-miR-92b-5p gene” or “hsa-miR-92b-5p” used herein includes the hsa-miR-92b-5p gene (miRBase Accession No. MIMAT0004792) described in SEQ ID NO: 181, a homolog or an ortholog of a different organism species, and the like. The hsa-miR-92b-5p gene can be obtained by a method described in Cummins J M et al., 2006, Proc Natl Acad Sci USA, Vol. 103, p. 3687-3692. Also, “hsa-mir-92b” (miRBase Accession No. M10003560, SEQ ID NO: 395) having a hairpin-like structure is known as a precursor of “hsa-miR-92b-5p”.

The term “hsa-miR-92a-3p gene” or “hsa-miR-92a-3p” used herein includes the hsa-miR-92a-3p gene (miRBase Accession No. MIMAT0000092) described in SEQ ID NO: 182, a homolog or an ortholog of a different organism species, and the like. The hsa-miR-92a-3p gene can be obtained by a method described in Mourelatos Z et al., 2002, Genes Dev, Vol. 16, p. 720-728. Also, “hsa-mir-92a-1” and “hsa-mir-92a-2” (miRBase Accession Nos. M10000093 and M10000094, SEQ ID NOs: 399 and 391) having a hairpin-like structure are known as precursors of “hsa-miR-92a-3p”.

The term “hsa-miR-663a gene” or “hsa-miR-663a” used herein includes the hsa-miR-663a gene (miRBase Accession No. MIMAT0003326) described in SEQ ID NO: 183, a homolog or an ortholog of a different organism species, and the like. The hsa-miR-663a gene can be obtained by a method described in Cummins J M et al., 2006, Proc Natl Acad Sci USA, Vol. 103, p. 3687-3692. Also, “hsa-mir-663a” (miRBase Accession No. M10003672, SEQ ID NO: 400) having a hairpin-like structure is known as a precursor of “hsa-miR-663a”.

The term “hsa-miR-4688 gene” or “hsa-miR-4688” used herein includes the hsa-miR-4688 gene (miRBase Accession No. MIMAT0019777) described in SEQ ID NO: 184, a homolog or an ortholog of a different organism species, and the like. The hsa-miR-4688 gene can be obtained by a method described in Persson H et al., 2011, Cancer Res, Vol. 71, p. 78-86. Also, “hsa-mir-4688” (miRBase Accession No. M10017321, SEQ ID NO: 401) having a hairpin-like structure is known as a precursor of “hsa-miR-4688”.

The term “hsa-miR-4648 gene” or “hsa-miR-4648” used herein includes the hsa-miR-4648 gene (miRBase Accession No. MIMAT0019710) described in SEQ ID NO: 185, a homolog or an ortholog of a different organism species, and the like. The hsa-miR-4648 gene can be obtained by a method described in Persson H et al., 2011, Cancer Res, Vol. 71, p. 78-86. Also, “hsa-mir-4648” (miRBase Accession No. M10017275, SEQ ID NO: 402) having a hairpin-like structure is known as a precursor of “hsa-miR-4648”.

The term “hsa-miR-6085 gene” or “hsa-miR-6085” used herein includes the hsa-miR-6085 gene (miRBase Accession No. MIMAT0023710) described in SEQ ID NO: 186, a homolog or an ortholog of a different organism species, and the like. The hsa-miR-6085 gene can be obtained by a method described in Voellenkle C et al., 2012, RNA, Vol. 18, p. 472-484. Also, “hsa-mir-6085” (miRBase Accession No. M10020362, SEQ ID NO: 403) having a hairpin-like structure is known as a precursor of “hsa-miR-6085”.

The term “hsa-miR-6126 gene” or “hsa-miR-6126” used herein includes the hsa-miR-6126 gene (miRBase Accession No. MIMAT0024599) described in SEQ ID NO: 187, a homolog or an ortholog of a different organism species, and the like. The hsa-miR-6126 gene can be obtained by a method described in Smith J L et al., 2012, J Virol, Vol. 86, p. 5278-5287. Also, “hsa-mir-6126” (miRBase Accession No. M10021260, SEQ ID NO: 404) having a hairpin-like structure is known as a precursor of “hsa-miR-6126”.

The term “hsa-miR-6880-5p gene” or “hsa-miR-6880-5p” used herein includes the hsa-miR-6880-5p gene (miRBase Accession No. MIMAT0027660) described in SEQ ID NO: 188, a homolog or an ortholog of a different organism species, and the like. The hsa-miR-6880-5p gene can be obtained by a method described in Ladewig E et al., 2012, Genome Res, Vol. 22, p. 1634-1645. Also, “hsa-mir-6880” (miRBase Accession No. M10022727, SEQ ID NO: 405) having a hairpin-like structure is known as a precursor of “hsa-miR-6880-5p”.

The term “hsa-miR-328-5p gene” or “hsa-miR-328-5p” used herein includes the hsa-miR-328-5p gene (miRBase Accession No. MIMAT0026486) described in SEQ ID NO: 189, a homolog or an ortholog of a different organism species, and the like. The hsa-miR-328-5p gene can be obtained by a method described in Kim J et al., 2004, Proc Natl Acad Sci USA, Vol. 101, p. 360-365. Also, “hsa-mir-328” (miRBase Accession No. M10000804, SEQ ID NO: 406) having a hairpin-like structure is known as a precursor of “hsa-miR-328-5p”.

The term “hsa-miR-6768-5p gene” or “hsa-miR-6768-5p” used herein includes the hsa-miR-6768-5p gene (miRBase Accession No. MIMAT0027436) described in SEQ ID NO: 190, a homolog or an ortholog of a different organism species, and the like. The hsa-miR-6768-5p gene can be obtained by a method described in Ladewig E et al., 2012, Genome Res, Vol. 22, p. 1634-1645. Also, “hsa-mir-6768” (miRBase Accession No. M10022613, SEQ ID NO: 407) having a hairpin-like structure is known as a precursor of “hsa-miR-6768-5p”.

The term “hsa-miR-3180 gene” or “hsa-miR-3180” used herein includes the hsa-miR-3180 gene (miRBase Accession No. MIMAT0018178) described in SEQ ID NO: 191, a homolog or an ortholog of a different organism species, and the like. The hsa-miR-3180 gene can be obtained by a method described in Creighton C J et al., 2010, PLoS One, Vol. 5, e9637. Also, “hsa-mir-3180-4” and “hsa-mir-3180-5” (miRBase Accession Nos. M10016408 and M10016409, SEQ ID NOs: 408 and 409) having a hairpin-like structure are known as precursors of “hsa-miR-3180”.

The term “hsa-miR-6087 gene” or “hsa-miR-6087” used herein includes the hsa-miR-6087 gene (miRBase Accession No. MIMAT0023712) described in SEQ ID NO: 192, a homolog or an ortholog of a different organism species, and the like. The hsa-miR-6087 gene can be obtained by a method described in Yoo J K et al., 2012, Stem Cells Dev, Vol. 21, p. 2049-2057. Also, “hsa-mir-6087” (miRBase Accession No. M10020364, SEQ ID NO: 410) having a hairpin-like structure is known as a precursor of “hsa-miR-6087”.

The term “hsa-miR-1273g-3p gene” or “hsa-miR-1273g-3p” used herein includes the hsa-miR-1273g-3p gene (miRBase Accession No. MIMAT0022742) described in SEQ ID NO: 193, a homolog or an ortholog of a different organism species, and the like. The hsa-miR-1273g-3p gene can be obtained by a method described in Reshmi G et al., 2011, Genomics, Vol. 97, p. 333-340. Also, “hsa-mir-1273g” (miRBase Accession No. M10018003, SEQ ID NO: 411) having a hairpin-like structure is known as a precursor of “hsa-miR-1273g-3p”.

The term “hsa-miR-1225-5p gene” or “hsa-miR-1225-5p” used herein includes the hsa-miR-1225-5p gene (miRBase Accession No. MIMAT0005572) described in SEQ ID NO: 194, a homolog or an ortholog of a different organism species, and the like. The hsa-miR-1225-5p gene can be obtained by a method described in Berezikov E et al., 2007, Mol Cell, Vol. 28, p. 328-336. Also, “hsa-mir-1225” (miRBase Accession No. M10006311, SEQ ID NO: 303) having a hairpin-like structure is known as a precursor of “hsa-miR-1225-5p”.

The term “hsa-miR-3196 gene” or “hsa-miR-3196” used herein includes the hsa-miR-3196 gene (miRBase Accession No. MIMAT0015080) described in SEQ ID NO: 195, a homolog or an ortholog of a different organism species, and the like. The hsa-miR-3196 gene can be obtained by a method described in Stark M S et al., 2010, PLoS One, Vol. 5, e9685. Also, “hsa-mir-3196” (miRBase Accession No. M10014241, SEQ ID NO: 412) having a hairpin-like structure is known as a precursor of “hsa-miR-3196”.

The term “hsa-miR-4695-5p gene” or “hsa-miR-4695-5p” used herein includes the hsa-miR-4695-5p gene (miRBase Accession No. MIMAT0019788) described in SEQ ID NO: 196, a homolog or an ortholog of a different organism species, and the like. The hsa-miR-4695-5p gene can be obtained by a method described in Persson H et al., 2011, Cancer Res, Vol. 71, p. 78-86. Also, “hsa-mir-4695” (miRBase Accession No. MI0017328, SEQ ID NO: 413) having a hairpin-like structure is known as a precursor of “hsa-miR-4695-5p”.

The term “hsa-miR-6732-5p gene” or “hsa-miR-6732-5p” used herein includes the hsa-miR-6732-5p gene (miRBase Accession No. MIMAT0027365) described in SEQ ID NO: 197, a homolog or an ortholog of a different organism species, and the like. The hsa-miR-6732-5p gene can be obtained by a method described in Ladewig E et al., 2012, Genome Res, Vol. 22, p. 1634-1645. Also, “hsa-mir-6732” (miRBase Accession No. M10022577, SEQ ID NO: 414) having a hairpin-like structure is known as a precursor of “hsa-miR-6732-5p”.

The term “hsa-miR-638 gene” or “hsa-miR-638” used herein includes the hsa-miR-638 gene (miRBase Accession No. MIMAT0003308) described in SEQ ID NO: 198, a homolog or an ortholog of a different organism species, and the like. The hsa-miR-638 gene can be obtained by a method described in Cummins J M et al., 2006, Proc Natl Acad Sci USA, Vol. 103, p. 3687-3692. Also, “hsa-mir-638” (miRBase Accession No. M10003653, SEQ ID NO: 415) having a hairpin-like structure is known as a precursor of “hsa-miR-638”.

The term “hsa-miR-6813-5p gene” or “hsa-miR-6813-5p” used herein includes the hsa-miR-6813-5p gene (miRBase Accession No. MIMAT0027526) described in SEQ ID NO: 199, a homolog or an ortholog of a different organism species, and the like. The hsa-miR-6813-5p p. 1634-1645. Also, “hsa-mir-6813” (miRBase Accession No. M10022658, SEQ ID NO: 416) having a hairpin-like structure is known as a precursor of “hsa-miR-6813-5p”.

The term “hsa-miR-665 gene” or “hsa-miR-665” used herein includes the hsa-miR-665 gene (miRBase Accession No. MIMAT0004952) described in SEQ ID NO: 200, a homolog or an ortholog of a different organism species, and the like. The hsa-miR-665 gene can be obtained by a method described in Berezikov E et al., 2006, Genome Res, Vol. 16, p. 1289-1298. Also, “hsa-mir-665” (miRBase Accession No. M10005563, SEQ ID NO: 417) having a hairpin-like structure is known as a precursor of “hsa-miR-665”.

The term “hsa-miR-486-3p gene” or “hsa-miR-486-3p” used herein includes the hsa-miR-486-3p gene (miRBase Accession No. MIMAT0004762) described in SEQ ID NO: 201, a homolog or an ortholog of a different organism species, and the like. The hsa-miR-486-3p gene can be obtained by a method described in Fu H et al., 2005, FEBS Lett, Vol. 579, p. 3849-3854. Also, “hsa-mir-486” and “hsa-mir-486-2” (miRBase Accession Nos. M10002470 and M10023622, SEQ ID NOs: 418 and 419) having a hairpin-like structure are known as precursors of “hsa-miR-486-3p”.

The term “hsa-miR-4466 gene” or “hsa-miR-4466” used herein includes the hsa-miR-4466 gene (miRBase Accession No. MIMAT0018993) described in SEQ ID NO: 202, a homolog or an ortholog of a different organism species, and the like. The hsa-miR-4466 gene can be obtained by a method described in Jima D D et al., 2010, Blood, Vol. 116, e118-e127. Also, “hsa-mir-4466” (miRBase Accession No. M10016817, SEQ ID NO: 420) having a hairpin-like structure is known as a precursor of “hsa-miR-4466”.

The term “hsa-miR-30c-1-3p gene” or “hsa-miR-30c-1-3p” used herein includes the hsa-miR-30c-1-3p gene (miRBase Accession No. MIMAT0004674) described in SEQ ID NO: 203, a homolog or an ortholog of a different organism species, and the like. The hsa-miR-30c-1-3p gene can be obtained by a method described in Lagos-Quintana M et al., 2002, Curr Biol, Vol. 12, p. 735-739. Also, “hsa-mir-30c-1” (miRBase Accession No. M10000736, SEQ ID NO: 421) having a hairpin-like structure is known as a precursor of “hsa-miR-30c-1-3p”.

The term “hsa-miR-3621 gene” or “hsa-miR-3621” used herein includes the hsa-miR-3621 gene (miRBase Accession No. MIMAT0018002) described in SEQ ID NO: 204, a homolog or an ortholog of a different organism species, and the like. The hsa-miR-3621 gene can be obtained by a method described in Witten D et al., 2010, BMC Biol, Vol. 8, p. 58. Also, “hsa-mir-3621” (miRBase Accession No. M10016012, SEQ ID NO: 422) having a hairpin-like structure is known as a precursor of “hsa-miR-3621”.

The term “hsa-miR-6743-5p gene” or “hsa-miR-6743-5p” used herein includes the hsa-miR-6743-5p gene (miRBase Accession No. MIMAT0027387) described in SEQ ID NO: 205, a homolog or an ortholog of a different organism species, and the like. The hsa-miR-6743-5p gene can be obtained by a method described in Ladewig E et al., 2012, Genome Res, Vol. 22, p. 1634-1645. Also, “hsa-mir-6743” (miRBase Accession No. M10022588, SEQ ID NO: 423) having a hairpin-like structure is known as a precursor of “hsa-miR-6743-5p”.

The term “hsa-miR-4298 gene” or “hsa-miR-4298” used herein includes the hsa-miR-4298 gene (miRBase Accession No. MIMAT0016852) described in SEQ ID NO: 206, a homolog or an ortholog of a different organism species, and the like. The hsa-miR-4298 gene can be obtained by a method described in Goff L A et al., 2009, PLoS One, Vol. 4, e7192. Also, “hsa-mir-4298” (miRBase Accession No. MI0015830, SEQ ID NO: 424) having a hairpin-like structure is known as a precursor of “hsa-miR-4298”.

The term “hsa-miR-4741 gene” or “hsa-miR-4741” used herein includes the hsa-miR-4741 gene (miRBase Accession No. MIMAT0019871) described in SEQ ID NO: 207, a homolog or an ortholog of a different organism species, and the like. The hsa-miR-4741 gene Also, “hsa-mir-4741” (miRBase Accession No. M10017379, SEQ ID NO: 425) having a hairpin-like structure is known as a precursor of “hsa-miR-4741”.

The term “hsa-miR-3619-3p gene” or “hsa-miR-3619-3p” used herein includes the hsa-miR-3619-3p gene (miRBase Accession No. MIMAT0019219) described in SEQ ID NO: 208, a homolog or an ortholog of a different organism species, and the like. The hsa-miR-3619-3p gene can be obtained by a method described in Witten D et al., 2010, BMC Biol, Vol. 8, p. 58. Also, “hsa-mir-3619” (miRBase Accession No. M10016009, SEQ ID NO: 426) having a hairpin-like structure is known as a precursor of “hsa-miR-3619-3p”.

The term “hsa-miR-6824-5p gene” or “hsa-miR-6824-5p” used herein includes the hsa-miR-6824-5p gene (miRBase Accession No. MIMAT0027548) described in SEQ ID NO: 209, a homolog or an ortholog of a different organism species, and the like. The hsa-miR-6824-5p gene can be obtained by a method described in Ladewig E et al., 2012, Genome Res, Vol. 22, p. 1634-1645. Also, “hsa-mir-6824” (miRBase Accession No. M10022669, SEQ ID NO: 427) having a hairpin-like structure is known as a precursor of “hsa-miR-6824-5p”.

The term “hsa-miR-5698 gene” or “hsa-miR-5698” used herein includes the hsa-miR-5698 gene (miRBase Accession No. MIMAT0022491) described in SEQ ID NO: 210, a homolog or an ortholog of a different organism species, and the like. The hsa-miR-5698 gene can be obtained by a method described in Watahiki A et al., 2011, PLoS One, Vol. 6, e24950. Also, “hsa-mir-5698” (miRBase Accession No. M10019305, SEQ ID NO: 428) having a hairpin-like structure is known as a precursor of “hsa-miR-5698”.

The term “hsa-miR-371a-5p gene” or “hsa-miR-371a-5p” used herein includes the hsa-miR-371a-5p gene (miRBase Accession No. MIMAT0004687) described in SEQ ID NO: 211, a homolog or an ortholog of a different organism species, and the like. The hsa-miR-371a-5p gene can be obtained by a method described in Suh M R et al., 2004, Dev Biol, Vol. 270, p. 488-498. Also, “hsa-mir-371a” (miRBase Accession No. M10000779, SEQ ID NO: 429) having a hairpin-like structure is known as a precursor of “hsa-miR-371a-5p”.

The term “hsa-miR-4488 gene” or “hsa-miR-4488” used herein includes the hsa-miR-4488 gene (miRBase Accession No. MIMAT0019022) described in SEQ ID NO: 212, a homolog or an ortholog of a different organism species, and the like. The hsa-miR-4488 gene can be obtained by a method described in Jima D D et al., 2010, Blood, Vol. 116, e118-e127. Also, “hsa-mir-4488” (miRBase Accession No. M10016849, SEQ ID NO: 430) having a hairpin-like structure is known as a precursor of “hsa-miR-4488”.

The term “hsa-miR-1233-5p gene” or “hsa-miR-1233-5p” used herein includes the hsa-miR-1233-5p gene (miRBase Accession No. MIMAT0022943) described in SEQ ID NO: 213, a homolog or an ortholog of a different organism species, and the like. The hsa-miR-1233-5p gene can be obtained by a method described in Berezikov E et al., 2007, Mol Cell, Vol. 28, p. 328-336. Also, “hsa-mir-1233-1” and “hsa-mir-1233-2” (miRBase Accession Nos. M10006323 and M10015973, SEQ ID NOs: 431 and 432) having a hairpin-like structure are known as precursors of “hsa-miR-1233-5p”.

The term “hsa-miR-4723-5p gene” or “hsa-miR-4723-5p” used herein includes the hsa-miR-4723-5p gene (miRBase Accession No. MIMAT0019838) described in SEQ ID NO: 214, a homolog or an ortholog of a different organism species, and the like. The hsa-miR-4723-5p gene can be obtained by a method described in Persson H et al., 2011, Cancer Res, Vol. 71, p. 78-86. Also, “hsa-mir-4723” (miRBase Accession No. M10017359, SEQ ID NO: 433) having a hairpin-like structure is known as a precursor of “hsa-miR-4723-5p”.

The term “hsa-miR-24-3p gene” or “hsa-miR-24-3p” used herein includes the hsa-miR-24-3p gene (miRBase Accession No. MIMAT0000080) described in SEQ ID NO: 215, a homolog or an ortholog of a different organism species, and the like. The hsa-miR-24-3p gene can be obtained by a method described in Lagos-Quintana M et al., 2001, Science, Vol. 294, p. 853-858. Also, “hsa-mir-24-1” and “hsa-mir-24-2” (miRBase Accession Nos. M10000080 and M10000081, SEQ ID NOs: 434 and 435) having a hairpin-like structure are known as precursors of “hsa-miR-24-3p”.

The term “hsa-miR-1238-5p gene” or “hsa-miR-1238-5p” used herein includes the hsa-miR-1238-5p gene (miRBase Accession No. MIMAT0022947) described in SEQ ID NO: 216, a homolog or an ortholog of a different organism species, and the like. The hsa-miR-1238-5p gene can be obtained by a method described in Berezikov E et al., 2007, Mol Cell, Vol. 28, p. 328-336. Also, “hsa-mir-1238” (miRBase Accession No. M10006328, SEQ ID NO: 436) having a hairpin-like structure is known as a precursor of “hsa-miR-1238-5p”.

The term “hsa-miR-4442 gene” or “hsa-miR-4442” used herein includes the hsa-miR-4442 gene (miRBase Accession No. MIMAT0018960) described in SEQ ID NO: 217, a homolog or an ortholog of a different organism species, and the like. The hsa-miR-4442 gene can be obtained by a method described in Jima D D et al., 2010, Blood, Vol. 116, e118-e127. Also, “hsa-mir-4442” (miRBase Accession No. M10016785, SEQ ID NO: 437) having a hairpin-like structure is known as a precursor of “hsa-miR-4442”.

The term “hsa-miR-3928-3p gene” or “hsa-miR-3928-3p” used herein includes the hsa-miR-3928-3p gene (miRBase Accession No. MIMAT0018205) described in SEQ ID NO: 218, a homolog or an ortholog of a different organism species, and the like. The hsa-miR-3928-3p gene can be obtained by a method described in Creighton C J et al., 2010, PLoS One, Vol. 5, e9637. Also, “hsa-mir-3928” (miRBase Accession No. M10016438, SEQ ID NO: 438) having a hairpin-like structure is known as a precursor of “hsa-miR-3928-3p”.

The term “hsa-miR-6716-5p gene” or “hsa-miR-6716-5p” used herein includes the hsa-miR-6716-5p gene (miRBase Accession No. MIMAT0025844) described in SEQ ID NO: 219, a homolog or an ortholog of a different organism species, and the like. The hsa-miR-6716-5p gene can be obtained by a method described in Li Y et al., 2012, Gene, Vol. 497, p. 330-335. Also, “hsa-mir-6716” (miRBase Accession No. M10022550, SEQ ID NO: 439) having a hairpin-like structure is known as a precursor of “hsa-miR-6716-5p”.

The term “hsa-miR-6089 gene” or “hsa-miR-6089” used herein includes the hsa-miR-6089 gene (miRBase Accession No. MIMAT0023714) described in SEQ ID NO: 220, a homolog or an ortholog of a different organism species, and the like. The hsa-miR-6089 gene can be obtained by a method described in Yoo J K et al., 2012, Stem Cells Dev, Vol. 21, p. 2049-2057. Also, “hsa-mir-6089-1” and “hsa-mir-6089-2” (miRBase Accession Nos. M10020366 and M10023563, SEQ ID NOs: 440 and 441) having a hairpin-like structure are known as precursors of “hsa-miR-6089”.

The term “hsa-miR-6124 gene” or “hsa-miR-6124” used herein includes the hsa-miR-6124 gene (miRBase Accession No. MIMAT0024597) described in SEQ ID NO: 221, a homolog or an ortholog of a different organism species, and the like. The hsa-miR-6124 gene can be obtained by a method described in Smith J L et al., 2012, J Virol, Vol. 86, p. 5278-5287. Also, “hsa-mir-6124” (miRBase Accession No. M10021258, SEQ ID NO: 442) having a hairpin-like structure is known as a precursor of “hsa-miR-6124”.

The term “hsa-miR-6778-5p gene” or “hsa-miR-6778-5p” used herein includes the hsa-miR-6778-5p gene (miRBase Accession No. MIMAT0027456) described in SEQ ID NO: 222, a homolog or an ortholog of a different organism species, and the like. The hsa-miR-6778-5p gene can be obtained by a method described in Ladewig E et al., 2012, Genome Res, Vol. 22, p. 1634-1645. Also, “hsa-mir-6778” (miRBase Accession No. M10022623, SEQ ID NO: 443) having a hairpin-like structure is known as a precursor of “hsa-miR-6778-5p”.

The term “hsa-miR-557 gene” or “hsa-miR-557” used herein includes the hsa-miR-557 gene (miRBase Accession No. MIMAT0003221) described in SEQ ID NO: 223, a homolog or an ortholog of a different organism species, and the like. The hsa-miR-557 gene can be 103, p. 3687-3692. Also, “hsa-mir-557” (miRBase Accession No. M10003563, SEQ ID NO: 444) having a hairpin-like structure is known as a precursor of “hsa-miR-557”.

The term “hsa-miR-6090 gene” or “hsa-miR-6090” used herein includes the hsa-miR-6090 gene (miRBase Accession No. MIMAT0023715) described in SEQ ID NO: 224, a homolog or an ortholog of a different organism species, and the like. The hsa-miR-6090 gene can be obtained by a method described in Yoo J K et al., 2012, Stem Cells Dev, Vol. 21, p. 2049-2057. Also, “hsa-mir-6090” (miRBase Accession No. M10020367, SEQ ID NO: 445) having a hairpin-like structure is known as a precursor of “hsa-miR-6090”.

The term “hsa-miR-6757-5p gene” or “hsa-miR-6757-5p” used herein includes the hsa-miR-6757-5p gene (miRBase Accession No. MIMAT0027414) described in SEQ ID NO: 714, a homolog or an ortholog of a different organism species, and the like. The hsa-miR-6757-5p gene can be obtained by a method described in Ladewig E et al., 2012, Genome Res., Vol. 22, p. 1634-1645. Also, “hsa-mir-6757” (miRBase Accession No. M10022602, SEQ ID NO: 730) having a hairpin-like structure is known as a precursor of “hsa-miR-6757-5p”.

The term “hsa-miR-4448 gene” or “hsa-miR-4448” used herein includes the hsa-miR-4448 gene (miRBase Accession No. MIMAT0018967) described in SEQ ID NO: 715, a homolog or an ortholog of a different organism species, and the like. The hsa-miR-4448 gene can be obtained by a method described in Jima D D et al., 2010, Blood, Vol. 116, e118-e127. Also, “hsa-mir-4448” (miRBase Accession No. M10016791, SEQ ID NO: 731) having a hairpin-like structure is known as a precursor of “hsa-miR-4448”.

The term “hsa-miR-671-5p gene” or “hsa-miR-671-5p” used herein includes the hsa-miR-671-5p gene (miRBase Accession No. MIMAT0003880) described in SEQ ID NO: 716, a homolog or an ortholog of a different organism species, and the like. The hsa-miR-671-5p gene can be obtained by a method described in Berezikov E et al., 2006, Genome Res, Vol. 16, p. 1289-1298. Also, “hsa-mir-671” (miRBase Accession No. M10003760, SEQ ID NO: 732) having a hairpin-like structure is known as a precursor of “hsa-miR-671-5p”.

The term “hsa-miR-3178 gene” or “hsa-miR-3178” used herein includes the hsa-miR-3178 gene (miRBase Accession No. MIMAT0015055) described in SEQ ID NO: 717, a homolog or an ortholog of a different organism species, and the like. The hsa-miR-3178 gene can be obtained by a method described in Stark M S et al., 2010, PLoS One, Vol. 5, e9685. Also, “hsa-mir-3178” (miRBase Accession No. M10014212, SEQ ID NO: 733) having a hairpin-like structure is known as a precursor of “hsa-miR-3178”.

The term “hsa-miR-4725-3p gene” or “hsa-miR-4725-3p” used herein includes the hsa-miR-4725-3p gene (miRBase Accession No. MIMAT0019844) described in SEQ ID NO: 718, a homolog or an ortholog of a different organism species, and the like. The hsa-miR-4725-3p gene can be obtained by a method described in Persson H et al., 2011, Cancer Res, Vol. 71, p. 78-86. Also, “hsa-mir-4725” (miRBase Accession No. MI0017362, SEQ ID NO: 734) having a hairpin-like structure is known as a precursor of “hsa-miR-4725-3p”.

The term “hsa-miR-940 gene” or “hsa-miR-940” used herein includes the hsa-miR-940 gene (miRBase Accession No. MIMAT0004983) described in SEQ ID NO: 719, a homolog or an ortholog of a different organism species, and the like. The hsa-miR-940 gene can be obtained by a method described in Lui W O et al., 2007, A Cancer Res., Vol. 67, p. 6031-6043. Also, “hsa-mir-940” (miRBase Accession No. M10005762, SEQ ID NO: 735) having a hairpin-like structure is known as a precursor of “hsa-miR-940”.

The term “hsa-miR-6789-5p gene” or “hsa-miR-6789-5p” used herein includes the hsa-miR-6789-5p gene (miRBase Accession No. MIMAT0027478) described in SEQ ID NO: 720, a homolog or an ortholog of a different organism species, and the like. The hsa-miR-6789-5p gene can be obtained by a method described in Ladewig E et al., 2012, Genome Res., Vol. 22, p. 1634-1645. Also, “hsa-mir-6789” (miRBase Accession No. M10022634, SEQ ID NO: 736) having a hairpin-like structure is known as a precursor of “hsa-miR-6789-5p”.

The term “hsa-miR-4484 gene” or “hsa-miR-4484” used herein includes the hsa-miR-4484 gene (miRBase Accession No. MIMAT0019018) described in SEQ ID NO: 721, a homolog or an ortholog of a different organism species, and the like. The hsa-miR-4484 gene can be obtained by a method described in Jima D D et al., 2010, Blood., Vol. 116, e118-e127. Also, “hsa-mir-4484” (miRBase Accession No. M10016845, SEQ ID NO: 737) having a hairpin-like structure is known as a precursor of “hsa-miR-4484”.

The term “hsa-miR-4634 gene” or “hsa-miR-4634” used herein includes the hsa-miR-4634 gene (miRBase Accession No. MIMAT0019691) described in SEQ ID NO: 722, a homolog or an ortholog of a different organism species, and the like. The hsa-miR-4634 gene can be obtained by a method described in Persson H et al., 2011, Cancer Res., Vol. 71, p. 78-86. Also, “hsa-mir-4634” (miRBase Accession No. M10017261, SEQ ID NO: 738) having a hairpin-like structure is known as a precursor of “hsa-miR-4634”.

The term “hsa-miR-4745-5p gene” or “hsa-miR-4745-5p” used herein includes the hsa-miR-4745-5p gene (miRBase Accession No. MIMAT0019878) described in SEQ ID NO: 723, a homolog or an ortholog of a different organism species, and the like. The hsa-miR-4745-5p gene can be obtained by a method described in Persson H et al., 2011, Cancer Res, Vol. 71, p. 78-86. Also, “hsa-mir-4745” (miRBase Accession No. M10017384, SEQ ID NO: 739) having a hairpin-like structure is known as a precursor of “hsa-miR-4745-5p”.

The term “hsa-miR-4730 gene” or “hsa-miR-4730” used herein includes the hsa-miR-4730 gene (miRBase Accession No. MIMAT0019852) described in SEQ ID NO: 724, a homolog or an ortholog of a different organism species, and the like. The hsa-miR-4730 gene Also, “hsa-mir-4730” (miRBase Accession No. M10017367, SEQ ID NO: 740) having a hairpin-like structure is known as a precursor of “hsa-miR-4730”.

The term “hsa-miR-6803-5p gene” or “hsa-miR-6803-5p” used herein includes the hsa-miR-6803-5p gene (miRBase Accession No. MIMAT0027506) described in SEQ ID NO: 725, a homolog or an ortholog of a different organism species, and the like. The hsa-miR-6803-5p gene can be obtained by a method described in Ladewig E et al., 2012, Genome Res, Vol. 22, p. 1634-1645. Also, “hsa-mir-6803” (miRBase Accession No. M10022648, SEQ ID NO: 741) having a hairpin-like structure is known as a precursor of “hsa-miR-6803-5p”.

The term “hsa-miR-6798-5p gene” or “hsa-miR-6798-5p” used herein includes the hsa-miR-6798-5p gene (miRBase Accession No. MIMAT0027496) described in SEQ ID NO: 726, a homolog or an ortholog of a different organism species, and the like. The hsa-miR-6798-5p gene can be obtained by a method described in Ladewig E et al., 2012, Genome Res, Vol. 22, p. 1634-1645. Also, “hsa-mir-6798” (miRBase Accession No. M10022643, SEQ ID NO: 742) having a hairpin-like structure is known as a precursor of “hsa-miR-6798-5p”.

The term “hsa-miR-3648 gene” or “hsa-miR-3648” used herein includes the hsa-miR-3648 gene (miRBase Accession No. MIMAT0018068) described in SEQ ID NO: 727, a homolog or an ortholog of a different organism species, and the like. The hsa-miR-3648 gene can be obtained by a method described in Meiri E et al., 2010, Nucleic Acids Res, Vol. 38, p. 6234-6246. Also, “hsa-mir-3648” (miRBase Accession No. M10016048, SEQ ID NO: 743) having a hairpin-like structure is known as a precursor of “hsa-miR-3648”.

The term “hsa-miR-4783-3p gene” or “hsa-miR-4783-3p” used herein includes the hsa-miR-4783-3p gene (miRBase Accession No. MIMAT0019947) described in SEQ ID NO: 728, a homolog or an ortholog of a different organism species, and the like. The hsa-miR-4783-3p 78-86. Also, “hsa-mir-4783” (miRBase Accession No. MI0017428, SEQ ID NO: 744) having a hairpin-like structure is known as a precursor of “hsa-miR-4783-3p”.

The term “hsa-miR-6836-3p gene” or “hsa-miR-6836-3p” used herein includes the hsa-miR-6836-3p gene (miRBase Accession No. MIMAT0027575) described in SEQ ID NO: 729, a homolog or an ortholog of a different organism species, and the like. The hsa-miR-6836-3p gene can be obtained by a method described in Ladewig E et al., 2012, Genome Res., Vol. 22, p. 1634-1645. Also, “hsa-mir-6836” (miRBase Accession No. MI0022682, SEQ ID NO: 745) having a hairpin-like structure is known as a precursor of “hsa-miR-6836-3p”.

A mature miRNA may become a variant due to the sequence cleaved shorter or longer by one to several flanking nucleotides, or nucleotide substitution, when cleaved as the mature miRNA from its RNA precursor having a hairpin-like structure. This variant is called isomiR (Morin R D. et al., 2008, Genome Res., Vol. 18, p. 610-621). miRBase Release 20 shows the nucleotide sequences represented by SEQ ID NOs: 1 to 224 and 714 to 729 as well as a large number of the nucleotide sequence variants and fragments represented by SEQ ID NOs: 446 to 713 and 746 to 765, called isomiRs. These variants can also be obtained as miRNAs having a nucleotide sequence represented by any of SEQ ID NOs: 1 to 224 and 714 to 729.

Specifically, among the variants of polynucleotides consisting of a nucleotide sequence represented by any of SEQ ID NOs: 1, 3, 4, 6, 7, 10, 11, 13, 14, 16, 17, 20, 22, 26, 29, 36, 38, 39, 40, 42, 43, 44, 46, 49, 52, 59, 60, 62, 63, 65, 66, 67, 72, 76, 77, 78, 81, 83, 84, 85, 86, 87, 88, 89, 90, 92, 93, 94, 96, 100, 103, 105, 106, 107, 113, 114, 115, 116, 117, 118, 119, 120, 121, 123, 124, 125, 126, 130, 132, 134, 136, 139, 140, 141, 142, 143, 144, 145, 147, 148, 150, 151, 152, 155, 157, 158, 159, 163, 164, 165, 167, 168, 169, 170, 171, 172, 173, 174, 175, 176, 177, 178, 179, 180, 181, 182, 183, 184, 185, 187, 189, 191, 192, 193, 195, 196, 198, 200, 201, 202, 203, 206, 207, 210, 211, 212, 213, 214, 215, 217, 218, 219, 220, 221, 715, 716, 717, 718, 719, 721, 723, 724, 727 and 728 or a nucleotide sequence derived from the nucleotide sequence by the replacement of u with t according to the present invention, examples of the longest variants registered in miRBase Release 20 include polynucleotides represented by SEQ ID NOs: 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 746, 748, 750, 752, 754, 756, 758, 760, 762 and 764, respectively.

Also, among the variants of polynucleotides consisting of a nucleotide sequence represented by any of SEQ ID NOs: 1, 3, 4, 6, 7, 10, 11, 13, 14, 16, 17, 20, 22, 26, 29, 36, 38, 39, 40, 42, 43, 44, 46, 49, 52, 59, 60, 62, 63, 65, 66, 67, 72, 76, 77, 78, 81, 83, 84, 85, 86, 87, 88, 89, 90, 92, 93, 94, 96, 100, 103, 105, 106, 107, 113, 114, 115, 116, 117, 118, 119, 120, 121, 123, 124, 125, 126, 130, 132, 134, 136, 139, 140, 141, 142, 143, 144, 145, 147, 148, 150, 151, 152, 155, 157, 158, 159, 163, 164, 165, 167, 168, 169, 170, 171, 172, 173, 174, 175, 176, 177, 178, 179, 180, 181, 182, 183, 184, 185, 187, 189, 191, 192, 193, 195, 196, 198, 200, 201, 202, 203, 206, 207, 210, 211, 212, 213, 214, 215, 217, 218, 219, 220, 221, 715, 716, 717, 718, 719, 721, 723, 724, 727 and 728 or a nucleotide sequence derived from the nucleotide sequence by the replacement of u with t according to the present invention, examples of the shortest variants registered in miRBase Release 20 include polynucleotides having sequences represented by SEQ ID NOs: 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 747, 749, 751, 753, 755, 757, 759, 761, 763 and 765, respectively.

In addition to these variants and fragments, examples thereof include a large number of isomiR polynucleotides of SEQ ID NOs: 1 to 224 and 714 to 729 registered in miRBase. Examples of the polynucleotide comprising a nucleotide sequence represented by any of SEQ ID NOs: 1 to 224 and 714 to 729 include a polynucleotide represented by any of SEQ ID NOs: 225 to 445 and 730 to 745, which are their respective precursors.

The names and miRBase Accession Nos. (registration numbers) of the genes represented by SEQ ID NOs: 1 to 765 are shown in Table 1.

As used herein, the term “capable of specifically binding” means that the nucleic acid probe or the primer used in the present invention binds to a particular target nucleic acid and cannot substantially bind to other nucleic acids.

TABLE 1

| SEQ ID | | miRBase |
|---|---|---|
| NO: | Gene name | registration No. |
| 1 | hsa-miR-1343-3p | MIMAT0019776 |
| 2 | hsa-miR-6726-5p | MIMAT0027353 |
| 3 | hsa-miR-6515-3p | MIMAT0025487 |
| 4 | hsa-miR-4651 | MIMAT0019715 |
| 5 | hsa-miR-4257 | MIMAT0016878 |
| 6 | hsa-miR-3188 | MIMAT0015070 |
| 7 | hsa-miR-6131 | MIMAT0024615 |
| 8 | hsa-miR-6766-3p | MIMAT0027433 |
| 9 | hsa-miR-7641 | MIMAT0029782 |
| 10 | hsa-miR-1249 | MIMAT0005901 |
| 11 | hsa-miR-3679-3p | MIMAT0018105 |
| 12 | hsa-miR-6787-5p | MIMAT0027474 |
| 13 | hsa-miR-4454 | MIMAT0018976 |
| 14 | hsa-miR-3135b | MIMAT0018985 |
| 15 | hsa-miR-6765-3p | MIMAT0027431 |
| 16 | hsa-miR-7975 | MIMAT0031178 |
| 17 | hsa-miR-204-3p | MIMAT0022693 |
| 18 | hsa-miR-7977 | MIMAT0031180 |
| 19 | hsa-miR-7110-5p | MIMAT0028117 |
| 20 | hsa-miR-6717-5p | MIMAT0025846 |
| 21 | hsa-miR-6870-5p | MIMAT0027640 |
| 22 | hsa-miR-663b | MIMAT0005867 |
| 23 | hsa-miR-6875-5p | MIMAT0027650 |
| 24 | hsa-miR-8072 | MIMAT0030999 |
| 25 | hsa-miR-6816-5p | MIMAT0027532 |
| 26 | hsa-miR-4281 | MIMAT0016907 |
| 27 | hsa-miR-6729-5p | MIMAT0027359 |
| 28 | hsa-miR-8069 | MIMAT0030996 |
| 29 | hsa-miR-4706 | MIMAT0019806 |
| 30 | hsa-miR-7108-5p | MIMAT0028113 |
| 31 | hsa-miR-4433b-3p | MIMAT0030414 |
| 32 | hsa-miR-6893-5p | MIMAT0027686 |
| 33 | hsa-miR-6857-5p | MIMAT0027614 |
| 34 | hsa-miR-1227-5p | MIMAT0022941 |
| 35 | hsa-miR-6741-5p | MIMAT0027383 |
| 36 | hsa-miR-451a | MIMAT0001631 |
| 37 | hsa-miR-8063 | MIMAT0030990 |
| 38 | hsa-miR-3622a-5p | MIMAT0018003 |
| 39 | hsa-miR-615-5p | MIMAT0004804 |
| 40 | hsa-miR-128-1-5p | MIMAT0026477 |
| 41 | hsa-miR-6825-5p | MIMAT0027550 |
| 42 | hsa-miR-1260b | MIMAT0015041 |
| 43 | hsa-miR-4433-3p | MIMAT0018949 |
| 44 | hsa-miR-4665-5p | MIMAT0019739 |
| 45 | hsa-miR-7845-5p | MIMAT0030420 |
| 46 | hsa-miR-1908-5p | MIMAT0007881 |
| 47 | hsa-miR-6840-3p | MIMAT0027583 |
| 48 | hsa-miR-6765-5p | MIMAT0027430 |
| 49 | hsa-miR-296-5p | MIMAT0000690 |
| 50 | hsa-miR-3675-3p | MIMAT0018099 |
| 51 | hsa-miR-6781-5p | MIMAT0027462 |
| 52 | hsa-miR-423-5p | MIMAT0004748 |
| 53 | hsa-miR-3663-3p | MIMAT0018085 |
| 54 | hsa-miR-6784-5p | MIMAT0027468 |
| 55 | hsa-miR-6749-5p | MIMAT0027398 |
| 56 | hsa-miR-1231 | MIMAT0005586 |
| 57 | hsa-miR-4746-3p | MIMAT0019881 |
| 58 | hsa-miR-6780b-5p | MIMAT0027572 |
| 59 | hsa-miR-4758-5p | MIMAT0019903 |
| 60 | hsa-miR-3679-5p | MIMAT0018104 |
| 61 | hsa-miR-3184-5p | MIMAT0015064 |
| 62 | hsa-miR-6125 | MIMAT0024598 |
| 63 | hsa-miR-6721-5p | MIMAT0025852 |
| 64 | hsa-miR-6791-5p | MIMAT0027482 |
| 65 | hsa-miR-3185 | MIMAT0015065 |
| 66 | hsa-miR-1260a | MIMAT0005911 |
| 67 | hsa-miR-3197 | MIMAT0015082 |
| 68 | hsa-miR-6845-5p | MIMAT0027590 |
| 69 | hsa-miR-6887-5p | MIMAT0027674 |
| 70 | hsa-miR-6738-5p | MIMAT0027377 |
| 71 | hsa-miR-6872-3p | MIMAT0027645 |
| 72 | hsa-miR-4497 | MIMAT0019032 |
| 73 | hsa-miR-1229-5p | MIMAT0022942 |
| 74 | hsa-miR-6820-5p | MIMAT0027540 |
| 75 | hsa-miR-6777-5p | MIMAT0027454 |
| 76 | hsa-miR-3917 | MIMAT0018191 |
| 77 | hsa-miR-5787 | MIMAT0023252 |
| 78 | hsa-miR-4286 | MIMAT0016916 |
| 79 | hsa-miR-6877-5p | MIMAT0027654 |
| 80 | hsa-miR-1225-3p | MIMAT0005573 |
| 81 | hsa-miR-6088 | MIMAT0023713 |
| 82 | hsa-miR-6800-5p | MIMAT0027500 |
| 83 | hsa-miR-1246 | MIMAT0005898 |
| 84 | hsa-miR-4467 | MIMAT0018994 |
| 85 | hsa-miR-4419b | MIMAT0019034 |
| 86 | hsa-miR-1914-3p | MIMAT0007890 |
| 87 | hsa-miR-4632-5p | MIMAT0022977 |
| 88 | hsa-miR-1915-5p | MIMAT0007891 |
| 89 | hsa-miR-3940-5p | MIMAT0019229 |
| 90 | hsa-miR-1185-2-3p | MIMAT0022713 |
| 91 | hsa-miR-6746-5p | MIMAT0027392 |
| 92 | hsa-miR-5001-5p | MIMAT0021021 |
| 93 | hsa-miR-1228-5p | MIMAT0005582 |
| 94 | hsa-miR-5572 | MIMAT0022260 |
| 95 | hsa-miR-4327 | MIMAT0016889 |
| 96 | hsa-miR-4638-5p | MIMAT0019695 |
| 97 | hsa-miR-6799-5p | MIMAT0027498 |
| 98 | hsa-miR-6861-5p | MIMAT0027623 |
| 99 | hsa-miR-6727-5p | MIMAT0027355 |
| 100 | hsa-miR-4513 | MIMAT0019050 |
| 101 | hsa-miR-6805-3p | MIMAT0027511 |
| 102 | hsa-miR-6808-5p | MIMAT0027516 |
| 103 | hsa-miR-4449 | MIMAT0018968 |
| 104 | hsa-miR-1199-5p | MIMAT0031119 |
| 105 | hsa-miR-1275 | MIMAT0005929 |
| 106 | hsa-miR-4792 | MIMAT0019964 |
| 107 | hsa-miR-4443 | MIMAT0018961 |
| 108 | hsa-miR-6891-5p | MIMAT0027682 |
| 109 | hsa-miR-6826-5p | MIMAT0027552 |
| 110 | hsa-miR-6807-5p | MIMAT0027514 |
| 111 | hsa-miR-7150 | MIMAT0028211 |
| 112 | hsa-miR-4534 | MIMAT0019073 |
| 113 | hsa-miR-4476 | MIMAT0019003 |
| 114 | hsa-miR-4649-5p | MIMAT0019711 |
| 115 | hsa-miR-4525 | MIMAT0019064 |
| 116 | hsa-miR-1915-3p | MIMAT0007892 |
| 117 | hsa-miR-4516 | MIMAT0019053 |
| 118 | hsa-miR-4417 | MIMAT0018929 |
| 119 | hsa-miR-642b-3p | MIMAT0018444 |
| 120 | hsa-miR-3141 | MIMAT0015010 |
| 121 | hsa-miR-5100 | MIMAT0022259 |
| 122 | hsa-miR-6848-5p | MIMAT0027596 |
| 123 | hsa-miR-4739 | MIMAT0019868 |
| 124 | hsa-miR-4459 | MIMAT0018981 |
| 125 | hsa-miR-1237-5p | MIMAT0022946 |
| 126 | hsa-miR-296-3p | MIMAT0004679 |
| 127 | hsa-miR-4665-3p | MIMAT0019740 |
| 128 | hsa-miR-6786-5p | MIMAT0027472 |
| 129 | hsa-miR-4258 | MIMAT0016879 |
| 130 | hsa-miR-6510-5p | MIMAT0025476 |
| 131 | hsa-miR-1343-5p | MIMAT0027038 |
| 132 | hsa-miR-1247-3p | MIMAT0022721 |
| 133 | hsa-miR-6805-5p | MIMAT0027510 |
| 134 | hsa-miR-4492 | MIMAT0019027 |
| 135 | hsa-miR-1469 | MIMAT0007347 |
| 136 | hsa-miR-1268b | MIMAT0018925 |
| 137 | hsa-miR-6858-5p | MIMAT0027616 |
| 138 | hsa-miR-3937 | MIMAT0018352 |
| 139 | hsa-miR-939-5p | MIMAT0004982 |
| 140 | hsa-miR-3656 | MIMAT0018076 |
| 141 | hsa-miR-744-5p | MIMAT0004945 |
| 142 | hsa-miR-4687-3p | MIMAT0019775 |
| 143 | hsa-miR-4763-3p | MIMAT0019913 |
| 144 | hsa-miR-3620-5p | MIMAT0022967 |
| 145 | hsa-miR-3195 | MIMAT0015079 |
| 146 | hsa-miR-6842-5p | MIMAT0027586 |
| 147 | hsa-miR-4707-5p | MIMAT0019807 |
| 148 | hsa-miR-642a-3p | MIMAT0020924 |
| 149 | hsa-miR-7113-3p | MIMAT0028124 |
| 150 | hsa-miR-4728-5p | MIMAT0019849 |
| 151 | hsa-miR-5195-3p | MIMAT0021127 |
| 152 | hsa-miR-1185-1-3p | MIMAT0022838 |
| 153 | hsa-miR-6774-5p | MIMAT0027448 |
| 154 | hsa-miR-8059 | MIMAT0030986 |
| 155 | hsa-miR-3131 | MIMAT0014996 |
| 156 | hsa-miR-7847-3p | MIMAT0030422 |
| 157 | hsa-miR-4463 | MIMAT0018987 |
| 158 | hsa-miR-128-2-5p | MIMAT0031095 |
| 159 | hsa-miR-4508 | MIMAT0019045 |
| 160 | hsa-miR-6806-5p | MIMAT0027512 |
| 161 | hsa-miR-7111-5p | MIMAT0028119 |
| 162 | hsa-miR-6782-5p | MIMAT0027464 |
| 163 | hsa-miR-4734 | MIMAT0019859 |
| 164 | hsa-miR-3162-5p | MIMAT0015036 |
| 165 | hsa-miR-887-3p | MIMAT0004951 |
| 166 | hsa-miR-6752-5p | MIMAT0027404 |
| 167 | hsa-miR-6724-5p | MIMAT0025856 |
| 168 | hsa-miR-23b-3p | MIMAT0000418 |
| 169 | hsa-miR-23a-3p | MIMAT0000078 |
| 170 | hsa-miR-625-3p | MIMAT0004808 |
| 171 | hsa-miR-1228-3p | MIMAT0005583 |
| 172 | hsa-miR-614 | MIMAT0003282 |
| 173 | hsa-miR-1913 | MIMAT0007888 |
| 174 | hsa-miR-92a-2-5p | MIMAT0004508 |
| 175 | hsa-miR-187-5p | MIMAT0004561 |
| 176 | hsa-miR-16-5p | MIMAT0000069 |
| 177 | hsa-miR-92b-3p | MIMAT0003218 |
| 178 | hsa-miR-150-3p | MIMAT0004610 |
| 179 | hsa-miR-564 | MIMAT0003228 |
| 180 | hsa-miR-125a-3p | MIMAT0004602 |
| 181 | hsa-miR-92b-5p | MIMAT0004792 |
| 182 | hsa-miR-92a-3p | MIMAT0000092 |
| 183 | hsa-miR-663a | MIMAT0003326 |
| 184 | hsa-miR-4688 | MIMAT0019777 |
| 185 | hsa-miR-4648 | MIMAT0019710 |
| 186 | hsa-miR-6085 | MIMAT0023710 |
| 187 | hsa-miR-6126 | MIMAT0024599 |
| 188 | hsa-miR-6880-5p | MIMAT0027660 |
| 189 | hsa-miR-328-5p | MIMAT0026486 |
| 190 | hsa-miR-6768-5p | MIMAT0027436 |
| 191 | hsa-miR-3180 | MIMAT0018178 |
| 192 | hsa-miR-6087 | MIMAT0023712 |
| 193 | hsa-miR-1273g-3p | MIMAT0022742 |
| 194 | hsa-miR-1225-5p | MIMAT0005572 |
| 195 | hsa-miR-3196 | MIMAT0015080 |
| 196 | hsa-miR-4695-5p | MIMAT0019788 |
| 197 | hsa-miR-6732-5p | MIMAT0027365 |
| 198 | hsa-miR-638 | MIMAT0003308 |
| 199 | hsa-miR-6813-5p | MIMAT0027526 |
| 200 | hsa-miR-665 | MIMAT0004952 |
| 201 | hsa-miR-486-3p | MIMAT0004762 |
| 202 | hsa-miR-4466 | MIMAT0018993 |
| 203 | hsa-miR-30c-1-3p | MIMAT0004674 |
| 204 | hsa-miR-3621 | MIMAT0018002 |
| 205 | hsa-miR-6743-5p | MIMAT0027387 |
| 206 | hsa-miR-4298 | MIMAT0016852 |
| 207 | hsa-miR-4741 | MIMAT0019871 |
| 208 | hsa-miR-3619-3p | MIMAT0019219 |
| 209 | hsa-miR-6824-5p | MIMAT0027548 |
| 210 | hsa-miR-5698 | MIMAT0022491 |
| 211 | hsa-miR-371a-5p | MIMAT0004687 |
| 212 | hsa-miR-4488 | MIMAT0019022 |
| 213 | hsa-miR-1233-5p | MIMAT0022943 |
| 214 | hsa-miR-4723-5p | MIMAT0019838 |
| 215 | hsa-miR-24-3p | MIMAT0000080 |
| 216 | hsa-miR-1238-5p | MIMAT0022947 |
| 217 | hsa-miR-4442 | MIMAT0018960 |
| 218 | hsa-miR-3928-3p | MIMAT0018205 |
| 219 | hsa-miR-6716-5p | MIMAT0025844 |
| 220 | hsa-miR-6089 | MIMAT0023714 |
| 221 | hsa-miR-6124 | MIMAT0024597 |
| 222 | hsa-miR-6778-5p | MIMAT0027456 |
| 223 | hsa-miR-557 | MIMAT0003221 |
| 224 | hsa-miR-6090 | MIMAT0023715 |
| 225 | hsa-mir-1343 | MI0017320 |
| 226 | hsa-mir-6726 | MI0022571 |
| 227 | hsa-mir-6515 | MI0022227 |
| 228 | hsa-mir-4651 | MI0017279 |
| 229 | hsa-mir-4257 | MI0015856 |
| 230 | hsa-mir-3188 | MI0014232 |
| 231 | hsa-mir-6131 | MI0021276 |
| 232 | hsa-mir-6766 | MI0022611 |
| 233 | hsa-mir-7641-1 | MI0024975 |
| 234 | hsa-mir-7641-2 | MI0024976 |
| 235 | hsa-mir-1249 | MI0006384 |
| 236 | hsa-mir-3679 | MI0016080 |
| 237 | hsa-mir-6787 | MI0022632 |
| 238 | hsa-mir-4454 | MI0016800 |
| 239 | hsa-mir-3135b | MI0016809 |
| 240 | hsa-mir-6765 | MI0022610 |
| 241 | hsa-mir-7975 | MI0025751 |
| 242 | hsa-mir-204 | MI0000284 |
| 243 | hsa-mir-7977 | MI0025753 |
| 244 | hsa-mir-7110 | MI0022961 |
| 245 | hsa-mir-6717 | MI0022551 |
| 246 | hsa-mir-6870 | MI0022717 |
| 247 | hsa-mir-663b | MI0006336 |
| 248 | hsa-mir-6875 | MI0022722 |
| 249 | hsa-mir-8072 | MI0025908 |
| 250 | hsa-mir-6816 | MI0022661 |
| 251 | hsa-mir-4281 | MI0015885 |
| 252 | hsa-mir-6729 | MI0022574 |
| 253 | hsa-mir-8069 | MI0025905 |
| 254 | hsa-mir-4706 | MI0017339 |
| 255 | hsa-mir-7108 | MI0022959 |
| 256 | hsa-mir-4433b | MI0025511 |
| 257 | hsa-mir-6893 | MI0022740 |
| 258 | hsa-mir-6857 | MI0022703 |
| 259 | hsa-mir-1227 | MI0006316 |
| 260 | hsa-mir-6741 | MI0022586 |
| 261 | hsa-mir-451a | MI0001729 |
| 262 | hsa-mir-8063 | MI0025899 |
| 263 | hsa-mir-3622a | MI0016013 |
| 264 | hsa-mir-615 | MI0003628 |
| 265 | hsa-mir-128-1 | MI0000447 |
| 266 | hsa-mir-6825 | MI0022670 |
| 267 | hsa-mir-1260b | MI0014197 |
| 268 | hsa-mir-4433 | MI0016773 |
| 269 | hsa-mir-4665 | MI0017295 |
| 270 | hsa-mir-7845 | MI0025515 |
| 271 | hsa-mir-1908 | MI0008329 |
| 272 | hsa-mir-6840 | MI0022686 |
| 240 | hsa-mir-6765 | MI0022610 |
| 273 | hsa-mir-296 | MI0000747 |
| 274 | hsa-mir-3675 | MI0016076 |
| 275 | hsa-mir-6781 | MI0022626 |
| 276 | hsa-mir-423 | MI0001445 |
| 277 | hsa-mir-3663 | MI0016064 |
| 278 | hsa-mir-6784 | MI0022629 |
| 279 | hsa-mir-6749 | MI0022594 |
| 280 | hsa-mir-1231 | MI0006321 |
| 281 | hsa-mir-4746 | MI0017385 |
| 282 | hsa-mir-6780b | MI0022681 |
| 283 | hsa-mir-4758 | MI0017399 |
| 236 | hsa-mir-3679 | MI0016080 |
| 284 | hsa-mir-3184 | MI0014226 |
| 285 | hsa-mir-6125 | MI0021259 |
| 286 | hsa-mir-6721 | MI0022556 |
| 287 | hsa-mir-6791 | MI0022636 |
| 288 | hsa-mir-3185 | MI0014227 |
| 289 | hsa-mir-1260a | MI0006394 |
| 290 | hsa-mir-3197 | MI0014245 |
| 291 | hsa-mir-6845 | MI0022691 |
| 292 | hsa-mir-6887 | MI0022734 |
| 293 | hsa-mir-6738 | MI0022583 |
| 294 | hsa-mir-6872 | MI0022719 |
| 295 | hsa-mir-4497 | MI0016859 |
| 296 | hsa-mir-1229 | MI0006319 |
| 297 | hsa-mir-6820 | MI0022665 |
| 298 | hsa-mir-6777 | MI0022622 |
| 299 | hsa-mir-3917 | MI0016423 |
| 300 | hsa-mir-5787 | MI0019797 |
| 301 | hsa-mir-4286 | MI0015894 |
| 302 | hsa-mir-6877 | MI0022724 |
| 303 | hsa-mir-1225 | MI0006311 |
| 304 | hsa-mir-6088 | MI0020365 |
| 305 | hsa-mir-6800 | MI0022645 |
| 306 | hsa-mir-1246 | MI0006381 |
| 307 | hsa-mir-4467 | MI0016818 |
| 308 | hsa-mir-4419b | MI0016861 |
| 309 | hsa-mir-1914 | MI0008335 |
| 310 | hsa-mir-4632 | MI0017259 |
| 311 | hsa-mir-1915 | MI0008336 |
| 312 | hsa-mir-3940 | MI0016597 |
| 313 | hsa-mir-1185-2 | MI0003821 |
| 314 | hsa-mir-6746 | MI0022591 |
| 315 | hsa-mir-5001 | MI0017867 |
| 316 | hsa-mir-1228 | MI0006318 |
| 317 | hsa-mir-5572 | MI0019117 |
| 318 | hsa-mir-4327 | MI0015867 |
| 319 | hsa-mir-4638 | MI0017265 |
| 320 | hsa-mir-6799 | MI0022644 |
| 321 | hsa-mir-6861 | MI0022708 |
| 322 | hsa-mir-6727 | MI0022572 |
| 323 | hsa-mir-4513 | MI0016879 |
| 324 | hsa-mir-6805 | MI0022650 |
| 325 | hsa-mir-6808 | MI0022653 |
| 326 | hsa-mir-4449 | MI0016792 |
| 327 | hsa-mir-1199 | MI0020340 |
| 328 | hsa-mir-1275 | MI0006415 |
| 329 | hsa-mir-4792 | MI0017439 |
| 330 | hsa-mir-4443 | MI0016786 |
| 331 | hsa-mir-6891 | MI0022738 |
| 332 | hsa-mir-6826 | MI0022671 |
| 333 | hsa-mir-6807 | MI0022652 |
| 334 | hsa-mir-7150 | MI0023610 |
| 335 | hsa-mir-4534 | MI0016901 |
| 336 | hsa-mir-4476 | MI0016828 |
| 337 | hsa-mir-4649 | MI0017276 |
| 338 | hsa-mir-4525 | MI0016892 |
| 311 | hsa-mir-1915 | MI0008336 |
| 339 | hsa-mir-4516 | MI0016882 |
| 340 | hsa-mir-4417 | MI0016753 |
| 341 | hsa-mir-642b | MI0016685 |
| 342 | hsa-mir-3141 | MI0014165 |
| 343 | hsa-mir-5100 | MI0019116 |
| 344 | hsa-mir-6848 | MI0022694 |
| 345 | hsa-mir-4739 | MI0017377 |
| 346 | hsa-mir-4459 | MI0016805 |
| 347 | hsa-mir-1237 | MI0006327 |
| 273 | hsa-mir-296 | MI0000747 |
| 269 | hsa-mir-4665 | MI0017295 |
| 348 | hsa-mir-6786 | MI0022631 |
| 349 | hsa-mir-4258 | MI0015857 |
| 350 | hsa-mir-6510 | MI0022222 |
| 225 | hsa-mir-1343 | MI0017320 |
| 351 | hsa-mir-1247 | MI0006382 |
| 324 | hsa-mir-6805 | MI0022650 |
| 352 | hsa-mir-4492 | MI0016854 |
| 353 | hsa-mir-1469 | MI0007074 |
| 354 | hsa-mir-1268b | MI0016748 |
| 355 | hsa-mir-6858 | MI0022704 |
| 356 | hsa-mir-3937 | MI0016593 |
| 357 | hsa-mir-939 | MI0005761 |
| 358 | hsa-mir-3656 | MI0016056 |
| 359 | hsa-mir-744 | MI0005559 |
| 360 | hsa-mir-4687 | MI0017319 |
| 361 | hsa-mir-4763 | MI0017404 |
| 362 | hsa-mir-3620 | MI0016011 |
| 363 | hsa-mir-3195 | MI0014240 |
| 364 | hsa-mir-6842 | MI0022688 |
| 365 | hsa-mir-4707 | MI0017340 |
| 366 | hsa-mir-642a | MI0003657 |
| 367 | hsa-mir-7113 | MI0022964 |
| 368 | hsa-mir-4728 | MI0017365 |
| 369 | hsa-mir-5195 | MI0018174 |
| 370 | hsa-mir-1185-1 | MI0003844 |
| 371 | hsa-mir-6774 | MI0022619 |
| 372 | hsa-mir-8059 | MI0025895 |
| 373 | hsa-mir-3131 | MI0014151 |
| 374 | hsa-mir-7847 | MI0025517 |
| 375 | hsa-mir-4463 | MI0016811 |
| 376 | hsa-mir-128-2 | MI0000727 |
| 377 | hsa-mir-4508 | MI0016872 |
| 378 | hsa-mir-6806 | MI0022651 |
| 379 | hsa-mir-7111 | MI0022962 |
| 380 | hsa-mir-6782 | MI0022627 |
| 381 | hsa-mir-4734 | MI0017371 |
| 382 | hsa-mir-3162 | MI0014192 |
| 383 | hsa-mir-887 | MI0005562 |
| 384 | hsa-mir-6752 | MI0022597 |
| 385 | hsa-mir-6724 | MI0022559 |
| 386 | hsa-mir-23b | MI0000439 |
| 387 | hsa-mir-23a | MI0000079 |
| 388 | hsa-mir-625 | MI0003639 |
| 316 | hsa-mir-1228 | MI0006318 |
| 389 | hsa-mir-614 | MI0003627 |
| 390 | hsa-mir-1913 | MI0008334 |
| 391 | hsa-mir-92a-2 | MI0000094 |
| 392 | hsa-mir-187 | MI0000274 |
| 393 | hsa-mir-16-1 | MI0000070 |
| 394 | hsa-mir-16-2 | MI0000115 |
| 395 | hsa-mir-92b | MI0003560 |
| 396 | hsa-mir-150 | MI0000479 |
| 397 | hsa-mir-564 | MI0003570 |
| 398 | hsa-mir-125a | MI0000469 |
| 395 | hsa-mir-92b | MI0003560 |
| 399 | hsa-mir-92a-1 | MI0000093 |
| 391 | hsa-mir-92a-2 | MI0000094 |
| 400 | hsa-mir-663a | MI0003672 |
| 401 | hsa-mir-4688 | MI0017321 |
| 402 | hsa-mir-4648 | MI0017275 |
| 403 | hsa-mir-6085 | MI0020362 |
| 404 | hsa-mir-6126 | MI0021260 |
| 405 | hsa-mir-6880 | MI0022727 |
| 406 | hsa-mir-328 | MI0000804 |
| 407 | hsa-mir-6768 | MI0022613 |
| 408 | hsa-mir-3180-4 | MI0016408 |
| 409 | hsa-mir-3180-5 | MI0016409 |
| 410 | hsa-mir-6087 | MI0020364 |
| 411 | hsa-mir-1273g | MI0018003 |
| 303 | hsa-mir-1225 | MI0006311 |
| 412 | hsa-mir-3196 | MI0014241 |
| 413 | hsa-mir-4695 | MI0017328 |
| 414 | hsa-mir-6732 | MI0022577 |
| 415 | hsa-mir-638 | MI0003653 |
| 416 | hsa-mir-6813 | MI0022658 |
| 417 | hsa-mir-665 | MI0005563 |
| 418 | hsa-mir-486 | MI0002470 |
| 419 | hsa-mir-486-2 | MI0023622 |
| 420 | hsa-mir-4466 | MI0016817 |
| 421 | hsa-mir-30c-1 | MI0000736 |
| 422 | hsa-mir-3621 | MI0016012 |
| 423 | hsa-mir-6743 | MI0022588 |
| 424 | hsa-mir-4298 | MI0015830 |
| 425 | hsa-mir-4741 | MI0017379 |
| 426 | hsa-mir-3619 | MI0016009 |
| 427 | hsa-mir-6824 | MI0022669 |
| 428 | hsa-mir-5698 | MI0019305 |
| 429 | hsa-mir-371a | MI0000779 |
| 430 | hsa-mir-4488 | MI0016849 |
| 431 | hsa-mir-1233-1 | MI0006323 |
| 432 | hsa-mir-1233-2 | MI0015973 |
| 433 | hsa-mir-4723 | MI0017359 |
| 434 | hsa-mir-24-1 | MI0000080 |
| 435 | hsa-mir-24-2 | MI0000081 |
| 436 | hsa-mir-1238 | MI0006328 |
| 437 | hsa-mir-4442 | MI0016785 |
| 438 | hsa-mir-3928 | MI0016438 |
| 439 | hsa-mir-6716 | MI0022550 |
| 440 | hsa-mir-6089-1 | MI0020366 |
| 441 | hsa-mir-6089-2 | MI0023563 |
| 442 | hsa-mir-6124 | MI0021258 |
| 443 | hsa-mir-6778 | MI0022623 |
| 444 | hsa-mir-557 | MI0003563 |
| 445 | hsa-mir-6090 | MI0020367 |
| 446 | isomiR example 1 of SEQ ID NO: 1 | — |
| 447 | isomiR example 2 of SEQ ID NO: 1 | — |
| 448 | isomiR example 1 of SEQ ID NO: 3 | — |
| 449 | isomiR example 2 of SEQ ID NO: 3 | — |
| 450 | isomiR example 1 of SEQ ID NO: 4 | — |
| 451 | isomiR example 2 of SEQ ID NO: 4 | — |
| 452 | isomiR example 1 of SEQ ID NO: 6 | — |
| 453 | isomiR example 2 of SEQ ID NO: 6 | — |
| 454 | isomiR example 1 of SEQ ID NO: 7 | — |
| 455 | isomiR example 2 of SEQ ID NO: 7 | — |
| 456 | isomiR example 1 of SEQ ID NO: 10 | — |
| 457 | isomiR example 2 of SEQ ID NO: 10 | — |
| 458 | isomiR example 1 of SEQ ID NO: 11 | — |
| 459 | isomiR example 2 of SEQ ID NO: 11 | — |
| 460 | isomiR example 1 of SEQ ID NO: 13 | — |
| 461 | isomiR example 2 of SEQ ID NO: 13 | — |
| 462 | isomiR example 1 of SEQ ID NO: 14 | — |
| 463 | isomiR example 2 of SEQ ID NO: 14 | — |
| 464 | isomiR example 1 of SEQ ID NO: 16 | — |
| 465 | isomiR example 2 of SEQ ID NO: 16 | — |
| 466 | isomiR example 1 of SEQ ID NO: 17 | — |
| 467 | isomiR example 2 of SEQ ID NO: 17 | — |
| 468 | isomiR example 1 of SEQ ID NO: 20 | — |
| 469 | isomiR example 2 of SEQ ID NO: 20 | — |
| 470 | isomiR example 1 of SEQ ID NO: 22 | — |
| 471 | isomiR example 2 of SEQ ID NO: 22 | — |
| 472 | isomiR example 1 of SEQ ID NO: 26 | — |
| 473 | isomiR example 2 of SEQ ID NO: 26 | — |
| 474 | isomiR example 1 of SEQ ID NO: 29 | — |
| 475 | isomiR example 2 of SEQ ID NO: 29 | — |
| 476 | isomiR example 1 of SEQ ID NO: 36 | — |
| 477 | isomiR example 2 of SEQ ID NO: 36 | — |
| 478 | isomiR example 1 of SEQ ID NO: 38 | — |
| 479 | isomiR example 2 of SEQ ID NO: 38 | — |
| 480 | isomiR example 1 of SEQ ID NO: 39 | — |
| 481 | isomiR example 2 of SEQ ID NO: 39 | — |
| 482 | isomiR example 1 of SEQ ID NO: 40 | — |
| 483 | isomiR example 2 of SEQ ID NO: 40 | — |
| 484 | isomiR example 1 of SEQ ID NO: 42 | — |
| 485 | isomiR example 2 of SEQ ID NO: 42 | — |
| 486 | isomiR example 1 of SEQ ID NO: 43 | — |
| 487 | isomiR example 2 of SEQ ID NO: 43 | — |
| 488 | isomiR example 1 of SEQ ID NO: 44 | — |
| 489 | isomiR example 2 of SEQ ID NO: 44 | — |
| 490 | isomiR example 1 of SEQ ID NO: 46 | — |
| 491 | isomiR example 2 of SEQ ID NO: 46 | — |
| 492 | isomiR example 1 of SEQ ID NO: 49 | — |
| 493 | isomiR example 2 of SEQ ID NO: 49 | — |
| 494 | isomiR example 1 of SEQ ID NO: 52 | — |
| 495 | isomiR example 2 of SEQ ID NO: 52 | — |
| 496 | isomiR example 1 of SEQ ID NO: 59 | — |
| 497 | isomiR example 2 of SEQ ID NO: 59 | — |
| 498 | isomiR example 1 of SEQ ID NO: 60 | — |
| 499 | isomiR example 2 of SEQ ID NO: 60 | — |
| 500 | isomiR example 1 of SEQ ID NO: 62 | — |
| 501 | isomiR example 2 of SEQ ID NO: 62 | — |
| 502 | isomiR example 1 of SEQ ID NO: 63 | — |
| 503 | isomiR example 2 of SEQ ID NO: 63 | — |
| 504 | isomiR example 1 of SEQ ID NO: 65 | — |
| 505 | isomiR example 2 of SEQ ID NO: 65 | — |
| 506 | isomiR example 1 of SEQ ID NO: 66 | — |
| 507 | isomiR example 2 of SEQ ID NO: 66 | — |
| 508 | isomiR example 1 of SEQ ID NO: 67 | — |
| 509 | isomiR example 2 of SEQ ID NO: 67 | — |
| 510 | isomiR example 1 of SEQ ID NO: 72 | — |
| 511 | isomiR example 2 of SEQ ID NO: 72 | — |
| 512 | isomiR example 1 of SEQ ID NO: 76 | — |
| 513 | isomiR example 2 of SEQ ID NO: 76 | — |
| 514 | isomiR example 1 of SEQ ID NO: 77 | — |
| 515 | isomiR example 2 of SEQ ID NO: 77 | — |
| 516 | isomiR example 1 of SEQ ID NO: 78 | — |
| 517 | isomiR example 2 of SEQ ID NO: 78 | — |
| 518 | isomiR example 1 of SEQ ID NO: 81 | — |
| 519 | isomiR example 2 of SEQ ID NO: 81 | — |
| 520 | isomiR example 1 of SEQ ID NO: 83 | — |
| 521 | isomiR example 2 of SEQ ID NO: 83 | — |
| 522 | isomiR example 1 of SEQ ID NO: 84 | — |
| 523 | isomiR example 2 of SEQ ID NO: 84 | — |
| 524 | isomiR example 1 of SEQ ID NO: 85 | — |
| 525 | isomiR example 2 of SEQ ID NO: 85 | — |
| 526 | isomiR example 1 of SEQ ID NO: 86 | — |
| 527 | isomiR example 2 of SEQ ID NO: 86 | — |
| 528 | isomiR example 1 of SEQ ID NO: 87 | — |
| 529 | isomiR example 2 of SEQ ID NO: 87 | — |
| 530 | isomiR example 1 of SEQ ID NO: 88 | — |
| 531 | isomiR example 2 of SEQ ID NO: 88 | — |
| 532 | isomiR example 1 of SEQ ID NO: 89 | — |
| 533 | isomiR example 2 of SEQ ID NO: 89 | — |
| 534 | isomiR example 1 of SEQ ID NO: 90 | — |
| 535 | isomiR example 2 of SEQ ID NO: 90 | — |
| 536 | isomiR example 1 of SEQ ID NO: 92 | — |
| 537 | isomiR example 2 of SEQ ID NO: 92 | — |
| 538 | isomiR example 1 of SEQ ID NO: 93 | — |
| 539 | isomiR example 2 of SEQ ID NO: 93 | — |
| 540 | isomiR example 1 of SEQ ID NO: 94 | — |
| 541 | isomiR example 2 of SEQ ID NO: 94 | — |
| 542 | isomiR example 1 of SEQ ID NO: 96 | — |
| 543 | isomiR example 2 of SEQ ID NO: 96 | — |
| 544 | isomiR example 1 of SEQ ID NO: 100 | — |
| 545 | isomiR example 2 of SEQ ID NO: 100 | — |
| 546 | isomiR example 1 of SEQ ID NO: 103 | — |
| 547 | isomiR example 2 of SEQ ID NO: 103 | — |
| 548 | isomiR example 1 of SEQ ID NO: 105 | — |
| 549 | isomiR example 2 of SEQ ID NO: 105 | — |
| 550 | isomiR example 1 of SEQ ID NO: 106 | — |
| 551 | isomiR example 2 of SEQ ID NO: 106 | — |
| 552 | isomiR example 1 of SEQ ID NO: 107 | — |
| 553 | isomiR example 2 of SEQ ID NO: 107 | — |
| 554 | isomiR example 1 of SEQ ID NO: 113 | — |
| 555 | isomiR example 2 of SEQ ID NO: 113 | — |
| 556 | isomiR example 1 of SEQ ID NO: 114 | — |
| 557 | isomiR example 2 of SEQ ID NO: 114 | — |
| 558 | isomiR example 1 of SEQ ID NO: 115 | — |
| 559 | isomiR example 2 of SEQ ID NO: 115 | — |
| 560 | isomiR example 1 of SEQ ID NO: 116 | — |
| 561 | isomiR example 2 of SEQ ID NO: 116 | — |
| 562 | isomiR example 1 of SEQ ID NO: 117 | — |
| 563 | isomiR example 2 of SEQ ID NO: 117 | — |
| 564 | isomiR example 1 of SEQ ID NO: 118 | — |
| 565 | isomiR example 2 of SEQ ID NO: 118 | — |
| 566 | isomiR example 1 of SEQ ID NO: 119 | — |
| 567 | isomiR example 2 of SEQ ID NO: 119 | — |
| 568 | isomiR example 1 of SEQ ID NO: 120 | — |
| 569 | isomiR example 2 of SEQ ID NO: 120 | — |
| 570 | isomiR example 1 of SEQ ID NO: 121 | — |
| 571 | isomiR example 2 of SEQ ID NO: 121 | — |
| 572 | isomiR example 1 of SEQ ID NO: 123 | — |
| 573 | isomiR example 2 of SEQ ID NO: 123 | — |
| 574 | isomiR example 1 of SEQ ID NO: 124 | — |
| 575 | isomiR example 2 of SEQ ID NO: 124 | — |
| 576 | isomiR example 1 of SEQ ID NO: 125 | — |
| 577 | isomiR example 2 of SEQ ID NO: 125 | — |
| 578 | isomiR example 1 of SEQ ID NO: 126 | — |
| 579 | isomiR example 2 of SEQ ID NO: 126 | — |
| 580 | isomiR example 1 of SEQ ID NO: 130 | — |
| 581 | isomiR example 2 of SEQ ID NO: 130 | — |
| 582 | isomiR example 1 of SEQ ID NO: 132 | — |
| 583 | isomiR example 2 of SEQ ID NO: 132 | — |
| 584 | isomiR example 1 of SEQ ID NO: 134 | — |
| 585 | isomiR example 2 of SEQ ID NO: 134 | — |
| 586 | isomiR example 1 of SEQ ID NO: 136 | — |
| 587 | isomiR example 2 of SEQ ID NO: 136 | — |
| 588 | isomiR example 1 of SEQ ID NO: 139 | — |
| 589 | isomiR example 2 of SEQ ID NO: 139 | — |
| 590 | isomiR example 1 of SEQ ID NO: 140 | — |
| 591 | isomiR example 2 of SEQ ID NO: 140 | — |
| 592 | isomiR example 1 of SEQ ID NO: 141 | — |
| 593 | isomiR example 2 of SEQ ID NO: 141 | — |
| 594 | isomiR example 1 of SEQ ID NO: 142 | — |
| 595 | isomiR example 2 of SEQ ID NO: 142 | — |
| 596 | isomiR example 1 of SEQ ID NO: 143 | — |
| 597 | isomiR example 2 of SEQ ID NO: 143 | — |
| 598 | isomiR example 1 of SEQ ID NO: 144 | — |
| 599 | isomiR example 2 of SEQ ID NO: 144 | — |
| 600 | isomiR example 1 of SEQ ID NO: 145 | — |
| 601 | isomiR example 2 of SEQ ID NO: 145 | — |
| 602 | isomiR example 1 of SEQ ID NO: 147 | — |
| 603 | isomiR example 2 of SEQ ID NO: 147 | — |
| 604 | isomiR example 1 of SEQ ID NO: 148 | — |
| 605 | isomiR example 2 of SEQ ID NO: 148 | — |
| 606 | isomiR example 1 of SEQ ID NO: 150 | — |
| 607 | isomiR example 2 of SEQ ID NO: 150 | — |
| 608 | isomiR example 1 of SEQ ID NO: 151 | — |
| 609 | isomiR example 2 of SEQ ID NO: 151 | — |
| 610 | isomiR example 1 of SEQ ID NO: 152 | — |
| 611 | isomiR example 2 of SEQ ID NO: 152 | — |
| 612 | isomiR example 1 of SEQ ID NO: 155 | — |
| 613 | isomiR example 2 of SEQ ID NO: 155 | — |
| 614 | isomiR example 1 of SEQ ID NO: 157 | — |
| 615 | isomiR example 2 of SEQ ID NO: 157 | — |
| 616 | isomiR example 1 of SEQ ID NO: 158 | — |
| 617 | isomiR example 2 of SEQ ID NO: 158 | — |
| 618 | isomiR example 1 of SEQ ID NO: 159 | — |
| 619 | isomiR example 2 of SEQ ID NO: 159 | — |
| 620 | isomiR example 1 of SEQ ID NO: 163 | — |
| 621 | isomiR example 2 of SEQ ID NO: 163 | — |
| 622 | isomiR example 1 of SEQ ID NO: 164 | — |
| 623 | isomiR example 2 of SEQ ID NO: 164 | — |
| 624 | isomiR example 1 of SEQ ID NO: 165 | — |
| 625 | isomiR example 2 of SEQ ID NO: 165 | — |
| 626 | isomiR example 1 of SEQ ID NO: 167 | — |
| 627 | isomiR example 2 of SEQ ID NO: 167 | — |
| 628 | isomiR example 1 of SEQ ID NO: 168 | — |
| 629 | isomiR example 2 of SEQ ID NO: 168 | — |
| 630 | isomiR example 1 of SEQ ID NO: 169 | — |
| 631 | isomiR example 2 of SEQ ID NO: 169 | — |
| 632 | isomiR example 1 of SEQ ID NO: 170 | — |
| 633 | isomiR example 2 of SEQ ID NO: 170 | — |
| 634 | isomiR example 1 of SEQ ID NO: 171 | — |
| 635 | isomiR example 2 of SEQ ID NO: 171 | — |
| 636 | isomiR example 1 of SEQ ID NO: 172 | — |
| 637 | isomiR example 2 of SEQ ID NO: 172 | — |
| 638 | isomiR example 1 of SEQ ID NO: 173 | — |
| 639 | isomiR example 2 of SEQ ID NO: 173 | — |
| 640 | isomiR example 1 of SEQ ID NO: 174 | — |
| 641 | isomiR example 2 of SEQ ID NO: 174 | — |
| 642 | isomiR example 1 of SEQ ID NO: 175 | — |
| 643 | isomiR example 2 of SEQ ID NO: 175 | — |
| 644 | isomiR example 1 of SEQ ID NO: 176 | — |
| 645 | isomiR example 2 of SEQ ID NO: 176 | — |
| 646 | isomiR example 1 of SEQ ID NO: 177 | — |
| 647 | isomiR example 2 of SEQ ID NO: 177 | — |
| 648 | isomiR example 1 of SEQ ID NO: 178 | — |
| 649 | isomiR example 2 of SEQ ID NO: 178 | — |
| 650 | isomiR example 1 of SEQ ID NO: 179 | — |
| 651 | isomiR example 2 of SEQ ID NO: 179 | — |
| 652 | isomiR example 1 of SEQ ID NO: 180 | — |
| 653 | isomiR example 2 of SEQ ID NO: 180 | — |
| 654 | isomiR example 1 of SEQ ID NO: 181 | — |
| 655 | isomiR example 2 of SEQ ID NO: 181 | — |
| 656 | isomiR example 1 of SEQ ID NO: 182 | — |
| 657 | isomiR example 2 of SEQ ID NO: 182 | — |
| 658 | isomiR example 1 of SEQ ID NO: 183 | — |
| 659 | isomiR example 2 of SEQ ID NO: 183 | — |
| 660 | isomiR example 1 of SEQ ID NO: 184 | — |
| 661 | isomiR example 2 of SEQ ID NO: 184 | — |
| 662 | isomiR example 1 of SEQ ID NO: 185 | — |
| 663 | isomiR example 2 of SEQ ID NO: 185 | — |
| 664 | isomiR example 1 of SEQ ID NO: 187 | — |
| 665 | isomiR example 2 of SEQ ID NO: 187 | — |
| 666 | isomiR example 1 of SEQ ID NO: 189 | — |
| 667 | isomiR example 2 of SEQ ID NO: 189 | — |
| 668 | isomiR example 1 of SEQ ID NO: 191 | — |
| 669 | isomiR example 2 of SEQ ID NO: 191 | — |
| 670 | isomiR example 1 of SEQ ID NO: 192 | — |
| 671 | isomiR example 2 of SEQ ID NO: 192 | — |
| 672 | isomiR example 1 of SEQ ID NO: 193 | — |
| 673 | isomiR example 2 of SEQ ID NO: 193 | — |
| 674 | isomiR example 1 of SEQ ID NO: 195 | — |
| 675 | isomiR example 2 of SEQ ID NO: 195 | — |
| 676 | isomiR example 1 of SEQ ID NO: 196 | — |
| 677 | isomiR example 2 of SEQ ID NO: 196 | — |
| 678 | isomiR example 1 of SEQ ID NO: 198 | — |
| 679 | isomiR example 2 of SEQ ID NO: 198 | — |
| 680 | isomiR example 1 of SEQ ID NO: 200 | — |
| 681 | isomiR example 2 of SEQ ID NO: 200 | — |
| 682 | isomiR example 1 of SEQ ID NO: 201 | — |
| 683 | isomiR example 2 of SEQ ID NO: 201 | — |
| 684 | isomiR example 1 of SEQ ID NO: 202 | — |
| 685 | isomiR example 2 of SEQ ID NO: 202 | — |
| 686 | isomiR example 1 of SEQ ID NO: 203 | — |
| 687 | isomiR example 2 of SEQ ID NO: 203 | — |
| 688 | isomiR example 1 of SEQ ID NO: 206 | — |
| 689 | isomiR example 2 of SEQ ID NO: 206 | — |
| 690 | isomiR example 1 of SEQ ID NO: 207 | — |
| 691 | isomiR example 2 of SEQ ID NO: 207 | — |
| 692 | isomiR example 1 of SEQ ID NO: 210 | — |
| 693 | isomiR example 2 of SEQ ID NO: 210 | — |
| 694 | isomiR example 1 of SEQ ID NO: 211 | — |
| 695 | isomiR example 2 of SEQ ID NO: 211 | — |
| 696 | isomiR example 1 of SEQ ID NO: 212 | — |
| 697 | isomiR example 2 of SEQ ID NO: 212 | — |
| 698 | isomiR example 1 of SEQ ID NO: 213 | — |
| 699 | isomiR example 2 of SEQ ID NO: 213 | — |
| 700 | isomiR example 1 of SEQ ID NO: 214 | — |
| 701 | isomiR example 2 of SEQ ID NO: 214 | — |
| 702 | isomiR example 1 of SEQ ID NO: 215 | — |
| 703 | isomiR example 2 of SEQ ID NO: 215 | — |
| 704 | isomiR example 1 of SEQ ID NO: 217 | — |
| 705 | isomiR example 2 of SEQ ID NO: 217 | — |
| 706 | isomiR example 1 of SEQ ID NO: 218 | — |
| 707 | isomiR example 2 of SEQ ID NO: 218 | — |
| 708 | isomiR example 1 of SEQ ID NO: 219 | — |
| 709 | isomiR example 2 of SEQ ID NO: 219 | — |
| 710 | isomiR example 1 of SEQ ID NO: 220 | — |
| 711 | isomiR example 2 of SEQ ID NO: 220 | — |
| 712 | isomiR example 1 of SEQ ID NO: 221 | — |
| 713 | isomiR example 2 of SEQ ID NO: 221 | — |
| 714 | hsa-miR-6757-5p | MIMAT0027414 |
| 715 | hsa-miR-4448 | MIMAT0018967 |
| 716 | hsa-miR-671-5p | MIMAT0003880 |
| 717 | hsa-miR-3178 | MIMAT0015055 |
| 718 | hsa-miR-4725-3p | MIMAT0019844 |
| 719 | hsa-miR-940 | MIMAT0004983 |
| 720 | hsa-miR-6789-5p | MIMAT0027478 |
| 721 | hsa-miR-4484 | MIMAT0019018 |
| 722 | hsa-miR-4634 | MIMAT0019691 |
| 723 | hsa-miR-4745-5p | MIMAT0019878 |
| 724 | hsa-miR-4730 | MIMAT0019852 |
| 725 | hsa-miR-6803-5p | MIMAT0027506 |
| 726 | hsa-miR-6798-5p | MIMAT0027496 |
| 727 | hsa-miR-3648 | MIMAT0018068 |
| 728 | hsa-miR-4783-3p | MIMAT0019947 |
| 729 | hsa-miR-6836-3p | MIMAT0027575 |
| 730 | hsa-mir-6757 | MI0022602 |
| 731 | hsa-mir-4448 | MI0016791 |
| 732 | hsa-mir-671 | MI0003760 |
| 733 | hsa-mir-3178 | MI0014212 |
| 734 | hsa-mir-4725 | MI0017362 |
| 735 | hsa-mir-940 | MI0005762 |
| 736 | hsa-mir-6789 | MI0022634 |
| 737 | hsa-mir-4484 | MI0016845 |
| 738 | hsa-mir-4634 | MI0017261 |
| 739 | hsa-mir-4745 | MI0017384 |
| 740 | hsa-mir-4730 | MI0017367 |
| 741 | hsa-mir-6803 | MI0022648 |
| 742 | hsa-mir-6798 | MI0022643 |
| 743 | hsa-mir-3648 | MI0016048 |
| 744 | hsa-mir-4783 | MI0017428 |
| 745 | hsa-mir-6836 | MI0022682 |
| 746 | isomiR example 1 of SEQ ID NO: 715 | — |
| 747 | isomiR example 2 of SEQ ID NO: 715 | — |
| 748 | isomiR example 1 of SEQ ID NO: 716 | — |
| 749 | isomiR example 2 of SEQ ID NO: 716 | — |
| 750 | isomiR example 1 of SEQ ID NO: 717 | — |
| 751 | isomiR example 2 of SEQ ID NO: 717 | — |
| 752 | isomiR example 1 of SEQ ID NO: 718 | — |
| 753 | isomiR example 2 of SEQ ID NO: 718 | — |
| 754 | isomiR example 1 of SEQ ID NO: 719 | — |
| 755 | isomiR example 2 of SEQ ID NO: 719 | — |
| 756 | isomiR example 1 of SEQ ID NO: 721 | — |
| 757 | isomiR example 2 of SEQ ID NO: 721 | — |
| 758 | isomiR example 1 of SEQ ID NO: 723 | — |
| 759 | isomiR example 2 of SEQ ID NO: 723 | — |
| 760 | isomiR example 1 of SEQ ID NO: 724 | — |
| 761 | isomiR example 2 of SEQ ID NO: 724 | — |
| 762 | isomiR example 1 of SEQ ID NO: 727 | — |
| 763 | isomiR example 2 of SEQ ID NO: 727 | — |
| 764 | isomiR example 1 of SEQ ID NO: 728 | — |
| 765 | isomiR example 2 of SEQ ID NO: 728 | — |

The present specification encompasses the contents described in the specifications and drawings of Japanese Patent Application Nos. 2014-124880 on which the priority of the present application is based.

### Advantageous Effects of Invention

According to the present invention, liver cancer can be detected easily and highly accurately. For example, the presence or absence of liver cancer in a patient can be easily detected by using, as an indicator, the measurement values of several miRNAs in blood, serum, and/or plasma of the patient, which can be collected with limited invasiveness.

### BRIEF DESCRIPTION OF DRAWINGS

FIG. 1 This figure shows the relationship between the nucleotide sequences of hsa-miR-1343-5p represented by SEQ ID NO: 131 and hsa-miR-1343-3p represented by SEQ ID NO: 1, which are produced from a precursor hsa-mir-1343 represented by SEQ ID NO: 225.

FIG. 2 Left diagram: the measurement values of hsa-miR-1343-3p (SEQ ID NO: 1) in healthy subjects (100 persons) and liver cancer patients (34 persons) selected as a training cohort were each plotted on the ordinate. The horizontal line in the diagram depicts a threshold (7.09) that was optimized by Fisher's linear discriminant analysis and discriminated between the two groups. Right diagram: the measurement values of hsa-miR-1343-3p (SEQ ID NO: 1) in healthy subjects (50 persons) and liver cancer patients (16 persons) selected as a validation cohort were each plotted on the ordinate. The horizontal line in the diagram depicts the threshold (7.09) that was set in the training cohort and discriminated between the two groups.

FIG. 3 Left diagram: the measurement values of hsa-miR-1343-3p (SEQ ID NO: 1) in healthy subjects (100 persons, circles) and liver cancer patients (34 persons, triangles) selected as a training cohort were each plotted on the abscissa against their measurement values of hsa-miR-6726-5p (SEQ ID NO: 2) on the ordinate. The line in the diagram depicts a discriminant function (0=0.77x+y−15.07) that was optimized by Fisher's linear discriminant analysis and discriminated between the two groups. Right diagram: the measurement values of hsa-miR-1343-3p (SEQ ID NO: 1) in healthy subjects (50 persons, circles) and liver cancer patients (16 persons, triangles) selected as a validation cohort were each plotted on the abscissa against their measurement values of hsa-miR-6726-5p (SEQ ID NO: 2) on the ordinate. The line in the diagram depicts the threshold (0=0.77x+y−15.07) that was set in the training cohorts and discriminated between the two groups.

FIG. 4 Upper diagram: a discriminant (0.88×hsa-miR-6131−1.58×hsa-miR-642a-3p+0.39×hsa-miR-7641−0.33×hsa-miR-6729−5p+5.19) was prepared by use of Fisher's linear discriminant analysis from the measurement values of hsa-miR-6131 (SEQ ID NO: 7), hsa-miR-642a-3p (SEQ ID NO: 148), hsa-miR-7641 (SEQ ID NO: 9), and hsa-miR-6729-5p (SEQ ID NO: 27) in 35 liver cancer patients, 99 healthy subjects, 72 pancreatic cancer patients, 61 bile duct cancer patients, 35 colorectal cancer patients, 38 stomach cancer patients, 25 esophageal cancer patients, and 16 benign pancreaticobiliary disease patients selected as a training cohort, and discriminant scores obtained from the discriminant were plotted on the ordinate against the sample groups on the abscissa. The dotted line in the diagram depicts a discriminant boundary that offered a discriminant score of 0 and discriminated between the groups. Lower diagram: discriminant scores obtained from the discriminant prepared from the training cohorts as to the measurement values of hsa-miR-6131 (SEQ ID NO: 7), hsa-miR-642a-3p (SEQ ID NO: 148), hsa-miR-7641 (SEQ ID NO: 9), and hsa-miR-6729-5p (SEQ ID NO: 27) in 17 liver cancer patients, 51 healthy subjects, 28 pancreatic cancer patients, 37 bile duct cancer patients, 15 colorectal cancer patients, 12 stomach cancer patients, 25 esophageal cancer patients, and 5 benign pancreaticobiliary disease patients selected as a validation cohort were plotted on the ordinate against the sample groups on the abscissa. The dotted line in the diagram depicts the discriminant boundary that offered a discriminant score of 0 and discriminated between the two groups.

### DESCRIPTION OF EMBODIMENTS

Hereinafter, the present invention will be further described specifically.

1. Target Nucleic Acid for Liver Cancer

As a primary target nucleic acid as a liver cancer marker for detecting the presence and/or absence of liver cancer or liver cancer cells using the nucleic acid probe or the primer for the detection of liver cancer defined above according to the present invention, at least one or more miRNA(s) selected from the group consisting of hsa-miR-1343-3p, hsa-miR-6726-5p, hsa-miR-6515-3p, hsa-miR-4651, hsa-miR-4257, hsa-miR-3188, hsa-miR-6131, hsa-miR-6766-3p, hsa-miR-7641, hsa-miR-1249, hsa-miR-3679-3p, hsa-miR-6787-5p, hsa-miR-4454, hsa-miR-3135b, hsa-miR-6765-3p, hsa-miR-7975, hsa-miR-204-3p, hsa-miR-7977, hsa-miR-7110-5p, hsa-miR-6717-5p, hsa-miR-6870-5p, hsa-miR-663b, hsa-miR-6875-5p, hsa-miR-8072, hsa-miR-6816-5p, hsa-miR-4281, hsa-miR-6729-5p, hsa-miR-8069, hsa-miR-4706, hsa-miR-7108-5p, hsa-miR-4433b-3p, hsa-miR-6893-5p, hsa-miR-6857-5p, hsa-miR-1227-5p, hsa-miR-6741-5p, hsa-miR-451a, hsa-miR-8063, hsa-miR-3622a-5p, hsa-miR-615-5p, hsa-miR-128-1-5p, hsa-miR-6825-5p, hsa-miR-1260b, hsa-miR-4433-3p, hsa-miR-4665-5p, hsa-miR-7845-5p, hsa-miR-1908-5p, hsa-miR-6840-3p, hsa-miR-6765-5p, hsa-miR-296-5p, hsa-miR-36′75-3p, hsa-miR-6781-5p, hsa-miR-423-5p, hsa-miR-3663-3p, hsa-miR-6784-5p, hsa-miR-6749-5p, hsa-miR-1231, hsa-miR-4746-3p, hsa-miR-6780b-5p, hsa-miR-4758-5p, hsa-miR-36′79-5p, hsa-miR-3184-5p, hsa-miR-6125, hsa-miR-6721-5p, hsa-miR-6791-5p, hsa-miR-3185, hsa-miR-1260a, hsa-miR-3197, hsa-miR-6845-5p, hsa-miR-6887-5p, hsa-miR-6738-5p, hsa-miR-6872-3p, hsa-miR-4497, hsa-miR-1229-5p, hsa-miR-6820-5p, hsa-miR-6777-5p, hsa-miR-3917, hsa-miR-5787, hsa-miR-4286, hsa-miR-6877-5p, hsa-miR-1225-3p, hsa-miR-6088, hsa-miR-6800-5p, hsa-miR-1246, hsa-miR-4467, hsa-miR-4419b, hsa-miR-1914-3p, hsa-miR-4632-5p, hsa-miR-1915-5p, hsa-miR-3940-5p, hsa-miR-1185-2-3p, hsa-miR-6746-5p, hsa-miR-5001-5p, hsa-miR-1228-5p, hsa-miR-5572, hsa-miR-4327, hsa-miR-4638-5p, hsa-miR-6799-5p, hsa-miR-6861-5p, hsa-miR-6727-5p, hsa-miR-4513, hsa-miR-6805-3p, hsa-miR-6808-5p, hsa-miR-4449, hsa-miR-1199-5p, hsa-miR-1275, hsa-miR-4792, hsa-miR-4443, hsa-miR-6891-5p, hsa-miR-6826-5p, hsa-miR-6807-5p, hsa-miR-7150, hsa-miR-4534, hsa-miR-4476, hsa-miR-4649-5p, hsa-miR-4525, hsa-miR-1915-3p, hsa-miR-4516, hsa-miR-4417, hsa-miR-642b-3p, hsa-miR-3141, hsa-miR-5100, hsa-miR-6848-5p, hsa-miR-4739, hsa-miR-4459, hsa-miR-1237-5p, hsa-miR-296-3p, hsa-miR-4665-3p, hsa-miR-6786-5p, hsa-miR-4258, hsa-miR-6510-5p, hsa-miR-1343-5p, hsa-miR-1247-3p, hsa-miR-6805-5p, hsa-miR-4492, hsa-miR-1469, hsa-miR-1268b, hsa-miR-6858-5p, hsa-miR-3937, hsa-miR-939-5p, hsa-miR-3656, hsa-miR-744-5p, hsa-miR-4687-3p, hsa-miR-4763-3p, hsa-miR-3620-5p, hsa-miR-3195, hsa-miR-6842-5p, hsa-miR-4707-5p, hsa-miR-642a-3p, hsa-miR-7113-3p, hsa-miR-4728-5p, hsa-miR-5195-3p, hsa-miR-1185-1-3p, hsa-miR-6774-5p, hsa-miR-8059, hsa-miR-3131, hsa-miR-7847-3p, hsa-miR-4463, hsa-miR-128-2-5p, hsa-miR-4508, hsa-miR-6806-5p, hsa-miR-7111-5p, hsa-miR-6782-5p, hsa-miR-4734, hsa-miR-3162-5p, hsa-miR-887-3p, hsa-miR-6752-5p, hsa-miR-6724-5p, hsa-miR-6757-5p, hsa-miR-4448, hsa-miR-671-5p, hsa-miR-3178, hsa-miR-4725-3p, hsa-miR-940, hsa-miR-6789-5p, hsa-miR-4484, hsa-miR-4634, hsa-miR-4745-5p, hsa-miR-4730, hsa-miR-6803-5p, hsa-miR-6798-5p, hsa-miR-3648, hsa-miR-4783-3p and hsa-miR-6836-3p can be used. Furthermore, at least one or more miRNA(s) selected from the group consisting of other liver cancer markers that can be combined with these miRNAs, i.e., hsa-miR-23b-3p, hsa-miR-23a-3p, hsa-miR-625-3p, hsa-miR-1228-3p, hsa-miR-614, hsa-miR-1913, hsa-miR-92a-2-5p, hsa-miR-187-5p, hsa-miR-16-5p, hsa-miR-92b-3p, hsa-miR-150-3p, hsa-miR-564, hsa-miR-125a-3p, hsa-miR-92b-5p, hsa-miR-92a-3p and hsa-miR-663a can also be preferably used as a target nucleic acid. Moreover, at least one or more miRNA(s) selected from the group consisting of other liver cancer markers that can be combined with these miRNAs, i.e., hsa-miR-4688, hsa-miR-4648, hsa-miR-6085, hsa-miR-6126, hsa-miR-6880-5p, hsa-miR-328-5p, hsa-miR-6768-5p, hsa-miR-3180, hsa-miR-6087, hsa-miR-1273g-3p, hsa-miR-1225-5p, hsa-miR-3196, hsa-miR-4695-5p, hsa-miR-6732-5p, hsa-miR-638, hsa-miR-6813-5p, hsa-miR-665, hsa-miR-486-3p, hsa-miR-4466, hsa-miR-30c-1-3p, hsa-miR-3621, hsa-miR-6743-5p, hsa-miR-4298, hsa-miR-4741, hsa-miR-3619-3p, hsa-miR-6824-5p, hsa-miR-5698, hsa-miR-371a-5p, hsa-miR-4488, hsa-miR-1233-5p, hsa-miR-4723-5p, hsa-miR-24-3p, hsa-miR-1238-5p, hsa-miR-4442, hsa-miR-3928-3p, hsa-miR-6716-5p, hsa-miR-6089, hsa-miR-6124, hsa-miR-6778-5p, hsa-miR-557 and hsa-miR-6090 can also be preferably used as a target nucleic acid.

These miRNAs include, for example, a human gene comprising a nucleotide sequence represented by any of SEQ ID NOs: 1 to 224 and 714 to 729 (i.e., hsa-miR-1343-3p, hsa-miR-6726-5p, hsa-miR-6515-3p, hsa-miR-4651, hsa-miR-4257, hsa-miR-3188, hsa-miR-6131, hsa-miR-6766-3p, hsa-miR-7641, hsa-miR-1249, hsa-miR-3679-3p, hsa-miR-6787-5p, hsa-miR-4454, hsa-miR-3135b, hsa-miR-6765-3p, hsa-miR-7975, hsa-miR-204-3p, hsa-miR-7977, hsa-miR-7110-5p, hsa-miR-6717-5p, hsa-miR-6870-5p, hsa-miR-663b, hsa-miR-6875-5p, hsa-miR-8072, hsa-miR-6816-5p, hsa-miR-4281, hsa-miR-6729-5p, hsa-miR-8069, hsa-miR-4706, hsa-miR-7108-5p, hsa-miR-4433b-3p, hsa-miR-6893-5p, hsa-miR-6857-5p, hsa-miR-1227-5p, hsa-miR-6741-5p, hsa-miR-451a, hsa-miR-8063, hsa-miR-3622a-5p, hsa-miR-615-5p, hsa-miR-128-1-5p, hsa-miR-6825-5p, hsa-miR-1260b, hsa-miR-4433-3p, hsa-miR-4665-5p, hsa-miR-7845-5p, hsa-miR-1908-5p, hsa-miR-6840-3p, hsa-miR-6765-5p, hsa-miR-296-5p, hsa-miR-36′75-3p, hsa-miR-6781-5p, hsa-miR-423-5p, hsa-miR-3663-3p, hsa-miR-6784-5p, hsa-miR-6749-5p, hsa-miR-1231, hsa-miR-4746-3p, hsa-miR-6780b-5p, hsa-miR-4758-5p, hsa-miR-36′79-5p, hsa-miR-3184-5p, hsa-miR-6125, hsa-miR-6721-5p, hsa-miR-6791-5p, hsa-miR-3185, hsa-miR-1260a, hsa-miR-3197, hsa-miR-6845-5p, hsa-miR-6887-5p, hsa-miR-6738-5p, hsa-miR-6872-3p, hsa-miR-4497, hsa-miR-1229-5p, hsa-miR-6820-5p, hsa-miR-6777-5p, hsa-miR-3917, hsa-miR-5787, hsa-miR-4286, hsa-miR-6877-5p, hsa-miR-1225-3p, hsa-miR-6088, hsa-miR-6800-5p, hsa-miR-1246, hsa-miR-4467, hsa-miR-4419b, hsa-miR-1914-3p, hsa-miR-4632-5p, hsa-miR-1915-5p, hsa-miR-3940-5p, hsa-miR-1185-2-3p, hsa-miR-6746-5p, hsa-miR-5001-5p, hsa-miR-1228-5p, hsa-miR-5572, hsa-miR-4327, hsa-miR-4638-5p, hsa-miR-6799-5p, hsa-miR-6861-5p, hsa-miR-6727-5p, hsa-miR-4513, hsa-miR-6805-3p, hsa-miR-6808-5p, hsa-miR-4449, hsa-miR-1199-5p, hsa-miR-1275, hsa-miR-4792, hsa-miR-4443, hsa-miR-6891-5p, hsa-miR-6826-5p, hsa-miR-6807-5p, hsa-miR-7150, hsa-miR-4534, hsa-miR-4476, hsa-miR-4649-5p, hsa-miR-4525, hsa-miR-1915-3p, hsa-miR-4516, hsa-miR-4417, hsa-miR-642b-3p, hsa-miR-3141, hsa-miR-5100, hsa-miR-6848-5p, hsa-miR-4739, hsa-miR-4459, hsa-miR-1237-5p, hsa-miR-296-3p, hsa-miR-4665-3p, hsa-miR-6786-5p, hsa-miR-4258, hsa-miR-6510-5p, hsa-miR-1343-5p, hsa-miR-1247-3p, hsa-miR-6805-5p, hsa-miR-4492, hsa-miR-1469, hsa-miR-1268b, hsa-miR-6858-5p, hsa-miR-3937, hsa-miR-939-5p, hsa-miR-3656, hsa-miR-744-5p, hsa-miR-4687-3p, hsa-miR-4763-3p, hsa-miR-3620-5p, hsa-miR-3195, hsa-miR-6842-5p, hsa-miR-4707-5p, hsa-miR-642a-3p, hsa-miR-7113-3p, hsa-miR-4728-5p, hsa-miR-5195-3p, hsa-miR-1185-1-3p, hsa-miR-6774-5p, hsa-miR-8059, hsa-miR-3131, hsa-miR-7847-3p, hsa-miR-4463, hsa-miR-128-2-5p, hsa-miR-4508, hsa-miR-6806-5p, hsa-miR-7111-5p, hsa-miR-6782-5p, hsa-miR-4734, hsa-miR-3162-5p, hsa-miR-887-3p, hsa-miR-6752-5p, hsa-miR-6724-5p, hsa-miR-6757-5p, hsa-miR-4448, hsa-miR-671-5p, hsa-miR-3178, hsa-miR-4725-3p, hsa-miR-940, hsa-miR-6789-5p, hsa-miR-4484, hsa-miR-4634, hsa-miR-4745-5p, hsa-miR-4730, hsa-miR-6803-5p, hsa-miR-6798-5p, hsa-miR-3648, hsa-miR-4783-3p, hsa-miR-6836-3p, hsa-miR-23b-3p, hsa-miR-23a-3p, hsa-miR-625-3p, hsa-miR-1228-3p, hsa-miR-614, hsa-miR-1913, hsa-miR-92a-2-5p, hsa-miR-187-5p, hsa-miR-16-5p, hsa-miR-92b-3p, hsa-miR-150-3p, hsa-miR-564, hsa-miR-125a-3p, hsa-miR-92b-5p, hsa-miR-92a-3p, hsa-miR-663a, hsa-miR-4688, hsa-miR-4648, hsa-miR-6085, hsa-miR-6126, hsa-miR-6880-5p, hsa-miR-328-5p, hsa-miR-6768-5p, hsa-miR-3180, hsa-miR-6087, hsa-miR-1273g-3p, hsa-miR-1225-5p, hsa-miR-3196, hsa-miR-4695-5p, hsa-miR-6732-5p, hsa-miR-638, hsa-miR-6813-5p, hsa-miR-665, hsa-miR-486-3p, hsa-miR-4466, hsa-miR-30c-1-3p, hsa-miR-3621, hsa-miR-6743-5p, hsa-miR-4298, hsa-miR-4741, hsa-miR-3619-3p, hsa-miR-6824-5p, hsa-miR-5698, hsa-miR-371a-5p, hsa-miR-4488, hsa-miR-1233-5p, hsa-miR-4723-5p, hsa-miR-24-3p, hsa-miR-1238-5p, hsa-miR-4442, hsa-miR-3928-3p, hsa-miR-6716-5p, hsa-miR-6089, hsa-miR-6124, hsa-miR-6778-5p, hsa-miR-557 and hsa-miR-6090, respectively), a congener thereof, a transcript thereof, or/and a variant or a derivative thereof. In this context, the gene, the congener, the transcript, the variant, and the derivative are as defined above.

The target nucleic acid is preferably a human gene comprising a nucleotide sequence represented by any of SEQ ID NOs: 1 to 765 or a transcript thereof, more preferably the transcript, i.e., a miRNA or its precursor RNA (pri-miRNA or pre-miRNA).

The first target gene is the hsa-miR-1343-3p gene, a congener thereof, a transcript thereof, or a variant or a derivative thereof. None of the previously known reports show that change in the expression of the gene or the transcript thereof can serve as a marker for liver cancer.

The second target gene is the hsa-miR-6726-5p gene, a congener thereof, a transcript thereof, or a variant or a derivative thereof. None of the previously known reports show that change in the expression of the gene or the transcript thereof can serve as a marker for liver cancer.

The third target gene is the hsa-miR-6515-3p gene, a congener thereof, a transcript thereof, or a variant or a derivative thereof. None of the previously known reports show that change in the expression of the gene or the transcript thereof can serve as a marker for liver cancer.

The fourth target gene is the hsa-miR-4651 gene, a congener thereof, a transcript thereof, or a variant or a derivative thereof. None of the previously known reports show that change in the expression of the gene or the transcript thereof can serve as a marker for liver cancer.

The fifth target gene is the hsa-miR-4257 gene, a congener thereof, a transcript thereof, or a variant or a derivative thereof. None of the previously known reports show that change in the expression of the gene or the transcript thereof can serve as a marker for liver cancer.

The sixth target gene is the hsa-miR-3188 gene, a congener thereof, a transcript thereof, or a variant or a derivative thereof. None of the previously known reports show that change in the expression of the gene or the transcript thereof can serve as a marker for liver cancer.

The seventh target gene is the hsa-miR-6131 gene, a congener thereof, a transcript thereof, or a variant or a derivative thereof. None of the previously known reports show that change in the expression of the gene or the transcript thereof can serve as a marker for liver cancer.

The eighth target gene is the hsa-miR-6766-3p gene, a congener thereof, a transcript thereof, or a variant or a derivative thereof. None of the previously known reports show that change in the expression of the gene or the transcript thereof can serve as a marker for liver cancer.

The ninth target gene is the hsa-miR-7641 gene, a congener thereof, a transcript thereof, or a variant or a derivative thereof. None of the previously known reports show that change in the expression of the gene or the transcript thereof can serve as a marker for liver cancer.

The 10th target gene is the hsa-miR-1249 gene, a congener thereof, a transcript thereof, or a variant or a derivative thereof. None of the previously known reports show that change in the expression of the gene or the transcript thereof can serve as a marker for liver cancer.

The 11th target gene is the hsa-miR-3679-3p gene, a congener thereof, a transcript thereof, or a variant or a derivative thereof. None of the previously known reports show that change in the expression of the gene or the transcript thereof can serve as a marker for liver cancer.

The 12th target gene is the hsa-miR-6787-5p gene, a congener thereof, a transcript thereof, or a variant or a derivative thereof. None of the previously known reports show that change in the expression of the gene or the transcript thereof can serve as a marker for liver cancer.

The 13th target gene is the hsa-miR-4454 gene, a congener thereof, a transcript thereof, or a variant or a derivative thereof. None of the previously known reports show that change in the expression of the gene or the transcript thereof can serve as a marker for liver cancer.

The 14th target gene is the hsa-miR-3135b gene, a congener thereof, a transcript thereof, or a variant or a derivative thereof. None of the previously known reports show that change in the expression of the gene or the transcript thereof can serve as a marker for liver cancer.

The 15th target gene is the hsa-miR-6765-3p gene, a congener thereof, a transcript thereof, or a variant or a derivative thereof. None of the previously known reports show that change in the expression of the gene or the transcript thereof can serve as a marker for liver cancer.

The 16th target gene is the hsa-miR-7975 gene, a congener thereof, a transcript thereof, or a variant or a derivative thereof. None of the previously known reports show that change in the expression of the gene or the transcript thereof can serve as a marker for liver cancer.

The 17th target gene is the hsa-miR-204-3p gene, a congener thereof, a transcript thereof, or a variant or a derivative thereof. None of the previously known reports show that change in the expression of the gene or the transcript thereof can serve as a marker for liver cancer.

The 18th target gene is the hsa-miR-7977 gene, a congener thereof, a transcript thereof, or a variant or a derivative thereof. None of the previously known reports show that change in the expression of the gene or the transcript thereof can serve as a marker for liver cancer.

The 19th target gene is the hsa-miR-7110-5p gene, a congener thereof, a transcript thereof, or a variant or a derivative thereof. None of the previously known reports show that change in the expression of the gene or the transcript thereof can serve as a marker for liver cancer.

The 20th target gene is the hsa-miR-6717-5p gene, a congener thereof, a transcript thereof, or a variant or a derivative thereof. None of the previously known reports show that change in the expression of the gene or the transcript thereof can serve as a marker for liver cancer.

The 21st target gene is the hsa-miR-6870-5p gene, a congener thereof, a transcript thereof, or a variant or a derivative thereof. None of the previously known reports show that change in the expression of the gene or the transcript thereof can serve as a marker for liver cancer.

The 22nd target gene is the hsa-miR-663b gene, a congener thereof, a transcript thereof, or a variant or a derivative thereof. None of the previously known reports show that change in the expression of the gene or the transcript thereof can serve as a marker for liver cancer.

The 23rd target gene is the hsa-miR-6875-5p gene, a congener thereof, a transcript thereof, or a variant or a derivative thereof. None of the previously known reports show that change in the expression of the gene or the transcript thereof can serve as a marker for liver cancer.

The 24th target gene is the hsa-miR-8072 gene, a congener thereof, a transcript thereof, or a variant or a derivative thereof. None of the previously known reports show that change in the expression of the gene or the transcript thereof can serve as a marker for liver cancer.

The 25th target gene is the hsa-miR-6816-5p gene, a congener thereof, a transcript thereof, or a variant or a derivative thereof. None of the previously known reports show that change in the expression of the gene or the transcript thereof can serve as a marker for liver cancer.

The 26th target gene is the hsa-miR-4281 gene, a congener thereof, a transcript thereof, or a variant or a derivative thereof. None of the previously known reports show that change in the expression of the gene or the transcript thereof can serve as a marker for liver cancer.

The 27th target gene is the hsa-miR-6729-5p gene, a congener thereof, a transcript thereof, or a variant or a derivative thereof. None of the previously known reports show that change in the expression of the gene or the transcript thereof can serve as a marker for liver cancer.

The 28th target gene is the hsa-miR-8069 gene, a congener thereof, a transcript thereof, or a variant or a derivative thereof. None of the previously known reports show that change in the expression of the gene or the transcript thereof can serve as a marker for liver cancer.

The 29th target gene is the hsa-miR-4706 gene, a congener thereof, a transcript thereof, or a variant or a derivative thereof. None of the previously known reports show that change in the expression of the gene or the transcript thereof can serve as a marker for liver cancer.

The 30th target gene is the hsa-miR-7108-5p gene, a congener thereof, a transcript thereof, or a variant or a derivative thereof. None of the previously known reports show that change in the expression of the gene or the transcript thereof can serve as a marker for liver cancer.

The 31st target gene is the hsa-miR-4433b-3p gene, a congener thereof, a transcript thereof, or a variant or a derivative thereof. None of the previously known reports show that change in the expression of the gene or the transcript thereof can serve as a marker for liver cancer.

The 32nd target gene is the hsa-miR-6893-5p gene, a congener thereof, a transcript thereof, or a variant or a derivative thereof. None of the previously known reports show that change in the expression of the gene or the transcript thereof can serve as a marker for liver cancer.

The 33rd target gene is the hsa-miR-6857-5p gene, a congener thereof, a transcript thereof, or a variant or a derivative thereof. None of the previously known reports show that change in the expression of the gene or the transcript thereof can serve as a marker for liver cancer.

The 34th target gene is the hsa-miR-1227-5p gene, a congener thereof, a transcript thereof, or a variant or a derivative thereof. None of the previously known reports show that change in the expression of the gene or the transcript thereof can serve as a marker for liver cancer.

The 35th target gene is the hsa-miR-6741-5p gene, a congener thereof, a transcript thereof, or a variant or a derivative thereof. None of the previously known reports show that change in the expression of the gene or the transcript thereof can serve as a marker for liver cancer.

The 36th target gene is the hsa-miR-451a gene, a congener thereof, a transcript thereof, or a variant or a derivative thereof. None of the previously known reports show that change in the expression of the gene or the transcript thereof can serve as a marker for liver cancer.

The 37th target gene is the hsa-miR-8063 gene, a congener thereof, a transcript thereof, or a variant or a derivative thereof. None of the previously known reports show that change in the expression of the gene or the transcript thereof can serve as a marker for liver cancer.

The 38th target gene is the hsa-miR-3622a-5p gene, a congener thereof, a transcript thereof, or a variant or a derivative thereof. None of the previously known reports show that change in the expression of the gene or the transcript thereof can serve as a marker for liver cancer.

The 39th target gene is the hsa-miR-615-5p gene, a congener thereof, a transcript thereof, or a variant or a derivative thereof. None of the previously known reports show that change in the expression of the gene or the transcript thereof can serve as a marker for liver cancer.

The 40th target gene is the hsa-miR-128-1-5p gene, a congener thereof, a transcript thereof, or a variant or a derivative thereof. None of the previously known reports show that change in the expression of the gene or the transcript thereof can serve as a marker for liver cancer.

The 41st target gene is the hsa-miR-6825-5p gene, a congener thereof, a transcript thereof, or a variant or a derivative thereof. None of the previously known reports show that change in the expression of the gene or the transcript thereof can serve as a marker for liver cancer.

The 42nd target gene is the hsa-miR-1260b gene, a congener thereof, a transcript thereof, or a variant or a derivative thereof. None of the previously known reports show that change in the expression of the gene or the transcript thereof can serve as a marker for liver cancer.

The 43rd target gene is the hsa-miR-4433-3p gene, a congener thereof, a transcript thereof, or a variant or a derivative thereof. None of the previously known reports show that change in the expression of the gene or the transcript thereof can serve as a marker for liver cancer.

The 44th target gene is the hsa-miR-4665-5p gene, a congener thereof, a transcript thereof, or a variant or a derivative thereof. None of the previously known reports show that change in the expression of the gene or the transcript thereof can serve as a marker for liver cancer.

The 45th target gene is the hsa-miR-7845-5p gene, a congener thereof, a transcript thereof, or a variant or a derivative thereof. None of the previously known reports show that change in the expression of the gene or the transcript thereof can serve as a marker for liver cancer.

The 46th target gene is the hsa-miR-1908-5p gene, a congener thereof, a transcript thereof, or a variant or a derivative thereof. None of the previously known reports show that change in the expression of the gene or the transcript thereof can serve as a marker for liver cancer.

The 47th target gene is the hsa-miR-6840-3p gene, a congener thereof, a transcript thereof, or a variant or a derivative thereof. None of the previously known reports show that change in the expression of the gene or the transcript thereof can serve as a marker for liver cancer.

The 48th target gene is the hsa-miR-6765-5p gene, a congener thereof, a transcript thereof, or a variant or a derivative thereof. None of the previously known reports show that change in the expression of the gene or the transcript thereof can serve as a marker for liver cancer.

The 49th target gene is the hsa-miR-296-5p gene, a congener thereof, a transcript thereof, or a variant or a derivative thereof. None of the previously known reports show that change in the expression of the gene or the transcript thereof can serve as a marker for liver cancer.

The 50th target gene is the hsa-miR-3675-3p gene, a congener thereof, a transcript thereof, or a variant or a derivative thereof. None of the previously known reports show that change in the expression of the gene or the transcript thereof can serve as a marker for liver cancer.

The 51st target gene is the hsa-miR-6781-5p gene, a congener thereof, a transcript thereof, or a variant or a derivative thereof. None of the previously known reports show that change in the expression of the gene or the transcript thereof can serve as a marker for liver cancer.

The 52nd target gene is the hsa-miR-423-5p gene, a congener thereof, a transcript thereof, or a variant or a derivative thereof. None of the previously known reports show that change in the expression of the gene or the transcript thereof can serve as a marker for liver cancer.

The 53rd target gene is the hsa-miR-3663-3p gene, a congener thereof, a transcript thereof, or a variant or a derivative thereof. None of the previously known reports show that change in the expression of the gene or the transcript thereof can serve as a marker for liver cancer.

The 54th target gene is the hsa-miR-6784-5p gene, a congener thereof, a transcript thereof, or a variant or a derivative thereof. None of the previously known reports show that change in the expression of the gene or the transcript thereof can serve as a marker for liver cancer.

The 55th target gene is the hsa-miR-6749-5p gene, a congener thereof, a transcript thereof, or a variant or a derivative thereof. None of the previously known reports show that change in the expression of the gene or the transcript thereof can serve as a marker for liver cancer.

The 56th target gene is the hsa-miR-1231 gene, a congener thereof, a transcript thereof, or a variant or a derivative thereof. None of the previously known reports show that change in the expression of the gene or the transcript thereof can serve as a marker for liver cancer.

The 57th target gene is the hsa-miR-4746-3p gene, a congener thereof, a transcript thereof, or a variant or a derivative thereof. None of the previously known reports show that change in the expression of the gene or the transcript thereof can serve as a marker for liver cancer.

The 58th target gene is the hsa-miR-6780b-5p gene, a congener thereof, a transcript thereof, or a variant or a derivative thereof. None of the previously known reports show that change in the expression of the gene or the transcript thereof can serve as a marker for liver cancer.

The 59th target gene is the hsa-miR-4758-5p gene, a congener thereof, a transcript thereof, or a variant or a derivative thereof. None of the previously known reports show that change in the expression of the gene or the transcript thereof can serve as a marker for liver cancer.

The 60th target gene is the hsa-miR-3679-5p gene, a congener thereof, a transcript thereof, or a variant or a derivative thereof. None of the previously known reports show that change in the expression of the gene or the transcript thereof can serve as a marker for liver cancer.

The 61st target gene is the hsa-miR-3184-5p gene, a congener thereof, a transcript thereof, or a variant or a derivative thereof. None of the previously known reports show that change in the expression of the gene or the transcript thereof can serve as a marker for liver cancer.

The 62nd target gene is the hsa-miR-6125 gene, a congener thereof, a transcript thereof, or a variant or a derivative thereof. None of the previously known reports show that change in the expression of the gene or the transcript thereof can serve as a marker for liver cancer.

The 63rd target gene is the hsa-miR-6721-5p gene, a congener thereof, a transcript thereof, or a variant or a derivative thereof. None of the previously known reports show that change in the expression of the gene or the transcript thereof can serve as a marker for liver cancer.

The 64th target gene is the hsa-miR-6791-5p gene, a congener thereof, a transcript thereof, or a variant or a derivative thereof. None of the previously known reports show that change in the expression of the gene or the transcript thereof can serve as a marker for liver cancer.

The 65th target gene is the hsa-miR-3185 gene, a congener thereof, a transcript thereof, or a variant or a derivative thereof. None of the previously known reports show that change in the expression of the gene or the transcript thereof can serve as a marker for liver cancer.

The 66th target gene is the hsa-miR-1260a gene, a congener thereof, a transcript thereof, or a variant or a derivative thereof. None of the previously known reports show that change in the expression of the gene or the transcript thereof can serve as a marker for liver cancer.

The 67th target gene is the hsa-miR-3197 gene, a congener thereof, a transcript thereof, or a variant or a derivative thereof. None of the previously known reports show that change in the expression of the gene or the transcript thereof can serve as a marker for liver cancer.

The 68th target gene is the hsa-miR-6845-5p gene, a congener thereof, a transcript thereof, or a variant or a derivative thereof. None of the previously known reports show that change in the expression of the gene or the transcript thereof can serve as a marker for liver cancer.

The 69th target gene is the hsa-miR-6887-5p gene, a congener thereof, a transcript thereof, or a variant or a derivative thereof. None of the previously known reports show that change in the expression of the gene or the transcript thereof can serve as a marker for liver cancer.

The 70th target gene is the hsa-miR-6738-5p gene, a congener thereof, a transcript thereof, or a variant or a derivative thereof. None of the previously known reports show that change in the expression of the gene or the transcript thereof can serve as a marker for liver cancer.

The 71st target gene is the hsa-miR-6872-3p gene, a congener thereof, a transcript thereof, or a variant or a derivative thereof. None of the previously known reports show that change in the expression of the gene or the transcript thereof can serve as a marker for liver cancer.

The 72nd target gene is the hsa-miR-4497 gene, a congener thereof, a transcript thereof, or a variant or a derivative thereof. None of the previously known reports show that change in the expression of the gene or the transcript thereof can serve as a marker for liver cancer.

The 73rd target gene is the hsa-miR-1229-5p gene, a congener thereof, a transcript thereof, or a variant or a derivative thereof. None of the previously known reports show that change in the expression of the gene or the transcript thereof can serve as a marker for liver cancer.

The 74th target gene is the hsa-miR-6820-5p gene, a congener thereof, a transcript thereof, or a variant or a derivative thereof. None of the previously known reports show that change in the expression of the gene or the transcript thereof can serve as a marker for liver cancer.

The 75th target gene is the hsa-miR-6777-5p gene, a congener thereof, a transcript thereof, or a variant or a derivative thereof. None of the previously known reports show that change in the expression of the gene or the transcript thereof can serve as a marker for liver cancer.

The 76th target gene is the hsa-miR-3917 gene, a congener thereof, a transcript thereof, or a variant or a derivative thereof. None of the previously known reports show that change in the expression of the gene or the transcript thereof can serve as a marker for liver cancer.

The 77th target gene is the hsa-miR-5787 gene, a congener thereof, a transcript thereof, or a variant or a derivative thereof. None of the previously known reports show that change in the expression of the gene or the transcript thereof can serve as a marker for liver cancer.

The 78th target gene is the hsa-miR-4286 gene, a congener thereof, a transcript thereof, or a variant or a derivative thereof. None of the previously known reports show that change in the expression of the gene or the transcript thereof can serve as a marker for liver cancer.

The 79th target gene is the hsa-miR-6877-5p gene, a congener thereof, a transcript thereof, or a variant or a derivative thereof. None of the previously known reports show that change in the expression of the gene or the transcript thereof can serve as a marker for liver cancer.

The 80th target gene is the hsa-miR-1225-3p gene, a congener thereof, a transcript thereof, or a variant or a derivative thereof. None of the previously known reports show that change in the expression of the gene or the transcript thereof can serve as a marker for liver cancer.

The 81st target gene is the hsa-miR-6088 gene, a congener thereof, a transcript thereof, or a variant or a derivative thereof. None of the previously known reports show that change in the expression of the gene or the transcript thereof can serve as a marker for liver cancer.

The 82nd target gene is the hsa-miR-6800-5p gene, a congener thereof, a transcript thereof, or a variant or a derivative thereof. None of the previously known reports show that change in the expression of the gene or the transcript thereof can serve as a marker for liver cancer.

The 83rd target gene is the hsa-miR-1246 gene, a congener thereof, a transcript thereof, or a variant or a derivative thereof. None of the previously known reports show that change in the expression of the gene or the transcript thereof can serve as a marker for liver cancer.

The 84th target gene is the hsa-miR-4467 gene, a congener thereof, a transcript thereof, or a variant or a derivative thereof. None of the previously known reports show that change in the expression of the gene or the transcript thereof can serve as a marker for liver cancer.

The 85th target gene is the hsa-miR-4419b gene, a congener thereof, a transcript thereof, or a variant or a derivative thereof. None of the previously known reports show that change in the expression of the gene or the transcript thereof can serve as a marker for liver cancer.

The 86th target gene is the hsa-miR-1914-3p gene, a congener thereof, a transcript thereof, or a variant or a derivative thereof. None of the previously known reports show that change in the expression of the gene or the transcript thereof can serve as a marker for liver cancer.

The 87th target gene is the hsa-miR-4632-5p gene, a congener thereof, a transcript thereof, or a variant or a derivative thereof. None of the previously known reports show that change in the expression of the gene or the transcript thereof can serve as a marker for liver cancer.

The 88th target gene is the hsa-miR-1915-5p gene, a congener thereof, a transcript thereof, or a variant or a derivative thereof. None of the previously known reports show that change in the expression of the gene or the transcript thereof can serve as a marker for liver cancer.

The 89th target gene is the hsa-miR-3940-5p gene, a congener thereof, a transcript thereof, or a variant or a derivative thereof. None of the previously known reports show that change in the expression of the gene or the transcript thereof can serve as a marker for liver cancer.

The 90th target gene is the hsa-miR-1185-2-3p gene, a congener thereof, a transcript thereof, or a variant or a derivative thereof. None of the previously known reports show that change in the expression of the gene or the transcript thereof can serve as a marker for liver cancer.

The 91st target gene is the hsa-miR-6746-5p gene, a congener thereof, a transcript thereof, or a variant or a derivative thereof. None of the previously known reports show that change in the expression of the gene or the transcript thereof can serve as a marker for liver cancer.

The 92nd target gene is the hsa-miR-5001-5p gene, a congener thereof, a transcript thereof, or a variant or a derivative thereof. None of the previously known reports show that change in the expression of the gene or the transcript thereof can serve as a marker for liver cancer.

The 93rd target gene is the hsa-miR-1228-5p gene, a congener thereof, a transcript thereof, or a variant or a derivative thereof. None of the previously known reports show that change in the expression of the gene or the transcript thereof can serve as a marker for liver cancer.

The 94th target gene is the hsa-miR-5572 gene, a congener thereof, a transcript thereof, or a variant or a derivative thereof. None of the previously known reports show that change in the expression of the gene or the transcript thereof can serve as a marker for liver cancer.

The 95th target gene is the hsa-miR-4327 gene, a congener thereof, a transcript thereof, or a variant or a derivative thereof. None of the previously known reports show that change in the expression of the gene or the transcript thereof can serve as a marker for liver cancer.

The 96th target gene is the hsa-miR-4638-5p gene, a congener thereof, a transcript thereof, or a variant or a derivative thereof. None of the previously known reports show that change in the expression of the gene or the transcript thereof can serve as a marker for liver cancer.

The 97th target gene is the hsa-miR-6799-5p gene, a congener thereof, a transcript thereof, or a variant or a derivative thereof. None of the previously known reports show that change in the expression of the gene or the transcript thereof can serve as a marker for liver cancer.

The 98th target gene is the hsa-miR-6861-5p gene, a congener thereof, a transcript thereof, or a variant or a derivative thereof. None of the previously known reports show that change in the expression of the gene or the transcript thereof can serve as a marker for liver cancer.

The 99th target gene is the hsa-miR-6727-5p gene, a congener thereof, a transcript thereof, or a variant or a derivative thereof. None of the previously known reports show that change in the expression of the gene or the transcript thereof can serve as a marker for liver cancer.

The 100th target gene is the hsa-miR-4513 gene, a congener thereof, a transcript thereof, or a variant or a derivative thereof. None of the previously known reports show that change in the expression of the gene or the transcript thereof can serve as a marker for liver cancer.

The 101st target gene is the hsa-miR-6805-3p gene, a congener thereof, a transcript thereof, or a variant or a derivative thereof. None of the previously known reports show that change in the expression of the gene or the transcript thereof can serve as a marker for liver cancer.

The 102nd target gene is the hsa-miR-6808-5p gene, a congener thereof, a transcript thereof, or a variant or a derivative thereof. None of the previously known reports show that change in the expression of the gene or the transcript thereof can serve as a marker for liver cancer.

The 103rd target gene is the hsa-miR-4449 gene, a congener thereof, a transcript thereof, or a variant or a derivative thereof. None of the previously known reports show that change in the expression of the gene or the transcript thereof can serve as a marker for liver cancer.

The 104th target gene is the hsa-miR-1199-5p gene, a congener thereof, a transcript thereof, or a variant or a derivative thereof. None of the previously known reports show that change in the expression of the gene or the transcript thereof can serve as a marker for liver cancer.

The 105th target gene is the hsa-miR-1275 gene, a congener thereof, a transcript thereof, or a variant or a derivative thereof. None of the previously known reports show that change in the expression of the gene or the transcript thereof can serve as a marker for liver cancer.

The 106th target gene is the hsa-miR-4792 gene, a congener thereof, a transcript thereof, or a variant or a derivative thereof. None of the previously known reports show that change in the expression of the gene or the transcript thereof can serve as a marker for liver cancer.

The 107th target gene is the hsa-miR-4443 gene, a congener thereof, a transcript thereof, or a variant or a derivative thereof. None of the previously known reports show that change in the expression of the gene or the transcript thereof can serve as a marker for liver cancer.

The 108th target gene is the hsa-miR-6891-5p gene, a congener thereof, a transcript thereof, or a variant or a derivative thereof. None of the previously known reports show that change in the expression of the gene or the transcript thereof can serve as a marker for liver cancer.

The 109th target gene is the hsa-miR-6826-5p gene, a congener thereof, a transcript thereof, or a variant or a derivative thereof. None of the previously known reports show that change in the expression of the gene or the transcript thereof can serve as a marker for liver cancer.

The 110th target gene is the hsa-miR-6807-5p gene, a congener thereof, a transcript thereof, or a variant or a derivative thereof. None of the previously known reports show that change in the expression of the gene or the transcript thereof can serve as a marker for liver cancer.

The 111th target gene is the hsa-miR-7150 gene, a congener thereof, a transcript thereof, or a variant or a derivative thereof. None of the previously known reports show that change in the expression of the gene or the transcript thereof can serve as a marker for liver cancer.

The 112th target gene is the hsa-miR-4534 gene, a congener thereof, a transcript thereof, or a variant or a derivative thereof. None of the previously known reports show that change in the expression of the gene or the transcript thereof can serve as a marker for liver cancer.

The 113th target gene is the hsa-miR-4476 gene, a congener thereof, a transcript thereof, or a variant or a derivative thereof. None of the previously known reports show that change in the expression of the gene or the transcript thereof can serve as a marker for liver cancer.

The 114th target gene is the hsa-miR-4649-5p gene, a congener thereof, a transcript thereof, or a variant or a derivative thereof. None of the previously known reports show that change in the expression of the gene or the transcript thereof can serve as a marker for liver cancer.

The 115th target gene is the hsa-miR-4525 gene, a congener thereof, a transcript thereof, or a variant or a derivative thereof. None of the previously known reports show that change in the expression of the gene or the transcript thereof can serve as a marker for liver cancer.

The 116th target gene is the hsa-miR-1915-3p gene, a congener thereof, a transcript thereof, or a variant or a derivative thereof. None of the previously known reports show that change in the expression of the gene or the transcript thereof can serve as a marker for liver cancer.

The 117th target gene is the hsa-miR-4516 gene, a congener thereof, a transcript thereof, or a variant or a derivative thereof. None of the previously known reports show that change in the expression of the gene or the transcript thereof can serve as a marker for liver cancer.

The 118th target gene is the hsa-miR-4417 gene, a congener thereof, a transcript thereof, or a variant or a derivative thereof. None of the previously known reports show that change in the expression of the gene or the transcript thereof can serve as a marker for liver cancer.

The 119th target gene is the hsa-miR-642b-3p gene, a congener thereof, a transcript thereof, or a variant or a derivative thereof. None of the previously known reports show that change in the expression of the gene or the transcript thereof can serve as a marker for liver cancer.

The 120th target gene is the hsa-miR-3141 gene, a congener thereof, a transcript thereof, or a variant or a derivative thereof. None of the previously known reports show that change in the expression of the gene or the transcript thereof can serve as a marker for liver cancer.

The 121st target gene is the hsa-miR-5100 gene, a congener thereof, a transcript thereof, or a variant or a derivative thereof. None of the previously known reports show that change in the expression of the gene or the transcript thereof can serve as a marker for liver cancer.

The 122nd target gene is the hsa-miR-6848-5p gene, a congener thereof, a transcript thereof, or a variant or a derivative thereof. None of the previously known reports show that change in the expression of the gene or the transcript thereof can serve as a marker for liver cancer.

The 123rd target gene is the hsa-miR-4739 gene, a congener thereof, a transcript thereof, or a variant or a derivative thereof. None of the previously known reports show that change in the expression of the gene or the transcript thereof can serve as a marker for liver cancer.

The 124th target gene is the hsa-miR-4459 gene, a congener thereof, a transcript thereof, or a variant or a derivative thereof. None of the previously known reports show that change in the expression of the gene or the transcript thereof can serve as a marker for liver cancer.

The 125th target gene is the hsa-miR-1237-5p gene, a congener thereof, a transcript thereof, or a variant or a derivative thereof. None of the previously known reports show that change in the expression of the gene or the transcript thereof can serve as a marker for liver cancer.

The 126th target gene is the hsa-miR-296-3p gene, a congener thereof, a transcript thereof, or a variant or a derivative thereof. None of the previously known reports show that change in the expression of the gene or the transcript thereof can serve as a marker for liver cancer.

The 127th target gene is the hsa-miR-4665-3p gene, a congener thereof, a transcript thereof, or a variant or a derivative thereof. None of the previously known reports show that change in the expression of the gene or the transcript thereof can serve as a marker for liver cancer.

The 128th target gene is the hsa-miR-6786-5p gene, a congener thereof, a transcript thereof, or a variant or a derivative thereof. None of the previously known reports show that change in the expression of the gene or the transcript thereof can serve as a marker for liver cancer.

The 129th target gene is the hsa-miR-4258 gene, a congener thereof, a transcript thereof, or a variant or a derivative thereof. None of the previously known reports show that change in the expression of the gene or the transcript thereof can serve as a marker for liver cancer.

The 130th target gene is the hsa-miR-6510-5p gene, a congener thereof, a transcript thereof, or a variant or a derivative thereof. None of the previously known reports show that change in the expression of the gene or the transcript thereof can serve as a marker for liver cancer.

The 131st target gene is the hsa-miR-1343-5p gene, a congener thereof, a transcript thereof, or a variant or a derivative thereof. None of the previously known reports show that change in the expression of the gene or the transcript thereof can serve as a marker for liver cancer.

The 132nd target gene is the hsa-miR-1247-3p gene, a congener thereof, a transcript thereof, or a variant or a derivative thereof. None of the previously known reports show that change in the expression of the gene or the transcript thereof can serve as a marker for liver cancer.

The 133rd target gene is the hsa-miR-6805-5p gene, a congener thereof, a transcript thereof, or a variant or a derivative thereof. None of the previously known reports show that change in the expression of the gene or the transcript thereof can serve as a marker for liver cancer.

The 134th target gene is the hsa-miR-4492 gene, a congener thereof, a transcript thereof, or a variant or a derivative thereof. None of the previously known reports show that change in the expression of the gene or the transcript thereof can serve as a marker for liver cancer.

The 135th target gene is the hsa-miR-1469 gene, a congener thereof, a transcript thereof, or a variant or a derivative thereof. None of the previously known reports show that change in the expression of the gene or the transcript thereof can serve as a marker for liver cancer.

The 136th target gene is the hsa-miR-1268b gene, a congener thereof, a transcript thereof, or a variant or a derivative thereof. None of the previously known reports show that change in the expression of the gene or the transcript thereof can serve as a marker for liver cancer.

The 137th target gene is the hsa-miR-6858-5p gene, a congener thereof, a transcript thereof, or a variant or a derivative thereof. None of the previously known reports show that change in the expression of the gene or the transcript thereof can serve as a marker for liver cancer.

The 138th target gene is the hsa-miR-3937 gene, a congener thereof, a transcript thereof, or a variant or a derivative thereof. None of the previously known reports show that change in the expression of the gene or the transcript thereof can serve as a marker for liver cancer.

The 139th target gene is the hsa-miR-939-5p gene, a congener thereof, a transcript thereof, or a variant or a derivative thereof. None of the previously known reports show that change in the expression of the gene or the transcript thereof can serve as a marker for liver cancer.

The 140th target gene is the hsa-miR-3656 gene, a congener thereof, a transcript thereof, or a variant or a derivative thereof. None of the previously known reports show that change in the expression of the gene or the transcript thereof can serve as a marker for liver cancer.

The 141st target gene is the hsa-miR-744-5p gene, a congener thereof, a transcript thereof, or a variant or a derivative thereof. None of the previously known reports show that change in the expression of the gene or the transcript thereof can serve as a marker for liver cancer.

The 142nd target gene is the hsa-miR-4687-3p gene, a congener thereof, a transcript thereof, or a variant or a derivative thereof. None of the previously known reports show that change in the expression of the gene or the transcript thereof can serve as a marker for liver cancer.

The 143rd target gene is the hsa-miR-4763-3p gene, a congener thereof, a transcript thereof, or a variant or a derivative thereof. None of the previously known reports show that change in the expression of the gene or the transcript thereof can serve as a marker for liver cancer.

The 144th target gene is the hsa-miR-3620-5p gene, a congener thereof, a transcript thereof, or a variant or a derivative thereof. None of the previously known reports show that change in the expression of the gene or the transcript thereof can serve as a marker for liver cancer.

The 145th target gene is the hsa-miR-3195 gene, a congener thereof, a transcript thereof, or a variant or a derivative thereof. None of the previously known reports show that change in the expression of the gene or the transcript thereof can serve as a marker for liver cancer.

The 146th target gene is the hsa-miR-6842-5p gene, a congener thereof, a transcript thereof, or a variant or a derivative thereof. None of the previously known reports show that change in the expression of the gene or the transcript thereof can serve as a marker for liver cancer.

The 147th target gene is the hsa-miR-4707-5p gene, a congener thereof, a transcript thereof, or a variant or a derivative thereof. None of the previously known reports show that change in the expression of the gene or the transcript thereof can serve as a marker for liver cancer.

The 148th target gene is the hsa-miR-642a-3p gene, a congener thereof, a transcript thereof, or a variant or a derivative thereof. None of the previously known reports show that change in the expression of the gene or the transcript thereof can serve as a marker for liver cancer.

The 149th target gene is the hsa-miR-7113-3p gene, a congener thereof, a transcript thereof, or a variant or a derivative thereof. None of the previously known reports show that change in the expression of the gene or the transcript thereof can serve as a marker for liver cancer.

The 150th target gene is the hsa-miR-4728-5p gene, a congener thereof, a transcript thereof, or a variant or a derivative thereof. None of the previously known reports show that change in the expression of the gene or the transcript thereof can serve as a marker for liver cancer.

The 151st target gene is the hsa-miR-5195-3p gene, a congener thereof, a transcript thereof, or a variant or a derivative thereof. None of the previously known reports show that change in the expression of the gene or the transcript thereof can serve as a marker for liver cancer.

The 152nd target gene is the hsa-miR-1185-1-3p gene, a congener thereof, a transcript thereof, or a variant or a derivative thereof. None of the previously known reports show that change in the expression of the gene or the transcript thereof can serve as a marker for liver cancer.

The 153rd target gene is the hsa-miR-6774-5p gene, a congener thereof, a transcript thereof, or a variant or a derivative thereof. None of the previously known reports show that change in the expression of the gene or the transcript thereof can serve as a marker for liver cancer.

The 154th target gene is the hsa-miR-8059 gene, a congener thereof, a transcript thereof, or a variant or a derivative thereof. None of the previously known reports show that change in the expression of the gene or the transcript thereof can serve as a marker for liver cancer.

The 155th target gene is the hsa-miR-3131 gene, a congener thereof, a transcript thereof, or a variant or a derivative thereof. None of the previously known reports show that change in the expression of the gene or the transcript thereof can serve as a marker for liver cancer.

The 156th target gene is the hsa-miR-7847-3p gene, a congener thereof, a transcript thereof, or a variant or a derivative thereof. None of the previously known reports show that change in the expression of the gene or the transcript thereof can serve as a marker for liver cancer.

The 157th target gene is the hsa-miR-4463 gene, a congener thereof, a transcript thereof, or a variant or a derivative thereof. None of the previously known reports show that change in the expression of the gene or the transcript thereof can serve as a marker for liver cancer.

The 158th target gene is the hsa-miR-128-2-5p gene, a congener thereof, a transcript thereof, or a variant or a derivative thereof. None of the previously known reports show that change in the expression of the gene or the transcript thereof can serve as a marker for liver cancer.

The 159th target gene is the hsa-miR-4508 gene, a congener thereof, a transcript thereof, or a variant or a derivative thereof. None of the previously known reports show that change in the expression of the gene or the transcript thereof can serve as a marker for liver cancer.

The 160th target gene is the hsa-miR-6806-5p gene, a congener thereof, a transcript thereof, or a variant or a derivative thereof. None of the previously known reports show that change in the expression of the gene or the transcript thereof can serve as a marker for liver cancer.

The 161st target gene is the hsa-miR-7111-5p gene, a congener thereof, a transcript thereof, or a variant or a derivative thereof. None of the previously known reports show that change in the expression of the gene or the transcript thereof can serve as a marker for liver cancer.

The 162nd target gene is the hsa-miR-6782-5p gene, a congener thereof, a transcript thereof, or a variant or a derivative thereof. None of the previously known reports show that change in the expression of the gene or the transcript thereof can serve as a marker for liver cancer.

The 163rd target gene is the hsa-miR-4734 gene, a congener thereof, a transcript thereof, or a variant or a derivative thereof. None of the previously known reports show that change in the expression of the gene or the transcript thereof can serve as a marker for liver cancer.

The 164th target gene is the hsa-miR-3162-5p gene, a congener thereof, a transcript thereof, or a variant or a derivative thereof. None of the previously known reports show that change in the expression of the gene or the transcript thereof can serve as a marker for liver cancer.

The 165th target gene is the hsa-miR-887-3p gene, a congener thereof, a transcript thereof, or a variant or a derivative thereof. None of the previously known reports show that change in the expression of the gene or the transcript thereof can serve as a marker for liver cancer.

The 166th target gene is the hsa-miR-6752-5p gene, a congener thereof, a transcript thereof, or a variant or a derivative thereof. None of the previously known reports show that change in the expression of the gene or the transcript thereof can serve as a marker for liver cancer.

The 167th target gene is the hsa-miR-6724-5p gene, a congener thereof, a transcript thereof, or a variant or a derivative thereof. None of the previously known reports show that change in the expression of the gene or the transcript thereof can serve as a marker for liver cancer.

The 168th target gene is the hsa-miR-23b-3p gene, a congener thereof, a transcript thereof, or a variant or a derivative thereof. The previously known reports show that change in the expression of the gene or the transcript thereof can serve as a marker for liver cancer (Patent Literatures 2 and 3).

The 169th target gene is the hsa-miR-23a-3p gene, a congener thereof, a transcript thereof, or a variant or a derivative thereof. The previously known report shows that change in the expression of the gene or the transcript thereof can serve as a marker for liver cancer (Patent Literature 2).

The 170th target gene is the hsa-miR-625-3p gene, a congener thereof, a transcript thereof, or a variant or a derivative thereof. The previously known report shows that change in the expression of the gene or the transcript thereof can serve as a marker for liver cancer (Patent Literature 4).

The 171st target gene is the hsa-miR-1228-3p gene, a congener thereof, a transcript thereof, or a variant or a derivative thereof. None of the previously known reports show that change in the expression of the gene or the transcript thereof can serve as a marker for liver cancer.

The 172nd target gene is the hsa-miR-614 gene, a congener thereof, a transcript thereof, or a variant or a derivative thereof. The previously known report shows that change in the expression of the gene or the transcript thereof can serve as a marker for liver cancer (Patent Literature 2).

The 173rd target gene is the hsa-miR-1913 gene, a congener thereof, a transcript thereof, or a variant or a derivative thereof. The previously known report shows that change in the expression of the gene or the transcript thereof can serve as a marker for liver cancer (Patent Literature 4).

The 174th target gene is the hsa-miR-92a-2-5p gene, a congener thereof, a transcript thereof, or a variant or a derivative thereof. The previously known report shows that change in the expression of the gene or the transcript thereof can serve as a marker for liver cancer (Patent Literature 1).

The 175th target gene is the hsa-miR-187-5p gene, a congener thereof, a transcript thereof, or a variant or a derivative thereof. The previously known report shows that change in the expression of the gene or the transcript thereof can serve as a marker for liver cancer (Patent Literature 5).

The 176th target gene is the hsa-miR-16-5p gene, a congener thereof, a transcript thereof, or a variant or a derivative thereof. The previously known report shows that change in the expression of the gene or the transcript thereof can serve as a marker for liver cancer (Patent Literatures 4 and 5).

The 177th target gene is the hsa-miR-92b-3p gene, a congener thereof, a transcript thereof, or a variant or a derivative thereof. The previously known report shows that change in the expression of the gene or the transcript thereof can serve as a marker for liver cancer (Patent Literature 1).

The 178th target gene is the hsa-miR-150-3p gene, a congener thereof, a transcript thereof, or a variant or a derivative thereof. The previously known report shows that change in the expression of the gene or the transcript thereof can serve as a marker for liver cancer (Patent Literature 2).

The 179th target gene is the hsa-miR-564 gene, a congener thereof, a transcript thereof, or a variant or a derivative thereof. The previously known report shows that change in the expression of the gene or the transcript thereof can serve as a marker for liver cancer (Patent Literature 2).

The 180th target gene is the hsa-miR-125a-3p gene, a congener thereof, a transcript thereof, or a variant or a derivative thereof. The previously known report shows that change in the expression of the gene or the transcript thereof can serve as a marker for liver cancer (Patent Literature 3).

The 181st target gene is the hsa-miR-92b-5p gene, a congener thereof, a transcript thereof, or a variant or a derivative thereof. The previously known report shows that change in the expression of the gene or the transcript thereof can serve as a marker for liver cancer (Patent Literature 1).

The 182nd target gene is the hsa-miR-92a-3p gene, a congener thereof, a transcript thereof, or a variant or a derivative thereof. The previously known report shows that change in the expression of the gene or the transcript thereof can serve as a marker for liver cancer (Patent Literatures 1, 4, and 5).

The 183rd target gene is the hsa-miR-663a gene, a congener thereof, a transcript thereof, or a variant or a derivative thereof. The previously known report shows that change in the expression of the gene or the transcript thereof can serve as a marker for liver cancer (Patent Literature 4).

The 184th target gene is the hsa-miR-4688 gene, a congener thereof, a transcript thereof, or a variant or a derivative thereof. None of the previously known reports show that change in the expression of the gene or the transcript thereof can serve as a marker for liver cancer.

The 185th target gene is the hsa-miR-4648 gene, a congener thereof, a transcript thereof, or a variant or a derivative thereof. None of the previously known reports show that change in the expression of the gene or the transcript thereof can serve as a marker for liver cancer.

The 186th target gene is the hsa-miR-6085 gene, a congener thereof, a transcript thereof, or a variant or a derivative thereof. None of the previously known reports show that change in the expression of the gene or the transcript thereof can serve as a marker for liver cancer.

The 187th target gene is the hsa-miR-6126 gene, a congener thereof, a transcript thereof, or a variant or a derivative thereof. None of the previously known reports show that change in the expression of the gene or the transcript thereof can serve as a marker for liver cancer.

The 188th target gene is the hsa-miR-6880-5p gene, a congener thereof, a transcript thereof, or a variant or a derivative thereof. None of the previously known reports show that change in the expression of the gene or the transcript thereof can serve as a marker for liver cancer.

The 189th target gene is the hsa-miR-328-5p gene, a congener thereof, a transcript thereof, or a variant or a derivative thereof. None of the previously known reports show that change in the expression of the gene or the transcript thereof can serve as a marker for liver cancer.

The 190th target gene is the hsa-miR-6768-5p gene, a congener thereof, a transcript thereof, or a variant or a derivative thereof. None of the previously known reports show that change in the expression of the gene or the transcript thereof can serve as a marker for liver cancer.

The 191st target gene is the hsa-miR-3180 gene, a congener thereof, a transcript thereof, or a variant or a derivative thereof. None of the previously known reports show that change in the expression of the gene or the transcript thereof can serve as a marker for liver cancer.

The 192nd target gene is the hsa-miR-6087 gene, a congener thereof, a transcript thereof, or a variant or a derivative thereof. None of the previously known reports show that change in the expression of the gene or the transcript thereof can serve as a marker for liver cancer.

The 193rd target gene is the hsa-miR-1273g-3p gene, a congener thereof, a transcript thereof, or a variant or a derivative thereof. None of the previously known reports show that change in the expression of the gene or the transcript thereof can serve as a marker for liver cancer.

The 194th target gene is the hsa-miR-1225-5p gene, a congener thereof, a transcript thereof, or a variant or a derivative thereof. None of the previously known reports show that change in the expression of the gene or the transcript thereof can serve as a marker for liver cancer.

The 195th target gene is the hsa-miR-3196 gene, a congener thereof, a transcript thereof, or a variant or a derivative thereof. None of the previously known reports show that change in the expression of the gene or the transcript thereof can serve as a marker for liver cancer.

The 196th target gene is the hsa-miR-4695-5p gene, a congener thereof, a transcript thereof, or a variant or a derivative thereof. None of the previously known reports show that change in the expression of the gene or the transcript thereof can serve as a marker for liver cancer.

The 197th target gene is the hsa-miR-6732-5p gene, a congener thereof, a transcript thereof, or a variant or a derivative thereof. None of the previously known reports show that change in the expression of the gene or the transcript thereof can serve as a marker for liver cancer.

The 198th target gene is the hsa-miR-638 gene, a congener thereof, a transcript thereof, or a variant or a derivative thereof. None of the previously known reports show that change in the expression of the gene or the transcript thereof can serve as a marker for liver cancer.

The 199th target gene is the hsa-miR-6813-5p gene, a congener thereof, a transcript thereof, or a variant or a derivative thereof. None of the previously known reports show that change in the expression of the gene or the transcript thereof can serve as a marker for liver cancer.

The 200th target gene is the hsa-miR-665 gene, a congener thereof, a transcript thereof, or a variant or a derivative thereof. None of the previously known reports show that change in the expression of the gene or the transcript thereof can serve as a marker for liver cancer.

The 201st target gene is the hsa-miR-486-3p gene, a congener thereof, a transcript thereof, or a variant or a derivative thereof. The previously known report shows that change in the expression of the gene or the transcript thereof can serve as a marker for liver cancer (Patent Literatures 2 and 3).

The 202nd target gene is the hsa-miR-4466 gene, a congener thereof, a transcript thereof, or a variant or a derivative thereof. None of the previously known reports show that change in the expression of the gene or the transcript thereof can serve as a marker for liver cancer.

The 203rd target gene is the hsa-miR-30c-1-3p gene, a congener thereof, a transcript thereof, or a variant or a derivative thereof. The previously known report shows that change in the expression of the gene or the transcript thereof can serve as a marker for liver cancer (Patent Literatures 3 and 5).

The 204th target gene is the hsa-miR-3621 gene, a congener thereof, a transcript thereof, or a variant or a derivative thereof. None of the previously known reports show that change in the expression of the gene or the transcript thereof can serve as a marker for liver cancer.

The 205th target gene is the hsa-miR-6743-5p gene, a congener thereof, a transcript thereof, or a variant or a derivative thereof. None of the previously known reports show that change in the expression of the gene or the transcript thereof can serve as a marker for liver cancer.

The 206th target gene is the hsa-miR-4298 gene, a congener thereof, a transcript thereof, or a variant or a derivative thereof. None of the previously known reports show that change in the expression of the gene or the transcript thereof can serve as a marker for liver cancer.

The 207th target gene is the hsa-miR-4741 gene, a congener thereof, a transcript thereof, or a variant or a derivative thereof. None of the previously known reports show that change in the expression of the gene or the transcript thereof can serve as a marker for liver cancer.

The 208th target gene is the hsa-miR-3619-3p gene, a congener thereof, a transcript thereof, or a variant or a derivative thereof. None of the previously known reports show that change in the expression of the gene or the transcript thereof can serve as a marker for liver cancer.

The 209th target gene is the hsa-miR-6824-5p gene, a congener thereof, a transcript thereof, or a variant or a derivative thereof. None of the previously known reports show that change in the expression of the gene or the transcript thereof can serve as a marker for liver cancer.

The 210th target gene is the hsa-miR-5698 gene, a congener thereof, a transcript thereof, or a variant or a derivative thereof. None of the previously known reports show that change in the expression of the gene or the transcript thereof can serve as a marker for liver cancer.

The 211th target gene is the hsa-miR-371a-5p gene, a congener thereof, a transcript thereof, or a variant or a derivative thereof. None of the previously known reports show that change in the expression of the gene or the transcript thereof can serve as a marker for liver cancer.

The 212th target gene is the hsa-miR-4488 gene, a congener thereof, a transcript thereof, or a variant or a derivative thereof. None of the previously known reports show that change in the expression of the gene or the transcript thereof can serve as a marker for liver cancer.

The 213th target gene is the hsa-miR-1233-5p gene, a congener thereof, a transcript thereof, or a variant or a derivative thereof. None of the previously known reports show that change in the expression of the gene or the transcript thereof can serve as a marker for liver cancer.

The 214th target gene is the hsa-miR-4723-5p gene, a congener thereof, a transcript thereof, or a variant or a derivative thereof. None of the previously known reports show that change in the expression of the gene or the transcript thereof can serve as a marker for liver cancer.

The 215th target gene is the hsa-miR-24-3p gene, a congener thereof, a transcript thereof, or a variant or a derivative thereof. The previously known report shows that change in the expression of the gene or the transcript thereof can serve as a marker for liver cancer (Patent Literature 2).

The 216th target gene is the hsa-miR-1238-5p gene, a congener thereof, a transcript thereof, or a variant or a derivative thereof. None of the previously known reports show that change in the expression of the gene or the transcript thereof can serve as a marker for liver cancer.

The 217th target gene is the hsa-miR-4442 gene, a congener thereof, a transcript thereof, or a variant or a derivative thereof. None of the previously known reports show that change in the expression of the gene or the transcript thereof can serve as a marker for liver cancer.

The 218th target gene is the hsa-miR-3928-3p gene, a congener thereof, a transcript thereof, or a variant or a derivative thereof. None of the previously known reports show that change in the expression of the gene or the transcript thereof can serve as a marker for liver cancer.

The 219th target gene is the hsa-miR-6716-5p gene, a congener thereof, a transcript thereof, or a variant or a derivative thereof. None of the previously known reports show that change in the expression of the gene or the transcript thereof can serve as a marker for liver cancer.

The 220th target gene is the hsa-miR-6089 gene, a congener thereof, a transcript thereof, or a variant or a derivative thereof. None of the previously known reports show that change in the expression of the gene or the transcript thereof can serve as a marker for liver cancer.

The 221st target gene is the hsa-miR-6124 gene, a congener thereof, a transcript thereof, or a variant or a derivative thereof. None of the previously known reports show that change in the expression of the gene or the transcript thereof can serve as a marker for liver cancer.

The 222nd target gene is the hsa-miR-6778-5p gene, a congener thereof, a transcript thereof, or a variant or a derivative thereof. None of the previously known reports show that change in the expression of the gene or the transcript thereof can serve as a marker for liver cancer.

The 223rd target gene is the hsa-miR-557 gene, a congener thereof, a transcript thereof, or a variant or a derivative thereof. The previously known report shows that change in the expression of the gene or the transcript thereof can serve as a marker for liver cancer (Patent Literature 2).

The 224th target gene is the hsa-miR-6090 gene, a congener thereof, a transcript thereof, or a variant or a derivative thereof. None of the previously known reports show that change in the expression of the gene or the transcript thereof can serve as a marker for liver cancer.

The 225th target gene is the hsa-miR-6757-5p gene, a congener thereof, a transcript thereof, or a variant or a derivative thereof. None of the previously known reports show that change in the expression of the gene or the transcript thereof can serve as a marker for liver cancer.

The 226th target gene is the hsa-miR-4448 gene, a congener thereof, a transcript thereof, or a variant or a derivative thereof. None of the previously known reports show that change in the expression of the gene or the transcript thereof can serve as a marker for liver cancer.

The 227th target gene is the hsa-miR-671-5p gene, a congener thereof, a transcript thereof, or a variant or a derivative thereof. None of the previously known reports show that change in the expression of the gene or the transcript thereof can serve as a marker for liver cancer.

The 228th target gene is the hsa-miR-3178 gene, a congener thereof, a transcript thereof, or a variant or a derivative thereof. None of the previously known reports show that change in the expression of the gene or the transcript thereof can serve as a marker for liver cancer.

The 229th target gene is the hsa-miR-4725-3p gene, a congener thereof, a transcript thereof, or a variant or a derivative thereof. None of the previously known reports show that change in the expression of the gene or the transcript thereof can serve as a marker for liver cancer.

The 230th target gene is the hsa-miR-940 gene, a congener thereof, a transcript thereof, or a variant or a derivative thereof. None of the previously known reports show that change in the expression of the gene or the transcript thereof can serve as a marker for liver cancer.

The 231st target gene is the hsa-miR-6789-5p gene, a congener thereof, a transcript thereof, or a variant or a derivative thereof. None of the previously known reports show that change in the expression of the gene or the transcript thereof can serve as a marker for liver cancer.

The 232nd target gene is the hsa-miR-4484 gene, a congener thereof, a transcript thereof, or a variant or a derivative thereof. None of the previously known reports show that change in the expression of the gene or the transcript thereof can serve as a marker for liver cancer.

The 233rd target gene is the hsa-miR-4634 gene, a congener thereof, a transcript thereof, or a variant or a derivative thereof. None of the previously known reports show that change in the expression of the gene or the transcript thereof can serve as a marker for liver cancer.

The 234th target gene is the hsa-miR-4745-5p gene, a congener thereof, a transcript thereof, or a variant or a derivative thereof. None of the previously known reports show that change in the expression of the gene or the transcript thereof can serve as a marker for liver cancer.

The 235th target gene is the hsa-miR-4730 gene, a congener thereof, a transcript thereof, or a variant or a derivative thereof. None of the previously known reports show that change in the expression of the gene or the transcript thereof can serve as a marker for liver cancer.

The 236th target gene is the hsa-miR-6803-5p gene, a congener thereof, a transcript thereof, or a variant or a derivative thereof. None of the previously known reports show that change in the expression of the gene or the transcript thereof can serve as a marker for liver cancer.

The 237th target gene is the hsa-miR-6798-5p gene, a congener thereof, a transcript thereof, or a variant or a derivative thereof. None of the previously known reports show that change in the expression of the gene or the transcript thereof can serve as a marker for liver cancer.

The 238th target gene is the hsa-miR-3648 gene, a congener thereof, a transcript thereof, or a variant or a derivative thereof. None of the previously known reports show that change in the expression of the gene or the transcript thereof can serve as a marker for liver cancer.

The 239th target gene is the hsa-miR-4783-3p gene, a congener thereof, a transcript thereof, or a variant or a derivative thereof. None of the previously known reports show that change in the expression of the gene or the transcript thereof can serve as a marker for liver cancer.

The 240th target gene is the hsa-miR-6836-3p gene, a congener thereof, a transcript thereof, or a variant or a derivative thereof. None of the previously known reports show that change in the expression of the gene or the transcript thereof can serve as a marker for liver cancer.

2. Nucleic Acid Probe or Primer for Detection of Liver Cancer

In the present invention, a nucleic acid capable of specifically binding to any of the target nucleic acids as the liver cancer markers described above can be used as a nucleic acid, for example, a nucleic acid probe or a primer, for the detection or diagnosis of liver cancer.

In the present invention, the nucleic acid probe or the primer that can be used for detecting liver cancer or for diagnosing liver cancer enables qualitative and/or quantitative measurement of the presence, expression level, or abundance of a target nucleic acid as the liver cancer marker described above, for example, human-derived hsa-miR-1343-3p, hsa-miR-6726-5p, hsa-miR-6515-3p, hsa-miR-4651, hsa-miR-4257, hsa-miR-3188, hsa-miR-6131, hsa-miR-6766-3p, hsa-miR-7641, hsa-miR-1249, hsa-miR-3679-3p, hsa-miR-6787-5p, hsa-miR-4454, hsa-miR-3135b, hsa-miR-6765-3p, hsa-miR-7975, hsa-miR-204-3p, hsa-miR-7977, hsa-miR-7110-5p, hsa-miR-6717-5p, hsa-miR-6870-5p, hsa-miR-663b, hsa-miR-6875-5p, hsa-miR-8072, hsa-miR-6816-5p, hsa-miR-4281, hsa-miR-6729-5p, hsa-miR-8069, hsa-miR-4706, hsa-miR-7108-5p, hsa-miR-4433b-3p, hsa-miR-6893-5p, hsa-miR-6857-5p, hsa-miR-1227-5p, hsa-miR-6741-5p, hsa-miR-451a, hsa-miR-8063, hsa-miR-3622a-5p, hsa-miR-615-5p, hsa-miR-128-1-5p, hsa-miR-6825-5p, hsa-miR-1260b, hsa-miR-4433-3p, hsa-miR-4665-5p, hsa-miR-7845-5p, hsa-miR-1908-5p, hsa-miR-6840-3p, hsa-miR-6765-5p, hsa-miR-296-5p, hsa-miR-36′75-3p, hsa-miR-6781-5p, hsa-miR-423-5p, hsa-miR-3663-3p, hsa-miR-6784-5p, hsa-miR-6749-5p, hsa-miR-1231, hsa-miR-4746-3p, hsa-miR-6780b-5p, hsa-miR-4758-5p, hsa-miR-36′79-5p, hsa-miR-3184-5p, hsa-miR-6125, hsa-miR-6721-5p, hsa-miR-6791-5p, hsa-miR-3185, hsa-miR-1260a, hsa-miR-3197, hsa-miR-6845-5p, hsa-miR-6887-5p, hsa-miR-6738-5p, hsa-miR-6872-3p, hsa-miR-4497, hsa-miR-1229-5p, hsa-miR-6820-5p, hsa-miR-6777-5p, hsa-miR-3917, hsa-miR-5787, hsa-miR-4286, hsa-miR-6877-5p, hsa-miR-1225-3p, hsa-miR-6088, hsa-miR-6800-5p, hsa-miR-1246, hsa-miR-4467, hsa-miR-4419b, hsa-miR-1914-3p, hsa-miR-4632-5p, hsa-miR-1915-5p, hsa-miR-3940-5p, hsa-miR-1185-2-3p, hsa-miR-6746-5p, hsa-miR-5001-5p, hsa-miR-1228-5p, hsa-miR-5572, hsa-miR-4327, hsa-miR-4638-5p, hsa-miR-6799-5p, hsa-miR-6861-5p, hsa-miR-6727-5p, hsa-miR-4513, hsa-miR-6805-3p, hsa-miR-6808-5p, hsa-miR-4449, hsa-miR-1199-5p, hsa-miR-1275, hsa-miR-4792, hsa-miR-4443, hsa-miR-6891-5p, hsa-miR-6826-5p, hsa-miR-6807-5p, hsa-miR-7150, hsa-miR-4534, hsa-miR-4476, hsa-miR-4649-5p, hsa-miR-4525, hsa-miR-1915-3p, hsa-miR-4516, hsa-miR-4417, hsa-miR-642b-3p, hsa-miR-3141, hsa-miR-5100, hsa-miR-6848-5p, hsa-miR-4739, hsa-miR-4459, hsa-miR-1237-5p, hsa-miR-296-3p, hsa-miR-4665-3p, hsa-miR-6786-5p, hsa-miR-4258, hsa-miR-6510-5p, hsa-miR-1343-5p, hsa-miR-1247-3p, hsa-miR-6805-5p, hsa-miR-4492, hsa-miR-1469, hsa-miR-1268b, hsa-miR-6858-5p, hsa-miR-3937, hsa-miR-939-5p, hsa-miR-3656, hsa-miR-744-5p, hsa-miR-4687-3p, hsa-miR-4763-3p, hsa-miR-3620-5p, hsa-miR-3195, hsa-miR-6842-5p, hsa-miR-4707-5p, hsa-miR-642a-3p, hsa-miR-7113-3p, hsa-miR-4728-5p, hsa-miR-5195-3p, hsa-miR-1185-1-3p, hsa-miR-6774-5p, hsa-miR-8059, hsa-miR-3131, hsa-miR-7847-3p, hsa-miR-4463, hsa-miR-128-2-5p, hsa-miR-4508, hsa-miR-6806-5p, hsa-miR-7111-5p, hsa-miR-6782-5p, hsa-miR-4734, hsa-miR-3162-5p, hsa-miR-887-3p, hsa-miR-6752-5p, hsa-miR-6724-5p, hsa-miR-6757-5p, hsa-miR-4448, hsa-miR-671-5p, hsa-miR-3178, hsa-miR-4725-3p, hsa-miR-940, hsa-miR-6789-5p, hsa-miR-4484, hsa-miR-4634, hsa-miR-4745-5p, hsa-miR-4730, hsa-miR-6803-5p, hsa-miR-6798-5p, hsa-miR-3648, hsa-miR-4783-3p, or hsa-miR-6836-3p, or a combination thereof, or a congener thereof, a transcript thereof, or a variant or derivative thereof; and, optionally in combination therewith, hsa-miR-23b-3p, hsa-miR-23a-3p, hsa-miR-625-3p, hsa-miR-1228-3p, hsa-miR-614, hsa-miR-1913, hsa-miR-92a-2-5p, hsa-miR-187-5p, hsa-miR-16-5p, hsa-miR-92b-3p, hsa-miR-150-3p, hsa-miR-564, hsa-miR-125a-3p, hsa-miR-92b-5p, hsa-miR-92a-3p, or hsa-miR-663a, or a combination thereof, a congener thereof, a transcript thereof, or a variant or derivative thereof; and optionally in combination therewith, hsa-miR-4688, hsa-miR-4648, hsa-miR-6085, hsa-miR-6126, hsa-miR-6880-5p, hsa-miR-328-5p, hsa-miR-6768-5p, hsa-miR-3180, hsa-miR-6087, hsa-miR-1273g-3p, hsa-miR-1225-5p, hsa-miR-3196, hsa-miR-4695-5p, hsa-miR-6732-5p, hsa-miR-638, hsa-miR-6813-5p, hsa-miR-665, hsa-miR-486-3p, hsa-miR-4466, hsa-miR-30c-1-3p, hsa-miR-3621, hsa-miR-6743-5p, hsa-miR-4298, hsa-miR-4741, hsa-miR-3619-3p, hsa-miR-6824-5p, hsa-miR-5698, hsa-miR-371a-5p, hsa-miR-4488, hsa-miR-1233-5p, hsa-miR-4723-5p, hsa-miR-24-3p, hsa-miR-1238-5p, hsa-miR-4442, hsa-miR-3928-3p, hsa-miR-6716-5p, hsa-miR-6089, hsa-miR-6124, hsa-miR-6778-5p, hsa-miR-557, and hsa-miR-6090, or a combination thereof, a congener thereof, a transcript thereof, or a variant or derivative thereof.

The expression level of each target nucleic acid described above is increased or decreased (hereinafter, referred to as “increased/decreased”) depending on the type of the target nucleic acid in a subject having liver cancer as compared with a healthy subject. Hence, the nucleic acid of the present invention can be effectively used for measuring the expression level of the target nucleic acid described above in a body fluid derived from a subject (e.g., a human) suspected of having liver cancer and a body fluid derived from a healthy subject and comparing them to detect liver cancer.

The nucleic acid probe or the primer that can be used in the present invention is a nucleic acid probe capable of specifically binding to a polynucleotide consisting of a nucleotide sequence represented by at least one of SEQ ID NOs: 1 to 167 and 714 to 729, or a primer for amplifying a polynucleotide consisting of a nucleotide sequence represented by at least one of SEQ ID NOs: 1 to 167 and 714 to 729.

The nucleic acid probe or the primer that can be further used in the present invention may comprise a nucleic acid probe capable of specifically binding to a polynucleotide consisting of a nucleotide sequence represented by at least one of SEQ ID NOs: 168 to 183, or a primer for amplifying a polynucleotide consisting of a nucleotide sequence represented by at least one of SEQ ID NOs: 168 to 183.

The nucleic acid probe or the primer that can be further used in the present invention may comprise a nucleic acid probe capable of specifically binding to a polynucleotide consisting of a nucleotide sequence represented by at least one of SEQ ID NOs: 184 to 224, or a primer for amplifying a polynucleotide consisting of a nucleotide sequence represented by at least one of SEQ ID NOs: 184 to 224.

Specifically, these nucleic acid probes or primers comprise a combination of one or more polynucleotides selected from a group of polynucleotides comprising nucleotide sequences represented by any of SEQ ID NOs: 1 to 765 or nucleotide sequences derived from the nucleotide sequences by the replacement of u with t, and a group of complementary polynucleotides thereof, a group of polynucleotides respectively hybridizing under stringent conditions (mentioned later) to DNAs consisting of nucleotide sequences complementary to these nucleotide sequences, and a group of complementary polynucleotides thereof, and a group of polynucleotides comprising 15 or more, preferably 17 or more consecutive nucleotides in the nucleotide sequences of these polynucleotide groups. These polynucleotides can be used as nucleic acid probes and primers for detecting the liver cancer markers as target nucleic acids.

More specifically, examples of the nucleic acid probe or the primer that can be used in the present invention include one or more polynucleotide(s) selected from the group consisting of the following polynucleotides (a) to (e):
(a) a polynucleotide consisting of a nucleotide sequence represented by any of SEQ ID NOs: 1 to 167 and 714 to 729 or a nucleotide sequence derived from the nucleotide sequence by the replacement of u with t, a variant thereof, a derivative thereof, or a fragment thereof comprising 15 or more consecutive nucleotides,
(b) a polynucleotide comprising a nucleotide sequence represented by any of SEQ ID NOs: 1 to 167 and 714 to 729,
(c) a polynucleotide consisting of a nucleotide sequence complementary to a nucleotide sequence represented by any of SEQ ID NOs: 1 to 167 and 714 to 729 or a nucleotide sequence derived from the nucleotide sequence by the replacement of u with t, a variant thereof, a derivative thereof, or a fragment thereof comprising 15 or more consecutive nucleotides,
(d) a polynucleotide comprising a nucleotide sequence complementary to a nucleotide sequence represented by any of SEQ ID NOs: 1 to 167 and 714 to 729 or a nucleotide sequence derived from the nucleotide sequence by the replacement of u with t, and
(e) a polynucleotide hybridizing under stringent conditions to any of the polynucleotides (a) to (d).

In addition to at least one or more polynucleotide(s) selected from the group consisting of the polynucleotides (a) to (e), the nucleic acid probe or the primer that can be further used in the present invention may comprise a polynucleotide selected from the group consisting of the following polynucleotides (f) to (j):
(f) a polynucleotide consisting of a nucleotide sequence represented by any of SEQ ID NOs: 168 to 183 or a nucleotide sequence derived from the nucleotide sequence by the replacement of u with t, a variant thereof, a derivative thereof, or a fragment thereof comprising 15 or more consecutive nucleotides,
(g) a polynucleotide comprising a nucleotide sequence represented by any of SEQ ID NOs: 168 to 183,
(h) a polynucleotide consisting of a nucleotide sequence complementary to a nucleotide sequence represented by any of SEQ ID NOs: 168 to 183 or a nucleotide sequence derived from the nucleotide sequence by the replacement of u with t, a variant thereof, a derivative thereof, or a fragment thereof comprising 15 or more consecutive nucleotides,
(i) a polynucleotide comprising a nucleotide sequence complementary to a nucleotide sequence represented by any of SEQ ID NOs: 168 to 183 or a nucleotide sequence derived from the nucleotide sequence by the replacement of u with t, and
(j) a polynucleotide hybridizing under stringent conditions to any of the polynucleotides (f) to (i).

In addition to at least one or more polynucleotide(s) selected from the group consisting of the polynucleotides (a) to (j), the nucleic acid probe or the primer that can be further used in the present invention may comprise a polynucleotide selected from the group consisting of the following polynucleotides (k) to (o):
(k) a polynucleotide consisting of a nucleotide sequence represented by any of SEQ ID NOs: 184 to 224 or a nucleotide sequence derived from the nucleotide sequence by the replacement of u with t, a variant thereof, a derivative thereof, or a fragment thereof comprising 15 or more consecutive nucleotides,
(l) a polynucleotide comprising a nucleotide sequence represented by any of SEQ ID NOs: 184 to 224,
(m) a polynucleotide consisting of a nucleotide sequence complementary to a nucleotide sequence represented by any of SEQ ID NOs: 184 to 224 or a nucleotide sequence derived from the nucleotide sequence by the replacement of u with t, a variant thereof, a derivative thereof, or a fragment thereof comprising 15 or more consecutive nucleotides,
(n) a polynucleotide comprising a nucleotide sequence complementary to a nucleotide sequence represented by any of SEQ ID NOs: 184 to 224 or a nucleotide sequence derived from the nucleotide sequence by the replacement of u with t, and
(o) a polynucleotide hybridizing under stringent conditions to any of the polynucleotides (k) to (n).

For these polynucleotides, the “fragment thereof comprising 15 or more consecutive nucleotides” can comprise the number of nucleotides in the range from, for example, 15 consecutive nucleotides to less than the total number of nucleotides of the sequence, 17 consecutive nucleotides to less than the total number of nucleotides of the sequence, or 19 consecutive nucleotides to less than the total number of nucleotides of the sequence, in the nucleotide sequence of each polynucleotide, though the fragment is not limited thereto.

These polynucleotides or fragments thereof used in the present invention may each be DNA or may each be RNA.

The polynucleotides that can be used in the present invention can each be prepared by use of a general technique such as a DNA recombination technique, PCR, or a method using an automatic DNA/RNA synthesizer.

The DNA recombination technique and the PCR can employ a technique described in, for example, Ausubel et al., Current Protocols in Molecular Biology, John Willey & Sons, US (1993); and Sambrook et al., Molecular Cloning—A Laboratory Manual, Cold Spring Harbor Laboratory Press, US (1989).

The human-derived hsa-miR-1343-3p, hsa-miR-6726-5p, hsa-miR-6515-3p, hsa-miR-4651, hsa-miR-4257, hsa-miR-3188, hsa-miR-6131, hsa-miR-6766-3p, hsa-miR-7641, hsa-miR-1249, hsa-miR-3679-3p, hsa-miR-6787-5p, hsa-miR-4454, hsa-miR-3135b, hsa-miR-6765-3p, hsa-miR-7975, hsa-miR-204-3p, hsa-miR-7977, hsa-miR-7110-5p, hsa-miR-6717-5p, hsa-miR-6870-5p, hsa-miR-663b, hsa-miR-6875-5p, hsa-miR-8072, hsa-miR-6816-5p, hsa-miR-4281, hsa-miR-6729-5p, hsa-miR-8069, hsa-miR-4706, hsa-miR-7108-5p, hsa-miR-4433b-3p, hsa-miR-6893-5p, hsa-miR-6857-5p, hsa-miR-1227-5p, hsa-miR-6741-5p, hsa-miR-451a, hsa-miR-8063, hsa-miR-3622a-5p, hsa-miR-615-5p, hsa-miR-128-1-5p, hsa-miR-6825-5p, hsa-miR-1260b, hsa-miR-4433-3p, hsa-miR-4665-5p, hsa-miR-7845-5p, hsa-miR-1908-5p, hsa-miR-6840-3p, hsa-miR-6765-5p, hsa-miR-296-5p, hsa-miR-3675-3p, hsa-miR-6781-5p, hsa-miR-423-5p, hsa-miR-3663-3p, hsa-miR-6784-5p, hsa-miR-6749-5p, hsa-miR-1231, hsa-miR-4746-3p, hsa-miR-6780b-5p, hsa-miR-4758-5p, hsa-miR-3679-5p, hsa-miR-3184-5p, hsa-miR-6125, hsa-miR-6721-5p, hsa-miR-6791-5p, hsa-miR-3185, hsa-miR-1260a, hsa-miR-3197, hsa-miR-6845-5p, hsa-miR-6887-5p, hsa-miR-6738-5p, hsa-miR-6872-3p, hsa-miR-4497, hsa-miR-1229-5p, hsa-miR-6820-5p, hsa-miR-6777-5p, hsa-miR-3917, hsa-miR-5787, hsa-miR-4286, hsa-miR-6877-5p, hsa-miR-1225-3p, hsa-miR-6088, hsa-miR-6800-5p, hsa-miR-1246, hsa-miR-4467, hsa-miR-4419b, hsa-miR-1914-3p, hsa-miR-4632-5p, hsa-miR-1915-5p, hsa-miR-3940-5p, hsa-miR-1185-2-3p, hsa-miR-6746-5p, hsa-miR-5001-5p, hsa-miR-1228-5p, hsa-miR-5572, hsa-miR-4327, hsa-miR-4638-5p, hsa-miR-6799-5p, hsa-miR-6861-5p, hsa-miR-6727-5p, hsa-miR-4513, hsa-miR-6805-3p, hsa-miR-6808-5p, hsa-miR-4449, hsa-miR-1199-5p, hsa-miR-1275, hsa-miR-4792, hsa-miR-4443, hsa-miR-6891-5p, hsa-miR-6826-5p, hsa-miR-6807-5p, hsa-miR-7150, hsa-miR-4534, hsa-miR-4476, hsa-miR-4649-5p, hsa-miR-4525, hsa-miR-1915-3p, hsa-miR-4516, hsa-miR-4417, hsa-miR-642b-3p, hsa-miR-3141, hsa-miR-5100, hsa-miR-6848-5p, hsa-miR-4739, hsa-miR-4459, hsa-miR-1237-5p, hsa-miR-296-3p, hsa-miR-4665-3p, hsa-miR-6786-5p, hsa-miR-4258, hsa-miR-6510-5p, hsa-miR-1343-5p, hsa-miR-1247-3p, hsa-miR-6805-5p, hsa-miR-4492, hsa-miR-1469, hsa-miR-1268b, hsa-miR-6858-5p, hsa-miR-3937, hsa-miR-939-5p, hsa-miR-3656, hsa-miR-744-5p, hsa-miR-4687-3p, hsa-miR-4763-3p, hsa-miR-3620-5p, hsa-miR-3195, hsa-miR-6842-5p, hsa-miR-4707-5p, hsa-miR-642a-3p, hsa-miR-7113-3p, hsa-miR-4728-5p, hsa-miR-5195-3p, hsa-miR-1185-1-3p, hsa-miR-6774-5p, hsa-miR-8059, hsa-miR-3131, hsa-miR-7847-3p, hsa-miR-4463, hsa-miR-128-2-5p, hsa-miR-4508, hsa-miR-6806-5p, hsa-miR-7111-5p, hsa-miR-6782-5p, hsa-miR-4734, hsa-miR-3162-5p, hsa-miR-887-3p, hsa-miR-6752-5p, hsa-miR-6724-5p, hsa-miR-6757-5p, hsa-miR-4448, hsa-miR-671-5p, hsa-miR-3178, hsa-miR-4725-3p, hsa-miR-940, hsa-miR-6789-5p, hsa-miR-4484, hsa-miR-4634, hsa-miR-4745-5p, hsa-miR-4730, hsa-miR-6803-5p, hsa-miR-6798-5p, hsa-miR-3648, hsa-miR-4783-3p, hsa-miR-6836-3p, hsa-miR-23b-3p, hsa-miR-23a-3p, hsa-miR-625-3p, hsa-miR-1228-3p, hsa-miR-614, hsa-miR-1913, hsa-miR-92a-2-5p, hsa-miR-187-5p, hsa-miR-16-5p, hsa-miR-92b-3p, hsa-miR-150-3p, hsa-miR-564, hsa-miR-125a-3p, hsa-miR-92b-5p, hsa-miR-92a-3p, hsa-miR-663a, hsa-miR-4688, hsa-miR-4648, hsa-miR-6085, hsa-miR-6126, hsa-miR-6880-5p, hsa-miR-328-5p, hsa-miR-6768-5p, hsa-miR-3180, hsa-miR-6087, hsa-miR-1273g-3p, hsa-miR-1225-5p, hsa-miR-3196, hsa-miR-4695-5p, hsa-miR-6732-5p, hsa-miR-638, hsa-miR-6813-5p, hsa-miR-665, hsa-miR-486-3p, hsa-miR-4466, hsa-miR-30c-1-3p, hsa-miR-3621, hsa-miR-6743-5p, hsa-miR-4298, hsa-miR-4741, hsa-miR-3619-3p, hsa-miR-6824-5p, hsa-miR-5698, hsa-miR-371a-5p, hsa-miR-4488, hsa-miR-1233-5p, hsa-miR-4723-5p, hsa-miR-24-3p, hsa-miR-1238-5p, hsa-miR-4442, hsa-miR-3928-3p, hsa-miR-6716-5p, hsa-miR-6089, hsa-miR-6124, hsa-miR-6778-5p, hsa-miR-557 and hsa-miR-6090 represented by SEQ ID NOs: 1 to 224 and 714 to 729 are known in the art, and their obtainment methods are also known as mentioned above. Therefore, each polynucleotide that can be used as a nucleic acid probe or a primer in the present invention can be prepared by cloning the gene.

Such a nucleic acid probe or a primer can be chemically synthesized using an automated DNA synthesizer. In general, a phosphoramidite method is used in this synthesis, and single-stranded DNA up to approximately 100 nucleotides can be automatically synthesized by this method. The automated DNA synthesizer is commercially available from, for example, Polygen GmbH, ABI, or Applied Biosystems, Inc.

Alternatively, the polynucleotide of the present invention can also be prepared by a cDNA cloning method. The cDNA cloning technique can employ, for example, microRNA Cloning Kit Wako.

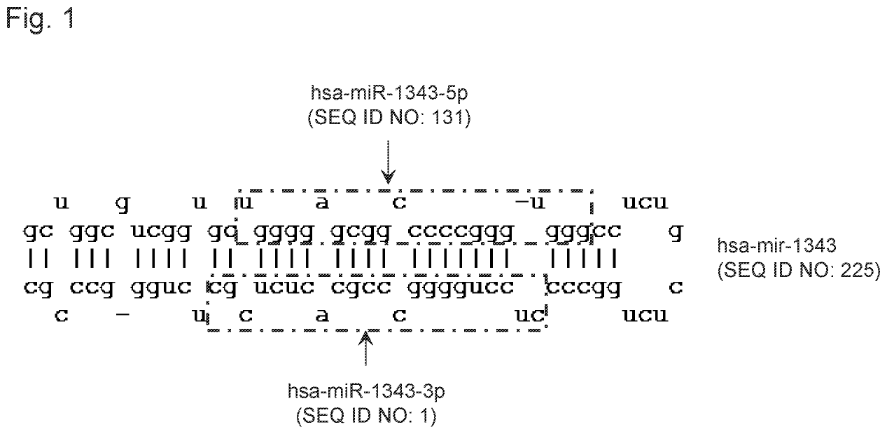

In this context, the sequences of the nucleic acid probe and the primer for detecting the polynucleotide consisting of a nucleotide sequence represented by any of SEQ ID NOs: 1 to 224 and 714 to 729 do not exist as miRNAs or precursors thereof in vivo. For example, the nucleotide sequences represented by SEQ ID NO: 1 and SEQ ID NO: 131 are produced from the precursor represented by SEQ ID NO: 225. This precursor has a hairpin-like structure as shown in FIG. 1, and the nucleotide sequences represented by SEQ ID NO: 1 and SEQ ID NO: 131 have mismatch sequences with each other. Therefore, a nucleotide sequence completely complementary to the nucleotide sequence represented by SEQ ID NO: 1 or SEQ ID NO: 131 is not naturally produced in vivo. Likewise, the nucleic acid probe and the primer for detecting the nucleotide sequence represented by any of SEQ ID NOs: 1 to 224 and 714 to 729 each have an artificial nucleotide sequence that does not exist in vivo.

3. Kit or Device for Detection of Liver Cancer

The present invention also provides a kit or a device for the detection of liver cancer, comprising one or more polynucleotide(s) (which may include a variant, a fragment, or a derivative thereof; hereinafter, also referred to as a polynucleotide for detection) that can be used as a nucleic acid probe or a primer in the present invention for measuring a target nucleic acid as a liver cancer marker.

The target nucleic acid as a liver cancer marker according to the present invention is preferably selected from the following group 1:

miR-1343-3p, miR-6726-5p, miR-6515-3p, miR-4651, miR-4257, miR-3188, miR-6131, miR-6766-3p, miR-7641, miR-1249, miR-3679-3p, miR-6787-5p, miR-4454, miR-3135b, miR-6765-3p, miR-7975, miR-204-3p, miR-7977, miR-7110-5p, miR-6717-5p, miR-6870-5p, miR-663b, miR-6875-5p, miR-8072, miR-6816-5p, miR-4281, miR-6729-5p, miR-8069, miR-4706, miR-7108-5p, miR-4433b-3p, miR-6893-5p, miR-6857-5p, miR-1227-5p, miR-6741-5p, miR-451a, miR-8063, miR-3622a-5p, miR-615-5p, miR-128-1-5p, miR-6825-5p, miR-1260b, miR-4433-3p, miR-4665-5p, miR-7845-5p, miR-1908-5p, miR-6840-3p, miR-6765-5p, miR-296-5p, miR-3675-3p, miR-6781-5p, miR-423-5p, miR-3663-3p, miR-6′784-5p, miR-6749-5p, miR-1231, miR-4746-3p, miR-6780b-5p, miR-4758-5p, miR-3679-5p, miR-3184-5p, miR-6125, miR-6721-5p, miR-6791-5p, miR-3185, miR-1260a, miR-3197, miR-6845-5p, miR-6887-5p, miR-6738-5p, miR-6872-3p, miR-4497, miR-1229-5p, miR-6820-5p, miR-6777-5p, miR-3917, miR-5787, miR-4286, miR-6877-5p, miR-1225-3p, miR-6088, miR-6800-5p, miR-1246, miR-4467, miR-4419b, miR-1914-3p, miR-4632-5p, miR-1915-5p, miR-3940-5p, miR-1185-2-3p, miR-6746-5p, miR-5001-5p, miR-1228-5p, miR-5572, miR-4327, miR-4638-5p, miR-6799-5p, miR-6861-5p, miR-6727-5p, miR-4513, miR-6805-3p, miR-6808-5p, miR-4449, miR-1199-5p, miR-1275, miR-4792, miR-4443, miR-6891-5p, miR-6826-5p, miR-6807-5p, miR-7150, miR-4534, miR-4476, miR-4649-5p, miR-4525, miR-1915-3p, miR-4516, miR-4417, miR-642b-3p, miR-3141, miR-5100, miR-6848-5p, miR-4739, miR-4459, miR-1237-5p, miR-296-3p, miR-4665-3p, miR-6786-5p, miR-4258, miR-6510-5p, miR-1343-5p, miR-1247-3p, miR-6805-5p, miR-4492, miR-1469, miR-1268b, miR-6858-5p, miR-3937, miR-939-5p, miR-3656, miR-744-5p, miR-4687-3p, miR-4763-3p, miR-3620-5p, miR-3195, miR-6842-5p, miR-4707-5p, miR-642a-3p, miR-7113-3p, miR-4728-5p, miR-5195-3p, miR-1185-1-3p, miR-6774-5p, miR-8059, miR-3131, miR-7847-3p, miR-4463, miR-128-2-5p, miR-4508, miR-6806-5p, miR-7111-5p, miR-6782-5p, miR-4734, miR-3162-5p, miR-887-3p, miR-6752-5p, miR-6724-5p, miR-6757-5p, miR-4448, miR-671-5p, miR-3178, miR-4725-3p, miR-940, miR-6789-5p, miR-4484, miR-4634, miR-4745-5p, miR-4730, miR-6803-5p, miR-6798-5p, miR-3648, miR-4783-3p and miR-6836-3p.

An additional target nucleic acid that may be optionally used in the measurement is preferably selected from the following group 2: miR-23b-3p, miR-23a-3p, miR-625-3p, miR-1228-3p, miR-614, miR-1913, miR-92a-2-5p, miR-187-5p, miR-16-5p, miR-92b-3p, miR-150-3p, miR-564, miR-125a-3p, miR-92b-5p, miR-92a-3p and miR-663a.

An additional target nucleic acid that can be optionally further used in the measurement is preferably selected from the following group 3: miR-4688, miR-4648, miR-6085, miR-6126, miR-6880-5p, miR-328-5p, miR-6768-5p, miR-3180, miR-6087, miR-1273g-3p, miR-1225-5p, miR-3196, miR-4695-5p, miR-6732-5p, miR-638, miR-6813-5p, miR-665, miR-486-3p, miR-4466, miR-30c-1-3p, miR-3621, miR-6743-5p, miR-4298, miR-4741, miR-3619-3p, miR-6824-5p, miR-5698, miR-371a-5p, miR-4488, miR-1233-5p, miR-4723-5p, miR-24-3p, miR-1238-5p, miR-4442, miR-3928-3p, miR-6716-5p, miR-6089, miR-6124, miR-6778-5p, miR-557 and miR-6090.

The kit or the device of the present invention comprises a nucleic acid capable of specifically binding to any of the target nucleic acids as the liver cancer markers described above, preferably one or more polynucleotide(s) selected from the nucleic acid probes or the primers described in Section 2 above, specifically, the polynucleotides described in Section 2 above, or variant(s) thereof.

Specifically, the kit or the device of the present invention may comprise at least one or more polynucleotide(s) comprising (or consisting of) a nucleotide sequence represented by any of SEQ ID NOs: 1 to 167 and 714 to 729 or a nucleotide sequence derived from the nucleotide sequence by the replacement of u with t, polynucleotide(s) comprising (or consisting of) a complementary sequence thereof, polynucleotide(s) hybridizing under stringent conditions to any of these polynucleotides, or variant(s) or fragment(s) comprising 15 or more consecutive nucleotides of any of these polynucleotide sequences.

The kit or the device of the present invention may further comprise one or more polynucleotide(s) comprising (or consisting of) a nucleotide sequence represented by any of SEQ ID NOs: 168 to 183 or a nucleotide sequence derived from the nucleotide sequence by the replacement of u with t, polynucleotide(s) comprising (or consisting of) a complementary sequence thereof, polynucleotide(s) hybridizing under stringent conditions to any of these polynucleotides, variant(s) or fragment(s) comprising 15 or more consecutive nucleotides of any of these polynucleotide sequences.

The kit or the device of the present invention may further comprise one or more polynucleotide(s) comprising (or consisting of) a nucleotide sequence represented by any of SEQ ID NOs: 184 to 224 or a nucleotide sequence derived from the nucleotide sequence by the replacement of u with t, polynucleotide(s) comprising (or consisting of) a complementary sequence thereof, polynucleotide(s) hybridizing under stringent conditions to any of these polynucleotides, variant(s) or fragment(s) comprising 15 or more consecutive nucleotides of any of these polynucleotide sequences.

The fragment that may be contained in the kit or the device of the present invention is, for example, one or more, preferably two or more polynucleotides selected from the group consisting of the following polynucleotides (1) to (3):
(1) a polynucleotide comprising 15 or more consecutive nucleotides in a nucleotide sequence derived from a nucleotide sequence represented by any of SEQ ID NOs: 1 to 167 and 714 to 729 by the replacement of u with t, or a complementary sequence thereof;
(2) a polynucleotide comprising 15 or more consecutive nucleotides in a nucleotide sequence derived from a nucleotide sequence represented by any of SEQ ID NOs: 168 to 183 by the replacement of u with t, or a complementary sequence thereof; and
(3) a polynucleotide comprising 15 or more consecutive nucleotides in a nucleotide sequence derived from a nucleotide sequence represented by any of SEQ ID NOs: 184 to 224 by the replacement of u with t, or a complementary sequence thereof.

In a preferred embodiment, the polynucleotide is a polynucleotide consisting of a nucleotide sequence represented by any of SEQ ID NOs: 1 to 167 and 714 to 729 or a nucleotide sequence derived from the nucleotide sequence by the replacement of u with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the polynucleotide is a polynucleotide consisting of a nucleotide sequence represented by any of SEQ ID NOs: 168 to 183 or a nucleotide sequence derived from the nucleotide sequence by the replacement of u with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the polynucleotide is a polynucleotide consisting of a nucleotide sequence represented by any of SEQ ID NOs: 184 to 224 or a nucleotide sequence derived from the nucleotide sequence by the replacement of u with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the fragment may be a polynucleotide comprising 15 or more, preferably 17 or more, more preferably 19 or more consecutive nucleotides.

In the present invention, the size of the polynucleotide fragment is the number of nucleotides in the range from, for example, 15 consecutive nucleotides to less than the total number of nucleotides of the sequence, 17 consecutive nucleotides to less than the total number of nucleotides of the sequence, or 19 consecutive nucleotides to less than the total number of nucleotides of the sequence, in the nucleotide sequence of each polynucleotide.

Specific examples of the aforementioned polynucleotide combination constituting the kit or the device of the present invention can include any combination of the polynucleotides consisting of nucleotide sequences represented by SEQ ID NOs shown in Table 1 (SEQ ID NOs: 1 to 224 and 714 to 729 corresponding to the miRNA markers in Table 1) or complementary sequences thereof. However, these are given merely for illustrative purposes, and all of various other possible combinations are included in the present invention.

The aforementioned combination constituting the kit or the device for discriminating a liver cancer patient from a healthy subject according to the present invention is desirably, for example, a combination of two or more of the aforementioned polynucleotides consisting of nucleotide sequences represented by SEQ ID NOs shown in Table 1. Usually, a combination of two of these polynucleotides can produce adequate performance.

The combination of two polynucleotides consisting of the nucleotide sequences or the complementary sequences thereof for specifically discriminating a liver cancer patient from a healthy subject is preferably a combination comprising at least one or more of newly found polynucleotides consisting of the nucleotide sequences represented by SEQ ID NOs: 1 to 167 and 714 to 729, among the combinations of two selected from the polynucleotides consisting of the nucleotide sequences represented by SEQ ID NOs: 1 to 224 and 714 to 729.

The combination of polynucleotides with cancer type specificity capable of discriminating a liver cancer patient not only from a healthy subject but also from other cancer patients is preferably, for example, a combination of a plurality of polynucleotides comprising at least one polynucleotide selected from the group consisting of polynucleotides consisting of the nucleotide sequences represented by SEQ ID NOs: 1, 2, 3, 5, 7, 9, 12, 17, 20, 22, 27, 28, 29, 38, 39, 44, 46, 48, 51, 54, 61, 76, 89, 93, 101, 109, 116, 123, 132, 134, 136, 148, 150, 151, 155, 157, 164, 166, 167, 172, 180, 186, 188, 189, 197, 198, 214, 216, 714, 715, 716, 717, 718, 719, 720, 721, 722, 723, 724, 725, 726, 727, 728 and 729 or complementary sequences thereof (hereinafter, this group is referred to as “cancer type-specific polynucleotide group 1”), with any of the polynucleotides of the other SEQ ID NOs.

The combination of polynucleotides with cancer type specificity capable of discriminating a liver cancer patient not only from a healthy subject but also from other cancer patients is more preferably a combination of a plurality of polynucleotides selected from cancer type-specific polynucleotide group 1.

The combination of polynucleotides with cancer type specificity capable of discriminating a liver cancer patient not only from a healthy subject but also from other cancer patients is more preferably a combination comprising at least one or more polynucleotide(s) selected from the group consisting of polynucleotides consisting of the nucleotide sequences represented by SEQ ID NOs: 1, 3, 7, 9, 22, 38, 44, 134, 148, 155, 157, 164, 167, 172, 214, 714, 715, 716 and 717 or complementary sequences thereof (hereinafter, this group is referred to as “cancer type-specific polynucleotide group 2”) included in the cancer type-specific polynucleotide group 1, among the combinations of a plurality of polynucleotides selected from the cancer type-specific polynucleotide group 1.

The number of the polynucleotides with cancer type specificity in the combination described above can be 1, 2, 3, 4, 5, 6, 7, 8, 9, 10 or more in the combination and is more preferably 4 or more in the combination. Usually, the combination of 4 of the polynucleotides can produce adequate performance.

Non-limiting examples of the combination of the polynucleotide consisting of the nucleotide sequence represented by SEQ ID NO: 1 or a complementary sequence thereof with polynucleotides consisting of nucleotide sequences represented by SEQ ID NOs of three polynucleotides selected from the cancer type-specific polynucleotide group 1 or complementary sequences thereof are listed below.
(1) a combination of SEQ ID NOs: 1, 7, 9, and 148 (markers: hsa-miR-1343-3p, hsa-miR-6131, hsa-miR-7641, and hsa-miR-642a-3p);
(2) a combination of SEQ ID NOs: 1, 9, 155, and 172 (markers: hsa-miR-1343-3p, hsa-miR-7641, hsa-miR-3131, and hsa-miR-614);
(3) a combination of SEQ ID NOs: 1, 9, 148, and 155 (markers: hsa-miR-1343-3p, hsa-miR-7641, hsa-miR-642a-3p, and hsa-miR-3131);
(4) a combination of SEQ ID NOs: 1, 155, 172, and 715 (markers: hsa-miR-1343-3p, hsa-miR-3131, hsa-miR-614, and hsa-miR-4448); and
(5) a combination of SEQ ID NOs: 1, 155, 164, and 715 (markers: hsa-miR-1343-3p, hsa-miR-3131, hsa-miR-3162-5p, and hsa-miR-4448).

Non-limiting examples of the combination of the polynucleotide consisting of the nucleotide sequence represented by SEQ ID NO: 3 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 3, 7, 9, and 148 (markers: hsa-miR-6515-3p, hsa-miR-6131, hsa-miR-7641, and hsa-miR-642a-3p);
(2) a combination of SEQ ID NOs: 3, 22, 27, and 46 (markers: hsa-miR-6515-3p, hsa-miR-663b, hsa-miR-6729-5p, and hsa-miR-1908-5p);
(3) a combination of SEQ ID NOs: 1, 3, 29, and 155 (markers: hsa-miR-1343-3p, hsa-miR-6515-3p, hsa-miR-4706, and hsa-miR-3131);
(4) a combination of SEQ ID NOs: 1, 3, 151, and 155 (markers: hsa-miR-1343-3p, hsa-miR-6515-3p, hsa-miR-5195-3p, and hsa-miR-3131); and
(5) a combination of SEQ ID NOs: 3, 7, 148, and 715 (markers: hsa-miR-6515-3p, hsa-miR-6131, hsa-miR-642a-3p, and hsa-miR-4448).

Non-limiting examples of the combination of the polynucleotide consisting of the nucleotide sequence represented by SEQ ID NO: 7 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 28, 148, and 717 (markers: hsa-miR-6131, hsa-miR-8069, hsa-miR-642a-3p, and hsa-miR-3178);
(2) a combination of SEQ ID NOs: 7, 9, 148, and 186 (markers: hsa-miR-6131, hsa-miR-7641, hsa-miR-642a-3p, and hsa-miR-6085);
(3) a combination of SEQ ID NOs: 7, 148, 172, and 715 (markers: hsa-miR-6131, hsa-miR-642a-3p, hsa-miR-614, and hsa-miR-4448);
(4) a combination of SEQ ID NOs: 7, 9, 148, and 723 (markers: hsa-miR-6131, hsa-miR-7641, hsa-miR-642a-3p, and hsa-miR-4745-5p); and
(5) a combination of SEQ ID NOs: 7, 9, 28, and 148 (markers: hsa-miR-6131, hsa-miR-7641, hsa-miR-8069, and hsa-miR-642a-3p).

Non-limiting examples of the combination of the polynucleotide consisting of the nucleotide sequence represented by SEQ ID NO: 9 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 9, 148, and 157 (markers: hsa-miR-6131, hsa-miR-7641, hsa-miR-642a-3p, and hsa-miR-4463);
(2) a combination of SEQ ID NOs: 7, 9, 148, and 722 (markers: hsa-miR-6131, hsa-miR-7641, hsa-miR-642a-3p, and hsa-miR-4634);
(3) a combination of SEQ ID NOs: 7, 9, 27, and 148 (markers: hsa-miR-6131, hsa-miR-7641, hsa-miR-6729-5p, and hsa-miR-642a-3p);
(4) a combination of SEQ ID NOs: 7, 9, 148, and 725 (markers: hsa-miR-6131, hsa-miR-7641, hsa-miR-642a-3p, and hsa-miR-6803-5p); and
(5) a combination of SEQ ID NOs: 7, 9, 148, and 729 (markers: hsa-miR-6131, hsa-miR-7641, hsa-miR-642a-3p, and hsa-miR-6836-3p).

Non-limiting examples of the combination of the polynucleotide consisting of the nucleotide sequence represented by SEQ ID NO: 22 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 9, 22, and 148 (markers: hsa-miR-6131, hsa-miR-7641, hsa-miR-663b, and hsa-miR-642a-3p);
(2) a combination of SEQ ID NOs: 7, 22, 28, and 148 (markers: hsa-miR-6131, hsa-miR-663b, hsa-miR-8069, and hsa-miR-642a-3p);
(3) a combination of SEQ ID NOs: 7, 22, 148, and 189 (markers: hsa-miR-6131, hsa-miR-663b, hsa-miR-642a-3p, and hsa-miR-328-5p);
(4) a combination of SEQ ID NOs: 2, 7, 22, and 148 (markers: hsa-miR-6726-5p, hsa-miR-6131, hsa-miR-663b, and hsa-miR-642a-3p); and
(5) a combination of SEQ ID NOs: 7, 22, 148, and 720 (markers: hsa-miR-6131, hsa-miR-663b, hsa-miR-642a-3p, and hsa-miR-6789-5p).

Non-limiting examples of the combination of the polynucleotide consisting of the nucleotide sequence represented by SEQ ID NO: 38 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 9, 38, and 148 (markers: hsa-miR-6131, hsa-miR-7641, hsa-miR-3622a-5p, and hsa-miR-642a-3p);
(2) a combination of SEQ ID NOs: 7, 38, 51, and 148 (markers: hsa-miR-6131, hsa-miR-3622a-5p, hsa-miR-6781-5p, and hsa-miR-642a-3p);
(3) a combination of SEQ ID NOs: 7, 38, 148, and 718 (markers: hsa-miR-6131, hsa-miR-3622a-5p, hsa-miR-642a-3p, and hsa-miR-4725-3p);
(4) a combination of SEQ ID NOs: 7, 38, 148, and 216 (markers: hsa-miR-6131, hsa-miR-3622a-5p, hsa-miR-642a-3p, and hsa-miR-1238-5p); and
(5) a combination of SEQ ID NOs: 7, 38, 148, and 728 (markers: hsa-miR-6131, hsa-miR-3622a-5p, hsa-miR-642a-3p, and hsa-miR-4783-3p).

Non-limiting examples of the combination of the polynucleotide consisting of the nucleotide sequence represented by SEQ ID NO: 44 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 9, 44, and 148 (markers: hsa-miR-6131, hsa-miR-7641, hsa-miR-4665-5p, and hsa-miR-642a-3p);
(2) a combination of SEQ ID NOs: 7, 44, 123, and 148 (markers: hsa-miR-6131, hsa-miR-4665-5p, hsa-miR-4739, and hsa-miR-642a-3p);
(3) a combination of SEQ ID NOs: 7, 38, 44, and 148 (markers: hsa-miR-6131, hsa-miR-3622a-5p, hsa-miR-4665-5p, and hsa-miR-642a-3p);
(4) a combination of SEQ ID NOs: 7, 44, 148, and 723 (markers: hsa-miR-6131, hsa-miR-4665-5p, hsa-miR-642a-3p, and hsa-miR-4745-5p); and
(5) a combination of SEQ ID NOs: 7, 44, 48, and 148 (markers: hsa-miR-6131, hsa-miR-4665-5p, hsa-miR-6765-5p, and hsa-miR-642a-3p).

Non-limiting examples of the combination of the polynucleotide consisting of the nucleotide sequence represented by SEQ ID NO: 134 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 9, 134, and 148 (markers: hsa-miR-6131, hsa-miR-7641, hsa-miR-4492, and hsa-miR-642a-3p);
(2) a combination of SEQ ID NOs: 7, 134, 148, and 724 (markers: hsa-miR-6131, hsa-miR-4492, hsa-miR-642a-3p, and hsa-miR-4730);
(3) a combination of SEQ ID NOs: 7, 22, 134, and 148 (markers: hsa-miR-6131, hsa-miR-663b, hsa-miR-4492, and hsa-miR-642a-3p);
(4) a combination of SEQ ID NOs: 7, 134, 148, and 189 (markers: hsa-miR-6131, hsa-miR-4492, hsa-miR-642a-3p, and hsa-miR-328-5p); and
(5) a combination of SEQ ID NOs: 7, 134, 148, and 714 (markers: hsa-miR-6131, hsa-miR-4492, hsa-miR-642a-3p, and hsa-miR-6757-5p).

Non-limiting examples of the combination of the polynucleotide consisting of the nucleotide sequence represented by SEQ ID NO: 148 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 9, 148, and 726 (markers: hsa-miR-6131, hsa-miR-7641, hsa-miR-642a-3p, and hsa-miR-6798-5p);
(2) a combination of SEQ ID NOs: 7, 9, 148, and 151 (markers: hsa-miR-6131, hsa-miR-7641, hsa-miR-642a-3p, and hsa-miR-5195-3p);
(3) a combination of SEQ ID NOs: 7, 9, 109, and 148 (markers: hsa-miR-6131, hsa-miR-7641, hsa-miR-6826-5p, and hsa-miR-642a-3p);
(4) a combination of SEQ ID NOs: 5, 7, 9, and 148 (markers: hsa-miR-4257, hsa-miR-6131, hsa-miR-7641, and hsa-miR-642a-3p); and
(5) a combination of SEQ ID NOs: 7, 9, 76, and 148 (markers: hsa-miR-6131, hsa-miR-7641, hsa-miR-3917, and hsa-miR-642a-3p).

Non-limiting examples of the combination of the polynucleotide consisting of the nucleotide sequence represented by SEQ ID NO: 155 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 9, 148, and 155 (markers: hsa-miR-6131, hsa-miR-7641, hsa-miR-642a-3p, and hsa-miR-3131);
(2) a combination of SEQ ID NOs: 7, 38, 148, and 155 (markers: hsa-miR-6131, hsa-miR-3622a-5p, hsa-miR-642a-3p, and hsa-miR-3131);
(3) a combination of SEQ ID NOs: 1, 9, 155, and 167 (markers: hsa-miR-1343-3p, hsa-miR-7641, hsa-miR-3131, and hsa-miR-6724-5p);
(4) a combination of SEQ ID NOs: 1, 3, 155, and 715 (markers: hsa-miR-1343-3p, hsa-miR-6515-3p, hsa-miR-3131, and hsa-miR-4448); and
(5) a combination of SEQ ID NOs: 1, 3, 38, and 155 (markers: hsa-miR-1343-3p, hsa-miR-6515-3p, hsa-miR-3622a-5p, and hsa-miR-3131).

Non-limiting examples of the combination of the polynucleotide consisting of the nucleotide sequence represented by SEQ ID NO: 157 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 48, 157, and 714 (markers: hsa-miR-6131, hsa-miR-6765-5p, hsa-miR-4463, and hsa-miR-6757-5p);
(2) a combination of SEQ ID NOs: 7, 38, 148, and 157 (markers: hsa-miR-6131, hsa-miR-3622a-5p, hsa-miR-642a-3p, and hsa-miR-4463);
(3) a combination of SEQ ID NOs: 1, 44, 155, and 157 (markers: hsa-miR-1343-3p, hsa-miR-4665-5p, hsa-miR-3131, and hsa-miR-4463);
(4) a combination of SEQ ID NOs: 7, 76, 157, and 714 (markers: hsa-miR-6131, hsa-miR-3917, hsa-miR-4463, and hsa-miR-6757-5p); and
(5) a combination of SEQ ID NOs: 7, 148, 157, and 189 (markers: hsa-miR-6131, hsa-miR-642a-3p, hsa-miR-4463, and hsa-miR-328-5p).

Non-limiting examples of the combination of the polynucleotide consisting of the nucleotide sequence represented by SEQ ID NO: 164 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 9, 148, and 164 (markers: hsa-miR-6131, hsa-miR-7641, hsa-miR-642a-3p, and hsa-miR-3162-5p);
(2) a combination of SEQ ID NOs: 7, 76, 164, and 714 (markers: hsa-miR-6131, hsa-miR-3917, hsa-miR-3162-5p, and hsa-miR-6757-5p);
(3) a combination of SEQ ID NOs: 7, 38, 164, and 714 (markers: hsa-miR-6131, hsa-miR-3622a-5p, hsa-miR-3162-5p, and hsa-miR-6757-5p);
(4) a combination of SEQ ID NOs: 7, 38, 148, and 164 (markers: hsa-miR-6131, hsa-miR-3622a-5p, hsa-miR-642a-3p, and hsa-miR-3162-5p); and
(5) a combination of SEQ ID NOs: 1, 7, 164, and 714 (markers: hsa-miR-1343-3p, hsa-miR-6131, hsa-miR-3162-5p, and hsa-miR-6757-5p).

Non-limiting examples of the combination of the polynucleotide consisting of the nucleotide sequence represented by SEQ ID NO: 167 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 9, 148, and 167 (markers: hsa-miR-6131, hsa-miR-7641, hsa-miR-642a-3p, and hsa-miR-6724-5p);
(2) a combination of SEQ ID NOs: 1, 7, 167, and 714 (markers: hsa-miR-1343-3p, hsa-miR-6131, hsa-miR-6724-5p, and hsa-miR-6757-5p);
(3) a combination of SEQ ID NOs: 7, 151, 167, and 714 (markers: hsa-miR-6131, hsa-miR-5195-3p, hsa-miR-6724-5p, and hsa-miR-6757-5p);
(4) a combination of SEQ ID NOs: 7, 148, 167, and 189 (markers: hsa-miR-6131, hsa-miR-642a-3p, hsa-miR-6724-5p, and hsa-miR-328-5p); and
(5) a combination of SEQ ID NOs: 7, 28, 167, and 714 (markers: hsa-miR-6131, hsa-miR-8069, hsa-miR-6724-5p, and hsa-miR-6757-5p).

Non-limiting examples of the combination of the polynucleotide consisting of the nucleotide sequence represented by SEQ ID NO: 172 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 9, 148, and 172 (markers: hsa-miR-6131, hsa-miR-7641, hsa-miR-642a-3p, and hsa-miR-614);
(2) a combination of SEQ ID NOs: 7, 150, 172, and 714 (markers: hsa-miR-6131, hsa-miR-4728-5p, hsa-miR-614, and hsa-miR-6757-5p);
(3) a combination of SEQ ID NOs: 7, 172, 714, and 715 (markers: hsa-miR-6131, hsa-miR-614, hsa-miR-6757-5p, and hsa-miR-4448);
(4) a combination of SEQ ID NOs: 7, 38, 155, and 172 (markers: hsa-miR-6131, hsa-miR-3622a-5p, hsa-miR-3131, and hsa-miR-614); and
(5) a combination of SEQ ID NOs: 1, 2, 155, and 172 (markers: hsa-miR-1343-3p, hsa-miR-6726-5p, hsa-miR-3131, and hsa-miR-614).

Non-limiting examples of the combination of the polynucleotide consisting of the nucleotide sequence represented by SEQ ID NO: 214 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 9, 148, and 214 (markers: hsa-miR-6131, hsa-miR-7641, hsa-miR-642a-3p, and hsa-miR-4723-5p);
(2) a combination of SEQ ID NOs: 7, 148, 189, and 214 (markers: hsa-miR-6131, hsa-miR-642a-3p, hsa-miR-328-5p, and hsa-miR-4723-5p);
(3) a combination of SEQ ID NOs: 2, 7, 148, and 214 (markers: hsa-miR-6726-5p, hsa-miR-6131, hsa-miR-642a-3p, and hsa-miR-4723-5p);
(4) a combination of SEQ ID NOs: 1, 7, 214, and 714 (markers: hsa-miR-1343-3p, hsa-miR-6131, hsa-miR-4723-5p, and hsa-miR-6757-5p); and
(5) a combination of SEQ ID NOs: 7, 39, 148, and 214 (markers: hsa-miR-6131, hsa-miR-615-5p, hsa-miR-642a-3p, and hsa-miR-4723-5p).

Non-limiting examples of the combination of the polynucleotide consisting of the nucleotide sequence represented by SEQ ID NO: 714 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 9, 148, and 714 (markers: hsa-miR-6131, hsa-miR-7641, hsa-miR-642a-3p, and hsa-miR-6757-5p);
(2) a combination of SEQ ID NOs: 7, 54, 148, and 714 (markers: hsa-miR-6131, hsa-miR-6784-5p, hsa-miR-642a-3p, and hsa-miR-6757-5p);
(3) a combination of SEQ ID NOs: 7, 148, 151, and 714 (markers: hsa-miR-6131, hsa-miR-642a-3p, hsa-miR-5195-3p, and hsa-miR-6757-5p);
(4) a combination of SEQ ID NOs: 7, 38, 148, and 714 (markers: hsa-miR-6131, hsa-miR-3622a-5p, hsa-miR-642a-3p, and hsa-miR-6757-5p); and
(5) a combination of SEQ ID NOs: 7, 28, 148, and 714 (markers: hsa-miR-6131, hsa-miR-8069, hsa-miR-642a-3p, and hsa-miR-6757-5p).

Non-limiting examples of the combination of the polynucleotide consisting of the nucleotide sequence represented by SEQ ID NO: 715 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 2, 7, 148, and 715 (markers: hsa-miR-6726-5p, hsa-miR-6131, hsa-miR-642a-3p, and hsa-miR-4448);
(2) a combination of SEQ ID NOs: 7, 9, 148, and 715 (markers: hsa-miR-6131, hsa-miR-7641, hsa-miR-642a-3p, and hsa-miR-4448);
(3) a combination of SEQ ID NOs: 7, 17, 148, and 715 (markers: hsa-miR-6131, hsa-miR-204-3p, hsa-miR-642a-3p, and hsa-miR-4448);
(4) a combination of SEQ ID NOs: 7, 38, 148, and 715 (markers: hsa-miR-6131, hsa-miR-3622a-5p, hsa-miR-642a-3p, and hsa-miR-4448); and
(5) a combination of SEQ ID NOs: 7, 148, 715, and 725 (markers: hsa-miR-6131, hsa-miR-642a-3p, hsa-miR-6803-5p, and hsa-miR-4448).

Non-limiting examples of the combination of the polynucleotide consisting of the nucleotide sequence represented by SEQ ID NO: 716 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 9, 148, and 716 (markers: hsa-miR-6131, hsa-miR-7641, hsa-miR-642a-3p, and hsa-miR-671-5p);
(2) a combination of SEQ ID NOs: 7, 148, 714, and 716 (markers: hsa-miR-6131, hsa-miR-642a-3p, hsa-miR-6757-5p, and hsa-miR-671-5p);
(3) a combination of SEQ ID NOs: 2, 7, 148, and 716 (markers: hsa-miR-6726-5p, hsa-miR-6131, hsa-miR-642a-3p, and hsa-miR-671-5p);
(4) a combination of SEQ ID NOs: 7, 38, 148, and 716 (markers: hsa-miR-6131, hsa-miR-3622a-5p, hsa-miR-642a-3p, and hsa-miR-671-5p); and
(5) a combination of SEQ ID NOs: 7, 148, 715, and 716 (markers: hsa-miR-6131, hsa-miR-642a-3p, hsa-miR-4448, and hsa-miR-671-5p).

Non-limiting examples of the combination of the polynucleotide consisting of the nucleotide sequence represented by SEQ ID NO: 717 or a complementary sequence thereof with polynucleotides consisting of nucleotide sequences represented by SEQ ID NOs of three polynucleotides selected from the cancer type-specific polynucleotide group 1 or complementary sequences thereof are further listed below.
(1) a combination of SEQ ID NOs: 7, 9, 148, and 717 (markers: hsa-miR-6131, hsa-miR-7641, hsa-miR-642a-3p, and hsa-miR-3178);
(2) a combination of SEQ ID NOs: 7, 38, 148, and 717 (markers: hsa-miR-6131, hsa-miR-3622a-5p, hsa-miR-642a-3p, and hsa-miR-3178);
(3) a combination of SEQ ID NOs: 7, 27, 148, and 717 (markers: hsa-miR-6131, hsa-miR-6729-5p, hsa-miR-642a-3p, and hsa-miR-3178);
(4) a combination of SEQ ID NOs: 7, 44, 148, and 717 (markers: hsa-miR-6131, hsa-miR-4665-5p, hsa-miR-642a-3p, and hsa-miR-3178); and
(5) a combination of SEQ ID NOs: 7, 148, 715, and 717 (markers: hsa-miR-6131, hsa-miR-642a-3p, hsa-miR-4448, and hsa-miR-3178).

The kit or the device of the present invention may also comprise a polynucleotide that is already known or that will be found in the future, to enable detection of liver cancer, in addition to the polynucleotide(s) (which can include variant(s), fragment(s), and derivative(s)) according to the present invention described above.

The kit of the present invention may also comprise an antibody for measuring a marker for liver cancer examination known in the art, such as AFP, CEA, CA19-9 and PIVKA-II, in addition to the polynucleotide(s) according to the present invention as described above.

These polynucleotides contained in the kit of the present invention may be packaged in different containers either individually or in any combination.

The kit of the present invention may comprise a kit for extracting a nucleic acid (e.g., total RNA) from body fluids, cells, or tissues, a fluorescent material for labeling, an enzyme and a medium for nucleic acid amplification, an instruction manual, etc.

The device of the present invention is a device for cancer marker measurement in which nucleic acids such as the polynucleotides according to the present invention described above are bonded or attached to, for example, a solid phase. Examples of the material for the solid phase include plastics, paper, glass, and silicon. The material for the solid phase is preferably a plastic from the viewpoint of easy processability. The solid phase has any shape and is, for example, square, round, reed-shaped, or film-shaped. The device of the present invention includes, for example, a device for measurement by a hybridization technique. Specific examples thereof include blotting devices and nucleic acid arrays (e.g., microarrays, DNA chips, and RNA chips).

The nucleic acid array technique is a technique which involves bonding or attaching the nucleic acids one by one by use of a method [e.g., a method of spotting the nucleic acids using a high-density dispenser called spotter or arrayer onto the surface of the solid phase surface-treated, if necessary, by coating with L-lysine or the introduction of a functional group such as an amino group or a carboxyl group, a method of spraying the nucleic acids onto the solid phase using an inkjet which injects very small liquid droplets by a piezoelectric element or the like from a nozzle, or a method of sequentially synthesizing nucleotides on the solid phase] to prepare an array such as a chip and measuring a target nucleic acid through the use of hybridization using this array.

The kit or the device of the present invention comprises nucleic acids capable of specifically binding to the polynucleotides of at least one or more, preferably at least two or more, more preferably at least three or more, most preferably at least five or more to all of the liver cancer marker miRNAs, respectively, of the group 1 described above. The kit or the device of the present invention may optionally further comprise nucleic acids capable of specifically binding to the polynucleotides of at least one or more, preferably at least two or more, more preferably at least three or more, most preferably at least five or more to all of the liver cancer marker miRNAs, respectively, of the group 2 described above. The kit or the device of the present invention may optionally further comprise nucleic acids capable of specifically binding to the polynucleotides of at least one or more, preferably at least two or more, more preferably at least three or more, most preferably at least five or more to all of the liver cancer marker miRNAs, respectively, of the group 3 described above.

The kit or the device of the present invention can be used for detecting liver cancer as described in Section 4 below.

4. Method for Detecting Liver Cancer

The present invention further provides a method for detecting liver cancer, comprising using the kit or the device of the present invention (comprising the above-mentioned nucleic acid(s) that can be used in the present invention) described in Section 3 above to measure expression level(s) of one or more liver cancer-derived gene(s) being an expression level of liver cancer-derived gene(s) selected from the following group: miR-1343-3p, miR-6726-5p, miR-6515-3p, miR-4651, miR-4257, miR-3188, miR-6131, miR-6766-3p, miR-7641, miR-1249, miR-3679-3p, miR-6787-5p, miR-4454, miR-3135b, miR-6765-3p, miR-7975, miR-204-3p, miR-7977, miR-7110-5p, miR-6717-5p, miR-6870-5p, miR-663b, miR-6875-5p, miR-8072, miR-6816-5p, miR-4281, miR-6729-5p, miR-8069, miR-4706, miR-7108-5p, miR-4433b-3p, miR-6893-5p, miR-6857-5p, miR-1227-5p, miR-6741-5p, miR-451a, miR-8063, miR-3622a-5p, miR-615-5p, miR-128-1-5p, miR-6825-5p, miR-1260b, miR-4433-3p, miR-4665-5p, miR-7845-5p, miR-1908-5p, miR-6840-3p, miR-6765-5p, miR-296-5p, miR-3675-3p, miR-6781-5p, miR-423-5p, miR-3663-3p, miR-6784-5p, miR-6749-5p, miR-1231, miR-4746-3p, miR-6780b-5p, miR-4758-5p, miR-3679-5p, miR-3184-5p, miR-6125, miR-6721-5p, miR-6791-5p, miR-3185, miR-1260a, miR-3197, miR-6845-5p, miR-6887-5p, miR-6738-5p, miR-6872-3p, miR-4497, miR-1229-5p, miR-6820-5p, miR-6777-5p, miR-3917, miR-5787, miR-4286, miR-6877-5p, miR-1225-3p, miR-6088, miR-6800-5p, miR-1246, miR-4467, miR-4419b, miR-1914-3p, miR-4632-5p, miR-1915-5p, miR-3940-5p, miR-1185-2-3p, miR-6746-5p, miR-5001-5p, miR-1228-5p, miR-5572, miR-4327, miR-4638-5p, miR-6799-5p, miR-6861-5p, miR-6727-5p, miR-4513, miR-6805-3p, miR-6808-5p, miR-4449, miR-1199-5p, miR-1275, miR-4792, miR-4443, miR-6891-5p, miR-6826-5p, miR-6807-5p, miR-7150, miR-4534, miR-4476, miR-4649-5p, miR-4525, miR-1915-3p, miR-4516, miR-4417, miR-642b-3p, miR-3141, miR-5100, miR-6848-5p, miR-4739, miR-4459, miR-1237-5p, miR-296-3p, miR-4665-3p, miR-6786-5p, miR-4258, miR-6510-5p, miR-1343-5p, miR-1247-3p, miR-6805-5p, miR-4492, miR-1469, miR-1268b, miR-6858-5p, miR-3937, miR-939-5p, miR-3656, miR-744-5p, miR-4687-3p, miR-4763-3p, miR-3620-5p, miR-3195, miR-6842-5p, miR-4707-5p, miR-642a-3p, miR-7113-3p, miR-4728-5p, miR-5195-3p, miR-1185-1-3p, miR-6774-5p, miR-8059, miR-3131, miR-7847-3p, miR-4463, miR-128-2-5p, miR-4508, miR-6806-5p, miR-7111-5p, miR-6782-5p, miR-4734, miR-3162-5p, miR-887-3p, miR-6752-5p, miR-6724-5p, miR-6757-5p, miR-4448, miR-671-5p, miR-3178, miR-4725-3p, miR-940, miR-6789-5p, miR-4484, miR-4634, miR-4745-5p, miR-4730, miR-6803-5p, miR-6798-5p, miR-3648, miR-4783-3p and miR-6836-3p; optionally an expression level of liver cancer-derived gene(s) selected from the following group: miR-23b-3p, miR-23a-3p, miR-625-3p, miR-1228-3p, miR-614, miR-1913, miR-92a-2-5p, miR-187-5p, miR-16-5p, miR-92b-3p, miR-150-3p, miR-564, miR-125a-3p, miR-92b-5p, miR-92a-3p and miR-663a; and optionally an expression level of liver cancer-derived gene(s) selected from miR-4688, miR-4648, miR-6085, miR-6126, miR-6880-5p, miR-328-5p, miR-6768-5p, miR-3180, miR-6087, miR-1273g-3p, miR-1225-5p, miR-3196, miR-4695-5p, miR-6732-5p, miR-638, miR-6813-5p, miR-665, miR-486-3p, miR-4466, miR-30c-1-3p, miR-3621, miR-6743-5p, miR-4298, miR-4741, miR-3619-3p, miR-6824-5p, miR-5698, miR-371a-5p, miR-4488, miR-1233-5p, miR-4723-5p, miR-24-3p, miR-1238-5p, miR-4442, miR-3928-3p, miR-6716-5p, miR-6089, miR-6124, miR-6778-5p, miR-557 and miR-6090 in a sample in vitro, further comparing, for example, the expression level(s) of the aforementioned gene(s) in the sample (e.g., blood, serum, or plasma) collected from a subject suspected of having liver cancer with a control expression level in the sample collected from a healthy subject (including a non-liver cancer patient), and evaluating the subject as having liver cancer when the expression level(s) of the target nucleic acid(s) is statistically significantly different between the samples.

This method of the present invention enables a limitedly invasive, early diagnosis of the cancer with high sensitivity and high specificity and thereby brings about early treatment and improved prognosis. In addition, exacerbation of the disease or the effectiveness of surgical, radiotherapeutic, and chemotherapeutic treatments can be monitored.

The method for extracting the liver cancer-derived gene from the sample such as blood, serum, or plasma according to the present invention is particularly preferably prepared by the addition of a reagent for RNA extraction in 3D-Gene® RNA extraction reagent from liquid sample kit (Toray Industries, Inc.). A general acidic phenol method (acid guanidinium-phenol-chloroform (AGPC)) may be used, or Trizol® (Life Technologies Corp.) may be used. The liver cancer-derived gene may be prepared by the addition of a reagent for RNA extraction containing acidic phenol, such as Trizol (Life Technologies Corp.) or Isogen (Nippon Gene Co., Ltd.). Alternatively, a kit such as miRNeasy® Mini Kit (Qiagen N. V.) can be used, though the method is not limited thereto.

The present invention also provides use of the kit or the device of the present invention for detecting in vitro an expression product of a liver cancer-derived miRNA gene in a sample derived from a subject.

In the method of the present invention, the kit or device described above comprising a single polynucleotide or any possible combination of the polynucleotides that can be used in the present invention as described above is used.

In the detection or (genetic) diagnosis of liver cancer according to the present invention, each polynucleotide contained in the kit or the device of the present invention can be used as a probe or a primer. In the case of using the polynucleotide as a primer, TaqMan® MicroRNA Assays from Life Technologies Corp., miScript PCR System from Qiagen N. V., or the like can be used, though the method is not limited thereto.

The polynucleotide contained in the kit or the device of the present invention can be used as a primer or a probe according to a routine method in a method known in the art for specifically detecting the particular gene, for example, a hybridization technique such as Northern blot, Southern blot, in situ hybridization, Northern hybridization, or Southern hybridization, or a quantitative amplification technique such as quantitative RT-PCR. A body fluid such as blood, serum, plasma, or urine of the subject is collected as a sample to be assayed according to the type of the detection method used. Alternatively, total RNA prepared from such a body fluid by the method described above may be used, and various polynucleotides including cDNA prepared on the basis of the RNA may be used.

The kit or the device of the present invention is useful for the diagnosis of liver cancer or the detection of the presence or absence of liver cancer. Specifically, the detection of liver cancer using the kit or the device can be performed by detecting in vitro an expression level of a gene using the nucleic acid probe or the primer contained in the kit or the device in a sample such as blood, serum, plasma, or urine from a subject suspected of having liver cancer. The subject suspected of having liver cancer can be evaluated as having liver cancer when the expression level of a target miRNA marker measured using polynucleotide(s) (including variant(s), fragment(s), and derivative(s) thereof) consisting of a nucleotide sequence represented by at least one or more of SEQ ID NOs: 1 to 167 and 714 to 729 or a complementary sequence thereof, optionally a nucleotide sequence represented by one or more of SEQ ID NOs: 168 to 183 or a complementary sequence thereof, and optionally a nucleotide sequence represented by one or more of SEQ ID NOs: 184 to 224 or a complementary sequence thereof in the sample such as blood, serum, plasma, or urine of the subject is statistically significantly different compared with the expression level thereof in the sample such as blood, serum, or plasma, or urine of a healthy subject.

The method of the present invention can be combined with a diagnostic imaging method such as ultrasonography, CT scanning, MRI scanning, or angiography examination. The method of the present invention is capable of specifically detecting liver cancer and can substantially discriminate liver cancer from the other cancers.

The method for detecting the absence of an expression product of a liver cancer-derived gene or the presence of the expression product of a liver cancer-derived gene in a sample using the kit or the device of the present invention comprises collecting a body fluid such as blood, serum, plasma, or urine of a subject, and measuring the expression level of the target gene contained therein using one or more polynucleotide(s) (including variant(s), fragment(s), or derivative(s)) selected from the polynucleotide group of the present invention, to evaluate the presence or absence of liver cancer or to detect liver cancer. Using the method for detecting liver cancer according to the present invention, for example, the presence or absence of amelioration of the disease or the degree of amelioration thereof in a liver cancer patient when a therapeutic drug is administered to the patient for amelioration of the disease can be also evaluated or diagnosed.

The method of the present invention may comprise, for example, the following steps (a), (b), and (c):
(a) a step of contacting in vitro a sample derived from a subject with a polynucleotide in the kit or the device of the present invention;
(b) a step of measuring an expression level of the target nucleic acid in the sample using the polynucleotide as a nucleic acid probe or a primer; and
(c) a step of evaluating the presence or absence of liver cancer (cells) in the subject on the basis of a measurement result obtained in the step (b).

Specifically, the present invention provides a method for detecting liver cancer, comprising measuring an expression level of a target nucleic acid in a sample of a subject using nucleic acid(s) capable of specifically binding to at least one or more (preferably at least two or more) polynucleotide(s) selected from the group consisting of miR-1343-3p, miR-6726-5p, miR-6515-3p, miR-4651, miR-4257, miR-3188, miR-6131, miR-6766-3p, miR-7641, miR-1249, miR-3679-3p, miR-6787-5p, miR-4454, miR-3135b, miR-6765-3p, miR-7975, miR-204-3p, miR-7977, miR-7110-5p, miR-6717-5p, miR-6870-5p, miR-663b, miR-6875-5p, miR-8072, miR-6816-5p, miR-4281, miR-6729-5p, miR-8069, miR-4706, miR-7108-5p, miR-4433b-3p, miR-6893-5p, miR-6857-5p, miR-1227-5p, miR-6741-5p, miR-451a, miR-8063, miR-3622a-5p, miR-615-5p, miR-128-1-5p, miR-6825-5p, miR-1260b, miR-4433-3p, miR-4665-5p, miR-7845-5p, miR-1908-5p, miR-6840-3p, miR-6765-5p, miR-296-5p, miR-3675-3p, miR-6781-5p, miR-423-5p, miR-3663-3p, miR-6784-5p, miR-6749-5p, miR-1231, miR-4746-3p, miR-6780b-5p, miR-4758-5p, miR-3679-5p, miR-3184-5p, miR-6125, miR-6721-5p, miR-6791-5p, miR-3185, miR-1260a, miR-3197, miR-6845-5p, miR-6887-5p, miR-6738-5p, miR-6872-3p, miR-4497, miR-1229-5p, miR-6820-5p, miR-6777-5p, miR-3917, miR-5787, miR-4286, miR-6877-5p, miR-1225-3p, miR-6088, miR-6800-5p, miR-1246, miR-4467, miR-4419b, miR-1914-3p, miR-4632-5p, miR-1915-5p, miR-3940-5p, miR-1185-2-3p, miR-6746-5p, miR-5001-5p, miR-1228-5p, miR-5572, miR-4327, miR-4638-5p, miR-6799-5p, miR-6861-5p, miR-6727-5p, miR-4513, miR-6805-3p, miR-6808-5p, miR-4449, miR-1199-5p, miR-1275, miR-4792, miR-4443, miR-6891-5p, miR-6826-5p, miR-6807-5p, miR-7150, miR-4534, miR-4476, miR-4649-5p, miR-4525, miR-1915-3p, miR-4516, miR-4417, miR-642b-3p, miR-3141, miR-5100, miR-6848-5p, miR-4739, miR-4459, miR-1237-5p, miR-296-3p, miR-4665-3p, miR-6786-5p, miR-4258, miR-6510-5p, miR-1343-5p, miR-1247-3p, miR-6805-5p, miR-4492, miR-1469, miR-1268b, miR-6858-5p, miR-3937, miR-939-5p, miR-3656, miR-744-5p, miR-4687-3p, miR-4763-3p, miR-3620-5p, miR-3195, miR-6842-5p, miR-4707-5p, miR-642a-3p, miR-7113-3p, miR-4728-5p, miR-5195-3p, miR-1185-1-3p, miR-6774-5p, miR-8059, miR-3131, miR-7847-3p, miR-4463, miR-128-2-5p, miR-4508, miR-6806-5p, miR-7111-5p, miR-6782-5p, miR-4734, miR-3162-5p, miR-887-3p, miR-6752-5p, miR-6724-5p, miR-6757-5p, miR-4448, miR-671-5p, miR-3178, miR-4725-3p, miR-940, miR-6789-5p, miR-4484, miR-4634, miR-4745-5p, miR-4730, miR-6803-5p, miR-6798-5p, miR-3648, miR-4783-3p and miR-6836-3p, and evaluating in vitro whether or not the subject has liver cancer using the measured expression level and a control expression level of a healthy subject measured in the same way as above.

The term “evaluation” used herein is evaluation support based on results of in vitro examination, not physician's judgment.

As described above, in a preferred embodiment of the method of the present invention, specifically, miR-1343-3p is hsa-miR-1343-3p, miR-6726-5p is hsa-miR-6726-5p, miR-6515-3p is hsa-miR-6515-3p, miR-4651 is hsa-miR-4651, miR-4257 is hsa-miR-4257, miR-3188 is hsa-miR-3188, miR-6131 is hsa-miR-6131, miR-6766-3p is hsa-miR-6766-3p, miR-7641 is hsa-miR-7641, miR-1249 is hsa-miR-1249, miR-3679-3p is hsa-miR-3679-3p, miR-6787-5p is hsa-miR-6787-5p, miR-4454 is hsa-miR-4454, miR-3135b is hsa-miR-3135b, miR-6765-3p is hsa-miR-6765-3p, miR-7975 is hsa-miR-7975, miR-204-3p is hsa-miR-204-3p, miR-7977 is hsa-miR-7977, miR-7110-5p is hsa-miR-7110-5p, miR-6717-5p is hsa-miR-6717-5p, miR-6870-5p is hsa-miR-6870-5p, miR-663b is hsa-miR-663b, miR-6875-5p is hsa-miR-6875-5p, miR-8072 is hsa-miR-8072, miR-6816-5p is hsa-miR-6816-5p, miR-4281 is hsa-miR-4281, miR-6729-5p is hsa-miR-6729-5p, miR-8069 is hsa-miR-8069, miR-4706 is hsa-miR-4706, miR-7108-5p is hsa-miR-7108-5p, miR-4433b-3p is hsa-miR-4433b-3p, miR-6893-5p is hsa-miR-6893-5p, miR-6857-5p is hsa-miR-6857-5p, miR-1227-5p is hsa-miR-1227-5p, miR-6741-5p is hsa-miR-6741-5p, miR-451a is hsa-miR-451a, miR-8063 is hsa-miR-8063, miR-3622a-5p is hsa-miR-3622a-5p, miR-615-5p is hsa-miR-615-5p, miR-128-1-5p is hsa-miR-128-1-5p, miR-6825-5p is hsa-miR-6825-5p, miR-1260b is hsa-miR-1260b, miR-4433-3p is hsa-miR-4433-3p, miR-4665-5p is hsa-miR-4665-5p, miR-7845-5p is hsa-miR-7845-5p, miR-1908-5p is hsa-miR-1908-5p, miR-6840-3p is hsa-miR-6840-3p, miR-6765-5p is hsa-miR-6765-5p, miR-296-5p is hsa-miR-296-5p, miR-3675-3p is hsa-miR-3675-3p, miR-6781-5p is hsa-miR-6781-5p, miR-423-5p is hsa-miR-423-5p, miR-3663-3p is hsa-miR-3663-3p, miR-6784-5p is hsa-miR-6784-5p, miR-6749-5p is hsa-miR-6749-5p, miR-1231 is hsa-miR-1231, miR-4746-3p is hsa-miR-4746-3p, miR-6780b-5p is hsa-miR-6780b-5p, miR-4758-5p is hsa-miR-4758-5p, miR-3679-5p is hsa-miR-3679-5p, miR-3184-5p is hsa-miR-3184-5p, miR-6125 is hsa-miR-6125, miR-6721-5p is hsa-miR-6721-5p, miR-6791-5p is hsa-miR-6791-5p, miR-3185 is hsa-miR-3185, miR-1260a is hsa-miR-1260a, miR-3197 is hsa-miR-3197, miR-6845-5p is hsa-miR-6845-5p, miR-6887-5p is hsa-miR-6887-5p, miR-6738-5p is hsa-miR-6738-5p, miR-6872-3p is hsa-miR-6872-3p, miR-4497 is hsa-miR-4497, miR-1229-5p is hsa-miR-1229-5p, miR-6820-5p is hsa-miR-6820-5p, miR-6777-5p is hsa-miR-6777-5p, miR-3917 is hsa-miR-3917, miR-5787 is hsa-miR-5787, miR-4286 is hsa-miR-4286, miR-6877-5p is hsa-miR-6877-5p, miR-1225-3p is hsa-miR-1225-3p, miR-6088 is hsa-miR-6088, miR-6800-5p is hsa-miR-6800-5p, miR-1246 is hsa-miR-1246, miR-4467 is hsa-miR-4467, miR-4419b is hsa-miR-4419b, miR-1914-3p is hsa-miR-1914-3p, miR-4632-5p is hsa-miR-4632-5p, miR-1915-5p is hsa-miR-1915-5p, miR-3940-5p is hsa-miR-3940-5p, miR-1185-2-3p is hsa-miR-1185-2-3p, miR-6746-5p is hsa-miR-6746-5p, miR-5001-5p is hsa-miR-5001-5p, miR-1228-5p is hsa-miR-1228-5p, miR-5572 is hsa-miR-5572, miR-4327 is hsa-miR-4327, miR-4638-5p is hsa-miR-4638-5p, miR-6799-5p is hsa-miR-6799-5p, miR-6861-5p is hsa-miR-6861-5p, miR-6727-5p is hsa-miR-6727-5p, miR-4513 is hsa-miR-4513, miR-6805-3p is hsa-miR-6805-3p, miR-6808-5p is hsa-miR-6808-5p, miR-4449 is hsa-miR-4449, miR-1199-5p is hsa-miR-1199-5p, miR-1275 is hsa-miR-1275, miR-4792 is hsa-miR-4792, miR-4443 is hsa-miR-4443, miR-6891-5p is hsa-miR-6891-5p, miR-6826-5p is hsa-miR-6826-5p, miR-6807-5p is hsa-miR-6807-5p, miR-7150 is hsa-miR-7150, miR-4534 is hsa-miR-4534, miR-4476 is hsa-miR-4476, miR-4649-5p is hsa-miR-4649-5p, miR-4525 is hsa-miR-4525, miR-1915-3p is hsa-miR-1915-3p, miR-4516 is hsa-miR-4516, miR-4417 is hsa-miR-4417, miR-642b-3p is hsa-miR-642b-3p, miR-3141 is hsa-miR-3141, miR-5100 is hsa-miR-5100, miR-6848-5p is hsa-miR-6848-5p, miR-4739 is hsa-miR-4739, miR-4459 is hsa-miR-4459, miR-123′7-5p is hsa-miR-1237-5p, miR-296-3p is hsa-miR-296-3p, miR-4665-3p is hsa-miR-4665-3p, miR-6786-5p is hsa-miR-6786-5p, miR-4258 is hsa-miR-4258, miR-6510-5p is hsa-miR-6510-5p, miR-1343-5p is hsa-miR-1343-5p, miR-1247-3p is hsa-miR-1247-3p, miR-6805-5p is hsa-miR-6805-5p, miR-4492 is hsa-miR-4492, miR-1469 is hsa-miR-1469, miR-1268b is hsa-miR-1268b, miR-6858-5p is hsa-miR-6858-5p, miR-3937 is hsa-miR-3937, miR-939-5p is hsa-miR-939-5p, miR-3656 is hsa-miR-3656, miR-744-5p is hsa-miR-744-5p, miR-4687-3p is hsa-miR-4687-3p, miR-4763-3p is hsa-miR-4763-3p, miR-3620-5p is hsa-miR-3620-5p, miR-3195 is hsa-miR-3195, miR-6842-5p is hsa-miR-6842-5p, miR-4707-5p is hsa-miR-4707-5p, miR-642a-3p is hsa-miR-642a-3p, miR-7113-3p is hsa-miR-7113-3p, miR-4728-5p is hsa-miR-4728-5p, miR-5195-3p is hsa-miR-5195-3p, miR-1185-1-3p is hsa-miR-1185-1-3p, miR-6774-5p is hsa-miR-6774-5p, miR-8059 is hsa-miR-8059, miR-3131 is hsa-miR-3131, miR-7847-3p is hsa-miR-7847-3p, miR-4463 is hsa-miR-4463, miR-128-2-5p is hsa-miR-128-2-5p, miR-4508 is hsa-miR-4508, miR-6806-5p is hsa-miR-6806-5p, miR-7111-5p is hsa-miR-7111-5p, miR-6782-5p is hsa-miR-6782-5p, miR-4734 is hsa-miR-4734, miR-3162-5p is hsa-miR-3162-5p, miR-887-3p is hsa-miR-887-3p, miR-6752-5p is hsa-miR-6752-5p, miR-6724-5p is hsa-miR-6724-5p, miR-6757-5p is hsa-miR-6757-5p, miR-4448 is hsa-miR-4448, miR-671-5p is hsa-miR-671-5p, miR-3178 is hsa-miR-3178, miR-4725-3p is hsa-miR-4725-3p, miR-940 is hsa-miR-940, miR-6789-5p is hsa-miR-6789-5p, miR-4484 is hsa-miR-4484, miR-4634 is hsa-miR-4634, miR-4745-5p is hsa-miR-4745-5p, miR-4730 is hsa-miR-4730, miR-6803-5p is hsa-miR-6803-5p, miR-6798-5p is hsa-miR-6798-5p, miR-3648 is hsa-miR-3648, miR-4783-3p is hsa-miR-4783-3p, and miR-6836-3p is hsa-miR-6836-3p.

In a preferred embodiment of the method of the present invention, specifically, the nucleic acid (specifically, probe or primer) is selected from the group consisting of the following polynucleotides (a) to (e):
(a) a polynucleotide consisting of a nucleotide sequence represented by any of SEQ ID NOs: 1 to 167 and 714 to 729 or a nucleotide sequence derived from the nucleotide sequence by the replacement of u with t, a variant thereof, a derivative thereof, or a fragment thereof comprising 15 or more consecutive nucleotides,
(b) a polynucleotide comprising a nucleotide sequence represented by any of SEQ ID NOs: 1 to 167 and 714 to 729,
(c) a polynucleotide consisting of a nucleotide sequence complementary to a nucleotide sequence represented by any of SEQ ID NOs: 1 to 167 and 714 to 729 or a nucleotide sequence derived from the nucleotide sequence by the replacement of u with t, a variant thereof, a derivative thereof, or a fragment thereof comprising 15 or more consecutive nucleotides,
(d) a polynucleotide comprising a nucleotide sequence complementary to a nucleotide sequence represented by any of SEQ ID NOs: 1 to 167 and 714 to 729 or a nucleotide sequence derived from the nucleotide sequence by the replacement of u with t, and
(e) a polynucleotide hybridizing under stringent conditions to any of the polynucleotides (a) to (d).

In the method of the present invention, nucleic acid(s) capable of specifically binding to at least one or more polynucleotide(s) selected from the followings: miR-23b-3p, miR-23a-3p, miR-625-3p, miR-1228-3p, miR-614, miR-1913, miR-92a-2-5p, miR-187-5p, miR-16-5p, miR-92b-3p, miR-150-3p, miR-564, miR-125a-3p, miR-92b-5p, miR-92a-3p and miR-663a may be further used.

In a preferred embodiment, as for such an additional nucleic acid, specifically, miR-23b-3p is hsa-miR-23b-3p, miR-23a-3p is hsa-miR-23a-3p, miR-625-3p is hsa-miR-625-3p, miR-1228-3p is hsa-miR-1228-3p, miR-614 is hsa-miR-614, miR-1913 is hsa-miR-1913, miR-92a-2-5p is hsa-miR-92a-2-5p, miR-187-5p is hsa-miR-187-5p, miR-16-5p is hsa-miR-16-5p, miR-92b-3p is hsa-miR-92b-3p, miR-150-3p is hsa-miR-150-3p, miR-564 is hsa-miR-564, miR-125a-3p is hsa-miR-125a-3p, miR-92b-5p is hsa-miR-92b-5p, miR-92a-3p is hsa-miR-92a-3p, and miR-663a is hsa-miR-663a.

In a preferred embodiment, such a nucleic acid is specifically selected from the group consisting of the following polynucleotides (f) to (j):
(f) a polynucleotide consisting of a nucleotide sequence represented by any of SEQ ID NOs: 168 to 183 or a nucleotide sequence derived from the nucleotide sequence by the replacement of u with t, a variant thereof, a derivative thereof, or a fragment thereof comprising 15 or more consecutive nucleotides,
(g) a polynucleotide comprising a nucleotide sequence represented by any of SEQ ID NOs: 168 to 183,
(h) a polynucleotide consisting of a nucleotide sequence complementary to a nucleotide sequence represented by any of SEQ ID NOs: 168 to 183 or a nucleotide sequence derived from the nucleotide sequence by the replacement of u with t, a variant thereof, a derivative thereof, or a fragment thereof comprising 15 or more consecutive nucleotides,
(i) a polynucleotide comprising a nucleotide sequence complementary to a nucleotide sequence represented by any of SEQ ID NOs: 168 to 183 or a nucleotide sequence derived from the nucleotide sequence by the replacement of u with t, and
(j) a polynucleotide hybridizing under stringent conditions to any of the polynucleotides (f) to (i).

In the method of the present invention, a nucleic acid capable of specifically binding to at least one or more polynucleotide(s) selected from the group consisting of miR-4688, miR-4648, miR-6085, miR-6126, miR-6880-5p, miR-328-5p, miR-6768-5p, miR-3180, miR-6087, miR-1273g-3p, miR-1225-5p, miR-3196, miR-4695-5p, miR-6732-5p, miR-638, miR-6813-5p, miR-665, miR-486-3p, miR-4466, miR-30c-1-3p, miR-3621, miR-6743-5p, miR-4298, miR-4741, miR-3619-3p, miR-6824-5p, miR-5698, miR-371a-5p, miR-4488, miR-1233-5p, miR-4723-5p, miR-24-3p, miR-1238-5p, miR-4442, miR-3928-3p, miR-6716-5p, miR-6089, miR-6124, miR-6778-5p, miR-557 and miR-6090 may be further used.

In a preferred embodiment, as for such an additional nucleic acid, specifically, miR-4688 is hsa-miR-4688, miR-4648 is hsa-miR-4648, miR-6085 is hsa-miR-6085, miR-6126 is hsa-miR-6126, miR-6880-5p is hsa-miR-6880-5p, miR-328-5p is hsa-miR-328-5p, miR-6768-5p is hsa-miR-6768-5p, miR-3180 is hsa-miR-3180, miR-6087 is hsa-miR-6087, miR-1273g-3p is hsa-miR-1273g-3p, miR-1225-5p is hsa-miR-1225-5p, miR-3196 is hsa-miR-3196, miR-4695-5p is hsa-miR-4695-5p, miR-6732-5p is hsa-miR-6732-5p, miR-638 is hsa-miR-638, miR-6813-5p is hsa-miR-6813-5p, miR-665 is hsa-miR-665, miR-486-3p is hsa-miR-486-3p, miR-4466 is hsa-miR-4466, miR-30c-1-3p is hsa-miR-30c-1-3p, miR-3621 is hsa-miR-3621, miR-6743-5p is hsa-miR-6743-5p, miR-4298 is hsa-miR-4298, miR-4741 is hsa-miR-4741, miR-3619-3p is hsa-miR-3619-3p, miR-6824-5p is hsa-miR-6824-5p, miR-5698 is hsa-miR-5698, miR-371a-5p is hsa-miR-371a-5p, miR-4488 is hsa-miR-4488, miR-1233-5p is hsa-miR-1233-5p, miR-4723-5p is hsa-miR-4723-5p, miR-24-3p is hsa-miR-24-3p, miR-1238-5p is hsa-miR-1238-5p, miR-4442 is hsa-miR-4442, miR-3928-3p is hsa-miR-3928-3p, miR-6716-5p is hsa-miR-6716-5p, miR-6089 is hsa-miR-6089, miR-6124 is hsa-miR-6124, miR-6778-5p is hsa-miR-6778-5p, miR-557 is hsa-miR-557, and miR-6090 is hsa-miR-6090.

In a preferred embodiment, such a nucleic acid is specifically a polynucleotide selected from the group consisting of the following polynucleotides (k) to (o):
(k) a polynucleotide consisting of a nucleotide sequence represented by any of SEQ ID NOs: 184 to 224 or a nucleotide sequence derived from the nucleotide sequence by the replacement of u with t, a variant thereof, a derivative thereof, or a fragment thereof comprising 15 or more consecutive nucleotides,
(l) a polynucleotide comprising a nucleotide sequence represented by any of SEQ ID NOs: 184 to 224,
(m) a polynucleotide consisting of a nucleotide sequence complementary to a nucleotide sequence represented by any of SEQ ID NOs: 184 to 224 or a nucleotide sequence derived from the nucleotide sequence by the replacement of u with t, a variant thereof, a derivative thereof, or a fragment thereof comprising 15 or more consecutive nucleotides,
(n) a polynucleotide comprising a nucleotide sequence complementary to a nucleotide sequence represented by any of SEQ ID NOs: 184 to 224 or a nucleotide sequence derived from the nucleotide sequence by the replacement of u with t, and
(o) a polynucleotide hybridizing under stringent conditions to any of the polynucleotides (k) to (n).

Examples of the sample used in the method of the present invention can include samples prepared from a living tissue (preferably a liver tissue) or a body fluid such as blood, serum, plasma, or urine of the subject. Specifically, for example, an RNA-containing sample prepared from the tissue, a polynucleotide-containing sample further prepared therefrom, a body fluid such as blood, serum, plasma, or urine, a portion or the whole of a living tissue collected from the subject by biopsy or the like, or a living tissue excised by surgery can be used, and the sample for measurement can be prepared therefrom.

The subject used herein refers to a mammal, for example, a human, a monkey, a mouse and a rat without any limitation, and is preferably a human.

The steps of the method of the present invention can be changed according to the type of the sample to be assayed.

In the case of using RNA as an analyte, the detection of liver cancer (cells) may comprise, for example, the following steps (a), (b), and (c):
(a) a step of binding RNA prepared from the sample of a subject or a complementary polynucleotide (cDNA) transcribed therefrom to a polynucleotide in the kit or the device of the present invention;
(b) a step of measuring the sample-derived RNA or the cDNA synthesized from the RNA, bound with the polynucleotide by hybridization using the polynucleotide as a nucleic acid probe or by quantitative RT-PCR using the polynucleotide as a primer; and
(c) a step of evaluating the presence or absence of liver cancer (or liver cancer-derived gene expression) on the basis of the measurement results of the step (b).

For example, various hybridization methods can be used for detecting, examining, evaluating, or diagnosing liver cancer (or liver cancer-derived gene expression) in vitro according to the present invention. For example, Northern blot, Southern blot, RT-PCR, DNA chip analysis, in situ hybridization, Northern hybridization, or Southern hybridization can be used as such a hybridization method.

In the case of using the Northern blot, the presence or absence of expression of each gene or the expression level thereof in the RNA can be detected or measured by use of the nucleic acid probe that can be used in the present invention. Specific examples thereof can include a method which comprises labeling the nucleic acid probe (a complementary strand) with a radioisotope (32P, 33P, 35 S, etc.), a fluorescent material, or the like, hybridizing the labeled product with the living tissue-derived RNA from the subject, which is transferred to a nylon membrane or the like according to a routine method, and then detecting and measuring a signal derived from the label (radioisotope or fluorescent material) on the formed DNA/RNA duplex using a radiation detector (examples thereof can include BAS-1800 II (Fujifilm Corp.)) or a fluorescence detector (examples thereof can include STORM 865 (GE Healthcare Japan Corp.)).

In the case of using the quantitative RT-PCR, the presence or absence of expression of each gene or the expression level thereof in the RNA can be detected or measured by use of the primer that can be used in the present invention. Specific examples thereof can include a method which comprises preparing cDNA from the living tissue-derived RNA of the subject according to a routine method, hybridizing a pair of primers (consisting of a plus strand and a reverse strand binding to the cDNA) of the present invention with the cDNA such that the region of each target gene can be amplified with the cDNA as a template, and performing PCR according to a routine method to detect the obtained double-stranded DNA. The method for detecting the double-stranded DNA can include a method of performing the PCR using the primers labeled in advance with a radioisotope or a fluorescent material, a method of electrophoresing the PCR product on an agarose gel and staining the double-stranded DNA with ethidium bromide or the like for detection, and a method of transferring the produced double-stranded DNA to a nylon membrane or the like according to a routine method and hybridizing the double-stranded DNA to a labeled nucleic acid probe for detection.

In the case of using the nucleic acid array analysis, an RNA chip or a DNA chip in which the nucleic acid probes (single-stranded or double-stranded) of the present invention are attached to a substrate (solid phase) is used. Regions having the attached nucleic acid probes are referred to as probe spots, and regions having no attached nucleic acid probe are referred to as blank spots. A group of genes immobilized on a solid-phase substrate is generally called a nucleic acid chip, a nucleic acid array, a microarray, or the like. The DNA or RNA array includes a DNA or RNA macroarray and a DNA or RNA microarray. The term “chip” used herein includes all of them. 3D-Gene® Human miRNA Oligo chip (Toray Industries, Inc.) can be used as the DNA chip, though the DNA chip is not limited thereto.

Examples of the measurement using the DNA chip can include, but are not limited to, a method of detecting and measuring a signal derived from the label on the nucleic acid probes using an image detector (examples thereof can include Typhoon 9410 (GE Healthcare Japan Corp.) and 3D-Gene® scanner (Toray Industries, Inc.)).

The “stringent conditions” used herein are, as mentioned above, conditions under which a nucleic acid probe hybridizes to its target sequence to a larger extent (e.g., a measurement value equal to or larger than a mean of background measurement values+a standard deviation of the background measurement values×2) than that for other sequences.

The stringent conditions are defined by hybridization and subsequent washing conditions. Examples of the hybridization conditions include, but not limited to, 30° C. to 60° C. for 1 to 24 hours in a solution containing SSC, a surfactant, formamide, dextran sulfate, a blocking agent, etc. In this context, 1×SSC is an aqueous solution (pH 7.0) containing 150 mM sodium chloride and 15 mM sodium citrate. The surfactant includes, for example, SDS (sodium dodecyl sulfate), Triton, or Tween. The hybridization conditions more preferably comprise 3 to 10×SSC and 0.1 to 1% SDS. Examples of the conditions for the washing, following the hybridization, which is another condition to define the stringent conditions, can include conditions comprising continuous washing at 30° C. in a solution containing 0.5×SSC and 0.1% SDS, at 30° C. in a solution containing 0.2×SSC and 0.1% SDS, and at 30° C. in a 0.05×SSC solution. It is desirable that the complementary strand should maintain its hybridized state with a target plus strand even by washing under such conditions. Specifically, examples of such a complementary strand can include a strand consisting of a nucleotide sequence in a completely complementary relationship with the nucleotide sequence of the target plus strand, and a strand consisting of a nucleotide sequence having at least 80%, preferably at least 85%, more preferably at least 90% or at least 95%, for example, at least 98% or at least 99% identity to the strand.

Other examples of the “stringent conditions” for the hybridization are described in, for example, Sambrook, J. & Russel, D., Molecular Cloning, A LABORATORY MANUAL, Cold Spring Harbor Laboratory Press, published on Jan. 15, 2001, Vol. 1, 7.42 to 7.45 and Vol. 2, 8.9 to 8.17, and can be used in the present invention.

Examples of the conditions for carrying out PCR using a polynucleotide fragment in the kit of the present invention as a primer include treatment for approximately 15 seconds to 1 minute at 5 to 10° C. plus a Tm value calculated from the sequence of the primer, using a PCR buffer with composition such as 10 mM Tris-HCL (pH 8.3), 50 mM KCL, and 1 to 2 mM MgCl 2. Examples of the method for calculating such a Tm value include Tm value=2×(the number of adenine residues+the number of thymine residues)+4×(the number of guanine residues+the number of cytosine residues).

In the case of using the quantitative RT-PCR, a commercially available kit for measurement specially designed for quantitatively measuring miRNA, such as TaqMan® MicroRNA Assays (Life Technologies Corp.); LNA®-based MicroRNA PCR (Exiqon); or Ncode® miRNA qRT-PCT kit (Invitrogen Corp.) may be used.

For the calculation of gene expression levels, statistical treatment described in, for example, Statistical analysis of gene expression microarray data (Speed T., Chapman and Hall/CRC), and A beginner's guide Microarray gene expression data analysis (Causton H. C. et al., Blackwell publishing) can be used in the present invention, though the calculation method is not limited thereto. For example, twice, preferably 3 times, more preferably 6 times the standard deviation of the measurement values of the blank spots are added to the average measurement value of the blank spots on the DNA chip, and probe spots having a signal value equal to or larger than the resulting value can be regarded as detection spots. Alternatively, the average measurement value of the blank spots is regarded as a background and can be subtracted from the measurement values of the probe spots to determine gene expression levels. A missing value for a gene expression level can be excluded from the analyte, preferably replaced with the smallest value of the gene expression level in each DNA chip, or more preferably replaced with a value obtained by subtracting 0.1 from a logarithmic value of the smallest value of the gene expression level. In order to eliminate low-signal genes, only a gene having a gene expression level of 2⁶, preferably 2⁸, more preferably 2¹⁰ or larger in 20% or more, preferably 50% or more, more preferably 80% or more of the number of measurement samples can be selected as the analyte. Examples of the normalization of the gene expression level include, but are not limited to, global normalization and quantile normalization (Bolstad, B. M. et al., 2003, Bioinformatics, Vol. 19, p. 185-193).

The present invention also provides a method comprising measuring a target gene or gene expression level in a sample derived from a subject using the polynucleotide, the kit, or the device (e.g., chip) for detection of the present invention, or a combination thereof, preparing a discriminant (discriminant function) with gene expression levels in a sample derived from a liver cancer patient and a sample derived from a healthy subject as supervising samples, and determining or evaluating the presence and/or absence of the liver cancer-derived gene in the sample.

Specifically, the present invention further provides the method comprising: a first step of measuring in vitro an expression level of a target gene (target nucleic acids) in multiple samples known to determine or evaluate the presence or absence of the liver cancer-derived gene in the samples, using the polynucleotide, the kit, or the device (e.g., chip) for detection of the present invention, or a combination thereof; a second step of constructing a discriminant with the measurement values of the expression level of the target gene obtained in the first step as supervising samples; a third step of measuring in vitro an expression level of the target gene in a sample derived from a subject in the same way as in the first step; and a fourth step of substituting the measurement value of the expression level of the target gene obtained in the third step into the discriminant obtained in the second step, and determining or evaluating the presence or absence of the liver cancer-derived gene in the sample on the basis of the results obtained from the discriminant, wherein the target gene can be detected using the polynucleotide or using a polynucleotide for detection contained in the kit or the device (e.g., chip). In this context, the discriminant can be prepared by use of Fisher's linear discriminant analysis, nonlinear discriminant analysis based on Mahalanobis' distance, neural network, Support Vector Machine (SVM), or the like, though the method is not limited thereto.

When a clustering boundary is a straight line or a hyperplane, the linear discriminant analysis is a method for determining the association of a cluster using Formula 1 as a discriminant. In this formula, x represents an explanatory variable, w represents a coefficient of the explanatory variable, and wo represents a constant term.

$$f ( x ) = {w}_{0} + {\sum }_{i = 1}^{n} {w}_{i} {x}_{i} \qquad Formula 1$$

Values obtained from the discriminant are referred to as discriminant scores. The measurement values of a newly offered data set can be substituted as explanatory variables into the discriminant to determine clusters on the basis of the signs of the discriminant scores.

The Fisher's linear discriminant analysis, one type of linear discriminant analysis, is a dimensionality reduction method for selecting a dimension suitable for discriminating classes, and constructs a highly discriminating synthetic variable by focusing on the variance of the synthetic variables and minimizing the variance of data having the same label (Venables, W. N. et al., Modern Applied Statistics with S. Fourth edition. Springer., 2002). In the Fisher's linear discriminant analysis, direction w of projection is determined so as to maximize Formula 2. In this formula, μ represents an average input, ng represents the number of data associated with class g, and μg represents an average input of the data associated with class g. The numerator and the denominator are interclass variance and intraclass variance, respectively, when each data is projected in the direction of the vector w. Discriminant coefficient wi is determined by maximizing this ratio (Takafumi Kanamori et al., “Pattern Recognition”, Kyoritsu Shuppan Co., Ltd. (2009); and Richard O. et al., Pattern Classification Second Edition., Wiley-Interscience, 2000).

$$J ( w ) = \frac{{\sum }_{g = 1}^{G} {n}_{g} ( {w}^{T} {μ}_{g} - {w}^{T} μ ) {( {w}^{T} {μ}_{g} - {w}^{T} μ )}^{T}}{{\sum }_{g = 1}^{G} {\sum }_{i : {y}_{i} = g} ( {w}^{T} {x}_{i} - {w}^{T} {μ}_{g} ) ( {w}^{T} {x}_{i} - {w}^{T} {μ}_{g} )} \qquad Formula 2$$

$$subject to μ = {\sum }_{i = 1}^{n} \frac{{x}_{i}}{n} , {μ}_{g} = {\sum }_{i : {μ}_{i} = g}^{n} \frac{{x}_{i}}{{n}_{g}}$$

The Mahalanobis' distance is calculated according to Formula 3 in consideration of data correlation and can be used as nonlinear discriminant analysis for determining an associated cluster, based on a closer Mahalanobis' distance from each cluster. In this Formula 3, μ represents a central vector of each cluster, and S⁻¹ represents an inverse matrix of the variance-covariance matrix of the cluster. The central vector is calculated from explanatory variable x, and an average vector, a median value vector, or the like can be used.

$$D ( x , μ ) = {\{ {( x - μ )}^{t} {S}^{- 1} ( x - μ ) \}}^{\frac{1}{2}} \qquad Formula 3$$

SVM is a discriminant analysis method devised by V. Vapnik (The Nature of Statistical Leaning Theory, Springer, 1995). Particular data points of a data set having known classes are defined as explanatory variables, and classes are defined as objective variables. A boundary plane called hyperplane for correctly classifying the data set into the known classes is determined, and a discriminant for data classification is determined using the boundary plane. Then, the measurement values of a newly offered data set can be substituted as explanatory variables into the discriminant to determine classes. In this respect, the result of the discriminant analysis may be classes, may be a probability of data to be classified into correct classes, or may be the distance from the hyperplane. In SVM, a method of nonlinearly converting a feature vector to a high dimension and performing linear discriminant analysis in the space is known as a method for tackling nonlinear problems. A formula in which an inner product of two factors in a nonlinearly mapped space is expressed only by inputs in their original spaces is called kernel. Examples of the kernel can include a linear kernel, a RBF (radial basis function) kernel, and a Gaussian kernel. While highly dimensional mapping is performed according to the kernel, the optimum discriminant, i.e., a discriminant, can be actually constructed by mere calculation according to the kernel, which avoids calculating features in the mapped space (e.g., Hideki Aso et al., Frontier of Statistical Science 6 “Statistics of pattern recognition and learning— New concepts and approaches”, Iwanami Shoten, Publishers (2004); Nello Cristianini et al., Introduction to SVM, Kyoritsu Shuppan Co., Ltd. (2008)).

C-support vector classification (C-SVC), one type of SVM, comprises preparing a hyperplane by supervising with the explanatory variables of two groups and classifying an unknown data set into either of the groups (C. Cortes et al., 1995, Machine Learning, Vol. 20, p. 273-297).

Exemplary calculation of the C-SVC discriminant that can be used in the method of the present invention will be given below. First, all subjects are divided into two groups, i.e., a liver cancer patient group and a healthy subject group. For example, liver tissue examination can be used for confirming each subject either as a liver cancer patient or as a healthy subject.

Next, a data set consisting of comprehensive gene expression levels of serum-derived samples of the two divided groups (hereinafter, this data set is referred to as a training cohort) is prepared, and a C-SVC discriminant is determined by using genes found to differ clearly in their gene expression levels between the two groups as explanatory variables and this grouping as objective variables (e.g., −1 and +1). An optimizing objective function is represented by Formula 4 wherein e represents all input vectors, y represents an objective variable, a represents a Lagrange's undetermined multiplier vector, Q represents a positive definite matrix, and C represents a parameter for adjusting constrained conditions.

$${min}_{a} \frac{1}{2} {a}^{T} Q a - {e}^{T} a \qquad Formula 4$$

$$subject to {y}^{T} a = 0 , 0 \leq {a}_{i} \leq C , i = 1 , … , l ,$$

Formula 5 is a finally obtained discriminant, and a group to which the data point is associated can be determined on the basis of the sign of a value obtained according to the discriminant. In this formula, x represents a support vector, y represents a label indicating the association of a group, a represents the corresponding coefficient, b represents a constant term, and K represents a kernel function.

$$f ( x ) = sgn ( {\sum }_{j = 1}^{l} {y}_{j} {a}_{j} K ( {x}_{j} , x ) + b ) \qquad Formula 5$$

For example, a RBF kernel defined by Formula 6 can be used as the kernel function. In this formula, x represents a support vector, and y represents a kernel parameter for adjusting the complexity of the hyperplane.

K(xᵢ,xⱼ)=exp(−r∥x,−x,∥²)r<0 Formula 6

In addition, an approach such as neural network, k-nearest neighbor algorithms, decision trees, or logistic regression analysis can be selected as a method for determining or evaluating the presence and/or absence of expression of a liver cancer-derived target gene in a sample derived from a subject, or for evaluating the expression level thereof by comparison with a control derived from a healthy subject.

The method of the present invention can comprise, for example, the following steps (a), (b), and (c):
(a) measuring an expression level of a target gene in tissues containing liver cancer-derived genes derived from liver cancer patients and/or samples that are already known to contain no liver cancer-derived gene derived from healthy subjects, using the polynucleotide, the kit, or the device (e.g., DNA chip) for detection according to the present invention;
(b) preparing the discriminants of Formulae 1 to 3, 5, and 6 described above from the measurement values of the expression level measured in the step (a); and
(c) measuring an expression level of the target gene in a sample derived from a subject using the polynucleotide, the kit, or the device (e.g., DNA chip) for detection according to the present invention, substituting the obtained measurement value into the discriminants prepared in the step (b), and determining or evaluating the presence and/or absence of the liver cancer-derived target gene in the sample, or evaluating the expression level thereof by comparison with a healthy subject-derived control, on the basis of the obtained results. In this context, in the discriminants of Formulae 1 to 3, 5, and 6, x represents an explanatory variable and includes a value obtained by measuring a polynucleotide selected from the polynucleotides described above in the Section 2, or a fragment thereof, etc. Specifically, the explanatory variable for discriminating a liver cancer patient from a healthy subject according to the present invention is a gene expression level selected from, for example, the following expression levels (1) to (3):
(1) a gene expression level in the serum of a pancreatic cancer patient or a healthy subject measured by any of DNA comprising 15 or more consecutive nucleotides in a nucleotide sequence represented by any of SEQ ID NOs: 1 to 167 and 714 to 729 or a complementary sequence thereof,
(2) a gene expression level in the serum of a pancreatic cancer patient or a healthy subject measured by any of DNA comprising 15 or more consecutive nucleotides in a nucleotide sequence represented by any of SEQ ID NOs: 168 to 183 or a complementary sequence thereof, and
(3) a gene expression level in the serum of a liver cancer patient or a healthy subject measured by any DNA comprising 15 or more consecutive nucleotides in a nucleotide sequence represented by any of SEQ ID NOs: 184 to 224 or a complementary sequence thereof.

As described above, for the method for determining or evaluating the presence and/or absence of a liver cancer-derived gene in a sample derived from a subject, the preparation of a discriminant requires a discriminant prepared in a training cohort. For enhancing the discriminant accuracy of the discriminant, it is necessary for the discriminant to use genes that show clear difference between two groups in the training cohort when preparing the discriminant.

Each gene that is used for an explanatory variable in a discriminant is preferably determined as follows. First, comprehensive gene expression levels of a liver cancer patient group and comprehensive gene expression levels of a healthy subject group, both of which are in a training cohort, are used as a data set, the degree of difference in the expression level of each gene between the two groups is determined through the use of, for example, the P value of t test, which is parametric analysis, or the P value of Mann-Whitney's U test or Wilcoxon test, which is nonparametric analysis.

The gene can be regarded as being statistically significant when the critical rate (significance level) as the P value obtained by the test is smaller than, for example, 5%, 1%, or 0.01%.

In order to correct an increased probability of type I error attributed to the repetition of a test, a method known in the art, for example, Bonferroni or Holm method, can be used for the correction (e.g., Yasushi Nagata et al., “Basics of statistical multiple comparison methods”, Scientist Press Co., Ltd. (2007)). As an example of the Bonferroni correction, for example, the P value obtained by a test is multiplied by the number of repetitions of the test, i.e., the number of genes used in the analysis, and the obtained value can be compared with a desired significance level to suppress a probability of causing type I error in the whole test.

Instead of the statistical test, the absolute value (fold change) of an expression ratio of a median value of each gene expression level between gene expression levels of a liver cancer patient group and gene expression levels of a healthy subject group may be calculated to select a gene that is used for an explanatory variable in a discriminant. Alternatively, ROC curves may be prepared using gene expression levels of a liver cancer patient group and a healthy subject group, and a gene that is used for an explanatory variable in a discriminant can be selected on the basis of an AUROC value.

Next, a discriminant that can be calculated by various methods described above is prepared using any number of genes having large difference in their gene expression levels determined here. Examples of the method for constructing a discriminant that produces the largest discriminant accuracy include a method of constructing a discriminant in every combination of genes that satisfy the significance level of P value, and a method of repetitively evaluating the genes for use in the preparation of a discriminant while increasing the number of genes one by one in a descending order of difference in gene expression level (Furey T S. et al., 2000, Bioinformatics., Vol. 16, p. 906-14). A gene expression level of another independent liver cancer patient or healthy subject is substituted as an explanatory variable into this discriminant to calculate discrimination results of the group to which this independent liver cancer patient or healthy subject belongs. Specifically, the found gene set for diagnosis and the discriminant constructed using the gene set for diagnosis can be evaluated in an independent sample cohort to find a more universal gene set for diagnosis capable of detecting liver cancer and a more universal method for discriminating liver cancer.

Split-sample method is preferably used for evaluating the discriminant performance (generality) of the discriminant. Specifically, a data set is divided into a training cohort and a validation cohort, and gene selection by a statistical test and discriminant preparation are performed using the training cohort. Accuracy, sensitivity, and specificity are calculated using results of discriminating a validation cohort according to the discriminant and a true group to which the validation cohort associates, to evaluate the discriminant performance. On the other hand, instead of dividing a data set, the gene selection by a statistical test and discriminant preparation may be performed using all of samples, and accuracy, sensitivity, and specificity can be calculated by the discriminant analysis using a newly prepared samples cohort for evaluation of the discriminant performance.

The present invention provides a polynucleotide for detection or for disease diagnosis useful in the diagnosis and treatment of liver cancer, a method for detecting liver cancer using the polynucleotide, and a kit and a device for the detection of liver cancer, comprising the polynucleotide. Particularly, in order to select a gene for diagnosis and prepare a discriminant so as to exhibit accuracy beyond a liver cancer diagnosis method using an existing tumor marker CEA, a gene set for diagnosis and a discriminant for the method of the present invention, that exhibit accuracy beyond AFP, CEA, CA19-9 and/or PIVKA-II, can be constructed, for example, by comparing expressed genes in serum derived from a patient confirmed to be negative using AFP, CEA, CA19-9, and/or PIVKA-II but finally found to have liver cancer by detailed examination such as computed tomography using a contrast medium, with genes expressed in serum derived from a patient having no liver cancer.

For example, the gene set for diagnosis is set to any combination selected from one or two or more of the polynucleotides based on a nucleotide sequence represented by any of SEQ ID NOs: 1 to 167 and 714 to 729 or a complementary sequence thereof as described above, optionally one or two or more of the polynucleotides based on a nucleotide sequence represented by any of SEQ ID NOs: 168 to 183 or a complementary sequence thereof, and optionally one or two or more of the polynucleotides based on a nucleotide sequence represented by any of SEQ ID NOs: 184 to 224 or a complementary sequence thereof. Further, a discriminant is constructed using expression levels of the gene set for diagnosis in samples derived from class I liver cancer patients as a result of tissue diagnosis and samples derived from class II healthy subjects as a result of tissue diagnosis. As a result, the presence or absence of liver cancer-derived genes in an unknown sample can be determined with 100% accuracy at the maximum by measuring expression levels of the gene set for diagnosis in an unknown sample.

### EXAMPLES

Hereinafter, the present invention is described further specifically with reference to Examples below. However, the scope of the present invention is not intended to be limited by these Examples.

### Reference Example 1

<Collection of Samples from Liver Cancer Patients and Healthy Subjects>

Sera were collected using VENOJECT II vacuum blood collecting tube VP-AS109K60 (Terumo Corp.) from 100 healthy subjects and 34 liver cancer patients (15 cases with stage I, 9 cases with stage II, 5 cases with stage IIIA, 2 cases with stage TIM, 1 case with stage IIIC, and 2 cases with stage IV) confirmed to have no primary cancer other than liver cancer after acquisition of informed consent, and used as a training cohort. Likewise, sera were collected using VENOJECT II vacuum blood collecting tube VP-AS109K60 (Terumo Corp.) from 50 healthy subjects and 16 liver cancer patients (9 cases with stage I, 5 cases with stage II, and 2 cases with stage IIIA) confirmed to have no primary cancer other than liver cancer after acquisition of informed consent, and used as a validation cohort.

<Extraction of Total RNA>

Total RNA was obtained from 300 μL of the serum sample obtained from each of 200 persons in total of 150 healthy subjects and 50 liver cancer patients included in the training cohort and the validation cohort, using a reagent for RNA extraction in 3D-Gene® RNA extraction reagent from liquid sample kit (Toray Industries, Inc.) according to the protocol provided by the manufacturer.

<Measurement of Gene Expression Level>

miRNAs in the total RNA obtained from the serum sample of each of 200 persons in total of 150 healthy subjects and 50 liver cancer patients included in the training cohort and the validation cohort were fluorescently labeled using 3D-Gene® miRNA Labeling kit (Toray Industries, Inc.) according to the protocol (ver 2.20) provided by the manufacturer. The oligo DNA chip used was 3D-Gene® Human miRNA Oligo chip (Toray Industries, Inc.) with attached probes having sequences complementary to 2,555 miRNAs among the miRNAs registered in miRBase Release 20. Hybridization between the miRNAs in the total RNA and the probes on the DNA chip under stringent conditions and washing following the hybridization were performed according to the protocol provided by the manufacturer. The DNA chip was scanned using 3D-Gene® scanner (Toray Industries, Inc.) to obtain images. Fluorescence intensity was digitized using 3D-Gene® Extraction (Toray Industries, Inc.). The digitized fluorescence intensity was converted to a logarithmic value having a base of 2 and used as a gene expression level, from which a blank value was subtracted. A missing value was replaced with a value obtained by subtracting 0.1 from a logarithmic value of the smallest value of the gene expression level in each DNA chip. As a result, the comprehensive gene expression levels of the miRNAs in the sera were obtained for the 150 liver cancer patients and the 150 healthy subjects. Calculation and statistical analysis using the digitized gene expression levels of the miRNAs were carried out using R language 3.0.2 (R Development Core Team (2013). R: A language and environment for statistical computing. R Foundation for Statistical Computing, URL http://www.R-project.org/.) and MASS package 7.3-30 (Venables, W. N. & Ripley, B. D. (2002) Modern Applied Statistics with S. Fourth Edition. Springer, New York. ISBN 0-387-95457-0).

### Reference Example 2

<Collection of Samples from Patients with Cancers Other than Liver Cancer>

Sera were collected using VENOJECT II vacuum blood collecting tube VP-AS109K60 (Terumo Corp.) from each of 72 pancreatic cancer patients, 61 bile duct cancer patients, 38 stomach cancer patients, 25 esophageal cancer patients, 35 colorectal cancer patients, and 16 benign pancreaticobiliary disease patients confirmed to have no cancer in other organs after acquisition of informed consent, and used as a training cohort together with the samples of 35 liver cancer patients and 99 healthy subjects of Reference Example 1. Likewise, sera were collected using VENOJECT II vacuum blood collecting tube VP-AS109K60 (Terumo Corp.) from each of 28 pancreatic cancer patients, 37 bile duct cancer patients, 12 stomach cancer patients, 25 esophageal cancer patients, 15 colorectal cancer patients, and 5 benign pancreaticobiliary disease patients confirmed to have no cancer in other organs after acquisition of informed consent, and used as a validation cohort together with the samples of 17 liver cancer patients confirmed to have no cancer in organs except for liver cancer and 51 healthy subjects of Reference Example 1. Subsequent operations were conducted in the same way as in Reference Example 1.

### Example 1

<Selection of Gene Marker Using Samples in the Training Cohort, and Method for Evaluating Liver Cancer Discriminant Performance of the Single Gene Marker Using Samples in the Validation Cohort>

In this Example, a gene marker for discriminating a liver cancer patient from a healthy subject was selected from the training cohort, and studied in samples of the validation cohort independent of the training cohort, for a method for evaluating liver cancer discriminant performance of each selected gene marker alone.

Specifically, first, the miRNA expression levels in the training cohort and the validation cohort obtained in the above-mentioned Reference Examples were combined and normalized by quantile normalization.

Next, genes for diagnosis were selected in the training cohort. Here, in order to acquire diagnostic markers with higher reliability, only genes having the gene expression level of 2⁶ or higher in 50% or more of the samples in either of the liver cancer patient group in the training cohort or the healthy subject group of the training cohort were selected. In order to further acquire statistically significant genes for discriminating a liver cancer patient group from a healthy subject group, the P value obtained by two-tailed t-test assuming equal variance as to each gene expression level was corrected by the Bonferroni method, and genes that satisfied p <0.01 were acquired as gene markers for use in explanatory variables of a discriminant and described in Table 2.

In this way, hsa-miR-1343-3p, hsa-miR-6726-5p, hsa-miR-6515-3p, hsa-miR-4651, hsa-miR-4257, hsa-miR-3188, hsa-miR-6131, hsa-miR-6766-3p, hsa-miR-7641, hsa-miR-1249, hsa-miR-3679-3p, hsa-miR-6787-5p, hsa-miR-4454, hsa-miR-3135b, hsa-miR-6765-3p, hsa-miR-7975, hsa-miR-204-3p, hsa-miR-7977, hsa-miR-7110-5p, hsa-miR-6717-5p, hsa-miR-6870-5p, hsa-miR-663b, hsa-miR-6875-5p, hsa-miR-8072, hsa-miR-6816-5p, hsa-miR-4281, hsa-miR-6729-5p, hsa-miR-8069, hsa-miR-4706, hsa-miR-7108-5p, hsa-miR-4433b-3p, hsa-miR-6893-5p, hsa-miR-6857-5p, hsa-miR-1227-5p, hsa-miR-6741-5p, hsa-miR-451a, hsa-miR-8063, hsa-miR-3622a-5p, hsa-miR-615-5p, hsa-miR-128-1-5p, hsa-miR-6825-5p, hsa-miR-1260b, hsa-miR-4433-3p, hsa-miR-4665-5p, hsa-miR-7845-5p, hsa-miR-1908-5p, hsa-miR-6840-3p, hsa-miR-6765-5p, hsa-miR-296-5p, hsa-miR-3675-3p, hsa-miR-6781-5p, hsa-miR-423-5p, hsa-miR-3663-3p, hsa-miR-6784-5p, hsa-miR-6749-5p, hsa-miR-1231, hsa-miR-4746-3p, hsa-miR-6780b-5p, hsa-miR-4758-5p, hsa-miR-3679-5p, hsa-miR-3184-5p, hsa-miR-6125, hsa-miR-6721-5p, hsa-miR-6791-5p, hsa-miR-3185, hsa-miR-1260a, hsa-miR-3197, hsa-miR-6845-5p, hsa-miR-6887-5p, hsa-miR-6738-5p, hsa-miR-6872-3p, hsa-miR-4497, hsa-miR-1229-5p, hsa-miR-6820-5p, hsa-miR-6777-5p, hsa-miR-3917, hsa-miR-5787, hsa-miR-4286, hsa-miR-6877-5p, hsa-miR-1225-3p, hsa-miR-6088, hsa-miR-6800-5p, hsa-miR-1246, hsa-miR-4467, hsa-miR-4419b, hsa-miR-1914-3p, hsa-miR-4632-5p, hsa-miR-1915-5p, hsa-miR-3940-5p, hsa-miR-1185-2-3p, hsa-miR-6746-5p, hsa-miR-5001-5p, hsa-miR-1228-5p, hsa-miR-5572, hsa-miR-4327, hsa-miR-4638-5p, hsa-miR-6799-5p, hsa-miR-6861-5p, hsa-miR-6727-5p, hsa-miR-4513, hsa-miR-6805-3p, hsa-miR-6808-5p, hsa-miR-4449, hsa-miR-1199-5p, hsa-miR-1275, hsa-miR-4792, hsa-miR-4443, hsa-miR-6891-5p, hsa-miR-6826-5p, hsa-miR-6807-5p, hsa-miR-7150, hsa-miR-4534, hsa-miR-4476, hsa-miR-4649-5p, hsa-miR-4525, hsa-miR-1915-3p, hsa-miR-4516, hsa-miR-4417, hsa-miR-642b-3p, hsa-miR-3141, hsa-miR-5100, hsa-miR-6848-5p, hsa-miR-4739, hsa-miR-4459, hsa-miR-1237-5p, hsa-miR-296-3p, hsa-miR-4665-3p, hsa-miR-6786-5p, hsa-miR-4258, hsa-miR-6510-5p, hsa-miR-1343-5p, hsa-miR-1247-3p, hsa-miR-6805-5p, hsa-miR-4492, hsa-miR-1469, hsa-miR-1268b, hsa-miR-6858-5p, hsa-miR-3937, hsa-miR-939-5p, hsa-miR-3656, hsa-miR-744-5p, hsa-miR-4687-3p, hsa-miR-4763-3p, hsa-miR-3620-5p, hsa-miR-3195, hsa-miR-6842-5p, hsa-miR-4707-5p, hsa-miR-642a-3p, hsa-miR-7113-3p, hsa-miR-4728-5p, hsa-miR-5195-3p, hsa-miR-1185-1-3p, hsa-miR-6774-5p, hsa-miR-8059, hsa-miR-3131, hsa-miR-7847-3p, hsa-miR-4463, hsa-miR-128-2-5p, hsa-miR-4508, hsa-miR-6806-5p, hsa-miR-7111-5p, hsa-miR-6782-5p, hsa-miR-4734, hsa-miR-3162-5p, hsa-miR-887-3p, hsa-miR-6752-5p, hsa-miR-6724-5p, hsa-miR-23b-3p, hsa-miR-23a-3p, hsa-miR-625-3p, hsa-miR-1228-3p, hsa-miR-614, hsa-miR-1913, hsa-miR-92a-2-5p, hsa-miR-187-5p, hsa-miR-16-5p, hsa-miR-92b-3p, hsa-miR-150-3p, hsa-miR-564, hsa-miR-125a-3p, hsa-miR-92b-5p, hsa-miR-92a-3p, and hsa-miR-663a genes represented by SEQ ID NOs: 1 to 183 were found as liver cancer markers relative to the healthy subjects.

Among them, genes newly found as markers for examining the presence or absence of liver cancer are polynucleotides consisting of the nucleotide sequences represented by SEQ ID NOs: 1 to 167.

A discriminant for determining the presence or absence of liver cancer was further prepared by Fisher's linear discriminant analysis with the expression levels of these genes as an indicator. Specifically, any newly found polynucleotide consisting of a nucleotide sequence represented by any of SEQ ID NOs: 1 to 183 in the training cohort was input to Formula 2 to construct a discriminant. Calculated accuracy, sensitivity, and specificity are shown in Table 3. In this respect, a discriminant coefficient and a constant term are shown in Table 4.

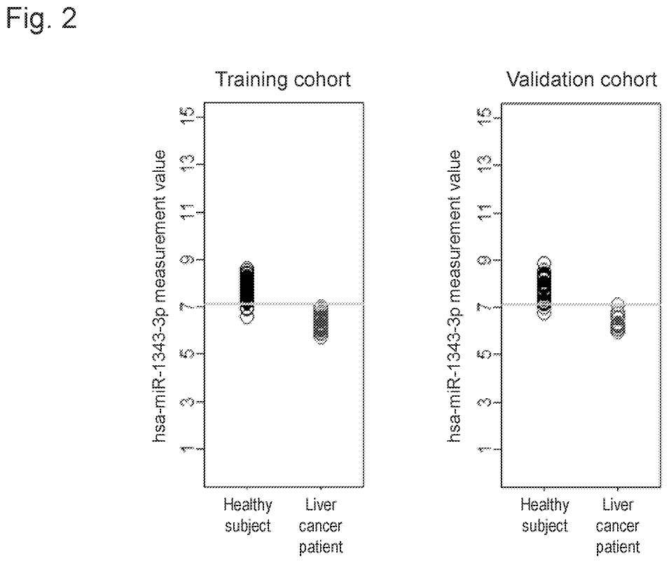

Accuracy, sensitivity, and specificity in the validation cohort were calculated using the discriminant thus prepared, and the discriminant performance of the selected polynucleotides was validated using independent samples (Table 3). For example, the expression level measurement value of the nucleotide sequence represented by SEQ ID NO: 1 was compared between the healthy subjects (100 persons) and the liver cancer patients (34 persons) in the training cohort. As a result, the gene expression level measurement values were found to be significantly lower in the liver cancer patient group than in the healthy subject group (see the left diagram of FIG. 2). These results were also reproducible for the healthy subjects (50 persons) and the liver cancer patients (16 persons) in the validation cohort (see the right diagram of FIG. 2). Likewise, the results obtained about the other polynucleotides shown in SEQ ID NOs: 2 to 183 showed that the gene expression level measurement values were significantly lower (−) or higher (+) in the liver cancer patient group than in the healthy subject group (Table 2). These results were able to be validated in the validation cohort. For example, as for this nucleotide sequence represented by SEQ ID NO: 1, the number of samples that were correctly identified in the detection of liver cancer was calculated using the threshold (7.09) that was set in the training cohort and discriminated between the two groups. As a result, 15 true positives, 49 true negatives, 1 false positive, and 1 false negatives were obtained in the validation cohort. From these values, 97% accuracy, 94% sensitivity, and 98% specificity were obtained as the detection performance. In this way, the detection performance was calculated as to all of the polynucleotides shown in SEQ ID NOs: 1 to 183, and described in Table 3.

Likewise, 72 polynucleotides consisting of the nucleotide sequences represented by SEQ ID NOs: 1, 2, 3, 4, 5, 6, 7, 8, 9, 10, 11, 12, 13, 14, 15, 16, 17, 19, 21, 22, 23, 24, 25, 27, 28, 29, 30, 31, 32, 33, 34, 35, 37, 39, 40, 41, 43, 44, 45, 46, 47, 48, 50, 51, 54, 55, 56, 57, 58, 60, 61, 62, 63, 64, 65, 68, 73, 80, 86, 88, 91, 93, 94, 99, 114, 117, 170, 171, 172, 173, 174 and 175 exhibited sensitivity of 93.8%, 93.8%, 93.8%, 87.5%, 75%, 87.5%, 62.5%, 81.2%, 93.8%, 93.8%, 75%, 93.8%, 62.5%, 93.8%, 56.2%, 56.2%, 56.2%, 93.8%, 68.8%, 87.5%, 93.8%, 81.2%, 87.5%, 62.5%, 56.2%, 68.8%, 81.2%, 81.2%, 62.5%, 87.5%, 68.8%, 75%, 75%, 75%, 62.5%, 93.8%, 75%, 56.2%, 62.5%, 62.5%, 68.8%, 87.5%, 75%, 62.5%, 75%, 68.8%, 62.5%, 68.8%, 68.8%, 68.8%, 62.5%, 62.5%, 75%, 62.5%, 75%, 68.8%, 56.2%, 81.2%, 68.8%, 56.2%, 62.5%, 56.2%, 56.2%, 68.8%, 56.2%, 62.5%, 87.5%, 87.5%, 75%, 68.8%, 62.5% and 81.2% respectively, in the validation cohort (Table 3). As seen from Comparative Example mentioned later, AFP, which had the highest sensitivity among four existing markers, had sensitivity of 53.3% in the validation cohort (Table 5), demonstrating that, for example, the 72 polynucleotides consisting of the nucleotide sequences represented by SEQ ID NOs: 1, 2, 3, 4, 5, 6, 7, 8, 9, 10, 11, 12, 13, 14, 15, 16, 17, 19, 21, 22, 23, 24, 25, 27, 28, 29, 30, 31, 32, 33, 34, 35, 37, 39, 40, 41, 43, 44, 45, 46, 47, 48, 50, 51, 54, 55, 56, 57, 58, 60, 61, 62, 63, 64, 65, 68, 73, 80, 86, 88, 91, 93, 94, 99, 114, 117, 170, 171, 172, 173, 174 and 175 can discriminate, each alone, liver cancer in the validation cohort with sensitivity beyond AFP.

Also, for example, 7 polynucleotides consisting of the nucleotide sequences represented by SEQ ID NOs: 1, 6, 15, 31, 46, 50, and 58 were able to correctly determine all of the nine stage 1 liver cancer samples contained in the validation cohort to have liver cancer. Thus, these polynucleotides can detect even early liver cancer and contribute to the early diagnosis of liver cancer.

### Example 2

<Method for Evaluating Liver Cancer Discriminant Performance by Combination of Multiple Gene Markers Using Samples in the Validation Cohort>

In this Example, a method for evaluating liver cancer discriminant performance by a combination of the gene markers selected in Example 1 was studied. Specifically, Fisher's linear discriminant analysis was conducted as to 16,533 combinations of two expression level measurement values comprising at least one or more of the expression level measurement values of the newly found polynucleotides consisting of the nucleotide sequences represented by SEQ ID NOs: 1 to 167 among the polynucleotides consisting of the nucleotide sequences represented by SEQ ID NOs: 1 to 183 selected in Example 1, to construct a discriminant for determining the presence or absence of liver cancer. Next, accuracy, sensitivity, and specificity in the validation cohort were calculated using the discriminant thus prepared, and the discriminant performance of the selected polynucleotides was validated using the independent samples.

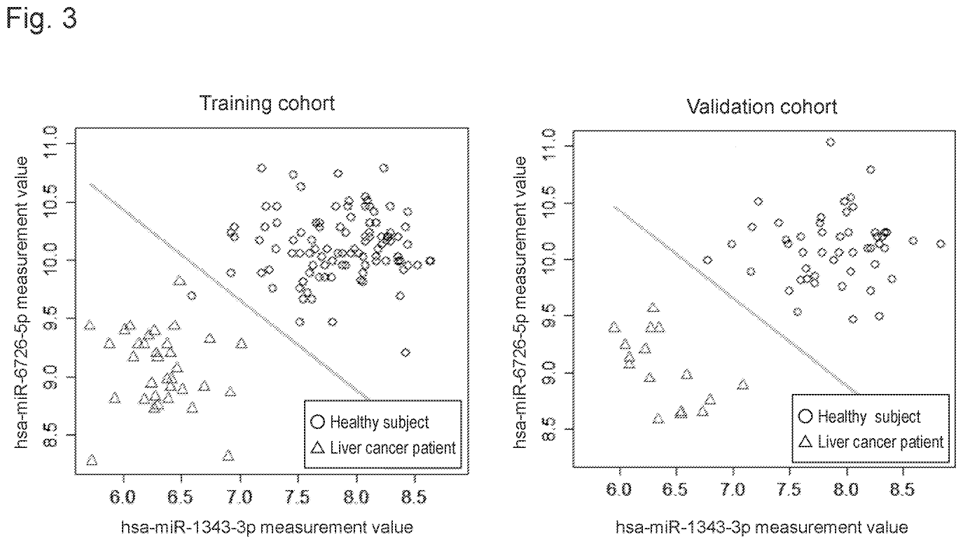

For example, the expression level measurement values of polynucleotides consisting of the nucleotide sequences represented by SEQ ID NO: 1 and SEQ ID NO: 2 were compared between the healthy subjects (100 persons) and the liver cancer patients (34 persons) in the training cohort. As a result, a scatter diagram that significantly separated the expression level measurement values of the liver cancer patient group from those of the healthy subject group was obtained (see the left diagram of FIG. 3). These results were also reproducible for the healthy subjects (50 persons) and the liver cancer patients (16 persons) in the validation cohort (see the right diagram of FIG. 3). Likewise, a scatter diagram that significantly separated the expression level measurement values of the liver cancer patient group from those of the healthy subject group was also obtained as to the other combinations of two expression level measurement values comprising at least one or more of the expression level measurement values of the newly found polynucleotides consisting of the nucleotide sequences represented by SEQ ID NOs: 1 to 167 among the polynucleotides consisting of the nucleotide sequences represented by SEQ ID NOs: 1 to 183. These results were able to be validated in the validation cohort. For example, as for these nucleotide sequences represented by SEQ ID NO: 1 and SEQ ID NO: 2, the number of correctly or incorrectly identified samples in the detection of liver cancer was calculated using the function (0=0.77x+y−15.07) that was set in the training cohort and discriminated between the two groups. As a result, 16 true positives, 50 true negatives, 0 false positives, and 0 false negatives were obtained. From these values, 100% accuracy, 100% sensitivity, and 100% specificity were obtained as the detection performance. In this way, the detection performance was calculated for all combinations of two expression level measurement values comprising at least one more of the expression level measurement values of any of the newly found polynucleotides consisting of the nucleotide sequences represented by SEQ ID NOs: 1 to 167 among the polynucleotides consisting of the nucleotide sequences represented by SEQ ID NOs: 1 to 183. Among them, 182 combinations comprising the expression level measurement value of the polynucleotide consisting of the nucleotide sequence represented by SEQ ID NO: 1 and the detection performance thereof were described in Table 6 as an example. For example, all of combinations of the expression level measurement values of the polynucleotides consisting of the nucleotide sequences represented by SEQ ID NOs: 1 and 2, SEQ ID NOs: 1 and 3, SEQ ID NOs: 1 and 4, and SEQ ID NOs: 1 and 5 exhibited sensitivity of 100%, 100%, 100%, 94%, and 94%, respectively, in the validation cohort. Likewise, the sensitivity was also calculated as to the combinations of two polynucleotides consisting of the nucleotide sequences represented by SEQ ID NO: 1 and any of SEQ ID NOs: 6 to 251. As a result, all of these combinations exhibited sensitivity of 88% or higher (Table 6), which was beyond the sensitivity (53.3%) of the existing liver cancer marker AFP (Table 5). Thus, a combination of two of the polynucleotides consisting of the nucleotide sequences represented by SEQ ID NOs: 1 to 183 also produced excellent liver cancer detection sensitivity.

In addition, markers for the detection of liver cancer with more excellent sensitivity are obtained by combining the expression level measurement values of 3, 4, 5, 6, 7, 8, 9, 10 or more of the polynucleotides consisting of the nucleotide sequences represented by SEQ ID NOs: 1 to 183. For example, the newly found polynucleotides consisting of the nucleotide sequences represented by SEQ ID NOs: 1 to 167 among the polynucleotides consisting of the nucleotide sequences represented by SEQ ID NOs: 1 to 183 selected in Example 1 were measured to obtain their expression levels of the healthy subject group and the liver cancer group in the validation cohort. All of the polynucleotides were ranked in the descending order of their P values based on the Student's t-test which indicate statistical significance of difference between groups (i.e., one having the lowest P value was ranked in the first place), and liver cancer detection sensitivity was evaluated for each of combinations of one or more polynucleotides to which the polynucleotides were added one by one from the top to the bottom according to the rank. In short, the order in terms of SEQ ID NOs in which the polynucleotides were combined in this evaluation is in reverse in terms of SEQ ID NO: 167 to SEQ ID NOs: 166, 165, . . . shown in Table 2 in order. As a result, the sensitivity in the validation cohort was 12.5% for 1 polynucleotide (SEQ ID NO: 167), 43.8% for 2 polynucleotides (SEQ ID NOs: 166 and 167), 68.8% for 4 polynucleotides (SEQ ID NOs: 164 to 167), 87.5% for 6 polynucleotides (SEQ ID NOs: 162 to 167), 93.8% for 10 polynucleotides (SEQ ID NOs: 158 to 167), 100% for 20 polynucleotides (SEQ ID NOs: 148 to 167), 100% for 30 polynucleotides (SEQ ID NOs: 138 to 167), 100% for 50 polynucleotides (SEQ ID NOs: 118 to 167), 100% for 80 polynucleotides (SEQ ID NOs: 88 to 167), 100% for 110 polynucleotides (SEQ ID NOs: 58 to 167), 100% for 150 polynucleotides (SEQ ID NOs: 18 to 167), and 100% for 167 polynucleotides (SEQ ID NOs: 1 to 167).

These results demonstrated that a combination of a plurality of polynucleotides can produce higher liver cancer discriminant performance than that of each polynucleotide alone or a combination of a fewer number of polynucleotides. In this context, the combinations of a plurality of polynucleotides are not limited to the combinations of the polynucleotides added in the order of statistically significant difference as described above, and any combination of a plurality of polynucleotides can be used in the detection of liver cancer.

From these results, it can be concluded that all of the polynucleotides consisting of the nucleotide sequences represented by SEQ ID NOs: 1 to 183 serve as excellent markers for the detection of liver cancer.

TABLE 2

| | | | Expression level in |
|---|---|---|---|
| | | P value after | liver cancer patient |
| | | Bonferroni | relative to healthy |
| SEQ ID NO: | Gene name | correction | subject |
| 1 | hsa-miR-1343-3p | 6.65.E−37 | − |
| 2 | hsa-miR-6726-5p | 2.01.E−34 | − |
| 3 | hsa-miR-6515-3p | 4.26.E−28 | + |
| 4 | hsa-miR-4651 | 1.83.E−27 | − |
| 5 | hsa-miR-4257 | 5.63.E−27 | − |
| 6 | hsa-miR-3188 | 1.06.E−25 | + |
| 7 | hsa-miR-6131 | 4.08.E−25 | − |
| 8 | hsa-miR-6766-3p | 1.86.E−24 | + |
| 9 | hsa-miR-7641 | 5.24.E−24 | − |
| 10 | hsa-miR-1249 | 1.67.E−23 | + |
| 11 | hsa-miR-3679-3p | 3.33.E−23 | + |
| 12 | hsa-miR-6787-5p | 5.69.E−23 | − |
| 13 | hsa-miR-4454 | 6.89.E−23 | − |
| 14 | hsa-miR-3135b | 3.83.E−21 | − |
| 15 | hsa-miR-6765-3p | 2.37.E−20 | − |
| 16 | hsa-miR-7975 | 1.57.E−19 | − |
| 17 | hsa-miR-204-3p | 2.58.E−19 | − |
| 18 | hsa-miR-7977 | 5.17.E−18 | − |
| 19 | hsa-miR-7110-5p | 1.34.E−16 | + |
| 20 | hsa-miR-6717-5p | 1.77.E−16 | − |
| 21 | hsa-miR-6870-5p | 1.86.E−16 | + |
| 22 | hsa-miR-663b | 1.91.E−16 | − |
| 23 | hsa-miR-6875-5p | 1.98.E−16 | + |
| 24 | hsa-miR-8072 | 2.20.E−16 | + |
| 25 | hsa-miR-6816-5p | 4.02.E−16 | + |
| 26 | hsa-miR-4281 | 1.18.E−15 | − |
| 27 | hsa-miR-6729-5p | 1.90.E−15 | + |
| 28 | hsa-miR-8069 | 4.12.E−15 | + |
| 29 | hsa-miR-4706 | 9.80.E−15 | − |
| 30 | hsa-miR-7108-5p | 1.34.E−14 | + |
| 31 | hsa-miR-4433b-3p | 1.44.E−14 | + |
| 32 | hsa-miR-6893-5p | 2.25.E−14 | − |
| 33 | hsa-miR-6857-5p | 3.37.E−14 | + |
| 34 | hsa-miR-1227-5p | 5.86.E−14 | + |
| 35 | hsa-miR-6741-5p | 1.52.E−13 | − |
| 36 | hsa-miR-451a | 1.99.E−13 | − |
| 37 | hsa-miR-8063 | 2.08.E−13 | − |
| 38 | hsa-miR-3622a-5p | 2.29.E−13 | − |
| 39 | hsa-miR-615-5p | 2.47.E−13 | − |
| 40 | hsa-miR-128-1-5p | 6.21.E−13 | + |
| 41 | hsa-miR-6825-5p | 1.19.E−12 | + |
| 42 | hsa-miR-1260b | 2.03.E−12 | − |
| 43 | hsa-miR-4433-3p | 2.67.E−12 | + |
| 44 | hsa-miR-4665-5p | 3.11.E−12 | − |
| 45 | hsa-miR-7845-5p | 3.97.E−12 | + |
| 46 | hsa-miR-1908-5p | 4.05.E−12 | + |
| 47 | hsa-miR-6840-3p | 5.71.E−12 | − |
| 48 | hsa-miR-6765-5p | 5.84.E−12 | + |
| 49 | hsa-miR-296-5p | 6.23.E−12 | + |
| 50 | hsa-miR-3675-3p | 1.58.E−11 | + |
| 51 | hsa-miR-6781-5p | 5.32.E−11 | + |
| 52 | hsa-miR-423-5p | 5.46.E−11 | − |
| 53 | hsa-miR-3663-3p | 5.53.E−11 | − |
| 54 | hsa-miR-6784-5p | 5.78.E−11 | + |
| 55 | hsa-miR-6749-5p | 7.92.E−11 | − |
| 56 | hsa-miR-1231 | 1.43.E−10 | + |
| 57 | hsa-miR-4746-3p | 1.47.E−10 | + |
| 58 | hsa-miR-6780b-5p | 1.80.E−10 | + |
| 59 | hsa-miR-4758-5p | 1.80.E−10 | − |
| 60 | hsa-miR-3679-5p | 2.45.E−10 | + |
| 61 | hsa-miR-3184-5p | 3.79.E−10 | + |
| 62 | hsa-miR-6125 | 4.04.E−10 | + |
| 63 | hsa-miR-6721-5p | 9.40.E−10 | + |
| 64 | hsa-miR-6791-5p | 1.05.E−09 | + |
| 65 | hsa-miR-3185 | 1.24.E−09 | + |
| 66 | hsa-miR-1260a | 1.37.E−09 | − |
| 67 | hsa-miR-3197 | 1.86.E−09 | + |
| 68 | hsa-miR-6845-5p | 2.23.E−09 | + |
| 69 | hsa-miR-6887-5p | 2.95.E−09 | − |
| 70 | hsa-miR-6738-5p | 5.06.E−09 | − |
| 71 | hsa-miR-6872-3p | 5.23.E−09 | − |
| 72 | hsa-miR-4497 | 5.30.E−09 | − |
| 73 | hsa-miR-1229-5p | 6.30.E−09 | + |
| 74 | hsa-miR-6820-5p | 6.66.E−09 | − |
| 75 | hsa-miR-6777-5p | 7.32.E−09 | − |
| 76 | hsa-miR-3917 | 7.71.E−09 | − |
| 77 | hsa-miR-5787 | 7.78.E−09 | + |
| 78 | hsa-miR-4286 | 1.22.E−08 | − |
| 79 | hsa-miR-6877-5p | 1.34.E−08 | − |
| 80 | hsa-miR-1225-3p | 1.56.E−08 | + |
| 81 | hsa-miR-6088 | 1.57.E−08 | − |
| 82 | hsa-miR-6800-5p | 1.94.E−08 | + |
| 83 | hsa-miR-1246 | 3.37.E−08 | − |
| 84 | hsa-miR-4467 | 4.44.E−08 | + |
| 85 | hsa-miR-4419b | 5.34.E−08 | − |
| 86 | hsa-miR-1914-3p | 6.12.E−08 | − |
| 87 | hsa-miR-4632-5p | 7.12.E−08 | + |
| 88 | hsa-miR-1915-5p | 7.21.E−08 | − |
| 89 | hsa-miR-3940-5p | 7.68.E−08 | + |
| 90 | hsa-miR-1185-2-3p | 8.95.E−08 | + |
| 91 | hsa-miR-6746-5p | 1.20.E−07 | − |
| 92 | hsa-miR-5001-5p | 1.89.E−07 | − |
| 93 | hsa-miR-1228-5p | 2.11.E−07 | + |
| 94 | hsa-miR-5572 | 2.20.E−07 | + |
| 95 | hsa-miR-4327 | 2.34.E−07 | + |
| 96 | hsa-miR-4638-5p | 2.46.E−07 | − |
| 97 | hsa-miR-6799-5p | 3.24.E−07 | + |
| 98 | hsa-miR-6861-5p | 5.31.E−07 | − |
| 99 | hsa-miR-6727-5p | 5.46.E−07 | − |
| 100 | hsa-miR-4513 | 7.37.E−07 | − |
| 101 | hsa-miR-6805-3p | 1.20.E−06 | + |
| 102 | hsa-miR-6808-5p | 1.48.E−06 | + |
| 103 | hsa-miR-4449 | 1.92.E−06 | + |
| 104 | hsa-miR-1199-5p | 1.96.E−06 | − |
| 105 | hsa-miR-1275 | 2.60.E−06 | + |
| 106 | hsa-miR-4792 | 3.93.E−06 | + |
| 107 | hsa-miR-4443 | 4.56.E−06 | + |
| 108 | hsa-miR-6891-5p | 4.68.E−06 | + |
| 109 | hsa-miR-6826-5p | 5.09.E−06 | − |
| 110 | hsa-miR-6807-5p | 5.61.E−06 | + |
| 111 | hsa-miR-7150 | 5.87.E−06 | + |
| 112 | hsa-miR-4534 | 6.23.E−06 | + |
| 113 | hsa-miR-4476 | 6.58.E−06 | − |
| 114 | hsa-miR-4649-5p | 6.78.E−06 | − |
| 115 | hsa-miR-4525 | 6.95.E−06 | − |
| 116 | hsa-miR-1915-3p | 7.86.E−06 | + |
| 117 | hsa-miR-4516 | 9.89.E−06 | − |
| 118 | hsa-miR-4417 | 1.02.E−05 | + |
| 119 | hsa-miR-642b-3p | 1.44.E−05 | − |
| 120 | hsa-miR-3141 | 1.52.E−05 | + |
| 121 | hsa-miR-5100 | 1.70.E−05 | − |
| 122 | hsa-miR-6848-5p | 2.10.E−05 | + |
| 123 | hsa-miR-4739 | 2.86.E−05 | + |
| 124 | hsa-miR-4459 | 3.57.E−05 | + |
| 125 | hsa-miR-1237-5p | 3.74.E−05 | + |
| 126 | hsa-miR-296-3p | 4.27.E−05 | − |
| 127 | hsa-miR-4665-3p | 4.37.E−05 | + |
| 128 | hsa-miR-6786-5p | 6.36.E−05 | + |
| 129 | hsa-miR-4258 | 7.87.E−05 | − |
| 130 | hsa-miR-6510-5p | 8.68.E−05 | + |
| 131 | hsa-miR-1343-5p | 8.90.E−05 | + |
| 132 | hsa-miR-1247-3p | 1.33.E−04 | + |
| 133 | hsa-miR-6805-5p | 1.34.E−04 | + |
| 134 | hsa-miR-4492 | 1.62.E−04 | + |
| 135 | hsa-miR-1469 | 1.93.E−04 | + |
| 136 | hsa-miR-1268b | 2.29.E−04 | + |
| 137 | hsa-miR-6858-5p | 2.37.E−04 | + |
| 138 | hsa-miR-3937 | 3.14.E−04 | + |
| 139 | hsa-miR-939-5p | 3.53.E−04 | + |
| 140 | hsa-miR-3656 | 3.91.E−04 | + |
| 141 | hsa-miR-744-5p | 4.32.E−04 | + |
| 142 | hsa-miR-4687-3p | 4.42.E−04 | + |
| 143 | hsa-miR-4763-3p | 4.53.E−04 | + |
| 144 | hsa-miR-3620-5p | 5.43.E−04 | + |
| 145 | hsa-miR-3195 | 6.21.E−04 | + |
| 146 | hsa-miR-6842-5p | 6.44.E−04 | + |
| 147 | hsa-miR-4707-5p | 7.50.E−04 | + |
| 148 | hsa-miR-642a-3p | 8.01.E−04 | + |
| 149 | hsa-miR-7113-3p | 8.81.E−04 | + |
| 150 | hsa-miR-4728-5p | 1.13.E−03 | − |
| 151 | hsa-miR-5195-3p | 1.39.E−03 | − |
| 152 | hsa-miR-1185-1-3p | 1.99.E−03 | + |
| 153 | hsa-miR-6774-5p | 2.01.E−03 | + |
| 154 | hsa-miR-8059 | 2.34.E−03 | − |
| 155 | hsa-miR-3131 | 2.51.E−03 | − |
| 156 | hsa-miR-7847-3p | 2.78.E−03 | − |
| 157 | hsa-miR-4463 | 3.86.E−03 | + |
| 158 | hsa-miR-128-2-5p | 4.01.E−03 | − |
| 159 | hsa-miR-4508 | 4.42.E−03 | + |
| 160 | hsa-miR-6806-5p | 4.85.E−03 | − |
| 161 | hsa-miR-7111-5p | 5.18.E−03 | + |
| 162 | hsa-miR-6782-5p | 5.20.E−03 | + |
| 163 | hsa-miR-4734 | 6.28.E−03 | + |
| 164 | hsa-miR-3162-5p | 8.46.E−03 | + |
| 165 | hsa-miR-887-3p | 8.47.E−03 | + |
| 166 | hsa-miR-6752-5p | 8.98.E−03 | + |
| 167 | hsa-miR-6724-5p | 9.90.E−03 | + |
| 168 | hsa-miR-23b-3p | 4.55.E−23 | − |
| 169 | hsa-miR-23a-3p | 4.37.E−21 | − |
| 170 | hsa-miR-625-3p | 8.87.E−20 | + |
| 171 | hsa-miR-1228-3p | 1.35.E−19 | + |
| 172 | hsa-miR-614 | 2.37.E−18 | − |
| 173 | hsa-miR-1913 | 5.84.E−18 | + |
| 174 | hsa-miR-92a-2-5p | 9.35.E−16 | + |
| 175 | hsa-miR-187-5p | 1.18.E−15 | − |
| 176 | hsa-miR-16-5p | 2.32.E−14 | − |
| 177 | hsa-miR-92b-3p | 2.82.E−12 | − |
| 178 | hsa-miR-150-3p | 8.73.E−11 | − |
| 179 | hsa-miR-564 | 1.08.E−09 | − |
| 180 | hsa-miR-125a-3p | 1.64.E−07 | − |
| 181 | hsa-miR-92b-5p | 5.34.E−07 | + |
| 182 | hsa-miR-92a-3p | 6.00.E−06 | − |
| 183 | hsa-miR-663a | 7.49.E−04 | + |

TABLE 3

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 1 | 95.5 | 97.1 | 95 | 97 | 93.8 | 98 |
| 2 | 97 | 97.1 | 97 | 95.5 | 93.8 | 96 |
| 3 | 91.8 | 82.4 | 95 | 90.9 | 93.8 | 90 |
| 4 | 96.3 | 91.2 | 98 | 95.5 | 87.5 | 98 |
| 5 | 96.3 | 88.2 | 99 | 92.4 | 75 | 98 |
| 6 | 94.8 | 88.2 | 97 | 95.5 | 87.5 | 98 |
| 7 | 92.5 | 73.5 | 99 | 90.9 | 62.5 | 100 |
| 8 | 94.8 | 88.2 | 97 | 92.4 | 81.2 | 96 |
| 9 | 91.8 | 82.4 | 95 | 95.5 | 93.8 | 96 |
| 10 | 94.7 | 94.1 | 94.9 | 92.4 | 93.8 | 92 |
| 11 | 94 | 91.2 | 95 | 86.4 | 75 | 90 |
| 12 | 91.8 | 76.5 | 97 | 93.9 | 93.8 | 94 |
| 13 | 91.8 | 70.6 | 99 | 89.4 | 62.5 | 98 |
| 14 | 97 | 91.2 | 99 | 97 | 93.8 | 98 |
| 15 | 91.8 | 73.5 | 98 | 87.9 | 56.2 | 98 |
| 16 | 90.3 | 64.7 | 99 | 87.9 | 56.2 | 98 |
| 17 | 90.3 | 67.6 | 98 | 81.8 | 56.2 | 90 |
| 18 | 88.1 | 58.8 | 98 | 84.8 | 43.8 | 98 |
| 19 | 88.1 | 76.5 | 92 | 90.9 | 93.8 | 90 |
| 20 | 92.5 | 73.5 | 99 | 86.4 | 50 | 98 |
| 21 | 92.5 | 79.4 | 97 | 92.4 | 68.8 | 100 |
| 22 | 88.8 | 58.8 | 99 | 97 | 87.5 | 100 |
| 23 | 91 | 73.5 | 97 | 90.9 | 93.8 | 90 |
| 24 | 91.8 | 79.4 | 96 | 84.8 | 81.2 | 86 |
| 25 | 89.6 | 82.4 | 92 | 93.9 | 87.5 | 96 |
| 26 | 88.8 | 76.5 | 93 | 84.8 | 50 | 96 |
| 27 | 91.8 | 73.5 | 98 | 89.4 | 62.5 | 98 |
| 28 | 83.6 | 50 | 95 | 86.4 | 56.2 | 96 |
| 29 | 88.8 | 73.5 | 94 | 87.9 | 68.8 | 94 |
| 30 | 85.8 | 64.7 | 93 | 86.4 | 81.2 | 88 |
| 31 | 88.8 | 76.5 | 93 | 83.3 | 81.2 | 84 |
| 32 | 89.6 | 61.8 | 99 | 89.4 | 62.5 | 98 |
| 33 | 89.6 | 79.4 | 93 | 92.4 | 87.5 | 94 |
| 34 | 86.6 | 64.7 | 94 | 84.8 | 68.8 | 90 |
| 35 | 88.1 | 64.7 | 96 | 87.9 | 75 | 92 |
| 36 | 86.6 | 50 | 99 | 80.3 | 31.2 | 96 |
| 37 | 84.3 | 64.7 | 91 | 89.4 | 75 | 94 |
| 38 | 85.8 | 50 | 98 | 86.4 | 43.8 | 100 |
| 39 | 87.3 | 52.9 | 99 | 92.4 | 75 | 98 |
| 40 | 85.1 | 64.7 | 92 | 78.8 | 62.5 | 84 |
| 41 | 94 | 85.3 | 97 | 93.9 | 93.8 | 94 |
| 42 | 85.8 | 52.9 | 97 | 84.8 | 50 | 96 |
| 43 | 82.1 | 64.7 | 88 | 86.4 | 75 | 90 |
| 44 | 82.1 | 50 | 93 | 80.3 | 56.2 | 88 |
| 45 | 88.1 | 70.6 | 94 | 84.8 | 62.5 | 92 |
| 46 | 82.8 | 52.9 | 93 | 86.4 | 62.5 | 94 |
| 47 | 86.6 | 55.9 | 97 | 89.4 | 68.8 | 96 |
| 48 | 88.1 | 67.6 | 95 | 92.4 | 87.5 | 94 |
| 49 | 82.8 | 50 | 94 | 72.7 | 25 | 88 |
| 50 | 94 | 85.3 | 97 | 89.4 | 75 | 94 |
| 51 | 84.3 | 55.9 | 94 | 83.3 | 62.5 | 90 |
| 52 | 83.6 | 41.2 | 98 | 86.4 | 43.8 | 100 |
| 53 | 85.8 | 52.9 | 97 | 84.8 | 43.8 | 98 |
| 54 | 91 | 79.4 | 95 | 87.9 | 75 | 92 |
| 55 | 86.6 | 58.8 | 96 | 90.9 | 68.8 | 98 |
| 56 | 83.6 | 55.9 | 93 | 84.8 | 62.5 | 92 |
| 57 | 86.6 | 67.6 | 93 | 89.4 | 68.8 | 96 |
| 58 | 85.1 | 55.9 | 95 | 92.4 | 68.8 | 100 |
| 59 | 85.1 | 47.1 | 98 | 81.8 | 31.2 | 98 |
| 60 | 82.1 | 50 | 93 | 89.4 | 68.8 | 96 |
| 61 | 86.6 | 67.6 | 93 | 86.4 | 62.5 | 94 |
| 62 | 85.8 | 61.8 | 94 | 87.9 | 62.5 | 96 |
| 63 | 82.1 | 58.8 | 90 | 84.8 | 75 | 88 |
| 64 | 83.6 | 61.8 | 91 | 89.4 | 62.5 | 98 |
| 65 | 85.1 | 64.7 | 92 | 89.4 | 75 | 94 |
| 66 | 85.8 | 52.9 | 97 | 78.8 | 31.2 | 94 |
| 67 | 84.3 | 58.8 | 93 | 83.3 | 50 | 94 |
| 68 | 84.3 | 47.1 | 97 | 90.9 | 68.8 | 98 |
| 69 | 80.6 | 26.5 | 99 | 80.3 | 18.8 | 100 |
| 70 | 86.6 | 55.9 | 97 | 83.3 | 50 | 94 |
| 71 | 83.6 | 38.2 | 99 | 84.8 | 37.5 | 100 |
| 72 | 79.1 | 41.2 | 92 | 74.2 | 31.2 | 88 |
| 73 | 85.1 | 55.9 | 95 | 86.4 | 56.2 | 96 |
| 74 | 85.8 | 47.1 | 99 | 81.8 | 31.2 | 98 |
| 75 | 82.1 | 32.4 | 99 | 83.3 | 31.2 | 100 |
| 76 | 82.1 | 32.4 | 99 | 81.8 | 37.5 | 96 |
| 77 | 81.3 | 32.4 | 98 | 87.9 | 50 | 100 |
| 78 | 82.1 | 38.2 | 97 | 78.8 | 25 | 96 |
| 79 | 79.1 | 41.2 | 92 | 78.8 | 37.5 | 92 |
| 80 | 88.8 | 64.7 | 97 | 95.5 | 81.2 | 100 |
| 81 | 79.1 | 47.1 | 90 | 80.3 | 43.8 | 92 |
| 82 | 84.3 | 52.9 | 95 | 81.8 | 50 | 92 |
| 83 | 82.1 | 41.2 | 96 | 78.8 | 31.2 | 94 |
| 84 | 76.1 | 41.2 | 88 | 84.8 | 50 | 96 |
| 85 | 79.9 | 32.4 | 96 | 78.8 | 18.8 | 98 |
| 86 | 83.6 | 55.9 | 93 | 83.3 | 68.8 | 88 |
| 87 | 86.6 | 50 | 99 | 80.3 | 18.8 | 100 |
| 88 | 82.1 | 41.2 | 96 | 86.4 | 56.2 | 96 |
| 89 | 82.1 | 38.2 | 97 | 80.3 | 37.5 | 94 |
| 90 | 83.6 | 50 | 95 | 80.3 | 43.8 | 92 |
| 91 | 78.4 | 44.1 | 90 | 84.8 | 62.5 | 92 |
| 92 | 88.1 | 64.7 | 96 | 81.8 | 37.5 | 96 |
| 93 | 82.8 | 50 | 94 | 84.8 | 56.2 | 94 |
| 94 | 88.1 | 67.6 | 95 | 84.8 | 56.2 | 94 |
| 95 | 82.8 | 50 | 94 | 77.3 | 31.2 | 92 |
| 96 | 82.1 | 35.3 | 98 | 80.3 | 18.8 | 100 |
| 97 | 84.3 | 50 | 96 | 77.3 | 18.8 | 96 |
| 98 | 79.1 | 41.2 | 92 | 78.8 | 37.5 | 92 |
| 99 | 83.6 | 55.9 | 93 | 90.9 | 68.8 | 98 |
| 100 | 76.1 | 14.7 | 97 | 81.8 | 31.2 | 98 |
| 101 | 78.4 | 44.1 | 90 | 78.8 | 31.2 | 94 |
| 102 | 79.9 | 32.4 | 96 | 77.3 | 31.2 | 92 |
| 103 | 81.3 | 41.2 | 95 | 75.8 | 12.5 | 96 |
| 104 | 82.1 | 44.1 | 95 | 84.8 | 50 | 96 |
| 105 | 77.6 | 32.4 | 93 | 77.3 | 25 | 94 |
| 106 | 84.3 | 50 | 96 | 86.4 | 50 | 98 |
| 107 | 85.1 | 50 | 97 | 86.4 | 50 | 98 |
| 108 | 82.1 | 47.1 | 94 | 87.9 | 50 | 100 |
| 109 | 79.9 | 26.5 | 98 | 77.3 | 6.2 | 100 |
| 110 | 79.1 | 35.3 | 94 | 78.8 | 31.2 | 94 |
| 111 | 84.3 | 44.1 | 98 | 83.3 | 31.2 | 100 |
| 112 | 80.6 | 35.3 | 96 | 75.8 | 12.5 | 96 |
| 113 | 78.4 | 20.6 | 98 | 81.8 | 25 | 100 |
| 114 | 83.6 | 47.1 | 96 | 86.4 | 56.2 | 96 |
| 115 | 79.1 | 38.2 | 93 | 80.3 | 25 | 98 |
| 116 | 82.1 | 44.1 | 95 | 78.8 | 31.2 | 94 |
| 117 | 84.3 | 50 | 96 | 87.9 | 62.5 | 96 |
| 118 | 82.8 | 41.2 | 97 | 83.3 | 43.8 | 96 |
| 119 | 82.8 | 41.2 | 97 | 83.3 | 31.2 | 100 |
| 120 | 79.1 | 23.5 | 98 | 75.8 | 18.8 | 94 |
| 121 | 82 | 39.4 | 96 | 74.2 | 12.5 | 94 |
| 122 | 77.6 | 32.4 | 93 | 74.2 | 31.2 | 88 |
| 123 | 82.1 | 38.2 | 97 | 80.3 | 31.2 | 96 |
| 124 | 80.6 | 32.4 | 97 | 83.3 | 37.5 | 98 |
| 125 | 76.9 | 20.6 | 96 | 78.8 | 18.8 | 98 |
| 126 | 77.6 | 20.6 | 97 | 78.8 | 25 | 96 |
| 127 | 82.8 | 35.3 | 99 | 83.3 | 37.5 | 98 |
| 128 | 79.9 | 32.4 | 96 | 71.2 | 37.5 | 82 |
| 129 | 82.8 | 38.2 | 98 | 81.8 | 31.2 | 98 |
| 130 | 82.1 | 32.4 | 99 | 83.3 | 31.2 | 100 |
| 131 | 83.6 | 44.1 | 97 | 83.3 | 37.5 | 98 |
| 132 | 85.8 | 44.1 | 100 | 84.8 | 43.8 | 98 |
| 133 | 78.4 | 26.5 | 96 | 81.8 | 43.8 | 94 |
| 134 | 79.9 | 35.3 | 95 | 77.3 | 31.2 | 92 |
| 135 | 78.4 | 14.7 | 100 | 72.7 | 0 | 96 |
| 136 | 69.4 | 8.8 | 90 | 68.2 | 6.2 | 88 |
| 137 | 77.6 | 14.7 | 99 | 72.7 | 0 | 96 |
| 138 | 77.6 | 29.4 | 94 | 78.8 | 25 | 96 |
| 139 | 82.1 | 32.4 | 99 | 80.3 | 31.2 | 96 |
| 140 | 75.4 | 20.6 | 94 | 77.3 | 12.5 | 98 |
| 141 | 76.9 | 20.6 | 96 | 83.3 | 31.2 | 100 |
| 142 | 74.6 | 20.6 | 93 | 81.8 | 31.2 | 98 |
| 143 | 77.6 | 23.5 | 96 | 80.3 | 25 | 98 |
| 144 | 78.4 | 29.4 | 95 | 77.3 | 31.2 | 92 |
| 145 | 76.9 | 23.5 | 95 | 74.2 | 12.5 | 94 |
| 146 | 81.3 | 29.4 | 99 | 86.4 | 50 | 98 |
| 147 | 73.1 | 8.8 | 95 | 72.7 | 0 | 96 |
| 148 | 79.9 | 26.5 | 98 | 77.3 | 12.5 | 98 |
| 149 | 78.4 | 17.6 | 99 | 75.8 | 12.5 | 96 |
| 150 | 74.6 | 23.5 | 92 | 74.2 | 18.8 | 92 |
| 151 | 73.9 | 8.8 | 96 | 75.8 | 6.2 | 98 |
| 152 | 79.9 | 29.4 | 97 | 74.2 | 12.5 | 94 |
| 153 | 73.9 | 11.8 | 95 | 72.7 | 0 | 96 |
| 154 | 75.4 | 14.7 | 96 | 75.8 | 12.5 | 96 |
| 155 | 79.1 | 23.5 | 98 | 77.3 | 12.5 | 98 |
| 156 | 75.4 | 5.9 | 99 | 77.3 | 6.2 | 100 |
| 157 | 76.1 | 20.6 | 95 | 77.3 | 18.8 | 96 |
| 158 | 80.6 | 29.4 | 98 | 78.8 | 12.5 | 100 |
| 159 | 73.9 | 11.8 | 95 | 75.8 | 31.2 | 90 |
| 160 | 76.1 | 5.9 | 100 | 75.8 | 0 | 100 |
| 161 | 79.1 | 23.5 | 98 | 78.8 | 12.5 | 100 |
| 162 | 79.1 | 17.6 | 100 | 77.3 | 18.8 | 96 |
| 163 | 72.4 | 8.8 | 94 | 78.8 | 31.2 | 94 |
| 164 | 75.4 | 14.7 | 96 | 72.7 | 6.2 | 94 |
| 165 | 70.9 | 2.9 | 94 | 68.2 | 0 | 90 |
| 166 | 76.1 | 14.7 | 97 | 72.7 | 6.2 | 94 |
| 167 | 76.9 | 23.5 | 95 | 74.2 | 12.5 | 94 |
| 168 | 88.8 | 64.7 | 97 | 81.8 | 43.8 | 94 |
| 169 | 87.3 | 58.8 | 97 | 80.3 | 37.5 | 94 |
| 170 | 91 | 76.5 | 96 | 90.9 | 87.5 | 92 |
| 171 | 91.8 | 85.3 | 94 | 89.4 | 87.5 | 90 |
| 172 | 87.3 | 79.4 | 90 | 89.4 | 75 | 94 |
| 173 | 88.8 | 79.4 | 92 | 87.7 | 68.8 | 93.9 |
| 174 | 89.6 | 76.5 | 94 | 84.8 | 62.5 | 92 |
| 175 | 90.3 | 70.6 | 97 | 93.9 | 81.2 | 98 |
| 176 | 85.8 | 55.9 | 96 | 83.3 | 43.8 | 96 |
| 177 | 86.6 | 52.9 | 98 | 83.3 | 37.5 | 98 |
| 178 | 83.6 | 38.2 | 99 | 81.8 | 50 | 92 |
| 179 | 82.8 | 41.2 | 97 | 84.8 | 43.8 | 98 |
| 180 | 84.3 | 41.2 | 99 | 87.9 | 50 | 100 |
| 181 | 82.1 | 32.4 | 99 | 75.8 | 0 | 100 |
| 182 | 82.1 | 32.4 | 99 | 78.8 | 18.8 | 98 |
| 183 | 76.9 | 14.7 | 98 | 77.3 | 6.2 | 100 |

TABLE 4

| | Discriminant | Constant |
|---|---|---|
| SEQ ID NO: | coefficient | term |
| 1 | 2.471 | 17.511 |
| 2 | 3.389 | 32.503 |
| 3 | 4.221 | 29.467 |
| 4 | 5.669 | 61.422 |
| 5 | 2.340 | 14.902 |
| 6 | 3.403 | 21.347 |
| 7 | 1.666 | 16.714 |
| 8 | 3.780 | 23.286 |
| 9 | 1.162 | 7.705 |
| 10 | 3.871 | 23.895 |
| 11 | 3.327 | 20.777 |
| 12 | 3.912 | 32.887 |
| 13 | 1.850 | 20.690 |
| 14 | 2.777 | 21.161 |
| 15 | 1.469 | 12.157 |
| 16 | 1.640 | 15.602 |
| 17 | 1.594 | 20.057 |
| 18 | 1.741 | 16.417 |
| 19 | 1.740 | 14.012 |
| 20 | 2.167 | 12.838 |
| 21 | 3.215 | 24.454 |
| 22 | 2.867 | 24.605 |
| 23 | 3.272 | 30.031 |
| 24 | 5.400 | 67.222 |
| 25 | 4.398 | 44.949 |
| 26 | 4.110 | 47.240 |
| 27 | 8.336 | 105.482 |
| 28 | 6.984 | 90.484 |
| 29 | 3.912 | 29.950 |
| 30 | 4.452 | 41.269 |
| 31 | 3.737 | 30.649 |
| 32 | 1.541 | 12.525 |
| 33 | 1.731 | 9.319 |
| 34 | 6.775 | 65.355 |
| 35 | 4.246 | 28.999 |
| 36 | 0.707 | 5.520 |
| 37 | 2.475 | 20.255 |
| 38 | 1.782 | 9.870 |
| 39 | 1.749 | 10.960 |
| 40 | 2.724 | 20.676 |
| 41 | 1.635 | 11.008 |
| 42 | 2.017 | 16.782 |
| 43 | 3.750 | 27.935 |
| 44 | 3.268 | 30.852 |
| 45 | 3.074 | 20.807 |
| 46 | 4.135 | 48.094 |
| 47 | 2.722 | 23.696 |
| 48 | 4.645 | 49.638 |
| 49 | 4.364 | 34.762 |
| 50 | 2.395 | 13.357 |
| 51 | 5.700 | 60.009 |
| 52 | 1.785 | 12.550 |
| 53 | 3.691 | 44.502 |
| 54 | 3.410 | 43.229 |
| 55 | 4.359 | 43.584 |
| 56 | 3.783 | 25.006 |
| 57 | 2.734 | 18.058 |
| 58 | 2.978 | 26.851 |
| 59 | 6.061 | 51.915 |
| 60 | 2.729 | 18.883 |
| 61 | 2.150 | 17.585 |
| 62 | 5.256 | 63.263 |
| 63 | 3.936 | 30.117 |
| 64 | 4.508 | 41.792 |
| 65 | 2.386 | 16.961 |
| 66 | 1.810 | 12.154 |
| 67 | 2.969 | 28.301 |
| 68 | 3.512 | 34.056 |
| 69 | 1.951 | 12.101 |
| 70 | 3.135 | 22.180 |
| 71 | 1.606 | 9.267 |
| 72 | 2.696 | 34.139 |
| 73 | 4.474 | 34.903 |
| 74 | 2.012 | 14.274 |
| 75 | 1.959 | 12.395 |
| 76 | 2.215 | 12.602 |
| 77 | 5.057 | 66.741 |
| 78 | 1.620 | 11.678 |
| 79 | 4.288 | 30.633 |
| 80 | 2.430 | 13.696 |
| 81 | 3.351 | 33.938 |
| 82 | 3.921 | 34.024 |
| 83 | 1.278 | 9.389 |
| 84 | 2.183 | 21.651 |
| 85 | 1.944 | 11.599 |
| 86 | 4.824 | 36.279 |
| 87 | 3.858 | 31.074 |
| 88 | 1.277 | 7.779 |
| 89 | 4.555 | 56.233 |
| 90 | 1.520 | 8.345 |
| 91 | 3.667 | 23.791 |
| 92 | 3.455 | 26.548 |
| 93 | 3.821 | 45.609 |
| 94 | 1.784 | 12.053 |
| 95 | 4.842 | 42.664 |
| 96 | 1.392 | 8.122 |
| 97 | 3.251 | 27.595 |
| 98 | 4.026 | 29.199 |
| 99 | 5.471 | 69.803 |
| 100 | 2.281 | 13.200 |
| 101 | 2.499 | 18.849 |
| 102 | 5.118 | 35.429 |
| 103 | 3.691 | 24.076 |
| 104 | 2.471 | 16.246 |
| 105 | 2.973 | 21.963 |
| 106 | 1.588 | 10.669 |
| 107 | 2.017 | 13.094 |
| 108 | 4.206 | 32.002 |
| 109 | 1.659 | 9.895 |
| 110 | 2.739 | 16.192 |
| 111 | 3.174 | 24.976 |
| 112 | 2.780 | 19.682 |
| 113 | 1.225 | 8.488 |
| 114 | 2.404 | 24.762 |
| 115 | 2.895 | 19.963 |
| 116 | 4.205 | 46.806 |
| 117 | 4.490 | 59.177 |
| 118 | 5.016 | 41.382 |
| 119 | 2.142 | 20.182 |
| 120 | 4.030 | 28.787 |
| 121 | 2.093 | 21.502 |
| 122 | 4.832 | 36.040 |
| 123 | 3.672 | 42.382 |
| 124 | 3.305 | 27.456 |
| 125 | 4.919 | 62.904 |
| 126 | 1.924 | 11.325 |
| 127 | 2.696 | 15.869 |
| 128 | 7.275 | 92.098 |
| 129 | 1.903 | 17.010 |
| 130 | 1.935 | 12.644 |
| 131 | 3.379 | 35.351 |
| 132 | 2.384 | 15.077 |
| 133 | 6.549 | 74.981 |
| 134 | 5.238 | 55.302 |
| 135 | 2.785 | 28.718 |
| 136 | 3.118 | 31.040 |
| 137 | 3.097 | 23.331 |
| 138 | 4.424 | 38.383 |
| 139 | 1.611 | 12.320 |
| 140 | 4.840 | 56.003 |
| 141 | 2.484 | 17.251 |
| 142 | 3.851 | 37.749 |
| 143 | 3.720 | 31.374 |
| 144 | 3.991 | 31.836 |
| 145 | 4.065 | 33.772 |
| 146 | 2.441 | 14.617 |
| 147 | 3.795 | 27.973 |
| 148 | 2.362 | 18.895 |
| 149 | 2.354 | 13.716 |
| 150 | 5.065 | 35.714 |
| 151 | 2.922 | 20.137 |
| 152 | 1.539 | 9.313 |
| 153 | 4.631 | 31.436 |
| 154 | 3.326 | 25.477 |
| 155 | 2.223 | 15.649 |
| 156 | 2.416 | 15.308 |
| 157 | 4.655 | 51.632 |
| 158 | 2.552 | 27.736 |
| 159 | 6.563 | 85.503 |
| 160 | 2.281 | 14.772 |
| 161 | 5.241 | 39.899 |
| 162 | 2.291 | 14.195 |
| 163 | 6.256 | 74.602 |
| 164 | 2.920 | 22.423 |
| 165 | 2.285 | 16.474 |
| 166 | 3.720 | 42.108 |
| 167 | 4.806 | 47.920 |
| 168 | 1.156 | 5.990 |
| 169 | 1.212 | 6.218 |
| 170 | 3.292 | 19.092 |
| 171 | 4.244 | 27.332 |
| 172 | 1.867 | 12.024 |
| 173 | 3.494 | 22.197 |
| 174 | 2.062 | 19.948 |
| 175 | 1.942 | 18.936 |
| 176 | 0.886 | 4.794 |
| 177 | 1.182 | 6.543 |
| 178 | 1.678 | 10.850 |
| 179 | 1.358 | 7.646 |
| 180 | 1.032 | 6.311 |
| 181 | 2.498 | 20.322 |
| 182 | 1.203 | 7.922 |
| 183 | 2.779 | 28.552 |

TABLE 5-1
Training cohort

| | Cancer | AFP | CEA | CA19-9 | PIVKA-II |
|---|---|---|---|---|---|
| Sample name | stage | (ng/mL) | (ng/mL) | (U/mL) | (mAU/mL) |
| HC03 | I | 13.2 | 3.1 | — | 99 |
| HC04 | I | 37210 | 1 | — | 13550 |
| HC05 | IV | 3 | — | — | 18 |
| HC06 | I | 26.1 | 5.7 | — | 136 |
| HC07 | III | 3.2 | 3.4 | — | 2452 |
| HC09 | II | 34.7 | 5 | 26.2 | 1932 |
| HC10 | I | 74 | 2.6 | — | 10 |
| HC12 | I | 3.4 | — | — | 39 |
| HC13 | III | — | 0.6 | 5.1 | — |
| HC15 | II | — | 1.9 | 0.1 | — |
| HC17 | II | 2.3 | — | — | 556 |
| HC18 | IV | 36145 | — | — | 167 |
| HC19 | I | 8.5 | 3.7 | — | 13 |
| HC20 | I | 4.6 | 3.2 | 6.4 | 344 |
| HC23 | III | 151.3 | 1.9 | — | 29521 |
| HC24 | III | 103299 | 1.9 | — | 55837 |
| HC25 | I | 179.7 | 12.1 | — | 220 |
| HC26 | I | 25.3 | 1.4 | — | 36 |
| HC27 | I | 8.5 | 4.7 | — | 28 |
| HC29 | I | 29.2 | — | — | 979 |
| HC30 | IIIB | 77.4 | — | — | 176940 |
| HC31 | II | 7 | — | — | 34 |
| HC32 | III | 2.2 | 1.8 | — | 40 |
| HC34 | II | 6.9 | — | — | 688 |
| HC36 | II | 25.3 | 1.9 | — | 3481 |
| HC38 | I | 5.4 | 4.8 | — | 92 |
| HC40 | IIIB | 5.7 | — | — | 95 |
| HC41 | II | 93.7 | 5.8 | 104.9 | 26 |
| HC42 | I | 1.9 | 6.5 | — | 25 |
| HC45 | II | 10.3 | — | — | 51 |
| HC47 | IIIC | 235.5 | — | — | 3601 |
| HC48 | I | 107.9 | — | — | 52 |
| HC49 | I | 4.5 | 4.3 | 26.7 | 22 |
| HC50 | II | 133338 | 2.9 | — | 829 |
| | Sensitivity | 56.3% | 18.2% | 16.7% | 65.6% |

TABLE 5-2
Validation cohort

| | Cancer | AFP | CEA | CA19-9 | PIVKA-II |
|---|---|---|---|---|---|
| Sample name | stage | (ng/mL) | (ng/mL) | (U/mL) | (mAU/mL) |
| HC01 | II | 10.8 | 2.8 | — | 678 |
| HC02 | I | 3.8 | 1.4 | 11.4 | 26 |
| HC08 | I | 13 | 3 | — | 245 |
| HC11 | I | 17.2 | 3.4 | — | 15 |
| HC14 | I | 1.8 | 5.7 | — | 18 |
| HC16 | I | 6 | — | — | 21 |
| HC21 | II | 5.3 | 5.3 | 14.8 | 22 |
| HC22 | I | 1.7 | — | — | 76 |
| HC28 | I | — | 4.4 | 11 | — |
| HC33 | III | 40 | 1.1 | — | 25 |
| HC35 | II | 4.2 | 5.2 | — | 20 |
| HC37 | III | 59992 | — | — | 14358 |
| HC39 | II | 555 | — | — | 194 |
| HC43 | I | 18 | — | — | 32 |
| HC44 | I | 7.5 | 1 | 32.7 | 462 |
| HC46 | II | 1075 | — | — | 46 |
| | Sensitivity | 53.3% | 30.0% | 0.0% | 46.7% |

The reference values of AFP, CEA, CA19-9, and PIVKA-II were 10 ng/mL, 5 ng/mL, 37 U/mL, and 40 mAU/mL, respectively. Each sample that exhibited a measurement value equal to or higher than the reference values was determined to be positive, and the sensitivity of each tumor marker was calculated.

TABLE 6

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 1_2 | 99.3 | 100 | 99 | 100 | 100 | 100 |
| 1_3 | 100 | 100 | 100 | 98.5 | 100 | 98 |
| 1_4 | 99.3 | 100 | 99 | 100 | 100 | 100 |
| 1_5 | 97.8 | 97.1 | 98 | 97 | 93.8 | 98 |
| 1_6 | 99.3 | 97.1 | 100 | 97 | 93.8 | 98 |
| 1_7 | 96.3 | 91.2 | 98 | 97 | 87.5 | 100 |
| 1_8 | 100 | 100 | 100 | 97 | 93.8 | 98 |
| 1_9 | 97.8 | 97.1 | 98 | 97 | 100 | 96 |
| 1_10 | 99.2 | 100 | 99 | 100 | 100 | 100 |
| 1_11 | 98.5 | 100 | 98 | 97 | 93.8 | 98 |
| 1_12 | 97.8 | 100 | 97 | 97 | 93.8 | 98 |
| 1_13 | 98.5 | 97.1 | 99 | 98.5 | 93.8 | 100 |
| 1_14 | 99.3 | 100 | 99 | 98.5 | 93.8 | 100 |
| 1_15 | 97.8 | 94.1 | 99 | 98.5 | 93.8 | 100 |
| 1_16 | 97.8 | 94.1 | 99 | 97 | 93.8 | 98 |
| 1_17 | 99.3 | 100 | 99 | 97 | 100 | 96 |
| 1_18 | 97.8 | 97.1 | 98 | 97 | 93.8 | 98 |
| 1_19 | 96.3 | 94.1 | 97 | 97 | 93.8 | 98 |
| 1_20 | 96.3 | 94.1 | 97 | 97 | 93.8 | 98 |
| 1_21 | 95.5 | 94.1 | 96 | 97 | 93.8 | 98 |
| 1_22 | 97 | 94.1 | 98 | 97 | 93.8 | 98 |
| 1_23 | 97.8 | 97.1 | 98 | 98.5 | 100 | 98 |
| 1_24 | 98.5 | 100 | 98 | 97 | 93.8 | 98 |
| 1_25 | 97.8 | 97.1 | 98 | 97 | 93.8 | 98 |
| 1_26 | 97 | 97.1 | 97 | 97 | 93.8 | 98 |
| 1_27 | 97.8 | 97.1 | 98 | 95.5 | 93.8 | 96 |
| 1_28 | 97.8 | 100 | 97 | 97 | 93.8 | 98 |
| 1_29 | 97.8 | 100 | 97 | 97 | 100 | 96 |
| 1_30 | 98.5 | 97.1 | 99 | 93.9 | 87.5 | 96 |
| 1_31 | 95.5 | 91.2 | 97 | 97 | 93.8 | 98 |
| 1_32 | 99.3 | 100 | 99 | 97 | 100 | 96 |
| 1_33 | 96.3 | 94.1 | 97 | 97 | 93.8 | 98 |
| 1_34 | 96.3 | 97.1 | 96 | 97 | 93.8 | 98 |
| 1_35 | 97.8 | 97.1 | 98 | 97 | 93.8 | 98 |
| 1_36 | 99.3 | 100 | 99 | 98.5 | 93.8 | 100 |
| 1_37 | 97 | 94.1 | 98 | 97 | 93.8 | 98 |
| 1_38 | 98.5 | 97.1 | 99 | 97 | 93.8 | 98 |
| 1_39 | 99.3 | 97.1 | 100 | 100 | 100 | 100 |
| 1_40 | 97 | 97.1 | 97 | 97 | 93.8 | 98 |
| 1_41 | 95.5 | 94.1 | 96 | 97 | 93.8 | 98 |
| 1_42 | 96.3 | 97.1 | 96 | 97 | 93.8 | 98 |
| 1_43 | 96.3 | 94.1 | 97 | 97 | 93.8 | 98 |
| 1_44 | 98.5 | 100 | 98 | 97 | 100 | 96 |
| 1_45 | 97.8 | 97.1 | 98 | 97 | 93.8 | 98 |
| 1_46 | 97 | 97.1 | 97 | 97 | 93.8 | 98 |
| 1_47 | 97 | 94.1 | 98 | 97 | 93.8 | 98 |
| 1_48 | 97.8 | 97.1 | 98 | 97 | 93.8 | 98 |
| 1_49 | 98.5 | 97.1 | 99 | 98.5 | 93.8 | 100 |
| 1_50 | 96.3 | 97.1 | 96 | 97 | 93.8 | 98 |
| 1_51 | 97 | 97.1 | 97 | 97 | 93.8 | 98 |
| 1_52 | 99.3 | 100 | 99 | 98.5 | 100 | 98 |
| 1_53 | 95.5 | 97.1 | 95 | 97 | 93.8 | 98 |
| 1_54 | 96.3 | 94.1 | 97 | 97 | 93.8 | 98 |
| 1_55 | 97.8 | 97.1 | 98 | 97 | 93.8 | 98 |
| 1_56 | 96.3 | 97.1 | 96 | 97 | 93.8 | 98 |
| 1_57 | 97 | 94.1 | 98 | 97 | 93.8 | 98 |
| 1_58 | 96.3 | 94.1 | 97 | 97 | 93.8 | 98 |
| 1_59 | 97 | 94.1 | 98 | 98.5 | 93.8 | 100 |
| 1_60 | 97 | 97.1 | 97 | 97 | 93.8 | 98 |
| 1_61 | 95.5 | 94.1 | 96 | 97 | 93.8 | 98 |
| 1_62 | 97 | 94.1 | 98 | 97 | 93.8 | 98 |
| 1_63 | 96.3 | 94.1 | 97 | 97 | 93.8 | 98 |
| 1_64 | 97.8 | 94.1 | 99 | 97 | 93.8 | 98 |
| 1_65 | 97.8 | 97.1 | 98 | 97 | 93.8 | 98 |
| 1_66 | 97.8 | 97.1 | 98 | 97 | 93.8 | 98 |
| 1_67 | 97 | 94.1 | 98 | 97 | 93.8 | 98 |
| 1_68 | 98.5 | 100 | 98 | 98.5 | 100 | 98 |
| 1_69 | 96.3 | 94.1 | 97 | 97 | 93.8 | 98 |
| 1_70 | 97.8 | 94.1 | 99 | 97 | 93.8 | 98 |
| 1_71 | 97.8 | 97.1 | 98 | 97 | 93.8 | 98 |
| 1_72 | 97.8 | 100 | 97 | 95.5 | 100 | 94 |
| 1_73 | 95.5 | 94.1 | 96 | 97 | 93.8 | 98 |
| 1_74 | 99.3 | 100 | 99 | 98.5 | 100 | 98 |
| 1_75 | 98.5 | 100 | 98 | 97 | 93.8 | 98 |
| 1_76 | 96.3 | 97.1 | 96 | 97 | 93.8 | 98 |
| 1_77 | 97.8 | 97.1 | 98 | 97 | 93.8 | 98 |
| 1_78 | 97 | 97.1 | 97 | 97 | 93.8 | 98 |
| 1_79 | 97 | 97.1 | 97 | 97 | 93.8 | 98 |
| 1_80 | 97 | 94.1 | 98 | 95.5 | 87.5 | 98 |
| 1_81 | 98.5 | 97.1 | 99 | 95.5 | 93.8 | 96 |
| 1_82 | 95.5 | 97.1 | 95 | 97 | 93.8 | 98 |
| 1_83 | 96.3 | 91.2 | 98 | 97 | 93.8 | 98 |
| 1_84 | 97.8 | 97.1 | 98 | 97 | 93.8 | 98 |
| 1_85 | 96.3 | 97.1 | 96 | 97 | 93.8 | 98 |
| 1_86 | 97 | 97.1 | 97 | 95.5 | 93.8 | 96 |
| 1_87 | 97 | 97.1 | 97 | 97 | 93.8 | 98 |
| 1_88 | 96.3 | 94.1 | 97 | 98.5 | 100 | 98 |
| 1_89 | 95.5 | 97.1 | 95 | 95.5 | 93.8 | 96 |
| 1_90 | 98.5 | 100 | 98 | 95.5 | 93.8 | 96 |
| 1_91 | 96.3 | 97.1 | 96 | 97 | 93.8 | 98 |
| 1_92 | 97 | 97.1 | 97 | 97 | 93.8 | 98 |
| 1_93 | 97 | 100 | 96 | 95.5 | 93.8 | 96 |
| 1_94 | 96.3 | 94.1 | 97 | 97 | 93.8 | 98 |
| 1_95 | 97 | 97.1 | 97 | 97 | 93.8 | 98 |
| 1_96 | 99.3 | 100 | 99 | 97 | 100 | 96 |
| 1_97 | 97 | 100 | 96 | 95.5 | 93.8 | 96 |
| 1_98 | 97 | 100 | 96 | 95.5 | 93.8 | 96 |
| 1_99 | 97 | 97.1 | 97 | 97 | 93.8 | 98 |
| 1_100 | 98.5 | 100 | 98 | 95.5 | 93.8 | 96 |
| 1_101 | 97.8 | 100 | 97 | 93.9 | 93.8 | 94 |
| 1_102 | 97.8 | 100 | 97 | 97 | 93.8 | 98 |
| 1_103 | 97 | 97.1 | 97 | 97 | 93.8 | 98 |
| 1_104 | 97.8 | 97.1 | 98 | 97 | 93.8 | 98 |
| 1_105 | 96.3 | 97.1 | 96 | 97 | 93.8 | 98 |
| 1_106 | 97 | 100 | 96 | 95.5 | 93.8 | 96 |
| 1_107 | 96.3 | 97.1 | 96 | 97 | 93.8 | 98 |
| 1_108 | 96.3 | 97.1 | 96 | 95.5 | 93.8 | 96 |
| 1_109 | 96.3 | 97.1 | 96 | 97 | 93.8 | 98 |
| 1_110 | 97 | 97.1 | 97 | 98.5 | 100 | 98 |
| 1_111 | 97.8 | 100 | 97 | 97 | 100 | 96 |
| 1_112 | 96.3 | 97.1 | 96 | 97 | 93.8 | 98 |
| 1_113 | 98.5 | 100 | 98 | 97 | 100 | 96 |
| 1_114 | 96.3 | 100 | 95 | 95.5 | 93.8 | 96 |
| 1_115 | 97.8 | 97.1 | 98 | 98.5 | 100 | 98 |
| 1_116 | 95.5 | 97.1 | 95 | 97 | 93.8 | 98 |
| 1_117 | 97 | 94.1 | 98 | 97 | 93.8 | 98 |
| 1_118 | 95.5 | 97.1 | 95 | 97 | 93.8 | 98 |
| 1_119 | 97 | 97.1 | 97 | 97 | 93.8 | 98 |
| 1_120 | 95.5 | 97.1 | 95 | 97 | 93.8 | 98 |
| 1_121 | 97 | 97 | 97 | 97 | 93.8 | 98 |
| 1_122 | 95.5 | 97.1 | 95 | 97 | 93.8 | 98 |
| 1_123 | 97 | 97.1 | 97 | 98.5 | 100 | 98 |
| 1_124 | 95.5 | 97.1 | 95 | 97 | 93.8 | 98 |
| 1_125 | 98.5 | 97.1 | 99 | 97 | 93.8 | 98 |
| 1_126 | 96.3 | 94.1 | 97 | 93.9 | 93.8 | 94 |
| 1_127 | 97 | 97.1 | 97 | 98.5 | 100 | 98 |
| 1_128 | 96.3 | 97.1 | 96 | 95.5 | 93.8 | 96 |
| 1_129 | 97 | 100 | 96 | 97 | 100 | 96 |
| 1_130 | 95.5 | 97.1 | 95 | 97 | 93.8 | 98 |
| 1_131 | 97 | 100 | 96 | 93.9 | 93.8 | 94 |
| 1_132 | 96.3 | 94.1 | 97 | 97 | 93.8 | 98 |
| 1_133 | 96.3 | 97.1 | 96 | 95.5 | 93.8 | 96 |
| 1_134 | 98.5 | 100 | 98 | 97 | 93.8 | 98 |
| 1_135 | 98.5 | 97.1 | 99 | 95.5 | 93.8 | 96 |
| 1_136 | 97 | 97.1 | 97 | 97 | 93.8 | 98 |
| 1_137 | 97 | 97.1 | 97 | 98.5 | 100 | 98 |
| 1_138 | 96.3 | 97.1 | 96 | 97 | 93.8 | 98 |
| 1_139 | 96.3 | 94.1 | 97 | 97 | 93.8 | 98 |
| 1_140 | 96.3 | 97.1 | 96 | 97 | 93.8 | 98 |
| 1_141 | 97.8 | 97.1 | 98 | 97 | 100 | 96 |
| 1_142 | 95.5 | 94.1 | 96 | 97 | 93.8 | 98 |
| 1_143 | 95.5 | 97.1 | 95 | 97 | 93.8 | 98 |
| 1_144 | 95.5 | 97.1 | 95 | 97 | 93.8 | 98 |
| 1_145 | 97 | 94.1 | 98 | 97 | 93.8 | 98 |
| 1_146 | 95.5 | 94.1 | 96 | 97 | 93.8 | 98 |
| 1_147 | 98.5 | 97.1 | 99 | 97 | 93.8 | 98 |
| 1_148 | 96.3 | 94.1 | 97 | 97 | 93.8 | 98 |
| 1_149 | 95.5 | 97.1 | 95 | 97 | 93.8 | 98 |
| 1_150 | 95.5 | 97.1 | 95 | 97 | 93.8 | 98 |
| 1_151 | 97.8 | 97.1 | 98 | 95.5 | 93.8 | 96 |
| 1_152 | 96.3 | 97.1 | 96 | 97 | 93.8 | 98 |
| 1_153 | 97.8 | 100 | 97 | 97 | 93.8 | 98 |
| 1_154 | 97.8 | 97.1 | 98 | 95.5 | 93.8 | 96 |
| 1_155 | 98.5 | 97.1 | 99 | 97 | 93.8 | 98 |
| 1_156 | 96.3 | 97.1 | 96 | 97 | 93.8 | 98 |
| 1_157 | 97 | 97.1 | 97 | 95.5 | 93.8 | 96 |
| 1_158 | 96.3 | 100 | 95 | 97 | 100 | 96 |
| 1_159 | 95.5 | 97.1 | 95 | 97 | 93.8 | 98 |
| 1_160 | 97 | 97.1 | 97 | 97 | 93.8 | 98 |
| 1_161 | 96.3 | 94.1 | 97 | 97 | 93.8 | 98 |
| 1_162 | 96.3 | 97.1 | 96 | 97 | 93.8 | 98 |
| 1_163 | 95.5 | 97.1 | 95 | 97 | 100 | 96 |
| 1_164 | 95.5 | 97.1 | 95 | 97 | 93.8 | 98 |
| 1_165 | 96.3 | 94.1 | 97 | 97 | 93.8 | 98 |
| 1_166 | 97 | 97.1 | 97 | 97 | 93.8 | 98 |
| 1_167 | 96.3 | 97.1 | 96 | 97 | 93.8 | 98 |
| 1_168 | 97 | 94.1 | 98 | 98.5 | 93.8 | 100 |
| 1_169 | 98.5 | 97.1 | 99 | 97 | 93.8 | 98 |
| 1_170 | 100 | 100 | 100 | 97 | 93.8 | 98 |
| 1_171 | 99.3 | 100 | 99 | 98.5 | 100 | 98 |
| 1_172 | 96.3 | 97.1 | 96 | 97 | 93.8 | 98 |
| 1_173 | 98.5 | 100 | 98 | 98.5 | 100 | 98 |
| 1_174 | 95.5 | 94.1 | 96 | 97 | 93.8 | 98 |
| 1_175 | 97 | 97.1 | 97 | 97 | 93.8 | 98 |
| 1_176 | 98.5 | 100 | 98 | 98.5 | 93.8 | 100 |
| 1_177 | 97.8 | 97.1 | 98 | 97 | 93.8 | 98 |
| 1_178 | 99.3 | 100 | 99 | 97 | 100 | 96 |
| 1_179 | 98.5 | 100 | 98 | 98.5 | 100 | 98 |
| 1_180 | 99.3 | 100 | 99 | 97 | 100 | 96 |
| 1_181 | 97.8 | 97.1 | 98 | 97 | 93.8 | 98 |
| 1_182 | 97 | 97.1 | 97 | 97 | 93.8 | 98 |
| 1_183 | 99.3 | 100 | 99 | 100 | 100 | 100 |

### Example 3

<Selection of Gene Marker Using all Samples and Method for Evaluating Liver Cancer Discriminant Performance of Acquired Gene Marker>

In this Example, the samples in the training cohort and the validation cohort used in Examples 1 and 2 were integrated, and selection of a gene marker and evaluation of its liver cancer discriminant performance were conducted using all of the samples.

Specifically, the miRNA expression levels in the serum of the 50 liver cancer patients and the 150 healthy subjects obtained in the above-mentioned Reference Examples were normalized by quantile normalization. In order to acquire diagnostic markers with higher reliability, only genes having a gene expression level of 2⁶ or higher in 50% or more of the samples in either of the liver cancer patient group or the healthy subject group were selected in the gene marker selection. In order to further acquire statistical significance for discriminating a liver cancer patient group from a healthy subject group, the P value obtained by two-tailed t-test assuming equal variance as to each gene expression level was corrected by the Bonferroni method, and genes that satisfied p<0.01 were selected as gene markers for use in explanatory variables of a discriminant. The acquired genes are described in Table 7. In this way, hsa-miR-4688, hsa-miR-4648, hsa-miR-6085, hsa-miR-6126, hsa-miR-6880-5p, hsa-miR-328-5p, hsa-miR-6768-5p, hsa-miR-3180, hsa-miR-6087, hsa-miR-1273g-3p, hsa-miR-1225-5p, hsa-miR-3196, hsa-miR-4695-5p, hsa-miR-6732-5p, hsa-miR-638, hsa-miR-6813-5p, hsa-miR-665, hsa-miR-486-3p, hsa-miR-4466, hsa-miR-30c-1-3p, hsa-miR-3621, hsa-miR-6743-5p, hsa-miR-4298, hsa-miR-4741, hsa-miR-3619-3p, hsa-miR-6824-5p, hsa-miR-5698, hsa-miR-371a-5p, hsa-miR-4488, hsa-miR-1233-5p, hsa-miR-4723-5p, hsa-miR-24-3p, hsa-miR-1238-5p, hsa-miR-4442, hsa-miR-3928-3p, hsa-miR-6716-5p, hsa-miR-6089, hsa-miR-6124, hsa-miR-6778-5p, hsa-miR-557 and hsa-miR-6090 genes represented by SEQ ID NOs: 184 to 224 were found as liver cancer markers relative to the healthy subjects, in addition to the genes described in Table 2. As with the polynucleotides shown in SEQ ID NOs: 1 to 183, the results obtained about the polynucleotides shown in SEQ ID NOs: 184 to 224 also showed that the expression level measurement values were significantly lower (−) or higher (+) in the liver cancer patient group than in the healthy subject group (Table 7). These results were able to be validated in the validation cohort. Thus, the presence or absence of liver cancer in the newly obtained samples can be determined by the methods described in Examples 1 and 2 by using the gene expression level measurement values described in Table 7 either alone or in combination with the gene expression level measurement values described in Table 2.

TABLE 7

| | | | Expression level in |
|---|---|---|---|
| | | P value after | liver cancer patient |
| | | Bonferroni | relative to healthy |
| SEQ ID NO: | Gene name | correction | subject |
| 1 | hsa-miR-1343-3p | 7.76.E−56 | − |
| 2 | hsa-miR-6726-5p | 1.12.E−51 | − |
| 3 | hsa-miR-6515-3p | 4.93.E−36 | + |
| 4 | hsa-miR-4651 | 9.12.E−42 | − |
| 5 | hsa-miR-4257 | 2.81.E−42 | − |
| 6 | hsa-miR-3188 | 1.06.E−41 | + |
| 7 | hsa-miR-6131 | 1.97.E−37 | − |
| 8 | hsa-miR-6766-3p | 4.59.E−35 | + |
| 9 | hsa-miR-7641 | 2.35.E−36 | − |
| 10 | hsa-miR-1249 | 2.50.E−34 | + |
| 11 | hsa-miR-3679-3p | 5.67.E−31 | + |
| 12 | hsa-miR-6787-5p | 9.25.E−36 | − |
| 13 | hsa-miR-4454 | 1.38.E−34 | − |
| 14 | hsa-miR-3135b | 3.23.E−23 | − |
| 15 | hsa-miR-6765-3p | 8.15.E−32 | − |
| 16 | hsa-miR-7975 | 4.38.E−28 | − |
| 17 | hsa-miR-204-3p | 2.40.E−25 | − |
| 18 | hsa-miR-7977 | 6.65.E−27 | − |
| 19 | hsa-miR-7110-5p | 2.91.E−28 | + |
| 20 | hsa-miR-6717-5p | 4.18.E−23 | − |
| 21 | hsa-miR-6870-5p | 2.08.E−27 | + |
| 22 | hsa-miR-663b | 1.18.E−29 | − |
| 23 | hsa-miR-6875-5p | 1.80.E−24 | + |
| 24 | hsa-miR-8072 | 1.13.E−21 | + |
| 25 | hsa-miR-6816-5p | 9.86.E−26 | + |
| 26 | hsa-miR-4281 | 1.18.E−24 | − |
| 27 | hsa-miR-6729-5p | 1.39.E−22 | + |
| 28 | hsa-miR-8069 | 9.35.E−19 | + |
| 29 | hsa-miR-4706 | 1.28.E−23 | − |
| 30 | hsa-miR-7108-5p | 3.30.E−21 | + |
| 31 | hsa-miR-4433b-3p | 1.04.E−21 | + |
| 32 | hsa-miR-6893-5p | 7.87.E−23 | − |
| 33 | hsa-miR-6857-5p | 1.05.E−22 | + |
| 34 | hsa-miR-1227-5p | 5.00.E−23 | + |
| 35 | hsa-miR-6741-5p | 2.98.E−21 | − |
| 36 | hsa-miR-451a | 1.60.E−19 | − |
| 37 | hsa-miR-8063 | 1.20.E−22 | − |
| 38 | hsa-miR-3622a-5p | 8.16.E−21 | − |
| 39 | hsa-miR-615-5p | 1.17.E−21 | − |
| 40 | hsa-miR-128-1-5p | 8.49.E−17 | + |
| 41 | hsa-miR-6825-5p | 4.10.E−25 | + |
| 42 | hsa-miR-1260b | 4.23.E−20 | − |
| 43 | hsa-miR-4433-3p | 7.63.E−20 | + |
| 44 | hsa-miR-4665-5p | 1.92.E−15 | − |
| 45 | hsa-miR-7845-5p | 9.71.E−18 | + |
| 46 | hsa-miR-1908-5p | 6.59.E−21 | + |
| 47 | hsa-miR-6840-3p | 1.70.E−20 | − |
| 48 | hsa-miR-6765-5p | 3.32.E−19 | + |
| 49 | hsa-miR-296-5p | 5.14.E−14 | + |
| 51 | hsa-miR-6781-5p | 6.41.E−18 | + |
| 52 | hsa-miR-423-5p | 1.91.E−15 | − |
| 53 | hsa-miR-3663-3p | 1.67.E−16 | − |
| 54 | hsa-miR-6784-5p | 8.43.E−18 | + |
| 55 | hsa-miR-6749-5p | 2.59.E−20 | − |
| 56 | hsa-miR-1231 | 1.33.E−14 | + |
| 57 | hsa-miR-4746-3p | 3.47.E−19 | + |
| 58 | hsa-miR-6780b-5p | 2.82.E−21 | + |
| 59 | hsa-miR-4758-5p | 4.87.E−15 | − |
| 60 | hsa-miR-3679-5p | 1.59.E−19 | + |
| 61 | hsa-miR-3184-5p | 6.75.E−18 | + |
| 62 | hsa-miR-6125 | 8.43.E−17 | + |
| 63 | hsa-miR-6721-5p | 3.93.E−15 | + |
| 64 | hsa-miR-6791-5p | 1.78.E−17 | + |
| 65 | hsa-miR-3185 | 5.38.E−17 | + |
| 66 | hsa-miR-1260a | 7.87.E−15 | − |
| 67 | hsa-miR-3197 | 1.51.E−14 | + |
| 68 | hsa-miR-6845-5p | 2.09.E−16 | + |
| 69 | hsa-miR-6887-5p | 3.08.E−15 | − |
| 70 | hsa-miR-6738-5p | 1.83.E−16 | − |
| 71 | hsa-miR-6872-3p | 5.80.E−14 | − |
| 72 | hsa-miR-4497 | 2.63.E−10 | − |
| 73 | hsa-miR-1229-5p | 1.21.E−14 | + |
| 74 | hsa-miR-6820-5p | 5.60.E−13 | − |
| 75 | hsa-miR-6777-5p | 7.03.E−15 | − |
| 76 | hsa-miR-3917 | 7.63.E−13 | − |
| 77 | hsa-miR-5787 | 5.42.E−15 | + |
| 78 | hsa-miR-4286 | 1.57.E−12 | − |
| 79 | hsa-miR-6877-5p | 1.83.E−14 | − |
| 80 | hsa-miR-1225-3p | 4.77.E−11 | + |
| 81 | hsa-miR-6088 | 4.12.E−13 | − |
| 82 | hsa-miR-6800-5p | 1.01.E−13 | + |
| 83 | hsa-miR-1246 | 1.20.E−10 | − |
| 84 | hsa-miR-4467 | 2.24.E−15 | + |
| 85 | hsa-miR-4419b | 3.03.E−12 | − |
| 86 | hsa-miR-1914-3p | 3.27.E−13 | − |
| 87 | hsa-miR-4632-5p | 6.04.E−12 | + |
| 88 | hsa-miR-1915-5p | 7.61.E−15 | − |
| 89 | hsa-miR-3940-5p | 7.23.E−12 | + |
| 91 | hsa-miR-6746-5p | 5.54.E−13 | − |
| 92 | hsa-miR-5001-5p | 2.14.E−13 | − |
| 93 | hsa-miR-1228-5p | 7.95.E−13 | + |
| 94 | hsa-miR-5572 | 5.18.E−16 | + |
| 95 | hsa-miR-4327 | 2.61.E−09 | + |
| 96 | hsa-miR-4638-5p | 1.48.E−10 | − |
| 97 | hsa-miR-6799-5p | 1.10.E−10 | + |
| 98 | hsa-miR-6861-5p | 8.44.E−11 | − |
| 99 | hsa-miR-6727-5p | 2.38.E−13 | − |
| 100 | hsa-miR-4513 | 8.83.E−12 | − |
| 101 | hsa-miR-6805-3p | 1.08.E−12 | + |
| 102 | hsa-miR-6808-5p | 3.32.E−10 | + |
| 103 | hsa-miR-4449 | 4.13.E−09 | + |
| 104 | hsa-miR-1199-5p | 1.45.E−11 | − |
| 105 | hsa-miR-1275 | 2.47.E−08 | + |
| 106 | hsa-miR-4792 | 9.54.E−13 | + |
| 107 | hsa-miR-4443 | 4.44.E−10 | + |
| 108 | hsa-miR-6891-5p | 3.67.E−12 | + |
| 109 | hsa-miR-6826-5p | 5.10.E−11 | − |
| 110 | hsa-miR-6807-5p | 1.03.E−09 | + |
| 111 | hsa-miR-7150 | 1.05.E−09 | + |
| 112 | hsa-miR-4534 | 1.61.E−09 | + |
| 113 | hsa-miR-4476 | 6.66.E−08 | − |
| 114 | hsa-miR-4649-5p | 1.12.E−10 | − |
| 115 | hsa-miR-4525 | 4.68.E−12 | − |
| 116 | hsa-miR-1915-3p | 1.92.E−10 | + |
| 117 | hsa-miR-4516 | 1.95.E−10 | − |
| 118 | hsa-miR-4417 | 3.89.E−10 | + |
| 119 | hsa-miR-642b-3p | 3.82.E−10 | − |
| 120 | hsa-miR-3141 | 1.02.E−08 | + |
| 121 | hsa-miR-5100 | 4.74.E−08 | − |
| 122 | hsa-miR-6848-5p | 7.00.E−10 | + |
| 123 | hsa-miR-4739 | 1.94.E−08 | + |
| 124 | hsa-miR-4459 | 1.30.E−08 | + |
| 125 | hsa-miR-1237-5p | 1.04.E−08 | + |
| 126 | hsa-miR-296-3p | 9.28.E−08 | − |
| 127 | hsa-miR-4665-3p | 9.58.E−12 | + |
| 128 | hsa-miR-6786-5p | 7.26.E−06 | + |
| 129 | hsa-miR-4258 | 4.38.E−08 | − |
| 130 | hsa-miR-6510-5p | 4.93.E−11 | + |
| 131 | hsa-miR-1343-5p | 1.77.E−10 | + |
| 132 | hsa-miR-1247-3p | 3.69.E−11 | + |
| 133 | hsa-miR-6805-5p | 1.78.E−09 | + |
| 134 | hsa-miR-4492 | 1.28.E−07 | + |
| 135 | hsa-miR-1469 | 8.04.E−06 | + |
| 136 | hsa-miR-1268b | 7.93.E−07 | + |
| 137 | hsa-miR-6858-5p | 2.19.E−06 | + |
| 138 | hsa-miR-3937 | 5.07.E−06 | + |
| 139 | hsa-miR-939-5p | 3.71.E−10 | + |
| 140 | hsa-miR-3656 | 9.45.E−10 | + |
| 141 | hsa-miR-744-5p | 6.81.E−08 | + |
| 142 | hsa-miR-4687-3p | 1.70.E−07 | + |
| 143 | hsa-miR-4763-3p | 1.79.E−06 | + |
| 144 | hsa-miR-3620-5p | 2.74.E−06 | + |
| 145 | hsa-miR-3195 | 1.35.E−04 | + |
| 146 | hsa-miR-6842-5p | 9.98.E−12 | + |
| 147 | hsa-miR-4707-5p | 7.25.E−06 | + |
| 148 | hsa-miR-642a-3p | 1.31.E−06 | + |
| 149 | hsa-miR-7113-3p | 2.95.E−07 | + |
| 150 | hsa-miR-4728-5p | 3.51.E−06 | − |
| 151 | hsa-miR-5195-3p | 9.06.E−07 | − |
| 152 | hsa-miR-1185-1-3p | 3.35.E−05 | + |
| 153 | hsa-miR-6774-5p | 5.14.E−04 | + |
| 154 | hsa-miR-8059 | 1.37.E−05 | − |
| 155 | hsa-miR-3131 | 6.97.E−08 | − |
| 156 | hsa-miR-7847-3p | 6.35.E−06 | − |
| 157 | hsa-miR-4463 | 1.04.E−07 | + |
| 158 | hsa-miR-128-2-5p | 3.84.E−06 | − |
| 159 | hsa-miR-4508 | 3.57.E−05 | + |
| 160 | hsa-miR-6806-5p | 2.04.E−06 | − |
| 161 | hsa-miR-7111-5p | 6.31.E−05 | + |
| 162 | hsa-miR-6782-5p | 2.11.E−07 | + |
| 163 | hsa-miR-4734 | 1.79.E−05 | + |
| 164 | hsa-miR-3162-5p | 7.73.E−04 | + |
| 165 | hsa-miR-887-3p | 7.67.E−05 | + |
| 166 | hsa-miR-6752-5p | 7.74.E−05 | + |
| 167 | hsa-miR-6724-5p | 4.17.E−05 | + |
| 168 | hsa-miR-23b-3p | 1.17.E−30 | − |
| 169 | hsa-miR-23a-3p | 5.61.E−28 | − |
| 170 | hsa-miR-625-3p | 1.19.E−16 | + |
| 171 | hsa-miR-1228-3p | 7.80.E−28 | + |
| 172 | hsa-miR-614 | 7.24.E−27 | − |
| 173 | hsa-miR-1913 | 1.52.E−26 | + |
| 174 | hsa-miR-92a-2-5p | 5.94.E−24 | + |
| 175 | hsa-miR-187-5p | 1.72.E−26 | − |
| 176 | hsa-miR-16-5p | 4.14.E−20 | − |
| 177 | hsa-miR-92b-3p | 1.09.E−17 | − |
| 178 | hsa-miR-150-3p | 1.47.E−13 | − |
| 179 | hsa-miR-564 | 2.36.E−15 | − |
| 180 | hsa-miR-125a-3p | 7.07.E−12 | − |
| 181 | hsa-miR-92b-5p | 8.01.E−10 | + |
| 182 | hsa-miR-92a-3p | 3.99.E−09 | − |
| 183 | hsa-miR-663a | 1.34.E−06 | + |
| 184 | hsa-miR-4688 | 4.97.E−07 | − |
| 185 | hsa-miR-4648 | 2.21.E−05 | + |
| 186 | hsa-miR-6085 | 2.31.E−05 | + |
| 187 | hsa-miR-6126 | 2.31.E−05 | + |
| 188 | hsa-miR-6880-5p | 2.44.E−05 | + |
| 189 | hsa-miR-328-5p | 2.90.E−05 | + |
| 190 | hsa-miR-6768-5p | 4.36.E−05 | + |
| 191 | hsa-miR-3180 | 6.14.E−05 | + |
| 192 | hsa-miR-6087 | 8.15.E−05 | − |
| 193 | hsa-miR-1273g-3p | 1.23.E−04 | − |
| 194 | hsa-miR-1225-5p | 1.23.E−04 | + |
| 195 | hsa-miR-3196 | 1.32.E−04 | + |
| 196 | hsa-miR-4695-5p | 1.47.E−04 | + |
| 197 | hsa-miR-6732-5p | 2.45.E−04 | + |
| 198 | hsa-miR-638 | 2.98.E−04 | − |
| 199 | hsa-miR-6813-5p | 3.27.E−04 | + |
| 200 | hsa-miR-665 | 3.46.E−04 | + |
| 201 | hsa-miR-486-3p | 4.04.E−04 | − |
| 202 | hsa-miR-4466 | 4.22.E−04 | − |
| 203 | hsa-miR-30c-1-3p | 5.71.E−04 | + |
| 204 | hsa-miR-3621 | 8.32.E−04 | − |
| 205 | hsa-miR-6743-5p | 8.89.E−04 | + |
| 206 | hsa-miR-4298 | 1.05.E−03 | − |
| 207 | hsa-miR-4741 | 1.07.E−03 | + |
| 208 | hsa-miR-3619-3p | 1.11.E−03 | + |
| 209 | hsa-miR-6824-5p | 1.17.E−03 | + |
| 210 | hsa-miR-5698 | 1.30.E−03 | − |
| 211 | hsa-miR-371a-5p | 1.51.E−03 | − |
| 212 | hsa-miR-4488 | 1.85.E−03 | − |
| 213 | hsa-miR-1233-5p | 1.90.E−03 | − |
| 214 | hsa-miR-4723-5p | 2.05.E−03 | + |
| 215 | hsa-miR-24-3p | 2.09.E−03 | − |
| 216 | hsa-miR-1238-5p | 2.18.E−03 | + |
| 217 | hsa-miR-4442 | 2.48.E−03 | − |
| 218 | hsa-miR-3928-3p | 2.71.E−03 | + |
| 219 | hsa-miR-6716-5p | 2.96.E−03 | + |
| 220 | hsa-miR-6089 | 3.43.E−03 | + |
| 221 | hsa-miR-6124 | 3.68.E−03 | + |
| 222 | hsa-miR-6778-5p | 4.10.E−03 | − |
| 223 | hsa-miR-557 | 6.88.E−03 | + |
| 224 | hsa-miR-6090 | 9.92.E−03 | + |

### Example 4

<Method for Evaluating Liver Cancer-Specific Discriminant Performance by Combination of Multiple Gene Markers Using Samples in the Validation Cohort>

In this Example, novel additional gene markers for diagnosis were selected by comparing gene expression levels of miRNAs in sera of liver cancer patients with those of a control group consisting of healthy subjects, pancreatic cancer patients, bile duct cancer patients, stomach cancer patients, esophageal cancer patients, colorectal cancer patients, and benign pancreaticobiliary disease patients, in the same way as the method described in Example 1, and targeting the training cohort as the sample group described in Reference Example 2. One or two or more markers selected from the group consisting of the polynucleotides consisting of the nucleotide sequences represented by SEQ ID NOs: 714 to 729 thus selected and the gene markers selected in Example 1 were used to evaluate liver cancer-specific discriminant performance.

Specifically, first, the miRNA expression levels in the training cohort and the validation cohort obtained in Reference Example 2 mentioned above were combined and normalized by quantile normalization. Next, Fisher's discriminant analysis was conducted as to combinations of 1 to 4 expression level measurement values comprising at least one or more of the expression level measurement values of the polynucleotides consisting of the nucleotide sequences represented by SEQ ID NOs: 1 to 167 and 714 to 729, to construct a discriminant for determining the presence or absence of liver cancer. Next, accuracy, sensitivity, and specificity in the validation cohort were calculated using the discriminant thus prepared, with the liver cancer patient group as a positive sample group and, on the other hand, the healthy subject group, the pancreatic cancer patient group, the bile duct cancer patient group, the stomach cancer patient group, the esophageal cancer patient group, the colorectal cancer patient group, and the benign pancreaticobiliary disease patient group as negative sample groups. The discriminant performance of the selected polynucleotides was validated using independent samples.

Most of polynucleotides consisting of the nucleotide sequences represented by these SEQ ID NOs (SEQ ID NOs: 1 to 224 and 714 to 729 corresponding to the miRNA markers of Table 1) or complementary sequences thereof mentioned above were able to provide relatively high accuracy, sensitivity, and specificity in the determination of the presence or absence of liver cancer, and furthermore, were able to specifically discriminate liver cancer from other cancers. For example, among the combinations of a plurality of polynucleotides selected from the group consisting of polynucleotides consisting of the nucleotide sequences represented by SEQ ID NOs: 1, 2, 3, 5, 7, 9, 12, 17, 20, 22, 27, 28, 29, 38, 39, 44, 46, 48, 51, 54, 61, 76, 89, 93, 101, 109, 116, 123, 132, 134, 136, 148, 150, 151, 155, 157, 164, 166, 167, 172, 180, 186, 188, 189, 197, 198, 214, 216, 714, 715, 716, 717, 718, 719, 720, 721, 722, 723, 724, 725, 726, 727, 728 and 729 or complementary sequences thereof (the cancer type-specific polynucleotide group 1) as polynucleotides capable of specifically binding to target markers, combinations comprising at least one or more polynucleotide(s) selected from the group consisting of polynucleotides consisting of the nucleotide sequences represented by SEQ ID NOs: 1, 3, 7, 9, 22, 38, 44, 134, 148, 155, 157, 164, 167, 172, 214, 714, 715, 716, and 717 or complementary sequences thereof (the cancer type-specific polynucleotide group 2) were able to specifically discriminate liver cancer from other cancers with high accuracy.

The number of the polynucleotides with cancer type specificity in the combination mentioned above can be 1, 2, 3, 4, 5, 6, 7, 8, 9, 10 or more for the combination. The combinations of 4 or more polynucleotides were able to exhibit discriminant accuracy of 90% or higher.

Specifically, the discriminant accuracy of the measurement using the polynucleotide consisting of the nucleotide sequence represented by SEQ ID NO: 1 or a complementary sequence thereof is shown in Table 8-1. In Table 8-1, “SEQ ID NO” represents one polynucleotide or a combination of a plurality of polynucleotides used with the number of SEQ ID NO: (the same holds true for Tables 8-2 to 8-19). The measurement using, alone (one), the polynucleotide consisting of the nucleotide sequence represented by SEQ ID NO: 1 or a complementary sequence thereof exhibited accuracy of 71.2% in the training cohort and accuracy of 73.2% in the validation cohort. Also, for example, the measurement using the combinations of two polynucleotides comprising at least one polynucleotide consisting of the nucleotide sequence represented by SEQ ID NO: 1 or a complementary sequence thereof exhibited the highest accuracy of 88.1% in the training cohort and accuracy of 90% in the validation cohort. Furthermore, for example, the measurement using the combinations of three polynucleotides comprising at least one polynucleotide consisting of the nucleotide sequence represented by SEQ ID NO: 1 or a complementary sequence thereof exhibited the highest accuracy of 90.2% in the training cohort and accuracy of 90.5% in the validation cohort. Furthermore, for example, the measurement using the combinations of four polynucleotides comprising at least one polynucleotide consisting of the nucleotide sequence represented by SEQ ID NO: 1 or a complementary sequence thereof exhibited the highest accuracy of 92.3% in the training cohort and accuracy of 93.2% in the validation cohort.

The discriminant accuracy of the measurement using the polynucleotide consisting of the nucleotide sequence represented by SEQ ID NO: 3 or a complementary sequence thereof is shown in Table 8-2. The measurement using, alone (one), the polynucleotide consisting of the nucleotide sequence represented by SEQ ID NO: 3 or a complementary sequence thereof exhibited accuracy of 78.7% in the training cohort and accuracy of 73.2% in the validation cohort. Also, for example, the measurement using the combinations of two polynucleotides comprising at least one polynucleotide consisting of the nucleotide sequence represented by SEQ ID NO: 3 or a complementary sequence thereof exhibited the highest accuracy of 88.7% in the training cohort and accuracy of 87.4% in the validation cohort. Furthermore, for example, the measurement using the combinations of three polynucleotides comprising at least one polynucleotide consisting of the nucleotide sequence represented by SEQ ID NO: 3 or a complementary sequence thereof exhibited the highest accuracy of 91.8% in the training cohort and accuracy of 87.9% in the validation cohort. Furthermore, for example, the measurement using the combinations of four polynucleotides comprising at least one polynucleotide consisting of the nucleotide sequence represented by SEQ ID NO: 3 or a complementary sequence thereof exhibited the highest accuracy of 92.9% in the training cohort and accuracy of 93.2% in the validation cohort.

The discriminant accuracy of the measurement using the polynucleotide consisting of the nucleotide sequence represented by SEQ ID NO: 7 or a complementary sequence thereof is shown in Table 8-3. The measurement using, alone (one), the polynucleotide consisting of the nucleotide sequence represented by SEQ ID NO: 7 or a complementary sequence thereof exhibited accuracy of 85.5% in the training cohort and accuracy of 84.7% in the validation cohort. Also, for example, the measurement using the combinations of two polynucleotides comprising at least one polynucleotide consisting of the nucleotide sequence represented by SEQ ID NO: 7 or a complementary sequence thereof exhibited the highest accuracy of 91.5% in the training cohort and accuracy of 90.5% in the validation cohort. Furthermore, for example, the measurement using the combinations of three polynucleotides comprising at least one polynucleotide consisting of the nucleotide sequence represented by SEQ ID NO: 7 or a complementary sequence thereof exhibited the highest accuracy of 93.7% in the training cohort and accuracy of 92.1% in the validation cohort. Furthermore, for example, the measurement using the combinations of four polynucleotides comprising at least one polynucleotide consisting of the nucleotide sequence represented by SEQ ID NO: 7 or a complementary sequence thereof exhibited the highest accuracy of 94.4% in the training cohort and accuracy of 92.6% in the validation cohort.

The discriminant accuracy of the measurement using the polynucleotide consisting of the nucleotide sequence represented by SEQ ID NO: 9 or a complementary sequence thereof is shown in Table 8-4. The measurement using, alone (one), the polynucleotide consisting of the nucleotide sequence represented by SEQ ID NO: 9 or a complementary sequence thereof exhibited accuracy of 59.7% in the training cohort and accuracy of 59.5% in the validation cohort. Also, for example, the measurement using the combinations of two polynucleotides comprising at least one polynucleotide consisting of the nucleotide sequence represented by SEQ ID NO: 9 or a complementary sequence thereof exhibited the highest accuracy of 86% in the training cohort and accuracy of 81.1% in the validation cohort. Furthermore, for example, the measurement using the combinations of three polynucleotides comprising at least one polynucleotide consisting of the nucleotide sequence represented by SEQ ID NO: 9 or a complementary sequence thereof exhibited the highest accuracy of 91.8% in the training cohort and accuracy of 84.7% in the validation cohort. Furthermore, for example, the measurement using the combinations of four polynucleotides comprising at least one polynucleotide consisting of the nucleotide sequence represented by SEQ ID NO: 9 or a complementary sequence thereof exhibited the highest accuracy of 94.7% in the training cohort and accuracy of 92.1% in the validation cohort.

The discriminant accuracy of the measurement using the polynucleotide consisting of the nucleotide sequence represented by SEQ ID NO: 22 or a complementary sequence thereof is shown in Table 8-5. The measurement using, alone (one), the polynucleotide consisting of the nucleotide sequence represented by SEQ ID NO: 22 or a complementary sequence thereof exhibited accuracy of 76.5% in the training cohort and accuracy of 78.9% in the validation cohort. Also, for example, the measurement using the combinations of two polynucleotides comprising at least one polynucleotide consisting of the nucleotide sequence represented by SEQ ID NO: 22 or a complementary sequence thereof exhibited the highest accuracy of 85.8% in the training cohort and accuracy of 84.7% in the validation cohort. Furthermore, for example, the measurement using the combinations of three polynucleotides comprising at least one polynucleotide consisting of the nucleotide sequence represented by SEQ ID NO: 22 or a complementary sequence thereof exhibited the highest accuracy of 91.3% in the training cohort and accuracy of 91.6% in the validation cohort. Furthermore, for example, the measurement using the combinations of four polynucleotides comprising at least one polynucleotide consisting of the nucleotide sequence represented by SEQ ID NO: 22 or a complementary sequence thereof exhibited the highest accuracy of 93.7% in the training cohort and accuracy of 93.7% in the validation cohort.

The discriminant accuracy of the measurement using the polynucleotide consisting of the nucleotide sequence represented by SEQ ID NO: 38 or a complementary sequence thereof is shown in Table 8-6. The measurement using, alone (one), the polynucleotide consisting of the nucleotide sequence represented by SEQ ID NO: 38 or a complementary sequence thereof exhibited accuracy of 65.5% in the training cohort and accuracy of 65.8% in the validation cohort. Also, for example, the measurement using the combinations of two polynucleotides SEQ ID NO: 38 or a complementary sequence thereof exhibited the highest accuracy of 86.3% in the training cohort and accuracy of 84.2% in the validation cohort. Furthermore, for example, the measurement using the combinations of three polynucleotides comprising at least one polynucleotide consisting of the nucleotide sequence represented by SEQ ID NO: 38 or a complementary sequence thereof exhibited the highest accuracy of 92.3% in the training cohort and accuracy of 91.6% in the validation cohort. Furthermore, for example, the measurement using the combinations of four polynucleotides comprising at least one polynucleotide consisting of the nucleotide sequence represented by SEQ ID NO: 38 or a complementary sequence thereof exhibited the highest accuracy of 94.2% in the training cohort and accuracy of 92.1% in the validation cohort.

The discriminant accuracy of the measurement using the polynucleotide consisting of the nucleotide sequence represented by SEQ ID NO: 44 or a complementary sequence thereof is shown in Table 8-7. The measurement using, alone (one), the polynucleotide consisting of the nucleotide sequence represented by SEQ ID NO: 44 or a complementary sequence thereof exhibited accuracy of 62.6% in the training cohort and accuracy of 62.1% in the validation cohort. Also, for example, the measurement using the combinations of two polynucleotides comprising at least one polynucleotide consisting of the nucleotide sequence represented by SEQ ID NO: 44 or a complementary sequence thereof exhibited the highest accuracy of 90.5% in the training cohort and accuracy of 86.3% in the validation cohort. Furthermore, for example, the measurement using the combinations of three polynucleotides comprising at least one polynucleotide consisting of the nucleotide sequence represented by SEQ ID NO: 44 or a complementary sequence thereof exhibited the highest accuracy of 92.9% in the training cohort and accuracy of 91.1% in the validation cohort. Furthermore, for example, the measurement using the combinations of four polynucleotides comprising at least one polynucleotide consisting of the nucleotide sequence represented by SEQ ID NO: 44 or a complementary sequence thereof exhibited the highest accuracy of 93.7% in the training cohort and accuracy of 91.6% in the validation cohort.

The discriminant accuracy of the measurement using the polynucleotide consisting of the nucleotide sequence represented by SEQ ID NO: 134 or a complementary sequence thereof is shown in Table 8-8. The measurement using, alone (one), the polynucleotide consisting of the nucleotide sequence represented by SEQ ID NO: 134 or a complementary sequence thereof exhibited accuracy of 53.4% in the training cohort and accuracy of 58.9% in the validation cohort. Also, for example, the measurement using the combinations of two polynucleotides comprising at least one polynucleotide consisting of the nucleotide sequence represented by SEQ ID NO: 134 or a complementary sequence thereof exhibited the highest accuracy of 87.3% in the training cohort and accuracy of 84.2% in the validation cohort. Furthermore, for example, the measurement using the combinations of three polynucleotides comprising at least one polynucleotide consisting of the nucleotide sequence represented by SEQ ID NO: 134 or a complementary sequence thereof exhibited the highest accuracy of 92.9% in the training cohort and accuracy of 91.1% in the validation cohort. Furthermore, for example, the measurement using the combinations of four polynucleotides comprising at least one polynucleotide consisting of the nucleotide sequence represented by SEQ ID NO: 134 or a complementary sequence thereof exhibited the highest accuracy of 93.7% in the training cohort and accuracy of 92.1% in the validation cohort.

The discriminant accuracy of the measurement using the polynucleotide consisting of the nucleotide sequence represented by SEQ ID NO: 148 or a complementary sequence thereof is shown in Table 8-9. The measurement using, alone (one), the polynucleotide consisting of the nucleotide sequence represented by SEQ ID NO: 148 or a complementary sequence thereof exhibited accuracy of 73.6% in the training cohort and accuracy of 75.3% in the validation cohort. Also, for example, the measurement using the combinations of two polynucleotides comprising at least one polynucleotide consisting of the nucleotide sequence represented by SEQ ID NO: 148 or a complementary sequence thereof exhibited the highest accuracy of 86.3% in the training cohort and accuracy of 85.3% in the validation cohort. Furthermore, for example, the measurement using the combinations of three polynucleotides SEQ ID NO: 148 or a complementary sequence thereof exhibited the highest accuracy of 93.7% in the training cohort and accuracy of 91.6% in the validation cohort. Furthermore, for example, the measurement using the combinations of four polynucleotides comprising at least one polynucleotide consisting of the nucleotide sequence represented by SEQ ID NO: 148 or a complementary sequence thereof exhibited the highest accuracy of 93.7% in the training cohort and accuracy of 92.1% in the validation cohort.

The discriminant accuracy of the measurement using the polynucleotide consisting of the nucleotide sequence represented by SEQ ID NO: 155 or a complementary sequence thereof is shown in Table 8-10. The measurement using, alone (one), the polynucleotide consisting of the nucleotide sequence represented by SEQ ID NO: 155 or a complementary sequence thereof exhibited accuracy of 60.8% in the training cohort and accuracy of 58.9% in the validation cohort. Also, for example, the measurement using the combinations of two polynucleotides comprising at least one polynucleotide consisting of the nucleotide sequence represented by SEQ ID NO: 155 or a complementary sequence thereof exhibited the highest accuracy of 86.5% in the training cohort and accuracy of 85.8% in the validation cohort. Furthermore, for example, the measurement using the combinations of three polynucleotides comprising at least one polynucleotide consisting of the nucleotide sequence represented by SEQ ID NO: 155 or a complementary sequence thereof exhibited the highest accuracy of 90.5% in the training cohort and accuracy of 91.6% in the validation cohort. Furthermore, for example, the measurement using the combinations of four polynucleotides comprising at least one polynucleotide consisting of the nucleotide sequence represented by SEQ ID NO: 155 or a complementary sequence thereof exhibited the highest accuracy of 93.4% in the training cohort and accuracy of 91.6% in the validation cohort.

The discriminant accuracy of the measurement using the polynucleotide consisting of the nucleotide sequence represented by SEQ ID NO: 157 or a complementary sequence thereof is shown in Table 8-11. The measurement using, alone (one), the polynucleotide consisting of the nucleotide sequence represented by SEQ ID NO: 157 or a complementary sequence thereof exhibited accuracy of 70.3% in the training cohort and accuracy of 68.9% in the validation cohort. Also, for example, the measurement using the combinations of two polynucleotides comprising at least one polynucleotide consisting of the nucleotide sequence represented by SEQ ID NO: 157 or a complementary sequence thereof exhibited the highest accuracy of 86.5% in the training cohort and accuracy of 83.2% in the validation cohort. Furthermore, for example, the measurement using the combinations of three polynucleotides comprising at least one polynucleotide consisting of the nucleotide sequence represented by SEQ ID NO: 157 or a complementary sequence thereof exhibited the highest accuracy of 91% in the training cohort and accuracy of 91.6% in the validation cohort. Furthermore, for example, the measurement using the combinations of four polynucleotides comprising at least one polynucleotide consisting of the nucleotide sequence represented by SEQ ID NO: 157 or a complementary sequence thereof exhibited the highest accuracy of 93.9% in the training cohort and accuracy of 92.6% in the validation cohort.

The discriminant accuracy of the measurement using the polynucleotide consisting of the nucleotide sequence represented by SEQ ID NO: 164 or a complementary sequence thereof is shown in Table 8-12. The measurement using, alone (one), the polynucleotide consisting of the nucleotide sequence represented by SEQ ID NO: 164 or a complementary sequence thereof exhibited accuracy of 72.4% in the training cohort and accuracy of 65.8% in the validation cohort. Also, for example, the measurement using the combinations of two polynucleotides comprising at least one polynucleotide consisting of the nucleotide sequence represented by SEQ ID NO: 164 or a complementary sequence thereof exhibited the highest accuracy of 87.6% in the training cohort and accuracy of 87.4% in the validation cohort. Furthermore, for example, the measurement using the combinations of three polynucleotides comprising at least one polynucleotide consisting of the nucleotide sequence represented by SEQ ID NO: 164 or a complementary sequence thereof exhibited the highest accuracy of 91.5% in the training cohort and accuracy of 92.1% in the validation cohort. Furthermore, for example, the measurement using the combinations of four polynucleotides comprising at least one polynucleotide consisting of the nucleotide sequence represented by SEQ ID NO: 164 or a complementary sequence thereof exhibited the highest accuracy of 92.6% in the training cohort and accuracy of 90.5% in the validation cohort.

The discriminant accuracy of the measurement using the polynucleotide consisting of the nucleotide sequence represented by SEQ ID NO: 167 or a complementary sequence thereof is shown in Table 8-13. The measurement using, alone (one), the polynucleotide consisting of the nucleotide sequence represented by SEQ ID NO: 167 or a complementary sequence thereof exhibited accuracy of 62.1% in the training cohort and accuracy of 57.4% in the validation cohort. Also, for example, the measurement using the combinations of two polynucleotides comprising at least one polynucleotide consisting of the nucleotide sequence represented by SEQ ID NO: 167 or a complementary sequence thereof exhibited the highest accuracy of 89.2% in the training cohort and accuracy of 87.4% in the validation cohort. Furthermore, for example, the measurement using the combinations of three polynucleotides comprising at least one polynucleotide consisting of the nucleotide sequence represented by SEQ ID NO: 167 or a complementary sequence thereof exhibited the highest accuracy of 92.1% in the training cohort and accuracy of 90% in the validation cohort. Furthermore, for example, the measurement using the combinations of four polynucleotides comprising at least one polynucleotide consisting of the nucleotide sequence represented by SEQ ID NO: 167 or a complementary sequence thereof exhibited the highest accuracy of 93.4% in the training cohort and accuracy of 91.1% in the validation cohort.

The discriminant accuracy of the measurement using the polynucleotide consisting of the nucleotide sequence represented by SEQ ID NO: 172 or a complementary sequence thereof is shown in Table 8-14. The measurement using, alone (one), the polynucleotide consisting of the nucleotide sequence represented by SEQ ID NO: 172 or a complementary sequence thereof exhibited accuracy of 76.8% in the training cohort and accuracy of 75.8% in the validation cohort. Also, for example, the measurement using the combinations of two polynucleotides comprising at least one polynucleotide consisting of the nucleotide sequence represented by SEQ ID NO: 172 or a complementary sequence thereof exhibited the highest accuracy of 86.3% in the training cohort and accuracy of 83.7% in the validation cohort. Furthermore, for example, the measurement using the combinations of three polynucleotides comprising at least one polynucleotide consisting of the nucleotide sequence represented by SEQ ID NO: 172 or a complementary sequence thereof exhibited the highest accuracy of 90.2% in the training cohort and accuracy of 90.5% in the validation cohort. Furthermore, for example, the measurement using the combinations of four polynucleotides comprising at least one polynucleotide consisting of the nucleotide sequence represented by SEQ ID NO: 172 or a complementary sequence thereof exhibited the highest accuracy of 92.1% in the training cohort and accuracy of 93.2% in the validation cohort.

The discriminant accuracy of the measurement using the polynucleotide consisting of the nucleotide sequence represented by SEQ ID NO: 214 or a complementary sequence thereof is shown in Table 8-15. The measurement using, alone (one), the polynucleotide consisting of the nucleotide sequence represented by SEQ ID NO: 214 or a complementary sequence thereof exhibited accuracy of 69.5% in the training cohort and accuracy of 67.4% in the validation cohort. Also, for example, the measurement using the combinations of two polynucleotides comprising at least one polynucleotide consisting of the nucleotide sequence represented by SEQ ID NO: 214 or a complementary sequence thereof exhibited the highest accuracy of 89.2% in the training cohort and accuracy of 87.9% in the validation cohort. Furthermore, for example, the measurement using the combinations of three polynucleotides comprising at least one polynucleotide consisting of the nucleotide sequence represented by SEQ ID NO: 214 or a complementary sequence thereof exhibited the highest accuracy of 91.5% in the training cohort and accuracy of 90.5% in the validation cohort. Furthermore, for example, the measurement using the combinations of four polynucleotides comprising at least one polynucleotide consisting of the nucleotide sequence represented by SEQ ID NO: 214 or a complementary sequence thereof exhibited the highest accuracy of 93.4% in the training cohort and accuracy of 92.6% in the validation cohort.

The discriminant accuracy of the measurement using the polynucleotide consisting of the nucleotide sequence represented by SEQ ID NO: 714 or a complementary sequence thereof is shown in Table 8-16. The measurement using, alone (one), the polynucleotide consisting of the nucleotide sequence represented by SEQ ID NO: 714 or a complementary sequence thereof exhibited accuracy of 44.7% in the training cohort and accuracy of 46.8% in the validation cohort. Also, for example, the measurement using the combinations of two polynucleotides comprising at least one polynucleotide consisting of the nucleotide sequence represented by SEQ ID NO: 714 or a complementary sequence thereof exhibited the highest accuracy of 90.2% in the training cohort and accuracy of 87.4% in the validation cohort. Furthermore, for example, the measurement using the combinations of three polynucleotides comprising at least one polynucleotide consisting of the nucleotide sequence represented by SEQ ID NO: 714 or a complementary sequence thereof exhibited the highest accuracy of 92.1% in the training cohort and accuracy of 91.1% in the validation cohort. Furthermore, for example, the measurement using the combinations of four polynucleotides comprising at least one polynucleotide consisting of the nucleotide sequence represented by SEQ ID NO: 714 or a complementary sequence thereof exhibited the highest accuracy of 94.4% in the training cohort and accuracy of 94.2% in the validation cohort.

The discriminant accuracy of the measurement using the polynucleotide consisting of the nucleotide sequence represented by SEQ ID NO: 715 or a complementary sequence thereof is shown in Table 8-17. The measurement using, alone (one), the polynucleotide consisting of the nucleotide sequence represented by SEQ ID NO: 715 or a complementary sequence thereof exhibited accuracy of 64.2% in the training cohort and accuracy of 65.8% in the validation cohort. Also, for example, the measurement using the combinations of two polynucleotides comprising at least one polynucleotide consisting of the nucleotide sequence represented by SEQ ID NO: 715 or a complementary sequence thereof exhibited the highest accuracy of 87.9% in the training cohort and accuracy of 86.8% in the validation cohort. Furthermore, for example, the measurement using the combinations of three polynucleotides SEQ ID NO: 715 or a complementary sequence thereof exhibited the highest accuracy of 91.8% in the training cohort and accuracy of 91.1% in the validation cohort. Furthermore, for example, the measurement using the combinations of four polynucleotides comprising at least one polynucleotide consisting of the nucleotide sequence represented by SEQ ID NO: 715 or a complementary sequence thereof exhibited the highest accuracy of 93.9% in the training cohort and accuracy of 93.2% in the validation cohort.

The discriminant accuracy of the measurement using the polynucleotide consisting of the nucleotide sequence represented by SEQ ID NO: 716 or a complementary sequence thereof is shown in Table 8-18. The measurement using, alone (one), the polynucleotide consisting of the nucleotide sequence represented by SEQ ID NO: 716 or a complementary sequence thereof exhibited accuracy of 62.6% in the training cohort and accuracy of 58.9% in the validation cohort. Also, for example, the measurement using the combinations of two polynucleotides comprising at least one polynucleotide consisting of the nucleotide sequence represented by SEQ ID NO: 716 or a complementary sequence thereof exhibited the highest accuracy of 90.2% in the training cohort and accuracy of 86.3% in the validation cohort. Furthermore, for example, the measurement using the combinations of three polynucleotides comprising at least one polynucleotide consisting of the nucleotide sequence represented by SEQ ID NO: 716 or a complementary sequence thereof exhibited the highest accuracy of 91.3% in the training cohort and accuracy of 91.6% in the validation cohort. Furthermore, for example, the measurement using the combinations of four polynucleotides comprising at least one polynucleotide consisting of the nucleotide sequence represented by SEQ ID NO: 716 or a complementary sequence thereof exhibited the highest accuracy of 93.7% in the training cohort and accuracy of 92.1% in the validation cohort.

The discriminant accuracy of the measurement using the polynucleotide consisting of the nucleotide sequence represented by SEQ ID NO: 717 or a complementary sequence thereof is shown in Table 8-19. The measurement using, alone (one), the polynucleotide consisting of the nucleotide sequence represented by SEQ ID NO: 717 or a complementary sequence thereof exhibited accuracy of 70.3% in the training cohort and accuracy of 66.3% in the validation cohort. Also, for example, the measurement using the combinations of two polynucleotides comprising at least one polynucleotide consisting of the nucleotide sequence represented by SEQ ID NO: 717 or a complementary sequence thereof exhibited the highest accuracy of 86.8% in the training cohort and accuracy of 84.7% in the validation cohort. Furthermore, for example, the measurement using the combinations of three polynucleotides comprising at least one polynucleotide consisting of the nucleotide sequence represented by SEQ ID NO: 717 or a complementary sequence thereof exhibited the highest accuracy of 92.3% in the training cohort and accuracy of 90.5% in the validation cohort. Furthermore, for example, the measurement using the combinations of four polynucleotides comprising at least one polynucleotide consisting of the nucleotide sequence represented by SEQ ID NO: 717 or a complementary sequence thereof exhibited the highest accuracy of 93.1% in the training cohort and accuracy of 92.6% in the validation cohort.

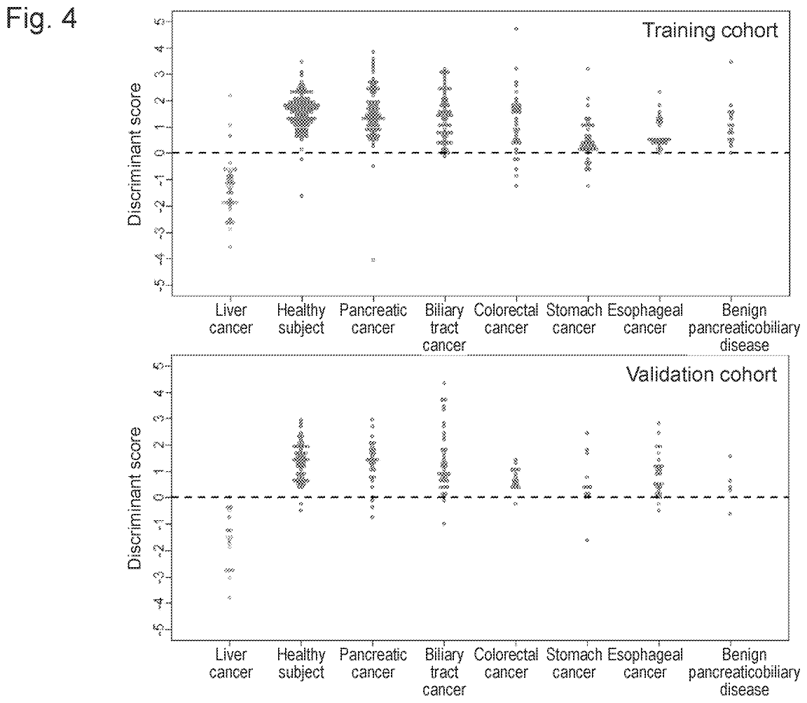

The expression level measurement values of the nucleotide sequences represented by SEQ ID NOs: 7, 9, 27, and 148 were compared among 35 liver cancer patients, 99 healthy subjects, 72 pancreatic cancer patients, 61 bile duct cancer patients, 38 stomach cancer patients, 25 esophageal cancer patients, 35 colorectal cancer patients, and 16 benign pancreaticobiliary disease patients in the training cohort. As a result, a scatter diagram that significantly separated the discriminant score of the liver cancer patient group from the discriminant scores of the other groups was obtained in the training cohort (see the upper diagram of FIG. 4). These results were also reproducible in the validation cohort (see the lower diagram of FIG. 4).

TABLE 8-1

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 1 | 71.2 | 94.3 | 68.9 | 73.2 | 100 | 70.5 |
| 1_155 | 88.1 | 91.4 | 87.8 | 90 | 88.2 | 90.2 |
| 1_7_155 | 90.2 | 88.6 | 90.4 | 90.5 | 88.2 | 90.8 |
| 1_7_9_148 | 92.3 | 91.4 | 92.4 | 93.2 | 100 | 92.5 |
| 1_9_155_172 | 91.3 | 94.3 | 91 | 91.6 | 94.1 | 91.3 |
| 1_9_148_155 | 90.2 | 91.4 | 90.1 | 90.5 | 100 | 89.6 |
| 1_155_172_715 | 91 | 91.4 | 91 | 93.2 | 100 | 92.5 |
| 1_155_164_715 | 90.8 | 94.3 | 90.4 | 93.7 | 100 | 93.1 |

TABLE 8-2

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 3 | 78.7 | 85.7 | 78 | 73.2 | 82.4 | 72.3 |
| 3_7 | 88.7 | 85.7 | 89 | 87.4 | 82.4 | 87.9 |
| 3_7_718 | 91.8 | 88.6 | 92.2 | 87.9 | 88.2 | 87.9 |
| 3_7_9_148 | 92.9 | 88.6 | 93.3 | 93.2 | 94.1 | 93.1 |
| 3_22_27_46 | 90.8 | 91.4 | 90.7 | 91.1 | 94.1 | 90.8 |
| 1_3_29_155 | 91 | 88.6 | 91.3 | 95.3 | 94.1 | 95.4 |
| 1_3_151_155 | 90.7 | 88.6 | 91 | 95.8 | 94.1 | 96 |
| 3_7_148_715 | 92.3 | 88.6 | 92.7 | 90 | 94.1 | 89.6 |

TABLE 8-3

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 7 | 85.5 | 85.7 | 85.5 | 84.7 | 82.4 | 85 |
| 7_148 | 91.5 | 85.7 | 92.1 | 90.5 | 88.2 | 90.8 |
| 7_9_148 | 93.7 | 91.4 | 93.9 | 92.1 | 100 | 91.3 |
| 7_28_148_717 | 94.2 | 91.4 | 94.5 | 92.1 | 100 | 91.3 |
| 7_9_148_186 | 93.4 | 91.4 | 93.6 | 91.6 | 94.1 | 91.3 |
| 7_148_172_715 | 92.1 | 88.6 | 92.4 | 92.6 | 100 | 91.9 |
| 7_9_148_723 | 93.4 | 91.4 | 93.6 | 92.1 | 100 | 91.3 |
| 7_9_28_148 | 94.4 | 91.4 | 94.8 | 92.6 | 100 | 91.9 |

TABLE 8-4

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 9 | 59.7 | 62.9 | 59.4 | 59.5 | 94.1 | 56.1 |
| 7_9 | 86 | 88.6 | 85.8 | 81.1 | 82.4 | 80.9 |
| 7_9_714 | 91.8 | 85.7 | 92.4 | 84.7 | 76.5 | 85.5 |
| 7_9_148_157 | 93.4 | 91.4 | 93.6 | 92.1 | 100 | 91.3 |
| 7_9_148_722 | 93.9 | 91.4 | 94.2 | 91.6 | 94.1 | 91.3 |
| 7_9_27_148 | 94.7 | 91.4 | 95 | 92.1 | 94.1 | 91.9 |
| 7_9_148_725 | 93.7 | 91.4 | 93.9 | 92.1 | 100 | 91.3 |
| 7_9_148_729 | 93.7 | 91.4 | 93.9 | 91.1 | 94.1 | 90.8 |

TABLE 8-5

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 22 | 76.5 | 77.1 | 76.5 | 78.9 | 76.5 | 79.2 |
| 3_22 | 85.8 | 88.6 | 85.5 | 84.7 | 88.2 | 84.4 |
| 7_22_148 | 91.3 | 88.6 | 91.5 | 91.6 | 88.2 | 91.9 |
| 7_9_22_148 | 93.7 | 91.4 | 93.9 | 93.7 | 100 | 93.1 |
| 7_22_28_148 | 93.7 | 91.4 | 93.9 | 92.6 | 94.1 | 92.5 |
| 7_22_148_189 | 91.8 | 85.7 | 92.4 | 92.1 | 88.2 | 92.5 |
| 2_7_22_148 | 92.1 | 91.4 | 92.1 | 92.6 | 100 | 91.9 |
| 7_22_148_720 | 92.3 | 82.9 | 93.3 | 93.2 | 88.2 | 93.6 |

TABLE 8-6

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 38 | 65.5 | 51.4 | 67 | 65.8 | 76.5 | 64.7 |
| 7_38 | 86.3 | 85.7 | 86.3 | 84.2 | 82.4 | 84.4 |
| 7_38_148 | 92.3 | 88.6 | 92.7 | 91.6 | 94.1 | 91.3 |
| 7_9_38_148 | 94.2 | 91.4 | 94.5 | 92.1 | 100 | 91.3 |
| 7_38_51_148 | 93.1 | 88.6 | 93.6 | 91.6 | 94.1 | 91.3 |
| 7_38_148_718 | 92.9 | 88.6 | 93.3 | 92.6 | 94.1 | 92.5 |
| 7_38_148_216 | 92.3 | 88.6 | 92.7 | 93.2 | 94.1 | 93.1 |
| 7_38_148_728 | 91.5 | 88.6 | 91.8 | 92.1 | 94.1 | 91.9 |

TABLE 8-7

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 44 | 62.6 | 62.9 | 62.6 | 62.1 | 94.1 | 59 |
| 7_44 | 90.5 | 85.7 | 91 | 86.3 | 88.2 | 86.1 |
| 7_44_148 | 92.9 | 91.4 | 93 | 91.1 | 100 | 90.2 |
| 7_9_44_148 | 93.7 | 91.4 | 93.9 | 91.6 | 100 | 90.8 |
| 7_44_123_148 | 93.4 | 91.4 | 93.6 | 91.1 | 100 | 90.2 |
| 7_38_44_148 | 92.9 | 91.4 | 93 | 91.1 | 100 | 90.2 |
| 7_44_148_723 | 93.1 | 91.4 | 93.3 | 91.1 | 100 | 90.2 |
| 7_44_48_148 | 93.7 | 91.4 | 93.9 | 92.1 | 100 | 91.3 |

TABLE 8-8

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 134 | 53.4 | 45.7 | 54.2 | 58.9 | 64.7 | 58.4 |
| 7_134 | 87.3 | 85.7 | 87.5 | 84.2 | 76.5 | 85 |
| 7_134_148 | 92.9 | 88.6 | 93.3 | 91.1 | 100 | 90.2 |
| 7_9_134_148 | 93.7 | 91.4 | 93.9 | 92.1 | 100 | 91.3 |
| 7_134_148_724 | 93.4 | 88.6 | 93.9 | 93.7 | 94.1 | 93.6 |
| 7_22_134_148 | 92.3 | 91.4 | 92.4 | 93.7 | 100 | 93.1 |
| 7_134_148_189 | 92.9 | 88.6 | 93.3 | 91.6 | 100 | 90.8 |
| 7_134_148_714 | 92.6 | 85.7 | 93.3 | 90 | 94.1 | 89.6 |

TABLE 8-9

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 148 | 73.6 | 85.7 | 72.4 | 75.3 | 82.4 | 74.6 |
| 48_148 | 86.3 | 88.6 | 86 | 85.3 | 88.2 | 85 |
| 7_28_148 | 93.7 | 85.7 | 94.5 | 91.6 | 94.1 | 91.3 |
| 7_9_148_726 | 93.7 | 91.4 | 93.9 | 92.1 | 100 | 91.3 |
| 7_9_148_151 | 93.6 | 91.4 | 93.9 | 93.7 | 94.1 | 93.6 |
| 7_9_109_148 | 93.7 | 91.4 | 93.9 | 92.1 | 100 | 91.3 |
| 5_7_9_148 | 92.9 | 91.4 | 93 | 93.2 | 100 | 92.5 |
| 7_9_76_148 | 93.4 | 91.4 | 93.6 | 91.6 | 100 | 90.8 |

TABLE 8-10

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 155 | 60.8 | 65.7 | 60.3 | 58.9 | 64.7 | 58.4 |
| 7_155 | 86.5 | 85.7 | 86.6 | 85.8 | 82.4 | 86.1 |
| 7_148_155 | 90.5 | 85.7 | 91 | 91.6 | 88.2 | 91.9 |
| 7_9_148_155 | 93.4 | 91.4 | 93.6 | 91.6 | 100 | 90.8 |
| 7_38_148_155 | 93.4 | 88.6 | 93.9 | 93.2 | 94.1 | 93.1 |
| 1_9_155_167 | 90 | 94.3 | 89.5 | 92.6 | 100 | 91.9 |
| 1_3_155_715 | 89.7 | 88.6 | 89.8 | 93.2 | 100 | 92.5 |
| 1_3_38_155 | 90 | 88.6 | 90.1 | 93.7 | 94.1 | 93.6 |

TABLE 8-11

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 157 | 70.3 | 71.4 | 70.1 | 68.9 | 94.1 | 66.5 |
| 7_157 | 86.5 | 85.7 | 86.6 | 83.2 | 82.4 | 83.2 |
| 7_148_157 | 91 | 88.6 | 91.3 | 91.6 | 94.1 | 91.3 |
| 7_48_157_714 | 93.9 | 88.6 | 94.5 | 92.6 | 94.1 | 92.5 |
| 7_38_148_157 | 92.3 | 88.6 | 92.7 | 92.6 | 94.1 | 92.5 |
| 1_44_155_157 | 89.4 | 94.3 | 89 | 90.5 | 100 | 89.6 |
| 7_76_157_714 | 92.9 | 82.9 | 93.9 | 90.5 | 94.1 | 90.2 |
| 7_148_157_189 | 91.8 | 88.6 | 92.1 | 92.1 | 94.1 | 91.9 |

TABLE 8-12

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 164 | 72.4 | 82.9 | 71.3 | 65.8 | 76.5 | 64.7 |
| 7_164 | 87.6 | 85.7 | 87.8 | 87.4 | 88.2 | 87.3 |
| 7_148_164 | 91.5 | 85.7 | 92.1 | 92.1 | 94.1 | 91.9 |
| 7_9_148_164 | 92.3 | 91.4 | 92.4 | 91.1 | 94.1 | 90.8 |
| 7_76_164_714 | 91.3 | 85.7 | 91.8 | 94.2 | 94.1 | 94.2 |
| 7_38_164_714 | 92.6 | 82.9 | 93.6 | 90.5 | 82.4 | 91.3 |
| 7_38_148_164 | 92.3 | 88.6 | 92.7 | 91.6 | 94.1 | 91.3 |
| 1_7_164_714 | 90.5 | 85.7 | 91 | 94.2 | 94.1 | 94.2 |

TABLE 8-13

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 167 | 62.1 | 68.6 | 61.4 | 57.4 | 70.6 | 56.1 |
| 7_167 | 89.2 | 85.7 | 89.5 | 87.4 | 82.4 | 87.9 |
| 7_148_167 | 92.1 | 85.7 | 92.7 | 90 | 88.2 | 90.2 |
| 7_9_148_167 | 93.1 | 91.4 | 93.3 | 92.6 | 100 | 91.9 |
| 1_7_167_714 | 92.6 | 85.7 | 93.3 | 94.7 | 100 | 94.2 |
| 7_151_167_714 | 92.9 | 85.7 | 93.6 | 92.1 | 88.2 | 92.5 |
| 7_148_167_189 | 92.9 | 85.7 | 93.6 | 92.6 | 88.2 | 93.1 |
| 7_28_167_714 | 93.4 | 85.7 | 94.2 | 91.1 | 88.2 | 91.3 |

TABLE 8-14

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 172 | 76.8 | 91.4 | 75.4 | 75.8 | 82.4 | 75.1 |
| 7_172 | 86.3 | 85.7 | 86.3 | 83.7 | 76.5 | 84.4 |
| 1_155_172 | 90.2 | 94.3 | 89.8 | 90.5 | 88.2 | 90.8 |
| 7_9_148_172 | 92.1 | 91.4 | 92.1 | 93.2 | 94.1 | 93.1 |
| 7_150_172_714 | 92.1 | 85.7 | 92.7 | 92.1 | 94.1 | 91.9 |
| 7_172_714_715 | 91.3 | 82.9 | 92.2 | 92.1 | 94.1 | 91.9 |
| 7_38_155_172 | 91.3 | 91.4 | 91.3 | 89.5 | 76.5 | 90.8 |
| 1_2_155_172 | 89.7 | 94.3 | 89.2 | 91.6 | 94.1 | 91.3 |

TABLE 8-15

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 214 | 69.5 | 77.1 | 68.7 | 67.4 | 64.7 | 67.6 |
| 7_214 | 89.2 | 85.7 | 89.5 | 87.9 | 82.4 | 88.4 |
| 7_148_214 | 91.5 | 85.7 | 92.1 | 90.5 | 88.2 | 90.8 |
| 7_9_148_214 | 93.4 | 91.4 | 93.6 | 92.6 | 100 | 91.9 |
| 7_148_189_214 | 92.6 | 85.7 | 93.3 | 92.1 | 88.2 | 92.5 |
| 2_7_148_214 | 92.1 | 91.4 | 92.1 | 93.7 | 100 | 93.1 |
| 1_7_214_714 | 91 | 88.6 | 91.3 | 94.7 | 94.1 | 94.8 |
| 7_39_148_214 | 92.1 | 88.6 | 92.4 | 90 | 88.2 | 90.2 |

TABLE 8-16

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 714 | 44.7 | 31.4 | 46.1 | 46.8 | 41.2 | 47.4 |
| 7_714 | 90.2 | 82.9 | 91 | 87.4 | 82.4 | 87.9 |
| 7_157_714 | 92.1 | 85.7 | 92.7 | 91.1 | 94.1 | 90.8 |
| 7_9_148_714 | 93.4 | 91.4 | 93.6 | 92.1 | 94.1 | 91.9 |
| 7_54_148_714 | 93.4 | 88.6 | 93.9 | 95.3 | 94.1 | 95.4 |
| 7_148_151_714 | 94.4 | 88.6 | 95 | 94.2 | 94.1 | 94.2 |
| 7_38_148_714 | 93.4 | 85.7 | 94.2 | 93.2 | 94.1 | 93.1 |
| 7_28_148_714 | 93.9 | 85.7 | 94.8 | 93.7 | 94.1 | 93.6 |

TABLE 8-17

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 715 | 64.2 | 71.4 | 63.5 | 65.8 | 76.5 | 64.7 |
| 7_715 | 87.9 | 85.7 | 88.1 | 86.8 | 94.1 | 86.1 |
| 7_148_715 | 91.8 | 88.6 | 92.1 | 91.1 | 100 | 90.2 |
| 2_7_148_715 | 93.1 | 91.4 | 93.3 | 91.6 | 100 | 90.8 |
| 7_9_148_715 | 93.9 | 91.4 | 94.2 | 93.2 | 100 | 92.5 |
| 7_17_148_715 | 93.7 | 91.4 | 93.9 | 91.1 | 100 | 90.2 |
| 7_38_148_715 | 92.6 | 88.6 | 93 | 91.1 | 100 | 90.2 |
| 7_148_715_725 | 92.3 | 88.6 | 92.7 | 91.6 | 100 | 90.8 |

TABLE 8-18

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 716 | 62.6 | 80 | 60.9 | 58.9 | 70.6 | 57.8 |
| 7_716 | 90.2 | 85.7 | 90.7 | 86.3 | 76.5 | 87.3 |
| 7_148_716 | 91.3 | 85.7 | 91.8 | 91.6 | 88.2 | 91.9 |
| 7_9_148_716 | 93.7 | 91.4 | 93.9 | 92.1 | 100 | 91.3 |
| 7_148_714_716 | 93.1 | 85.7 | 93.9 | 92.1 | 88.2 | 92.5 |
| 2_7_148_716 | 91.8 | 91.4 | 91.8 | 92.6 | 100 | 91.9 |
| 7_38_148_716 | 92.6 | 88.6 | 93 | 92.1 | 94.1 | 91.9 |
| 7_148_715_716 | 91.8 | 88.6 | 92.1 | 91.6 | 100 | 90.8 |

TABLE 8-19

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 717 | 70.3 | 85.7 | 68.7 | 66.3 | 82.4 | 64.7 |
| 7_717 | 86.8 | 85.7 | 86.9 | 84.7 | 82.4 | 85 |
| 7_148_717 | 92.3 | 85.7 | 93 | 90.5 | 88.2 | 90.8 |
| 7_9_148_717 | 93.1 | 91.4 | 93.3 | 92.6 | 100 | 91.9 |
| 7_38_148_717 | 92.3 | 88.6 | 92.7 | 91.6 | 94.1 | 91.3 |
| 7_27_148_717 | 93.1 | 85.7 | 93.9 | 91.6 | 88.2 | 91.9 |
| 7_44_148_717 | 93.1 | 91.4 | 93.3 | 92.1 | 100 | 91.3 |
| 7_148_715_717 | 92.6 | 88.6 | 93 | 91.1 | 100 | 90.2 |

### Comparative Example 1

<Liver Cancer Discriminant Performance of Existing Tumor Marker in Blood>

The concentrations of the existing tumor markers AFP, CEA, CA19-9, and PIVKA-II for detecting liver cancer in blood were measured in the training cohort and the validation cohort obtained in Reference Example 1. When the concentrations of these tumor markers in blood are higher than the reference values described in Non-Patent Literature 5 (AFP: 10 ng/mL, CEA: 5 ng/mL, CA19-9: 37 U/mL, PIVKA-II: 40 mAU/mL), subjects are usually suspected of having cancer. Thus, whether or not the concentration of each tumor marker in blood exceeded its reference value was determined for each sample, and the results were assessed for the ability of these tumor markers to detect cancer in liver cancer patients. The sensitivity of each existing marker in the training cohort and the validation cohort was calculated. The results are shown in Table 5. The sensitivity of AFP, which had the highest sensitivity among the 4 existing tumor markers measured, was as low as 56.3% in the training cohort, and was as low as 53.3% in the validation cohort, demonstrating that neither of the markers are useful in the detection of liver cancer (Table 5).

On the other hand, as shown above in Tables 3 and 6 of Examples 1 and 2, it can be concluded that all of the polynucleotides consisting of the nucleotide sequences represented by SEQ ID NOs: 1 to 183 have combinations of 1 or 2 polynucleotides exhibiting sensitivity beyond the existing liver cancer markers and thus serve as excellent diagnosis markers.

As shown in these Examples and Comparative Example, the kit, etc., and the method of the present invention can detect liver cancer with higher sensitivity than the existing tumor markers and therefore permit early detection of liver cancer. As a result, surgical resection having high potentiality of radical cure can be applied, leading to drastic improvement in survival rate.

### INDUSTRIAL APPLICABILITY

According to the present invention, liver cancer can be effectively detected by a simple and inexpensive method. This enables early detection, diagnosis and treatment of liver cancer. The method of the present invention can detect liver cancer with limited invasiveness using the blood of a patient and therefore allows liver cancer to be detected conveniently and rapidly.

All publications, patents, and patent applications cited herein are incorporated herein by reference in their entirety.

1. A method for detecting liver cancer in a human subject, comprising:
measuring an expression level of hsa-miR-5787 in a blood, serum or plasma sample from the subject;
comparing the measured expression level of hsa-miR-5787 to a control expression level of hsa-miR-5787 for a healthy subject;
detecting an increased level of hsa-miR-5787 in the sample from the subject as compared to the control expression level from the sample from the healthy subject, wherein the increased level of hsa-miR-5787 indicates that the subject has liver cancer; and
wherein the method further comprises treating the subject for the liver cancer or performing a diagnostic procedure on the subject with the liver cancer;
wherein the treating comprises: surgical resection and/or liver transplantation; local therapy which involves injecting a drug through centesis or performing cauterization to kill cancer; or hepatic arterial embolization; optionally in combination with a drug therapy or radiotherapy, and
wherein the diagnostic procedure comprises a palpation or imaging test.

2. The method according to claim 1, wherein the expression level of hsa-miR-5787 in the sample is measured by using a kit or device comprising a nucleic acid(s) specifically binds to hsa-miR-5787.

3. The method according to claim 2, wherein the kit or device further comprises nucleic acid(s) capable of specifically binding to one or more polynucleotide(s) selected from the group consisting of other liver cancer markers: miR-6726-5p, miR-6515-3p, miR-4651, miR-4257, miR-3188, miR-6131, miR-6766-3p, miR-7641, miR-1249, miR-3679-3p, miR-6787-5p, miR-4454, miR-3135b, miR-6765-3p, miR-7975, miR-204-3p, miR-7977, miR-7110-5p, miR-6717-5p, miR-6870-5p, miR-663b, miR-6875-5p, miR-8072, miR-6816-5p, miR-4281, miR-6729-5p, miR-8069, miR-4706, miR-7108-5p, miR-4433b-3p, miR-6893-5p, miR-6857-5p, miR-1227-5p, miR-6741-5p, miR-451a, miR-8063, miR-3622a-5p, miR-615-5p, miR-128-1-5p, miR-6825-5p, miR-1260b, miR-4433-3p, miR-4665-5p, miR-7845-5p, miR-1908-5p, miR-6840-3p, miR-6765-5p, miR-296-5p, miR-3675-3p, miR-6781-5p, miR-423-5p, miR-3663-3p, miR-6749-5p, miR-1231, miR-4746-3p, miR-6780b-5p, miR-4758-5p, miR-3679-5p, miR-3184-5p, miR-6125, miR-6721-5p, miR-6791-5p, miR-3185, miR-1260a, miR-3197, miR-6845-5p, miR-6887-5p, miR-6738-5p, miR-6872-3p, miR-4497, miR-1229-5p, miR-6820-5p, miR-6777-5p, miR-3917, miR-5787, miR-4286, miR-6877-5p, miR-1225-3p, miR-6088, miR-6800-5p, miR-1246, miR-4467, miR-4419b, miR-1914-3p, miR-4632-5p, miR-1915-5p, miR-3940-5p, miR-1185-2-3p, miR-6746-5p, miR-5001-5p, miR-1228-5p, miR-5572, miR-4327, miR-4638-5p, miR-6799-5p, miR-6861-5p, miR-6727-5p, miR-4513, miR-6805-3p, miR-6808-5p, miR-4449, miR-1199-5p, miR-1275, miR-4792, miR-4443, miR-6891-5p, miR-6826-5p, miR-6807-5p, miR-7150, miR-4534, miR-4476, miR-4649-5p, miR-4525, miR-1915-3p, miR-4516, miR-4417, miR-642b-3p, miR-3141, miR-5100, miR-6848-5p, miR-4739, miR-4459, miR-1237-5p, miR-296-3p, miR-4665-3p, miR-6786-5p, miR-4258, miR-6510-5p, miR-1343-5p, miR-1247-3p, miR-6805-5p, miR-4492, miR-1469, miR-1268b, miR-6858-5p, miR-3937, miR-939-5p, miR-3656, miR-744-5p, miR-4687-3p, miR-4763-3p, miR-3620-5p, miR-3195, miR-6842-5p, miR-4707-5p, miR-642a-3p, miR-7113-3p, miR-4728-5p, miR-5195-3p, miR-1185-1-3p, miR-6774-5p, miR-8059, miR-3131, miR-7847-3p, miR-4463, miR-128-2-5p, miR-4508, miR-6806-5p, miR-7111-5p, miR-6782-5p, miR-4734, miR-3162-5p, miR-887-3p, miR-6752-5p, miR-6757-5p, miR-4448, miR-671-5p, miR-3178, miR-4725-3p, miR-940, miR-6789-5p, miR-4484, miR-4634, miR-4745-5p, miR-4730, miR-6803-5p, miR-6798-5p, miR-3648, miR-4783-3p, and miR-6836-3p; and/or miR-23b-3p, miR-23a-3p, miR-625-3p, miR-1228-3p, miR-614, miR-1913, miR-92a-2-5p, miR-187-5p, miR-16-5p, miR-92b-3p, miR-150-3p, miR-564, miR-125a-3p, miR-92b-5p, miR-92a-3p, miR-663a, miR-4688, miR-4648, miR-6085, miR-6126, miR-6880-5p, miR-328-5p, miR-6768-5p, miR-3180, miR-6087, miR-1273g-3p, miR-1225-5p, miR-3196, miR-4695-5p, miR-6732-5p, miR-638, miR-6813-5p, miR-665, miR-486-3p, miR-4466, miR-30c-1-3p, miR-3621, miR-6743-5p, miR-4298, miR-4741, miR-3619-3p, miR-6824-5p, miR-5698, miR-371a-5p, miR-4488, miR-1233-5p, miR-4723-5p, miR-24-3p, miR-1238-5p, miR-4442, miR-3928-3p, miR-6716-5p, miR-6089, miR-6124, miR-6778-5p, miR-557 and miR-6090.

4. The method according to claim 1, comprising performing the diagnostic procedure on the subject.


SEQUENCE LISTING (765 sequences)

SEQ ID NO: 1 - RNA, 22 nt, Homo sapiens
  source 1..22: mol_type = transcribed RNA
ctcctggggcccgcactctcgc

SEQ ID NO: 2 - RNA, 21 nt, Homo sapiens
  source 1..21: mol_type = transcribed RNA
cgggagctggggtctgcaggt

SEQ ID NO: 3 - RNA, 20 nt, Homo sapiens
  source 1..20: mol_type = transcribed RNA
tctcttcatctaccccccag

SEQ ID NO: 4 - RNA, 20 nt, Homo sapiens
  source 1..20: mol_type = transcribed RNA
cggggtgggtgaggtcgggc

SEQ ID NO: 5 - RNA, 18 nt, Homo sapiens
  source 1..18: mol_type = transcribed RNA
ccagaggtggggactgag

SEQ ID NO: 6 - RNA, 23 nt, Homo sapiens
  source 1..23: mol_type = transcribed RNA
agaggctttgtgcggatacgggg

SEQ ID NO: 7 - RNA, 19 nt, Homo sapiens
  source 1..19: mol_type = transcribed RNA
ggctggtcagatgggagtg

SEQ ID NO: 8 - RNA, 22 nt, Homo sapiens
  source 1..22: mol_type = transcribed RNA
tgattgtcttcccccaccctca

SEQ ID NO: 9 - RNA, 19 nt, Homo sapiens
  source 1..19: mol_type = transcribed RNA
ttgatctcggaagctaagc

SEQ ID NO: 10 - RNA, 22 nt, Homo sapiens
  source 1..22: mol_type = transcribed RNA
acgcccttcccccccttcttca

SEQ ID NO: 11 - RNA, 22 nt, Homo sapiens
  source 1..22: mol_type = transcribed RNA
cttccccccagtaatcttcatc

SEQ ID NO: 12 - RNA, 22 nt, Homo sapiens
  source 1..22: mol_type = transcribed RNA
tggcgggggtagagctggctgc

SEQ ID NO: 13 - RNA, 20 nt, Homo sapiens
  source 1..20: mol_type = transcribed RNA
ggatccgagtcacggcacca

SEQ ID NO: 14 - RNA, 22 nt, Homo sapiens
  source 1..22: mol_type = transcribed RNA
ggctggagcgagtgcagtggtg

SEQ ID NO: 15 - RNA, 21 nt, Homo sapiens
  source 1..21: mol_type = transcribed RNA
tcacctggctggcccgcccag

SEQ ID NO: 16 - RNA, 18 nt, Homo sapiens
  source 1..18: mol_type = transcribed RNA
atcctagtcacggcacca

SEQ ID NO: 17 - RNA, 21 nt, Homo sapiens
  source 1..21: mol_type = transcribed RNA
gctgggaaggcaaagggacgt

SEQ ID NO: 18 - RNA, 18 nt, Homo sapiens
  source 1..18: mol_type = transcribed RNA
ttcccagccaacgcacca

SEQ ID NO: 19 - RNA, 21 nt, Homo sapiens
  source 1..21: mol_type = transcribed RNA
tgggggtgtggggagagagag

SEQ ID NO: 20 - RNA, 22 nt, Homo sapiens
  source 1..22: mol_type = transcribed RNA
aggcgatgtggggatgtagaga

SEQ ID NO: 21 - RNA, 19 nt, Homo sapiens
  source 1..19: mol_type = transcribed RNA
tgggggagatgggggttga

SEQ ID NO: 22 - RNA, 22 nt, Homo sapiens
  source 1..22: mol_type = transcribed RNA
ggtggcccggccgtgcctgagg

SEQ ID NO: 23 - RNA, 21 nt, Homo sapiens
  source 1..21: mol_type = transcribed RNA
tgagggacccaggacaggaga

SEQ ID NO: 24 - RNA, 20 nt, Homo sapiens
  source 1..20: mol_type = transcribed RNA
ggcggcggggaggtaggcag

SEQ ID NO: 25 - RNA, 21 nt, Homo sapiens
  source 1..21: mol_type = transcribed RNA
tggggcggggcaggtccctgc

SEQ ID NO: 26 - RNA, 18 nt, Homo sapiens
  source 1..18: mol_type = transcribed RNA
gggtcccggggagggggg

SEQ ID NO: 27 - RNA, 22 nt, Homo sapiens
  source 1..22: mol_type = transcribed RNA
tgggcgagggcggctgagcggc

SEQ ID NO: 28 - RNA, 23 nt, Homo sapiens
  source 1..23: mol_type = transcribed RNA
ggatggttgggggcggtcggcgt

SEQ ID NO: 29 - RNA, 25 nt, Homo sapiens
  source 1..25: mol_type = transcribed RNA
agcggggaggaagtgggcgctgctt

SEQ ID NO: 30 - RNA, 21 nt, Homo sapiens
  source 1..21: mol_type = transcribed RNA
gtgtggccggcaggcgggtgg

SEQ ID NO: 31 - RNA, 21 nt, Homo sapiens
  source 1..21: mol_type = transcribed RNA
caggagtggggggtgggacgt

SEQ ID NO: 32 - RNA, 21 nt, Homo sapiens
  source 1..21: mol_type = transcribed RNA
caggcaggtgtagggtggagc

SEQ ID NO: 33 - RNA, 22 nt, Homo sapiens
  source 1..22: mol_type = transcribed RNA
ttggggattgggtcaggccagt

SEQ ID NO: 34 - RNA, 17 nt, Homo sapiens
  source 1..17: mol_type = transcribed RNA
gtggggccaggcggtgg

SEQ ID NO: 35 - RNA, 22 nt, Homo sapiens
  source 1..22: mol_type = transcribed RNA
gtgggtgctggtgggagccgtg

SEQ ID NO: 36 - RNA, 22 nt, Homo sapiens
  source 1..22: mol_type = transcribed RNA
aaaccgttaccattactgagtt

SEQ ID NO: 37 - RNA, 22 nt, Homo sapiens
  source 1..22: mol_type = transcribed RNA
tcaaaatcaggagtcggggctt

SEQ ID NO: 38 - RNA, 22 nt, Homo sapiens
  source 1..22: mol_type = transcribed RNA
caggcacgggagctcaggtgag

SEQ ID NO: 39 - RNA, 22 nt, Homo sapiens
  source 1..22: mol_type = transcribed RNA
gggggtccccggtgctcggatc

SEQ ID NO: 40 - RNA, 23 nt, Homo sapiens
  source 1..23: mol_type = transcribed RNA
cggggccgtagcactgtctgaga

SEQ ID NO: 41 - RNA, 22 nt, Homo sapiens
  source 1..22: mol_type = transcribed RNA
tggggaggtgtggagtcagcat

SEQ ID NO: 42 - RNA, 19 nt, Homo sapiens
  source 1..19: mol_type = transcribed RNA
atcccaccactgccaccat

SEQ ID NO: 43 - RNA, 21 nt, Homo sapiens
  source 1..21: mol_type = transcribed RNA
acaggagtgggggtgggacat

SEQ ID NO: 44 - RNA, 23 nt, Homo sapiens
  source 1..23: mol_type = transcribed RNA
ctgggggacgcgtgagcgcgagc

SEQ ID NO: 45 - RNA, 21 nt, Homo sapiens
  source 1..21: mol_type = transcribed RNA
aagggacagggagggtcgtgg

SEQ ID NO: 46 - RNA, 21 nt, Homo sapiens
  source 1..21: mol_type = transcribed RNA
cggcggggacggcgattggtc

SEQ ID NO: 47 - RNA, 22 nt, Homo sapiens
  source 1..22: mol_type = transcribed RNA
gcccaggactttgtgcggggtg

SEQ ID NO: 48 - RNA, 25 nt, Homo sapiens
  source 1..25: mol_type = transcribed RNA
gtgaggcggggccaggagggtgtgt

SEQ ID NO: 49 - RNA, 21 nt, Homo sapiens
  source 1..21: mol_type = transcribed RNA
agggccccccctcaatcctgt

SEQ ID NO: 50 - RNA, 22 nt, Homo sapiens
  source 1..22: mol_type = transcribed RNA
catctctaaggaactcccccaa

SEQ ID NO: 51 - RNA, 21 nt, Homo sapiens
  source 1..21: mol_type = transcribed RNA
cgggccggaggtcaagggcgt

SEQ ID NO: 52 - RNA, 23 nt, Homo sapiens
  source 1..23: mol_type = transcribed RNA
tgaggggcagagagcgagacttt

SEQ ID NO: 53 - RNA, 23 nt, Homo sapiens
  source 1..23: mol_type = transcribed RNA
tgagcaccacacaggccgggcgc

SEQ ID NO: 54 - RNA, 20 nt, Homo sapiens
  source 1..20: mol_type = transcribed RNA
gccggggctttgggtgaggg

SEQ ID NO: 55 - RNA, 22 nt, Homo sapiens
  source 1..22: mol_type = transcribed RNA
tcgggcctggggttgggggagc

SEQ ID NO: 56 - RNA, 20 nt, Homo sapiens
  source 1..20: mol_type = transcribed RNA
gtgtctgggcggacagctgc

SEQ ID NO: 57 - RNA, 21 nt, Homo sapiens
  source 1..21: mol_type = transcribed RNA
agcggtgctcctgcgggccga

SEQ ID NO: 58 - RNA, 23 nt, Homo sapiens
  source 1..23: mol_type = transcribed RNA
tggggaaggcttggcagggaaga

SEQ ID NO: 59 - RNA, 23 nt, Homo sapiens
  source 1..23: mol_type = transcribed RNA
gtgagtgggagccggtggggctg

SEQ ID NO: 60 - RNA, 23 nt, Homo sapiens
  source 1..23: mol_type = transcribed RNA
tgaggatatggcagggaagggga

SEQ ID NO: 61 - RNA, 24 nt, Homo sapiens
  source 1..24: mol_type = transcribed RNA
tgaggggcctcagaccgagctttt

SEQ ID NO: 62 - RNA, 20 nt, Homo sapiens
  source 1..20: mol_type = transcribed RNA
gcggaaggcggagcggcgga

SEQ ID NO: 63 - RNA, 23 nt, Homo sapiens
  source 1..23: mol_type = transcribed RNA
tgggcaggggcttattgtaggag

SEQ ID NO: 64 - RNA, 22 nt, Homo sapiens
  source 1..22: mol_type = transcribed RNA
cccctggggctgggcaggcgga

SEQ ID NO: 65 - RNA, 23 nt, Homo sapiens
  source 1..23: mol_type = transcribed RNA
agaagaaggcggtcggtctgcgg

SEQ ID NO: 66 - RNA, 18 nt, Homo sapiens
  source 1..18: mol_type = transcribed RNA
atcccacctctgccacca

SEQ ID NO: 67 - RNA, 23 nt, Homo sapiens
  source 1..23: mol_type = transcribed RNA
ggaggcgcaggctcggaaaggcg

SEQ ID NO: 68 - RNA, 19 nt, Homo sapiens
  source 1..19: mol_type = transcribed RNA
cggggccagagcagagagc

SEQ ID NO: 69 - RNA, 23 nt, Homo sapiens
  source 1..23: mol_type = transcribed RNA
tggggggacagatggagaggaca

SEQ ID NO: 70 - RNA, 23 nt, Homo sapiens
  source 1..23: mol_type = transcribed RNA
cgaggggtagaagagcacagggg

SEQ ID NO: 71 - RNA, 21 nt, Homo sapiens
  source 1..21: mol_type = transcribed RNA
cccatgcctcctgccgcggtc

SEQ ID NO: 72 - RNA, 17 nt, Homo sapiens
  source 1..17: mol_type = transcribed RNA
ctccgggacggctgggc

SEQ ID NO: 73 - RNA, 24 nt, Homo sapiens
  source 1..24: mol_type = transcribed RNA
gtgggtagggtttgggggagagcg

SEQ ID NO: 74 - RNA, 19 nt, Homo sapiens
  source 1..19: mol_type = transcribed RNA
tgcggcagagctggggtca

SEQ ID NO: 75 - RNA, 23 nt, Homo sapiens
  source 1..23: mol_type = transcribed RNA
acggggagtcaggcagtggtgga

SEQ ID NO: 76 - RNA, 20 nt, Homo sapiens
  source 1..20: mol_type = transcribed RNA
gctcggactgagcaggtggg

SEQ ID NO: 77 - RNA, 20 nt, Homo sapiens
  source 1..20: mol_type = transcribed RNA
gggctggggcgcggggaggt

SEQ ID NO: 78 - RNA, 17 nt, Homo sapiens
  source 1..17: mol_type = transcribed RNA
accccactcctggtacc

SEQ ID NO: 79 - RNA, 22 nt, Homo sapiens
  source 1..22: mol_type = transcribed RNA
agggccgaagggtggaagctgc

SEQ ID NO: 80 - RNA, 22 nt, Homo sapiens
  source 1..22: mol_type = transcribed RNA
tgagcccctgtgccgcccccag

SEQ ID NO: 81 - RNA, 20 nt, Homo sapiens
  source 1..20: mol_type = transcribed RNA
agagatgaagcgggggggcg

SEQ ID NO: 82 - RNA, 21 nt, Homo sapiens
  source 1..21: mol_type = transcribed RNA
gtaggtgacagtcaggggcgg

SEQ ID NO: 83 - RNA, 19 nt, Homo sapiens
  source 1..19: mol_type = transcribed RNA
aatggatttttggagcagg

SEQ ID NO: 84 - RNA, 22 nt, Homo sapiens
  source 1..22: mol_type = transcribed RNA
tggcggcggtagttatgggctt

SEQ ID NO: 85 - RNA, 18 nt, Homo sapiens
  source 1..18: mol_type = transcribed RNA
gaggctgaaggaagatgg

SEQ ID NO: 86 - RNA, 22 nt, Homo sapiens
  source 1..22: mol_type = transcribed RNA
ggaggggtcccgcactgggagg

SEQ ID NO: 87 - RNA, 23 nt, Homo sapiens
  source 1..23: mol_type = transcribed RNA
gagggcagcgtgggtgtggcgga

SEQ ID NO: 88 - RNA, 22 nt, Homo sapiens
  source 1..22: mol_type = transcribed RNA
accttgccttgctgcccgggcc

SEQ ID NO: 89 - RNA, 20 nt, Homo sapiens
  source 1..20: mol_type = transcribed RNA
gtgggttggggcgggctctg

SEQ ID NO: 90 - RNA, 22 nt, Homo sapiens
  source 1..22: mol_type = transcribed RNA
atatacagggggagactctcat

SEQ ID NO: 91 - RNA, 22 nt, Homo sapiens
  source 1..22: mol_type = transcribed RNA
ccgggagaaggaggtggcctgg

SEQ ID NO: 92 - RNA, 24 nt, Homo sapiens
  source 1..24: mol_type = transcribed RNA
agggctggactcagcggcggagct

SEQ ID NO: 93 - RNA, 21 nt, Homo sapiens
  source 1..21: mol_type = transcribed RNA
gtgggcgggggcaggtgtgtg

SEQ ID NO: 94 - RNA, 21 nt, Homo sapiens
  source 1..21: mol_type = transcribed RNA
gttggggtgcaggggtctgct

SEQ ID NO: 95 - RNA, 19 nt, Homo sapiens
  source 1..19: mol_type = transcribed RNA
ggcttgcatgggggactgg

SEQ ID NO: 96 - RNA, 21 nt, Homo sapiens
  source 1..21: mol_type = transcribed RNA
actcggctgcggtggacaagt

SEQ ID NO: 97 - RNA, 20 nt, Homo sapiens
  source 1..20: mol_type = transcribed RNA
ggggaggtgtgcagggctgg

SEQ ID NO: 98 - RNA, 22 nt, Homo sapiens
  source 1..22: mol_type = transcribed RNA
actgggtaggtggggctccagg

SEQ ID NO: 99 - RNA, 23 nt, Homo sapiens
  source 1..23: mol_type = transcribed RNA
ctcggggcaggcggctgggagcg

SEQ ID NO: 100 - RNA, 22 nt, Homo sapiens
  source 1..22: mol_type = transcribed RNA
agactgacggctggaggcccat

SEQ ID NO: 101 - RNA, 23 nt, Homo sapiens
  source 1..23: mol_type = transcribed RNA
ttgctctgctcccccgcccccag

SEQ ID NO: 102 - RNA, 22 nt, Homo sapiens
  source 1..22: mol_type = transcribed RNA
caggcagggaggtgggaccatg

SEQ ID NO: 103 - RNA, 22 nt, Homo sapiens
  source 1..22: mol_type = transcribed RNA
cgtcccggggctgcgcgaggca

SEQ ID NO: 104 - RNA, 20 nt, Homo sapiens
  source 1..20: mol_type = transcribed RNA
cctgagcccgggccgcgcag

SEQ ID NO: 105 - RNA, 17 nt, Homo sapiens
  source 1..17: mol_type = transcribed RNA
gtgggggagaggctgtc

SEQ ID NO: 106 - RNA, 18 nt, Homo sapiens
  source 1..18: mol_type = transcribed RNA
cggtgagcgctcgctggc

SEQ ID NO: 107 - RNA, 17 nt, Homo sapiens
  source 1..17: mol_type = transcribed RNA
ttggaggcgtgggtttt

SEQ ID NO: 108 - RNA, 19 nt, Homo sapiens
  source 1..19: mol_type = transcribed RNA
taaggagggggatgagggg

SEQ ID NO: 109 - RNA, 23 nt, Homo sapiens
  source 1..23: mol_type = transcribed RNA
tcaataggaaagaggtgggacct

SEQ ID NO: 110 - RNA, 22 nt, Homo sapiens
  source 1..22: mol_type = transcribed RNA
gtgagccagtggaatggagagg

SEQ ID NO: 111 - RNA, 18 nt, Homo sapiens
  source 1..18: mol_type = transcribed RNA
ctggcagggggagaggta

SEQ ID NO: 112 - RNA, 17 nt, Homo sapiens
  source 1..17: mol_type = transcribed RNA
ggatggaggaggggtct

SEQ ID NO: 113 - RNA, 22 nt, Homo sapiens
  source 1..22: mol_type = transcribed RNA
caggaaggatttagggacaggc

SEQ ID NO: 114 - RNA, 24 nt, Homo sapiens
  source 1..24: mol_type = transcribed RNA
tgggcgaggggtgggctctcagag

SEQ ID NO: 115 - RNA, 21 nt, Homo sapiens
  source 1..21: mol_type = transcribed RNA
ggggggatgtgcatgctggtt

SEQ ID NO: 116 - RNA, 20 nt, Homo sapiens
  source 1..20: mol_type = transcribed RNA
ccccagggcgacgcggcggg

SEQ ID NO: 117 - RNA, 17 nt, Homo sapiens
  source 1..17: mol_type = transcribed RNA
gggagaagggtcggggc

SEQ ID NO: 118 - RNA, 18 nt, Homo sapiens
  source 1..18: mol_type = transcribed RNA
ggtgggcttcccggaggg

SEQ ID NO: 119 - RNA, 22 nt, Homo sapiens
  source 1..22: mol_type = transcribed RNA
agacacatttggagagggaccc

SEQ ID NO: 120 - RNA, 19 nt, Homo sapiens
  source 1..19: mol_type = transcribed RNA
gagggcgggtggaggagga

SEQ ID NO: 121 - RNA, 22 nt, Homo sapiens
  source 1..22: mol_type = transcribed RNA
ttcagatcccagcggtgcctct

SEQ ID NO: 122 - RNA, 23 nt, Homo sapiens
  source 1..23: mol_type = transcribed RNA
tgggggctgggatgggccatggt

SEQ ID NO: 123 - RNA, 25 nt, Homo sapiens
  source 1..25: mol_type = transcribed RNA
aagggaggaggagcggaggggccct

SEQ ID NO: 124 - RNA, 22 nt, Homo sapiens
  source 1..22: mol_type = transcribed RNA
ccaggaggcggaggaggtggag

SEQ ID NO: 125 - RNA, 21 nt, Homo sapiens
  source 1..21: mol_type = transcribed RNA
cgggggcggggccgaagcgcg

SEQ ID NO: 126 - RNA, 22 nt, Homo sapiens
  source 1..22: mol_type = transcribed RNA
gagggttgggtggaggctctcc

SEQ ID NO: 127 - RNA, 26 nt, Homo sapiens
  source 1..26: mol_type = transcribed RNA
ctcggccgcggcgcgtagcccccgcc

SEQ ID NO: 128 - RNA, 21 nt, Homo sapiens
  source 1..21: mol_type = transcribed RNA
gcggtggggccggaggggcgt

SEQ ID NO: 129 - RNA, 17 nt, Homo sapiens
  source 1..17: mol_type = transcribed RNA
ccccgccaccgccttgg

SEQ ID NO: 130 - RNA, 22 nt, Homo sapiens
  source 1..22: mol_type = transcribed RNA
cagcaggggagagagaggagtc

SEQ ID NO: 131 - RNA, 22 nt, Homo sapiens
  source 1..22: mol_type = transcribed RNA
tggggagcggcccccgggtggg

SEQ ID NO: 132 - RNA, 24 nt, Homo sapiens
  source 1..24: mol_type = transcribed RNA
ccccgggaacgtcgagactggagc

SEQ ID NO: 133 - RNA, 22 nt, Homo sapiens
  source 1..22: mol_type = transcribed RNA
tagggggcggcttgtggagtgt

SEQ ID NO: 134 - RNA, 17 nt, Homo sapiens
  source 1..17: mol_type = transcribed RNA
ggggctgggcgcgcgcc

SEQ ID NO: 135 - RNA, 22 nt, Homo sapiens
  source 1..22: mol_type = transcribed RNA
ctcggcgcggggcgcgggctcc

SEQ ID NO: 136 - RNA, 20 nt, Homo sapiens
  source 1..20: mol_type = transcribed RNA
cgggcgtggtggtgggggtg

SEQ ID NO: 137 - RNA, 22 nt, Homo sapiens
  source 1..22: mol_type = transcribed RNA
gtgaggaggggctggcagggac

SEQ ID NO: 138 - RNA, 23 nt, Homo sapiens
  source 1..23: mol_type = transcribed RNA
acaggcggctgtagcaatggggg

SEQ ID NO: 139 - RNA, 24 nt, Homo sapiens
  source 1..24: mol_type = transcribed RNA
tggggagctgaggctctgggggtg

SEQ ID NO: 140 - RNA, 17 nt, Homo sapiens
  source 1..17: mol_type = transcribed RNA
ggcgggtgcgggggtgg

SEQ ID NO: 141 - RNA, 22 nt, Homo sapiens
  source 1..22: mol_type = transcribed RNA
tgcggggctagggctaacagca

SEQ ID NO: 142 - RNA, 21 nt, Homo sapiens
  source 1..21: mol_type = transcribed RNA
tggctgttggagggggcaggc

SEQ ID NO: 143 - RNA, 24 nt, Homo sapiens
  source 1..24: mol_type = transcribed RNA
aggcaggggctggtgctgggcggg

SEQ ID NO: 144 - RNA, 22 nt, Homo sapiens
  source 1..22: mol_type = transcribed RNA
gtgggctgggctgggctgggcc

SEQ ID NO: 145 - RNA, 17 nt, Homo sapiens
  source 1..17: mol_type = transcribed RNA
cgcgccgggcccgggtt

SEQ ID NO: 146 - RNA, 22 nt, Homo sapiens
  source 1..22: mol_type = transcribed RNA
tgggggtggtctctagccaagg

SEQ ID NO: 147 - RNA, 23 nt, Homo sapiens
  source 1..23: mol_type = transcribed RNA
gccccggcgcgggcgggttctgg

SEQ ID NO: 148 - RNA, 22 nt, Homo sapiens
  source 1..22: mol_type = transcribed RNA
agacacatttggagagggaacc

SEQ ID NO: 149 - RNA, 23 nt, Homo sapiens
  source 1..23: mol_type = transcribed RNA
cctccctgcccgcctctctgcag

SEQ ID NO: 150 - RNA, 23 nt, Homo sapiens
  source 1..23: mol_type = transcribed RNA
tgggaggggagaggcagcaagca

SEQ ID NO: 151 - RNA, 21 nt, Homo sapiens
  source 1..21: mol_type = transcribed RNA
atccagttctctgagggggct

SEQ ID NO: 152 - RNA, 22 nt, Homo sapiens
  source 1..22: mol_type = transcribed RNA
atatacagggggagactcttat

SEQ ID NO: 153 - RNA, 25 nt, Homo sapiens
  source 1..25: mol_type = transcribed RNA
acttgggcaggagggaccctgtatg

SEQ ID NO: 154 - RNA, 22 nt, Homo sapiens
  source 1..22: mol_type = transcribed RNA
ggggaactgtagatgaaaaggc

SEQ ID NO: 155 - RNA, 23 nt, Homo sapiens
  source 1..23: mol_type = transcribed RNA
tcgaggactggtggaagggcctt

SEQ ID NO: 156 - RNA, 21 nt, Homo sapiens
  source 1..21: mol_type = transcribed RNA
cgtggaggacgaggaggaggc

SEQ ID NO: 157 - RNA, 17 nt, Homo sapiens
  source 1..17: mol_type = transcribed RNA
gagactggggtggggcc

SEQ ID NO: 158 - RNA, 23 nt, Homo sapiens
  source 1..23: mol_type = transcribed RNA
gggggccgatacactgtacgaga

SEQ ID NO: 159 - RNA, 17 nt, Homo sapiens
  source 1..17: mol_type = transcribed RNA
gcggggctgggcgcgcg

SEQ ID NO: 160 - RNA, 24 nt, Homo sapiens
  source 1..24: mol_type = transcribed RNA
tgtaggcatgaggcagggcccagg

SEQ ID NO: 161 - RNA, 22 nt, Homo sapiens
  source 1..22: mol_type = transcribed RNA
tgggggaggaaggacaggccat

SEQ ID NO: 162 - RNA, 25 nt, Homo sapiens
  source 1..25: mol_type = transcribed RNA
taggggtgggggaattcaggggtgt

SEQ ID NO: 163 - RNA, 22 nt, Homo sapiens
  source 1..22: mol_type = transcribed RNA
gctgcgggctgcggtcagggcg

SEQ ID NO: 164 - RNA, 23 nt, Homo sapiens
  source 1..23: mol_type = transcribed RNA
ttagggagtagaagggtggggag

SEQ ID NO: 165 - RNA, 22 nt, Homo sapiens
  source 1..22: mol_type = transcribed RNA
gtgaacgggcgccatcccgagg

SEQ ID NO: 166 - RNA, 22 nt, Homo sapiens
  source 1..22: mol_type = transcribed RNA
ggggggtgtggagccagggggc

SEQ ID NO: 167 - RNA, 23 nt, Homo sapiens
  source 1..23: mol_type = transcribed RNA
ctgggcccgcggcgggcgtgggg

SEQ ID NO: 168 - RNA, 21 nt, Homo sapiens
  source 1..21: mol_type = transcribed RNA
atcacattgccagggattacc

SEQ ID NO: 169 - RNA, 21 nt, Homo sapiens
  source 1..21: mol_type = transcribed RNA
atcacattgccagggatttcc

SEQ ID NO: 170 - RNA, 22 nt, Homo sapiens
  source 1..22: mol_type = transcribed RNA
gactatagaactttccccctca

SEQ ID NO: 171 - RNA, 20 nt, Homo sapiens
  source 1..20: mol_type = transcribed RNA
tcacacctgcctcgcccccc

SEQ ID NO: 172 - RNA, 23 nt, Homo sapiens
  source 1..23: mol_type = transcribed RNA
gaacgcctgttcttgccaggtgg

SEQ ID NO: 173 - RNA, 22 nt, Homo sapiens
  source 1..22: mol_type = transcribed RNA
tctgccccctccgctgctgcca

SEQ ID NO: 174 - RNA, 22 nt, Homo sapiens
  source 1..22: mol_type = transcribed RNA
gggtggggatttgttgcattac

SEQ ID NO: 175 - RNA, 22 nt, Homo sapiens
  source 1..22: mol_type = transcribed RNA
ggctacaacacaggacccgggc

SEQ ID NO: 176 - RNA, 22 nt, Homo sapiens
  source 1..22: mol_type = transcribed RNA
tagcagcacgtaaatattggcg

SEQ ID NO: 177 - RNA, 22 nt, Homo sapiens
  source 1..22: mol_type = transcribed RNA
tattgcactcgtcccggcctcc

SEQ ID NO: 178 - RNA, 22 nt, Homo sapiens
  source 1..22: mol_type = transcribed RNA
ctggtacaggcctgggggacag

SEQ ID NO: 179 - RNA, 19 nt, Homo sapiens
  source 1..19: mol_type = transcribed RNA
aggcacggtgtcagcaggc

SEQ ID NO: 180 - RNA, 22 nt, Homo sapiens
  source 1..22: mol_type = transcribed RNA
acaggtgaggttcttgggagcc

SEQ ID NO: 181 - RNA, 22 nt, Homo sapiens
  source 1..22: mol_type = transcribed RNA
agggacgggacgcggtgcagtg

SEQ ID NO: 182 - RNA, 22 nt, Homo sapiens
  source 1..22: mol_type = transcribed RNA
tattgcacttgtcccggcctgt

SEQ ID NO: 183 - RNA, 22 nt, Homo sapiens
  source 1..22: mol_type = transcribed RNA
aggcggggcgccgcgggaccgc

SEQ ID NO: 184 - RNA, 22 nt, Homo sapiens
  source 1..22: mol_type = transcribed RNA
taggggcagcagaggacctggg

SEQ ID NO: 185 - RNA, 20 nt, Homo sapiens
  source 1..20: mol_type = transcribed RNA
tgtgggactgcaaatgggag

SEQ ID NO: 186 - RNA, 19 nt, Homo sapiens
  source 1..19: mol_type = transcribed RNA
aaggggctgggggagcaca

SEQ ID NO: 187 - RNA, 18 nt, Homo sapiens
  source 1..18: mol_type = transcribed RNA
gtgaaggcccggcggaga

SEQ ID NO: 188 - RNA, 22 nt, Homo sapiens
  source 1..22: mol_type = transcribed RNA
tggtggaggaagagggcagctc

SEQ ID NO: 189 - RNA, 23 nt, Homo sapiens
  source 1..23: mol_type = transcribed RNA
gggggggcaggaggggctcaggg

SEQ ID NO: 190 - RNA, 23 nt, Homo sapiens
  source 1..23: mol_type = transcribed RNA
cacacaggaaaagcggggccctg

SEQ ID NO: 191 - RNA, 19 nt, Homo sapiens
  source 1..19: mol_type = transcribed RNA
tggggcggagcttccggag

SEQ ID NO: 192 - RNA, 18 nt, Homo sapiens
  source 1..18: mol_type = transcribed RNA
tgaggcgggggggcgagc

SEQ ID NO: 193 - RNA, 21 nt, Homo sapiens
  source 1..21: mol_type = transcribed RNA
accactgcactccagcctgag

SEQ ID NO: 194 - RNA, 22 nt, Homo sapiens
  source 1..22: mol_type = transcribed RNA
gtgggtacggcccagtgggggg

SEQ ID NO: 195 - RNA, 18 nt, Homo sapiens
  source 1..18: mol_type = transcribed RNA
cggggcggcaggggcctc

SEQ ID NO: 196 - RNA, 22 nt, Homo sapiens
  source 1..22: mol_type = transcribed RNA
caggaggcagtgggcgagcagg

SEQ ID NO: 197 - RNA, 20 nt, Homo sapiens
  source 1..20: mol_type = transcribed RNA
tagggggtggcaggctggcc

SEQ ID NO: 198 - RNA, 25 nt, Homo sapiens
  source 1..25: mol_type = transcribed RNA
agggatcgcgggcgggtggcggcct

SEQ ID NO: 199 - RNA, 23 nt, Homo sapiens
  source 1..23: mol_type = transcribed RNA
caggggctggggtttcaggttct

SEQ ID NO: 200 - RNA, 20 nt, Homo sapiens
  source 1..20: mol_type = transcribed RNA
accaggaggctgaggcccct

SEQ ID NO: 201 - RNA, 21 nt, Homo sapiens
  source 1..21: mol_type = transcribed RNA
cggggcagctcagtacaggat

SEQ ID NO: 202 - RNA, 18 nt, Homo sapiens
  source 1..18: mol_type = transcribed RNA
gggtgcgggccggcgggg

SEQ ID NO: 203 - RNA, 22 nt, Homo sapiens
  source 1..22: mol_type = transcribed RNA
ctgggagagggttgtttactcc

SEQ ID NO: 204 - RNA, 20 nt, Homo sapiens
  source 1..20: mol_type = transcribed RNA
cgcgggtcggggtctgcagg

SEQ ID NO: 205 - RNA, 22 nt, Homo sapiens
  source 1..22: mol_type = transcribed RNA
aaggggcagggacgggtggccc

SEQ ID NO: 206 - RNA, 22 nt, Homo sapiens
  source 1..22: mol_type = transcribed RNA
ctgggacaggaggaggaggcag

SEQ ID NO: 207 - RNA, 23 nt, Homo sapiens
  source 1..23: mol_type = transcribed RNA
cgggctgtccggaggggtcggct

SEQ ID NO: 208 - RNA, 22 nt, Homo sapiens
  source 1..22: mol_type = transcribed RNA
gggaccatcctgcctgctgtgg

SEQ ID NO: 209 - RNA, 22 nt, Homo sapiens
  source 1..22: mol_type = transcribed RNA
gtaggggaggttgggccaggga

SEQ ID NO: 210 - RNA, 22 nt, Homo sapiens
  source 1..22: mol_type = transcribed RNA
tgggggagtgcagtgattgtgg

SEQ ID NO: 211 - RNA, 20 nt, Homo sapiens
  source 1..20: mol_type = transcribed RNA
actcaaactgtgggggcact

SEQ ID NO: 212 - RNA, 18 nt, Homo sapiens
  source 1..18: mol_type = transcribed RNA
agggggcgggctccggcg

SEQ ID NO: 213 - RNA, 22 nt, Homo sapiens
  source 1..22: mol_type = transcribed RNA
agtgggaggccagggcacggca

SEQ ID NO: 214 - RNA, 24 nt, Homo sapiens
  source 1..24: mol_type = transcribed RNA
tgggggagccatgagataagagca

SEQ ID NO: 215 - RNA, 22 nt, Homo sapiens
  source 1..22: mol_type = transcribed RNA
tggctcagttcagcaggaacag

SEQ ID NO: 216 - RNA, 23 nt, Homo sapiens
  source 1..23: mol_type = transcribed RNA
gtgagtgggagccccagtgtgtg

SEQ ID NO: 217 - RNA, 17 nt, Homo sapiens
  source 1..17: mol_type = transcribed RNA
gccggacaagagggagg

SEQ ID NO: 218 - RNA, 22 nt, Homo sapiens
  source 1..22: mol_type = transcribed RNA
ggaggaaccttggagcttcggc

SEQ ID NO: 219 - RNA, 20 nt, Homo sapiens
  source 1..20: mol_type = transcribed RNA
tgggaatgggggtaagggcc

SEQ ID NO: 220 - RNA, 24 nt, Homo sapiens
  source 1..24: mol_type = transcribed RNA
ggaggccggggtggggcggggcgg

SEQ ID NO: 221 - RNA, 20 nt, Homo sapiens
  source 1..20: mol_type = transcribed RNA
gggaaaaggaagggggagga

SEQ ID NO: 222 - RNA, 22 nt, Homo sapiens
  source 1..22: mol_type = transcribed RNA
agtgggaggacaggaggcaggt

SEQ ID NO: 223 - RNA, 23 nt, Homo sapiens
  source 1..23: mol_type = transcribed RNA
gtttgcacgggtgggccttgtct

SEQ ID NO: 224 - RNA, 19 nt, Homo sapiens
  source 1..19: mol_type = transcribed RNA
ggggagcgaggggcggggc

SEQ ID NO: 225 - RNA, 84 nt, Homo sapiens
  source 1..84: mol_type = transcribed RNA
gctggcgtcggtgctggggagcggcccccgggtgggcctctgctctggcccctcctggggcccgcactctcgctctgggcccgc

SEQ ID NO: 226 - RNA, 61 nt, Homo sapiens
  source 1..61: mol_type = transcribed RNA
gggggcgggagctggggtctgcaggttcgcactgatgcctgctcgccctgtctcccgctag

SEQ ID NO: 227 - RNA, 57 nt, Homo sapiens
  source 1..57: mol_type = transcribed RNA
cattggagggtgtggaagacatctgggccaactctgatctcttcatctaccccccag

SEQ ID NO: 228 - RNA, 73 nt, Homo sapiens
  source 1..73: mol_type = transcribed RNA
cggcgacggcggggtgggtgaggtcgggccccaagactcggggtttgccgggcgcctcagttcaccgcggccg

SEQ ID NO: 229 - RNA, 86 nt, Homo sapiens
  source 1..86: mol_type = transcribed RNA
ggcttagaaacagtccctaggtaggatttggggaggagctaagaagcccctacagggcccagaggtggggactgagccttagttgg

SEQ ID NO: 230 - RNA, 85 nt, Homo sapiens
  source 1..85: mol_type = transcribed RNA
ggcgcctcctgctctgctgtgccgccagggcctcccctagcgcgccttctggagaggctttgtgcggatacggggctggaggcct

SEQ ID NO: 231 - RNA, 109 nt, Homo sapiens
  source 1..109: mol_type = transcribed RNA
tcccgcattccctctgctttggtcaggtggtgccctccttccatgggtagagccagagatggtgggttctggctggtcagatgggagtggacagagacccggggtcctc

SEQ ID NO: 232 - RNA, 72 nt, Homo sapiens
  source 1..72: mol_type = transcribed RNA
atgagcgggtgggagcagatcttattgagagttccttctcctgctcctgattgtcttcccccaccctcacag

SEQ ID NO: 233 - RNA, 61 nt, Homo sapiens
  source 1..61: mol_type = transcribed RNA
tctcgtttgatctcggaagctaagcagggttgggcctggttagtacttggatgggaaactt

SEQ ID NO: 234 - RNA, 53 nt, Homo sapiens
  source 1..53: mol_type = transcribed RNA
gtttgatctcggaagctaagcagggtcgggcctggttagtacttggatgggag

SEQ ID NO: 235 - RNA, 66 nt, Homo sapiens
  source 1..66: mol_type = transcribed RNA
gggaggagggaggagatgggccaagttccctctggctggaacgcccttcccccccttcttcacctg

SEQ ID NO: 236 - RNA, 68 nt, Homo sapiens
  source 1..68: mol_type = transcribed RNA
cgtggtgaggatatggcagggaaggggagtttccctctattcccttccccccagtaatcttcatcatg

SEQ ID NO: 237 - RNA, 61 nt, Homo sapiens
  source 1..61: mol_type = transcribed RNA
tcggctggcgggggtagagctggctgcaggcccggcccctctcagctgctgccctctccag

SEQ ID NO: 238 - RNA, 55 nt, Homo sapiens
  source 1..55: mol_type = transcribed RNA
ccggatccgagtcacggcaccaaatttcatgcgtgtccgtgtgaagagaccacca

SEQ ID NO: 239 - RNA, 68 nt, Homo sapiens
  source 1..68: mol_type = transcribed RNA
tgcccaggctggagcgagtgcagtggtgcagtcagtcctagctcactgcagcctcgaactcctgggct

SEQ ID NO: 240 - RNA, 87 nt, Homo sapiens
  source 1..87: mol_type = transcribed RNA
gtgaggcggggccaggagggtgtgtggcgtgggtgctgcggggccgtcagggtgcctgcgggacgctcacctggctggcccgcccag

SEQ ID NO: 241 - RNA, 68 nt, Homo sapiens
  source 1..68: mol_type = transcribed RNA
gtgcaaagagcaggaggacaggggatttatctcccaagggaggtcccctgatcctagtcacggcacca

SEQ ID NO: 242 - RNA, 110 nt, Homo sapiens
  source 1..110: mol_type = transcribed RNA
ggctacagtctttcttcatgtgactcgtggacttccctttgtcatcctatgcctgagaatatatgaaggaggctgggaaggcaaagggacgttcaattgtcatcactggc

SEQ ID NO: 243 - RNA, 49 nt, Homo sapiens
  source 1..49: mol_type = transcribed RNA
ttcccagccaacgcaccaaaaatgatatgggtctgttgtctggagaaac

SEQ ID NO: 244 - RNA, 86 nt, Homo sapiens
  source 1..86: mol_type = transcribed RNA
ggggctgggggtgtggggagagagagtgcacagccagctcagggattaaagctctttctctctctctctctcccacttccctgcag

SEQ ID NO: 245 - RNA, 73 nt, Homo sapiens
  source 1..73: mol_type = transcribed RNA
ctggtgtttgaggcgatgtggggatgtagagacaacttcccagtctcatttcctcatcctgccaggccaccat

SEQ ID NO: 246 - RNA, 60 nt, Homo sapiens
  source 1..60: mol_type = transcribed RNA
caaggtgggggagatgggggttgaacttcatttctcatgctcatccccatctcctttcag

SEQ ID NO: 247 - RNA, 115 nt, Homo sapiens
  source 1..115: mol_type = transcribed RNA
ggtgccgagggccgtccggcatcctaggcgggtcgctgcggtacctccctcctgtctgtggcggtgggatcccgtggccgtgttttcctggtggcccggccgtgcctgaggtttc

SEQ ID NO: 248 - RNA, 72 nt, Homo sapiens
  source 1..72: mol_type = transcribed RNA
gagtctgagggacccaggacaggagaaggcctatggtgatttgcattcttcctgccctggctccatcctcag

SEQ ID NO: 249 - RNA, 80 nt, Homo sapiens
  source 1..80: mol_type = transcribed RNA
gcgtcaagatggcggcggggaggtaggcagagcaggacgccgctgctgccgccgccaccgccgcctccgctccagtcgcc

SEQ ID NO: 250 - RNA, 66 nt, Homo sapiens
  source 1..66: mol_type = transcribed RNA
ccgagtggggcggggcaggtccctgcagggactgtgacactgaaggacctgcaccttcgcccacag

SEQ ID NO: 251 - RNA, 62 nt, Homo sapiens
  source 1..62: mol_type = transcribed RNA
gctgggggtcccccgacagtgtggagctggggccgggtcccggggaggggggttctgggcag

SEQ ID NO: 252 - RNA, 65 nt, Homo sapiens
  source 1..65: mol_type = transcribed RNA
gagggtgggcgagggcggctgagcggctccatcccccggcctgctcatccccctcgccctctcag

SEQ ID NO: 253 - RNA, 86 nt, Homo sapiens
  source 1..86: mol_type = transcribed RNA
cgcctgagcgtgcagcaggacatcttcctgacctggtaataattaggtgagaaggatggttgggggcggtcggcgtaactcaggga

SEQ ID NO: 254 - RNA, 82 nt, Homo sapiens
  source 1..82: mol_type = transcribed RNA
gctacggggagcggggaggaagtgggcgctgcttctgcgttatctggaaggagcagcccactcctgtcctgggctctgtggt

SEQ ID NO: 255 - RNA, 87 nt, Homo sapiens
  source 1..87: mol_type = transcribed RNA
gtgtggccggcaggcgggtgggcgggggcggccggtgggaaccccgccccgccccgcgcccgcactcacccgcccgtctccccacag

SEQ ID NO: 256 - RNA, 102 nt, Homo sapiens
  source 1..102: mol_type = transcribed RNA
tgtgttccctatcctccttatgtcccacccccactcctgtttgaatatttcaccagaaacaggagtggggggtgggacgtaaggaggatgggggaaagaaca

SEQ ID NO: 257 - RNA, 69 nt, Homo sapiens
  source 1..69: mol_type = transcribed RNA
ccgggcaggcaggtgtagggtggagcccactgtggctcctgactcagccctgctgccttcacctgccag

SEQ ID NO: 258 - RNA, 93 nt, Homo sapiens
  source 1..93: mol_type = transcribed RNA
gcttgttggggattgggtcaggccagtgttcaagggcccctcctctagtactccctgtttgtgttctgccactgactgagcttctccccacag

SEQ ID NO: 259 - RNA, 88 nt, Homo sapiens
  source 1..88: mol_type = transcribed RNA
gtggggccaggcggtggtgggcactgctggggtgggcacagcagccatgcagagcgggcatttgaccccgtgccacccttttccccag

SEQ ID NO: 260 - RNA, 63 nt, Homo sapiens
  source 1..63: mol_type = transcribed RNA
aatgggtgggtgctggtgggagccgtgccctggccactcattcggctctctccctcaccctag

SEQ ID NO: 261 - RNA, 72 nt, Homo sapiens
  source 1..72: mol_type = transcribed RNA
cttgggaatggcaaggaaaccgttaccattactgagtttagtaatggtaatggttctcttgctatacccaga

SEQ ID NO: 262 - RNA, 81 nt, Homo sapiens
  source 1..81: mol_type = transcribed RNA
tagaggcagtttcaacagatgtgtagacttttgatatgagaaattggtttcaaaatcaggagtcggggctttactgctttt

SEQ ID NO: 263 - RNA, 83 nt, Homo sapiens
  source 1..83: mol_type = transcribed RNA
aatagagggtgcacaggcacgggagctcaggtgaggcagggagctgagctcacctgacctcccatgcctgtgcaccctctatt

SEQ ID NO: 264 - RNA, 96 nt, Homo sapiens
  source 1..96: mol_type = transcribed RNA
ctcgggaggggcgggaggggggtccccggtgctcggatctcgagggtgcttattgttcggtccgagcctgggtctccctcttccccccaacccccc

SEQ ID NO: 265 - RNA, 82 nt, Homo sapiens
  source 1..82: mol_type = transcribed RNA
tgagctgttggattcggggccgtagcactgtctgagaggtttacatttctcacagtgaaccggtctctttttcagctgcttc

SEQ ID NO: 266 - RNA, 66 nt, Homo sapiens
  source 1..66: mol_type = transcribed RNA
gggcatggggaggtgtggagtcagcatggggctaggaggccccgcgctgacccgccttctccgcag

SEQ ID NO: 267 - RNA, 89 nt, Homo sapiens
  source 1..89: mol_type = transcribed RNA
tctccgtttatcccaccactgccaccattattgctactgttcagcaggtgctgctggtggtgatggtgatagtctggtgggggcggtgg

SEQ ID NO: 268 - RNA, 81 nt, Homo sapiens
  source 1..81: mol_type = transcribed RNA
catcctccttacgtcccaccccccactcctgtttctggtgaaatattcaaacaggagtgggggtgggacataaggaggata

SEQ ID NO: 269 - RNA, 79 nt, Homo sapiens
  source 1..79: mol_type = transcribed RNA
ctcgaggtgctgggggacgcgtgagcgcgagccgcttcctcacggctcggccgcggcgcgtagcccccgccacatcggg

SEQ ID NO: 270 - RNA, 99 nt, Homo sapiens
  source 1..99: mol_type = transcribed RNA
gcaagggacagggagggtcgtggcgacactcgcgccagctcccgggacggctgggctcgggctggtcgccgacctccgaccctccactagatgcctggc

SEQ ID NO: 271 - RNA, 80 nt, Homo sapiens
  source 1..80: mol_type = transcribed RNA
cgggaatgccgcggcggggacggcgattggtccgtatgtgtggtgccaccggccgccggctccgccccggcccccgcccc

SEQ ID NO: 272 - RNA, 71 nt, Homo sapiens
  source 1..71: mol_type = transcribed RNA
tgaccacccccgggcaaagacctgcagatcccctgttagagacgggcccaggactttgtgcggggtgccca

SEQ ID NO: 273 - RNA, 80 nt, Homo sapiens
  source 1..80: mol_type = transcribed RNA
aggacccttccagagggccccccctcaatcctgttgtgcctaattcagagggttgggtggaggctctcctgaagggctct

SEQ ID NO: 274 - RNA, 73 nt, Homo sapiens
  source 1..73: mol_type = transcribed RNA
ggatgataagttatggggcttctgtagagatttctatgagaacatctctaaggaactcccccaaactgaattc

SEQ ID NO: 275 - RNA, 64 nt, Homo sapiens
  source 1..64: mol_type = transcribed RNA
aaccccgggccggaggtcaagggcgtcgcttctccctaatgttgcctcttttccacggcctcag

SEQ ID NO: 276 - RNA, 94 nt, Homo sapiens
  source 1..94: mol_type = transcribed RNA
ataaaggaagttaggctgaggggcagagagcgagacttttctattttccaaaagctcggtctgaggcccctcagtcttgcttcctaacccgcgc

SEQ ID NO: 277 - RNA, 97 nt, Homo sapiens
  source 1..97: mol_type = transcribed RNA
cccgggaccttggtccaggcgctggtctgcgtggtgctcgggtggataagtctgatctgagcaccacacaggccgggcgccgggaccaagggggctc

SEQ ID NO: 278 - RNA, 67 nt, Homo sapiens
  source 1..67: mol_type = transcribed RNA
tacaggccggggctttgggtgagggacccccggagtctgtcacggtctcaccccaactctgccccag

SEQ ID NO: 279 - RNA, 69 nt, Homo sapiens
  source 1..69: mol_type = transcribed RNA
ggccctcgggcctggggttgggggagctctgtcctgtctcactcattgctcctcccctgcctggcccag

SEQ ID NO: 280 - RNA, 92 nt, Homo sapiens
  source 1..92: mol_type = transcribed RNA
gtcagtgtctgggcggacagctgcaggaaagggaagaccaaggcttgctgtctgtccagtctgccaccctaccctgtctgttcttgccacag

SEQ ID NO: 281 - RNA, 71 nt, Homo sapiens
  source 1..71: mol_type = transcribed RNA
gtgtctgtgccggtcccaggagaacctgcagaggcatcgggtcagcggtgctcctgcgggccgacactcac

SEQ ID NO: 282 - RNA, 79 nt, Homo sapiens
  source 1..79: mol_type = transcribed RNA
cagcctggggaaggcttggcagggaagacacatgagcagtgcctccacttcacgcctctcccttgtctcctttccctag

SEQ ID NO: 283 - RNA, 71 nt, Homo sapiens
  source 1..71: mol_type = transcribed RNA
ggtgagtgggagccggtggggctggagtaagggcacgcccggggctgccccacctgctgaccaccctcccc

SEQ ID NO: 284 - RNA, 75 nt, Homo sapiens
  source 1..75: mol_type = transcribed RNA
aagcaagactgaggggcctcagaccgagcttttggaaaatagaaaagtctcgctctctgcccctcagcctaactt

SEQ ID NO: 285 - RNA, 96 nt, Homo sapiens
  source 1..96: mol_type = transcribed RNA
gctctggggcgtgccgccgccgtcgctgccacctcccctaccgctagtggaagaagatggcggaaggcggagcggcggatctggacacccagcggt

SEQ ID NO: 286 - RNA, 87 nt, Homo sapiens
  source 1..87: mol_type = transcribed RNA
ccctcatctctgggcaggggcttattgtaggagtctctgaagagagctgtggactgacctgctttaacccttccccaggttcccatt

SEQ ID NO: 287 - RNA, 67 nt, Homo sapiens
  source 1..67: mol_type = transcribed RNA
ccagacccctggggctgggcaggcggaaagaggtctgaactgcctctgcctccttggtctccggcag

SEQ ID NO: 288 - RNA, 68 nt, Homo sapiens
  source 1..68: mol_type = transcribed RNA
gaatggaagaagaaggcggtcggtctgcgggagccaggccgcagagccatccgccttctgtccatgtc

SEQ ID NO: 289 - RNA, 73 nt, Homo sapiens
  source 1..73: mol_type = transcribed RNA
acctttccagctcatcccacctctgccaccaaaacactcatcgcggggtcagagggagtgccaaaaaaggtaa

SEQ ID NO: 290 - RNA, 73 nt, Homo sapiens
  source 1..73: mol_type = transcribed RNA
ggcgaggggaggcgcaggctcggaaaggcgcgcgaggctccaggctccttcccgatccaccgctctcctcgct

SEQ ID NO: 291 - RNA, 61 nt, Homo sapiens
  source 1..61: mol_type = transcribed RNA
aactgcggggccagagcagagagcccttgcacaccaccagcctctcctccctgtgccccag

SEQ ID NO: 292 - RNA, 65 nt, Homo sapiens
  source 1..65: mol_type = transcribed RNA
gagaatggggggacagatggagaggacacaggctggcactgaggtcccctccactttcctcctag

SEQ ID NO: 293 - RNA, 64 nt, Homo sapiens
  source 1..64: mol_type = transcribed RNA
gaaggcgaggggtagaagagcacaggggttctgataaacccttctgcctgcattctactcccag

SEQ ID NO: 294 - RNA, 62 nt, Homo sapiens
  source 1..62: mol_type = transcribed RNA
gtgggtctcgcatcaggaggcaaggccaggacccgctgacccatgcctcctgccgcggtcag

SEQ ID NO: 295 - RNA, 89 nt, Homo sapiens
  source 1..89: mol_type = transcribed RNA
acctccgggacggctgggcgccggcggccgggagatccgcgcttcctgaatcccggccggcccgcccggcgcccgtccgcccgcgggtc

SEQ ID NO: 296 - RNA, 69 nt, Homo sapiens
  source 1..69: mol_type = transcribed RNA
gtgggtagggtttgggggagagcgtgggctggggttcagggacaccctctcaccactgccctcccacag

SEQ ID NO: 297 - RNA, 62 nt, Homo sapiens
  source 1..62: mol_type = transcribed RNA
ccttctgcggcagagctggggtcaccagccctcatgtacttgtgacttctcccctgccacag

SEQ ID NO: 298 - RNA, 66 nt, Homo sapiens
  source 1..66: mol_type = transcribed RNA
tcaagacggggagtcaggcagtggtggagatggagagccctgagcctccactctcctggcccccag

SEQ ID NO: 299 - RNA, 93 nt, Homo sapiens
  source 1..93: mol_type = transcribed RNA
ggcgcttttgtgcgcgcccgggtctgttggtgctcagagtgtggtcaggcggctcggactgagcaggtgggtgcggggctcggaggaggcggc

SEQ ID NO: 300 - RNA, 55 nt, Homo sapiens
  source 1..55: mol_type = transcribed RNA
gggggctggggcgcggggaggtgctaggtcggcctcggctcccgcgccgcacccc

SEQ ID NO: 301 - RNA, 93 nt, Homo sapiens
  source 1..93: mol_type = transcribed RNA
tacttatggcaccccactcctggtaccatagtcataagttaggagatgttagagctgtgagtaccatgacttaagtgtggtggcttaaacatg

SEQ ID NO: 302 - RNA, 64 nt, Homo sapiens
  source 1..64: mol_type = transcribed RNA
agttcagggccgaagggtggaagctgctggtgctcatctcagcctctgcccttggcctccccag

SEQ ID NO: 303 - RNA, 90 nt, Homo sapiens
  source 1..90: mol_type = transcribed RNA
gtgggtacggcccagtgggggggagagggacacgccctgggctctgcccagggtgcagccggactgactgagcccctgtgccgcccccag

SEQ ID NO: 304 - RNA, 51 nt, Homo sapiens
  source 1..51: mol_type = transcribed RNA
agagatgaagcgggggggcggggtcttgctctattgcctacgctgatctca

SEQ ID NO: 305 - RNA, 82 nt, Homo sapiens
  source 1..82: mol_type = transcribed RNA
acctgtaggtgacagtcaggggcggggtgtggtggggctggggctggccccctcctcacacctctcctggcatcgcccccag

SEQ ID NO: 306 - RNA, 73 nt, Homo sapiens
  source 1..73: mol_type = transcribed RNA
tgtatccttgaatggatttttggagcaggagtggacacctgacccaaaggaaatcaatccataggctagcaat

SEQ ID NO: 307 - RNA, 63 nt, Homo sapiens
  source 1..63: mol_type = transcribed RNA
tggtggcggcggtagttatgggcttctctttctcaccagcagcccctgggccgccgcctccct

SEQ ID NO: 308 - RNA, 68 nt, Homo sapiens
  source 1..68: mol_type = transcribed RNA
ctcaggctcagtggtgcatgcttatagtcccagccactctggaggctgaaggaagatggcttgagcct

SEQ ID NO: 309 - RNA, 80 nt, Homo sapiens
  source 1..80: mol_type = transcribed RNA
cgtgtgagcccgccctgtgcccggcccacttctgcttcctcttagcgcaggaggggtcccgcactgggaggggccctcac

SEQ ID NO: 310 - RNA, 61 nt, Homo sapiens
  source 1..61: mol_type = transcribed RNA
gagggcagcgtgggtgtggcggaggcaggcgtgaccgtttgccgccctctcgctgctctag

SEQ ID NO: 311 - RNA, 80 nt, Homo sapiens
  source 1..80: mol_type = transcribed RNA
tgagaggccgcaccttgccttgctgcccgggccgtgcacccgtgggccccagggcgacgcggcgggggcggccctagcga

SEQ ID NO: 312 - RNA, 102 nt, Homo sapiens
  source 1..102: mol_type = transcribed RNA
gcttatcgaggaaaagatcgaggtgggttggggcgggctctggggatttggtctcacagcccggatcccagcccacttaccttggttactctccttccttct

SEQ ID NO: 313 - RNA, 86 nt, Homo sapiens
  source 1..86: mol_type = transcribed RNA
tttggtacttaaagagaggataccctttgtatgttcacttgattaatggcgaatatacagggggagactctcatttgcgtatcaaa

SEQ ID NO: 314 - RNA, 63 nt, Homo sapiens
  source 1..63: mol_type = transcribed RNA
cttgcccgggagaaggaggtggcctggagagctgctgtctccagccgccgcctgtctccacag

SEQ ID NO: 315 - RNA, 100 nt, Homo sapiens
  source 1..100: mol_type = transcribed RNA
agctcagggcggctgcgcagagggctggactcagcggcggagctggctgctggcctcagttctgcctctgtccaggtccttgtgacccgcccgctctcct

SEQ ID NO: 316 - RNA, 73 nt, Homo sapiens
  source 1..73: mol_type = transcribed RNA
gtgggcgggggcaggtgtgtggtgggtggtggcctgcggtgagcagggccctcacacctgcctcgccccccag

SEQ ID NO: 317 - RNA, 137 nt, Homo sapiens
  source 1..137: mol_type = transcribed RNA
agccagacaagagggtcatggggagtcactgtcaacccagagcaggcactgcccctgcgaccagcctggggcatcggttggggtgcaggggtctgctggtgatgctttccatctctttgctttgtcctgattgtagc

SEQ ID NO: 318 - RNA, 85 nt, Homo sapiens
  source 1..85: mol_type = transcribed RNA
ggcctgggtaggcttgcatgggggactgggaagagaccatgaacaggttagtccagggagttctcatcaagcctttactcagtag

SEQ ID NO: 319 - RNA, 68 nt, Homo sapiens
  source 1..68: mol_type = transcribed RNA
gactcggctgcggtggacaagtccggctccagaacctggacaccgctcagccggccgcggcaggggtc

SEQ ID NO: 320 - RNA, 69 nt, Homo sapiens
  source 1..69: mol_type = transcribed RNA
gaggaggggaggtgtgcagggctggggtcactgactctgcttcccctgccctgcatggtgtccccacag

SEQ ID NO: 321 - RNA, 64 nt, Homo sapiens
  source 1..64: mol_type = transcribed RNA
gaggcactgggtaggtggggctccagggctcctgacacctggacctctcctccccaggcccaca

SEQ ID NO: 322 - RNA, 65 nt, Homo sapiens
  source 1..65: mol_type = transcribed RNA
gggtgctcggggcaggcggctgggagcggccctcacattgatggctcctgccacctcctccgcag

SEQ ID NO: 323 - RNA, 86 nt, Homo sapiens
  source 1..86: mol_type = transcribed RNA
attctaggtggggagactgacggctggaggcccataagctgtctaaaacttcggcccccagatttctggtctccccacttcagaac

SEQ ID NO: 324 - RNA, 62 nt, Homo sapiens
  source 1..62: mol_type = transcribed RNA
tggcctagggggcggcttgtggagtgtatgggctgagccttgctctgctcccccgcccccag

SEQ ID NO: 325 - RNA, 59 nt, Homo sapiens
  source 1..59: mol_type = transcribed RNA
ggggccaggcagggaggtgggaccatgggggccttgctgtgtgaccaccgttcctgcag

SEQ ID NO: 326 - RNA, 66 nt, Homo sapiens
  source 1..66: mol_type = transcribed RNA
agcagccctcggcggcccggggggcgggcggcggtgcccgtcccggggctgcgcgaggcacaggcg

SEQ ID NO: 327 - RNA, 119 nt, Homo sapiens
  source 1..119: mol_type = transcribed RNA
agcctgcgccggagccggggcctgagcccgggccgcgcaggccgtgaactcgtcgagctgcgcgtgcggccggtgctcaacctgccgggtcctggccccgcgctcccgcgcgccctgga

SEQ ID NO: 328 - RNA, 80 nt, Homo sapiens
  source 1..80: mol_type = transcribed RNA
cctctgtgagaaagggtgtgggggagaggctgtcttgtgtctgtaagtatgccaaacttattttccccaaggcagaggga

SEQ ID NO: 329 - RNA, 74 nt, Homo sapiens
  source 1..74: mol_type = transcribed RNA
gcagcccggtgagcgctcgctggcctggcagtgcgtcggaagaacagggcgggtggggccgcgcacatctctgc

SEQ ID NO: 330 - RNA, 53 nt, Homo sapiens
  source 1..53: mol_type = transcribed RNA
ggtgggggttggaggcgtgggttttagaacctatccctttctagccctgagca

SEQ ID NO: 331 - RNA, 93 nt, Homo sapiens
  source 1..93: mol_type = transcribed RNA
gtaaggagggggatgaggggtcatatctcttctcagggaaagcaggagcccttcagcagggtcagggcccctcatcttcccctcctttcccag

SEQ ID NO: 332 - RNA, 98 nt, Homo sapiens
  source 1..98: mol_type = transcribed RNA
cttggtcaataggaaagaggtgggacctcctggcttttcctctgcagcatggctcggacctagtgcaatgtttaagctcccctctctttcctgttcag

SEQ ID NO: 333 - RNA, 92 nt, Homo sapiens
  source 1..92: mol_type = transcribed RNA
gtgagccagtggaatggagaggctgtgggcagggggagatgtgaaggaaagaactaggacccattcatccactgcattcctgcttggcccag

SEQ ID NO: 334 - RNA, 94 nt, Homo sapiens
  source 1..94: mol_type = transcribed RNA
cacggtgtcccctggtggaacctggcagggggagaggtaaggtctttcagcctctccaaagcccatggtcaggtactcaggtgggggagccctg

SEQ ID NO: 335 - RNA, 60 nt, Homo sapiens
  source 1..60: mol_type = transcribed RNA
tgtgaatgacccccttccagagccaaaatcaccagggatggaggaggggtcttgggtact

SEQ ID NO: 336 - RNA, 70 nt, Homo sapiens
  source 1..70: mol_type = transcribed RNA
aaaagcctgtccctaagtccctcccagccttccagagttggtgccaggaaggatttagggacaggctttg

SEQ ID NO: 337 - RNA, 64 nt, Homo sapiens
  source 1..64: mol_type = transcribed RNA
tctgggcgaggggtgggctctcagaggggctggcagtactgctctgaggcctgcctctccccag

SEQ ID NO: 338 - RNA, 75 nt, Homo sapiens
  source 1..75: mol_type = transcribed RNA
gtcagaggggggatgtgcatgctggttggggtgggctgcctgtggaccaatcagcgtgcacttccccaccctgaa

SEQ ID NO: 339 - RNA, 86 nt, Homo sapiens
  source 1..86: mol_type = transcribed RNA
agggagaagggtcggggcagggagggcagggcaggctctggggtggggggtctgtgagtcagccacggctctgcccacgtctcccc

SEQ ID NO: 340 - RNA, 73 nt, Homo sapiens
  source 1..73: mol_type = transcribed RNA
gaaaacaaccaggtgggcttcccggagggcggaacacccagccccagcatccagggctcacctaccacgtttg

SEQ ID NO: 341 - RNA, 77 nt, Homo sapiens
  source 1..77: mol_type = transcribed RNA
gagttgggaggttccctctccaaatgtgtcttgatcccccaccccaagacacatttggagagggaccctcccaactc

SEQ ID NO: 342 - RNA, 61 nt, Homo sapiens
  source 1..61: mol_type = transcribed RNA
tcacccggtgagggcgggtggaggaggagggtccccaccatcagccttcactgggacggga

SEQ ID NO: 343 - RNA, 119 nt, Homo sapiens
  source 1..119: mol_type = transcribed RNA
ccatgaggagctggcagtgggatggcctgggggtaggagcgtggcttctggagctagaccacatgggttcagatcccagcggtgcctctaactggccacaggaccttgggcagtcagct

SEQ ID NO: 344 - RNA, 70 nt, Homo sapiens
  source 1..70: mol_type = transcribed RNA
gtccctgggggctgggatgggccatggtgtgctctgatccccctgtggtctcttggcccccaggaactcc

SEQ ID NO: 345 - RNA, 74 nt, Homo sapiens
  source 1..74: mol_type = transcribed RNA
gggaggaagaagggaggaggagcggaggggcccttgtcttcccagagcctctcccttcctcccctccccctccc

SEQ ID NO: 346 - RNA, 66 nt, Homo sapiens
  source 1..66: mol_type = transcribed RNA
acccaggaggcggaggaggtggaggttgcagtgagccaagatcgtggcactgactccagcctgggg

SEQ ID NO: 347 - RNA, 102 nt, Homo sapiens
  source 1..102: mol_type = transcribed RNA
gtgggagggcccaggcgcgggcaggggtgggggtggcagagcgctgtcccgggggcggggccgaagcgcggcgaccgtaactccttctgctccgtcccccag

SEQ ID NO: 348 - RNA, 113 nt, Homo sapiens
  source 1..113: mol_type = transcribed RNA
gccgggtggggcggggcggcctcaggaggggcccagctcccctggatgtgctgcggtggggccggaggggcgtcacgtgcacccaagtgacgccccttctgattctgcctcag

SEQ ID NO: 349 - RNA, 91 nt, Homo sapiens
  source 1..91: mol_type = transcribed RNA
acgccccccgccccgccaccgccttggaggctgacctcttactttcggtcggtcttcttccctgggcttggtttgggggcgggggagtgtc

SEQ ID NO: 350 - RNA, 54 nt, Homo sapiens
  source 1..54: mol_type = transcribed RNA
agcagcaggggagagagaggagtcctctagacaccgactctgtctcctgcagat

SEQ ID NO: 351 - RNA, 136 nt, Homo sapiens
  source 1..136: mol_type = transcribed RNA
ccgcttgcctcgcccagcgcagccccggccgctgggcgcacccgtcccgttcgtccccggacgttgctctctaccccgggaacgtcgagactggagcgcccgaactgagccaccttcgcggaccccgagagcggcg

SEQ ID NO: 352 - RNA, 80 nt, Homo sapiens
  source 1..80: mol_type = transcribed RNA
ctgcagcgtgcttctccaggccccgcgcgcggacagacacacggacaagtcccgccaggggctgggcgcgcgccagccgg

SEQ ID NO: 353 - RNA, 47 nt, Homo sapiens
  source 1..47: mol_type = transcribed RNA
ctcggcgcggggcgcgggctccgggttggggcgagccaacgccgggg

SEQ ID NO: 354 - RNA, 50 nt, Homo sapiens
  source 1..50: mol_type = transcribed RNA
acccgggcgtggtggtgggggtgggtgcctgtaattccagctagttggga

SEQ ID NO: 355 - RNA, 67 nt, Homo sapiens
  source 1..67: mol_type = transcribed RNA
gtgaggaggggctggcagggacccctccaagttggggacggcagccagcccctgctcacccctcgcc

SEQ ID NO: 356 - RNA, 106 nt, Homo sapiens
  source 1..106: mol_type = transcribed RNA
agaagaatgcccaaccagccctcagttgctacagttccctgttgtttcagctcgacaacaacaggcggctgtagcaatggggggctggatgggcatctcaatgtgc

SEQ ID NO: 357 - RNA, 82 nt, Homo sapiens
  source 1..82: mol_type = transcribed RNA
tgtgggcagggccctggggagctgaggctctgggggtggccggggctgaccctgggcctctgctccccagtgtctgaccgcg

SEQ ID NO: 358 - RNA, 69 nt, Homo sapiens
  source 1..69: mol_type = transcribed RNA
ctttcggccagcgggacggcatccgaggtgggctaggctcgggcccgtggcgggtgcgggggtgggagg

SEQ ID NO: 359 - RNA, 98 nt, Homo sapiens
  source 1..98: mol_type = transcribed RNA
ttgggcaaggtgcggggctagggctaacagcagtcttactgaaggtttcctggaaaccacgcacatgctgttgccactaacctcaaccttactcggtc

SEQ ID NO: 360 - RNA, 80 nt, Homo sapiens
  source 1..80: mol_type = transcribed RNA
acctgaggagccagccctcctcccgcacccaaacttggagcacttgacctttggctgttggagggggcaggctcgcgggt

SEQ ID NO: 361 - RNA, 92 nt, Homo sapiens
  source 1..92: mol_type = transcribed RNA
cctgtccctcctgccctgcgcctgcccagccctcctgctctggtgactgaggaccgccaggcaggggctggtgctgggcggggggcggcggg

SEQ ID NO: 362 - RNA, 79 nt, Homo sapiens
  source 1..79: mol_type = transcribed RNA
gtgaggtgggggccagcagggagtgggctgggctgggctgggccaaggtacaaggcctcaccctgcatcccgcacccag

SEQ ID NO: 363 - RNA, 84 nt, Homo sapiens
  source 1..84: mol_type = transcribed RNA
ccgcagccgccgcgccgggcccgggttggccgctgacccccgcggggcccccggcggccggggcgggggcgggggctgccccgg

SEQ ID NO: 364 - RNA, 65 nt, Homo sapiens
  source 1..65: mol_type = transcribed RNA
agccctgggggtggtctctagccaaggctctggggtctcacccttggctggtctctgctccgcag

SEQ ID NO: 365 - RNA, 80 nt, Homo sapiens
  source 1..80: mol_type = transcribed RNA
ggttccggagccccggcgcgggcgggttctggggtgtagacgctgctggccagcccgccccagccgaggttctcggcacc

SEQ ID NO: 366 - RNA, 97 nt, Homo sapiens
  source 1..97: mol_type = transcribed RNA
atctgagttgggagggtccctctccaaatgtgtcttggggtgggggatcaagacacatttggagagggaacctcccaactcggcctctgccatcatt

SEQ ID NO: 367 - RNA, 59 nt, Homo sapiens
  source 1..59: mol_type = transcribed RNA
ctccagggagacagtgtgtgaggcctcttgccatggcctccctgcccgcctctctgcag

SEQ ID NO: 368 - RNA, 67 nt, Homo sapiens
  source 1..67: mol_type = transcribed RNA
gtgggaggggagaggcagcaagcacacagggcctgggactagcatgctgacctccctcctgccccag

SEQ ID NO: 369 - RNA, 115 nt, Homo sapiens
  source 1..115: mol_type = transcribed RNA
gagcaaaaaccagagaacaacatgggagcgttcctaacccctaaggcaactggatgggagacctgacccatccagttctctgagggggctcttgtgtgttctacaaggttgttca

SEQ ID NO: 370 - RNA, 86 nt, Homo sapiens
  source 1..86: mol_type = transcribed RNA
tttggtacttgaagagaggataccctttgtatgttcacttgattaatggcgaatatacagggggagactcttatttgcgtatcaaa

SEQ ID NO: 371 - RNA, 70 nt, Homo sapiens
  source 1..70: mol_type = transcribed RNA
tgtgcacttgggcaggagggaccctgtatgtctccccgcagcaccgtcatcgtgtccctcttgtccacag

SEQ ID NO: 372 - RNA, 81 nt, Homo sapiens
  source 1..81: mol_type = transcribed RNA
tacaggtgcaggggaactgtagatgaaaaggcttggcacttgagggaaagcctcagttcattctcattttgctcacctgtt

SEQ ID NO: 373 - RNA, 63 nt, Homo sapiens
  source 1..63: mol_type = transcribed RNA
gagtcgaggactggtggaagggcctttcccctcagaccaaggccctggccccagcttcttctc

SEQ ID NO: 374 - RNA, 103 nt, Homo sapiens
  source 1..103: mol_type = transcribed RNA
gtgtcggctgtggcgtgactgtccctctgtgtcccccactaggcccactgctcagtggagcgtggaggacgaggaggaggccgtccacgagcaatgccagcat

SEQ ID NO: 375 - RNA, 67 nt, Homo sapiens
  source 1..67: mol_type = transcribed RNA
aatagattattggtcaccacctccagtttctgaatttgtgagactggggtggggcctgagaatttgc

SEQ ID NO: 376 - RNA, 84 nt, Homo sapiens
  source 1..84: mol_type = transcribed RNA
tgtgcagtgggaaggggggccgatacactgtacgagagtgagtagcaggtctcacagtgaaccggtctctttccctactgtgtc

SEQ ID NO: 377 - RNA, 70 nt, Homo sapiens
  source 1..70: mol_type = transcribed RNA
aggacccagcggggctgggcgcgcggagcagcgctgggtgcagcgcctgcgccggcagctgcaagggccg

SEQ ID NO: 378 - RNA, 64 nt, Homo sapiens
  source 1..64: mol_type = transcribed RNA
tgctctgtaggcatgaggcagggcccaggttccatgtgatgctgaagctctgacattcctgcag

SEQ ID NO: 379 - RNA, 72 nt, Homo sapiens
  source 1..72: mol_type = transcribed RNA
ctgggggaggaaggacaggccatctgctattcgtccaccaacctgacttgatcctctcttccctcctcccag

SEQ ID NO: 380 - RNA, 69 nt, Homo sapiens
  source 1..69: mol_type = transcribed RNA
tggggtaggggtgggggaattcaggggtgtcgaactcatggctgccacctttgtgtccccatcctgcag

SEQ ID NO: 381 - RNA, 70 nt, Homo sapiens
  source 1..70: mol_type = transcribed RNA
ctcgggcccgaccgcgccggcccgcacctcccggcccggagctgcgggctgcggtcagggcgatcccggg

SEQ ID NO: 382 - RNA, 82 nt, Homo sapiens
  source 1..82: mol_type = transcribed RNA
ctgacttttttagggagtagaagggtggggagcatgaacaatgtttctcactccctacccctccactccccaaaaaagtcag

SEQ ID NO: 383 - RNA, 79 nt, Homo sapiens
  source 1..79: mol_type = transcribed RNA
gtgcagatccttgggagccctgttagactctggattttacacttggagtgaacgggcgccatcccgaggctttgcacag

SEQ ID NO: 384 - RNA, 71 nt, Homo sapiens
  source 1..71: mol_type = transcribed RNA
atggaggggggtgtggagccagggggcccaggtctacagcttctccccgctccctgcccccatactcccag

SEQ ID NO: 385 - RNA, 92 nt, Homo sapiens
  source 1..92: mol_type = transcribed RNA
cgctgcgcttctgggcccgcggcgggcgtggggctgcccgggccggtcgaccagcgcgccgtagctcccgaggcccgagccgcgacccgcgg

SEQ ID NO: 386 - RNA, 97 nt, Homo sapiens
  source 1..97: mol_type = transcribed RNA
ctcaggtgctctggctgcttgggttcctggcatgctgatttgtgacttaagattaaaatcacattgccagggattaccacgcaaccacgaccttggc

SEQ ID NO: 387 - RNA, 73 nt, Homo sapiens
  source 1..73: mol_type = transcribed RNA
ggccggctggggttcctggggatgggatttgcttcctgtcacaaatcacattgccagggatttccaaccgacc

SEQ ID NO: 388 - RNA, 85 nt, Homo sapiens
  source 1..85: mol_type = transcribed RNA
agggtagagggatgagggggaaagttctatagtcctgtaattagatctcaggactatagaactttccccctcatccctctgccct

SEQ ID NO: 389 - RNA, 90 nt, Homo sapiens
  source 1..90: mol_type = transcribed RNA
tctaagaaacgcagtggtctctgaagcctgcaggggcaggccagccctgcactgaacgcctgttcttgccaggtggcagaaggttgctgc

SEQ ID NO: 390 - RNA, 80 nt, Homo sapiens
  source 1..80: mol_type = transcribed RNA
acctctacctcccggcagaggaggctgcagaggctggctttccaaaactctgccccctccgctgctgccaagtggctggt

SEQ ID NO: 391 - RNA, 75 nt, Homo sapiens
  source 1..75: mol_type = transcribed RNA
tcatccctgggtggggatttgttgcattacttgtgttctatataaagtattgcacttgtcccggcctgtggaaga

SEQ ID NO: 392 - RNA, 109 nt, Homo sapiens
  source 1..109: mol_type = transcribed RNA
ggtcgggctcaccatgacacagtgtgagacctcgggctacaacacaggacccgggcgctgctctgacccctcgtgtcttgtgttgcagccggagggacgcaggtccgca

SEQ ID NO: 393 - RNA, 89 nt, Homo sapiens
  source 1..89: mol_type = transcribed RNA
gtcagcagtgccttagcagcacgtaaatattggcgttaagattctaaaattatctccagtattaactgtgctgctgaagtaaggttgac

SEQ ID NO: 394 - RNA, 81 nt, Homo sapiens
  source 1..81: mol_type = transcribed RNA
gttccactctagcagcacgtaaatattggcgtagtgaaatatatattaaacaccaatattactgtgctgctttagtgtgac

SEQ ID NO: 395 - RNA, 96 nt, Homo sapiens
  source 1..96: mol_type = transcribed RNA
cgggccccgggcgggcgggagggacgggacgcggtgcagtgttgttttttcccccgccaatattgcactcgtcccggcctccggcccccccggccc

SEQ ID NO: 396 - RNA, 84 nt, Homo sapiens
  source 1..84: mol_type = transcribed RNA
ctccccatggccctgtctcccaacccttgtaccagtgctgggctcagaccctggtacaggcctgggggacagggacctggggac

SEQ ID NO: 397 - RNA, 94 nt, Homo sapiens
  source 1..94: mol_type = transcribed RNA
cgggcagcgggtgccaggcacggtgtcagcaggcaacatggccgagaggccggggcctccgggcggcgccgtgtccgcgaccgcgtaccctgac

SEQ ID NO: 398 - RNA, 86 nt, Homo sapiens
  source 1..86: mol_type = transcribed RNA
tgccagtctctaggtccctgagaccctttaacctgtgaggacatccagggtcacaggtgaggttcttgggagcctggcgtctggcc

SEQ ID NO: 399 - RNA, 78 nt, Homo sapiens
  source 1..78: mol_type = transcribed RNA
ctttctacacaggttgggatcggttgcaatgctgtgtttctgtatggtattgcacttgtcccggcctgttgagtttgg

SEQ ID NO: 400 - RNA, 93 nt, Homo sapiens
  source 1..93: mol_type = transcribed RNA
ccttccggcgtcccaggcggggcgccgcgggaccgccctcgtgtctgtggcggtgggatcccgcggccgtgttttcctggtggcccggccatg

SEQ ID NO: 401 - RNA, 83 nt, Homo sapiens
  source 1..83: mol_type = transcribed RNA
gtctactcccagggtgccaagctgtttcgtgttccctccctaggggatcccaggtaggggcagcagaggacctgggcctggac

SEQ ID NO: 402 - RNA, 72 nt, Homo sapiens
  source 1..72: mol_type = transcribed RNA
tgtgggactgcaaatgggagctcagcacctgcctgccacccacgcagaccagcccctgctctgttcccacag

SEQ ID NO: 403 - RNA, 110 nt, Homo sapiens
  source 1..110: mol_type = transcribed RNA
gtctaccaggtgtgggcccagctttacatagttcatgctgaggccgggatttcatgcagaaaactggttgcaaaaggtgctgaaggggctgggggagcacaagggagaag

SEQ ID NO: 404 - RNA, 89 nt, Homo sapiens
  source 1..89: mol_type = transcribed RNA
agcctgtgggaaagagaagagcagggcagggtgaaggcccggcggagacactctgcccaccccacaccctgcctatgggccacacagct

SEQ ID NO: 405 - RNA, 62 nt, Homo sapiens
  source 1..62: mol_type = transcribed RNA
gagggtggtggaggaagagggcagctcccatgactgcctgaccgccttctctcctcccccag

SEQ ID NO: 406 - RNA, 75 nt, Homo sapiens
  source 1..75: mol_type = transcribed RNA
tggagtgggggggcaggaggggctcagggagaaagtgcatacagcccctggccctctctgcccttccgtcccctg

SEQ ID NO: 407 - RNA, 72 nt, Homo sapiens
  source 1..72: mol_type = transcribed RNA
ccaggcacacaggaaaagcggggccctgggttcggctgctaccccaaaggccacattctcctgtgcacacag

SEQ ID NO: 408 - RNA, 153 nt, Homo sapiens
  source 1..153: mol_type = transcribed RNA
gctccgccccacgtcgcatgcgccccgggaacgcgtggggcggagcttccggaggccccgctctgctgccgaccctgtggagcggagggtgaagcctccggatgccagtccctcatcgctggcctggtcgcgctgtggcgaagggggcggagc

SEQ ID NO: 409 - RNA, 153 nt, Homo sapiens
  source 1..153: mol_type = transcribed RNA
gctccgccccacgtcgcatgcgccccgggaacgcgtggggcggagcttccggaggccccgccctgctgccgaccctgtggagcggagggtgaagcctccggatgccagtccctcatcgctggcccggtcgcgctgtggcgaagggggcggagc

SEQ ID NO: 410 - RNA, 49 nt, Homo sapiens
  source 1..49: mol_type = transcribed RNA
ggtgaggcgggggggcgagccctgaggggctctcgcttctggcgccaag

SEQ ID NO: 411 - RNA, 100 nt, Homo sapiens
  source 1..100: mol_type = transcribed RNA
gaggtgggaggattgcttgagtcagggtggttgaggctgcagtaagttgtgatcataccactgcactccagcctgagtgacagagcaagaccttgtctca

SEQ ID NO: 412 - RNA, 64 nt, Homo sapiens
  source 1..64: mol_type = transcribed RNA
gggtgggggcggggcggcaggggcctcccccagtgccaggccccattctgcttctctcccagct

SEQ ID NO: 413 - RNA, 74 nt, Homo sapiens
  source 1..74: mol_type = transcribed RNA
cctgcaggaggcagtgggcgagcaggcggggcagcccaatgccatgggcctgatctcaccgctgcctccttccc

SEQ ID NO: 414 - RNA, 60 nt, Homo sapiens
  source 1..60: mol_type = transcribed RNA
aggcctagggggtggcaggctggccatcagtgtgggctaaccctgtcctctccctcccag

SEQ ID NO: 415 - RNA, 100 nt, Homo sapiens
  source 1..100: mol_type = transcribed RNA
gtgagcgggcgcggcagggatcgcgggcgggtggcggcctagggcgcggagggcggaccgggaatggcgcgccgtgcgccgccggcgtaactgcggcgct

SEQ ID NO: 416 - RNA, 56 nt, Homo sapiens
  source 1..56: mol_type = transcribed RNA
gtaggcaggggctggggtttcaggttctcagtcagaaccttggcccctctccccag

SEQ ID NO: 417 - RNA, 72 nt, Homo sapiens
  source 1..72: mol_type = transcribed RNA
tctcctcgaggggtctctgcctctacccaggactctttcatgaccaggaggctgaggcccctcacaggcggc

SEQ ID NO: 418 - RNA, 68 nt, Homo sapiens
  source 1..68: mol_type = transcribed RNA
gcatcctgtactgagctgccccgaggcccttcatgctgcccagctcggggcagctcagtacaggatac

SEQ ID NO: 419 - RNA, 64 nt, Homo sapiens
  source 1..64: mol_type = transcribed RNA
tcctgtactgagctgccccgagctgggcagcatgaagggcctcggggcagctcagtacaggatg

SEQ ID NO: 420 - RNA, 54 nt, Homo sapiens
  source 1..54: mol_type = transcribed RNA
acgcgggtgcgggccggcggggtagaagccacccggcccggcccggcccggcga

SEQ ID NO: 421 - RNA, 89 nt, Homo sapiens
  source 1..89: mol_type = transcribed RNA
accatgctgtagtgtgtgtaaacatcctacactctcagctgtgagctcaaggtggctgggagagggttgtttactccttctgccatgga

SEQ ID NO: 422 - RNA, 85 nt, Homo sapiens
  source 1..85: mol_type = transcribed RNA
gtgagctgctggggacgcgggtcggggtctgcagggcggtgcggcagccgccacctgacgccgcgcctttgtctgtgtcccacag

SEQ ID NO: 423 - RNA, 71 nt, Homo sapiens
  source 1..71: mol_type = transcribed RNA
gggtaaaggggcagggacgggtggccccaggaagaagggcctggtggagccgctcttctccctgcccacag

SEQ ID NO: 424 - RNA, 73 nt, Homo sapiens
  source 1..73: mol_type = transcribed RNA
ggggaggtacctgggacaggaggaggaggcagccttgcctcagaaaccaaactgtcaaaagtgtaggttccac

SEQ ID NO: 425 - RNA, 90 nt, Homo sapiens
  source 1..90: mol_type = transcribed RNA
cgggcggggcgggtccggccgcctccgagcccggccggcagcccccggccttaaagcgcgggctgtccggaggggtcggctttcccaccg

SEQ ID NO: 426 - RNA, 83 nt, Homo sapiens
  source 1..83: mol_type = transcribed RNA
acggcatctttgcactcagcaggcaggctggtgcagcccgtggtgggggaccatcctgcctgctgtggggtaaggacggctgt

SEQ ID NO: 427 - RNA, 63 nt, Homo sapiens
  source 1..63: mol_type = transcribed RNA
gaggtgtaggggaggttgggccagggatgccttcactgtgtctctctggtcttgccaccccag

SEQ ID NO: 428 - RNA, 72 nt, Homo sapiens
  source 1..72: mol_type = transcribed RNA
ctgtgcacctgggggagtgcagtgattgtggaatgcaaagtcccacaatcactgtactccccaggtgcacag

SEQ ID NO: 429 - RNA, 67 nt, Homo sapiens
  source 1..67: mol_type = transcribed RNA
gtggcactcaaactgtgggggcactttctgctctctggtgaaagtgccgccatcttttgagtgttac

SEQ ID NO: 430 - RNA, 62 nt, Homo sapiens
  source 1..62: mol_type = transcribed RNA
ggtagggggcgggctccggcgctgggaccccactagggtggcgccttggccccgccccgccc

SEQ ID NO: 431 - RNA, 82 nt, Homo sapiens
  source 1..82: mol_type = transcribed RNA
gtgagtgggaggccagggcacggcagggggagctgcagggctatgggaggggccccagcgtctgagccctgtcctcccgcag

SEQ ID NO: 432 - RNA, 82 nt, Homo sapiens; identical to SEQ ID NO: 431
  source 1..82: mol_type = transcribed RNA

SEQ ID NO: 433 - RNA, 81 nt, Homo sapiens
  source 1..81: mol_type = transcribed RNA
agttggtgggggagccatgagataagagcacctcctagagaatgttgaactaaaggtgccctctctggctcctccccaaag

SEQ ID NO: 434 - RNA, 68 nt, Homo sapiens
  source 1..68: mol_type = transcribed RNA
ctccggtgcctactgagctgatatcagttctcattttacacactggctcagttcagcaggaacaggag

SEQ ID NO: 435 - RNA, 73 nt, Homo sapiens
  source 1..73: mol_type = transcribed RNA
ctctgcctcccgtgcctactgagctgaaacacagttggtttgtgtacactggctcagttcagcaggaacaggg

SEQ ID NO: 436 - RNA, 83 nt, Homo sapiens
  source 1..83: mol_type = transcribed RNA
gtgagtgggagccccagtgtgtggttggggccatggcgggtgggcagcccagcctctgagccttcctcgtctgtctgccccag

SEQ ID NO: 437 - RNA, 67 nt, Homo sapiens
  source 1..67: mol_type = transcribed RNA
gcgccctccctctctccccggtgtgcaaatgtgtgtgtgcggtgttatgccggacaagagggaggtg

SEQ ID NO: 438 - RNA, 58 nt, Homo sapiens
  source 1..58: mol_type = transcribed RNA
gctgaagctctaaggttccgcctgcgggcaggaagcggaggaaccttggagcttcggc

SEQ ID NO: 439 - RNA, 80 nt, Homo sapiens
  source 1..80: mol_type = transcribed RNA
gagaggccaagaccttgggaatgggggtaagggccttctgagcccaggtccgaactctccattcctctgcagagcgctct

SEQ ID NO: 440 - RNA, 64 nt, Homo sapiens
  source 1..64: mol_type = transcribed RNA
ccccgggcccggcgttccctccccttccgtgcgccagtggaggccggggtggggcggggcgggg

SEQ ID NO: 441 - RNA, 64 nt, Homo sapiens; identical to SEQ ID NO: 440
  source 1..64: mol_type = transcribed RNA

SEQ ID NO: 442 - RNA, 85 nt, Homo sapiens
  source 1..85: mol_type = transcribed RNA
ggggaggtagggaaaaggaagggggaggagaaggtgagaccaatgtcctgggtgccactcctgcccagtgcctcccttcctcgtt

SEQ ID NO: 443 - RNA, 73 nt, Homo sapiens
  source 1..73: mol_type = transcribed RNA
gttcaagtgggaggacaggaggcaggtgtggttggaggaagcagcctgaacctgcctccctgacattccacag

SEQ ID NO: 444 - RNA, 98 nt, Homo sapiens
  source 1..98: mol_type = transcribed RNA
agaatgggcaaatgaacagtaaatttggaggcctggggccctccctgctgctggagaagtgtttgcacgggtgggccttgtctttgaaaggaggtgga

SEQ ID NO: 445 - RNA, 60 nt, Homo sapiens
  source 1..60: mol_type = transcribed RNA
cgctgggtccgcgcgccctgggccgggcgatgtccgcttgggggagcgaggggcggggcg

SEQ ID NO: 446 - RNA, 23 nt, Homo sapiens
  source 1..23: mol_type = transcribed RNA
ctcctggggcccgcactctcgct

SEQ ID NO: 447 - RNA, 18 nt, Homo sapiens
  source 1..18: mol_type = transcribed RNA
ctcctggggcccgcactc

SEQ ID NO: 448 - RNA, 21 nt, Homo sapiens
  source 1..21: mol_type = transcribed RNA
caactctgatctcttcatcta

SEQ ID NO: 449 - RNA, 20 nt, Homo sapiens
  source 1..20: mol_type = transcribed RNA
tctcttcatctaccccccag

SEQ ID NO: 450 - RNA, 23 nt, Homo sapiens
  source 1..23: mol_type = transcribed RNA
ggtgggtgaggtcgggccccaag

SEQ ID NO: 451 - RNA, 20 nt, Homo sapiens
  source 1..20: mol_type = transcribed RNA
cggggtgggtgaggtcgggc

SEQ ID NO: 452 - RNA, 26 nt, Homo sapiens
  source 1..26: mol_type = transcribed RNA
ccttctggagaggctttgtgcggata

SEQ ID NO: 453 - RNA, 15 nt, Homo sapiens
  source 1..15: mol_type = transcribed RNA
ccttctggagaggct

SEQ ID NO: 454 - RNA, 20 nt, Homo sapiens
  source 1..20: mol_type = transcribed RNA
ggctggtcagatgggagtgg

SEQ ID NO: 455 - RNA, 20 nt, Homo sapiens
  source 1..20: mol_type = transcribed RNA
ggctggtcagatgggagtgg

SEQ ID NO: 456 - RNA, 26 nt, Homo sapiens
  source 1..26: mol_type = transcribed RNA
aggagggaggagatgggccaagttcc

SEQ ID NO: 457 - RNA, 15 nt, Homo sapiens
  source 1..15: mol_type = transcribed RNA
gggaggagggaggag

SEQ ID NO: 458 - RNA, 21 nt, Homo sapiens
  source 1..21: mol_type = transcribed RNA
cttccccccagtaatcttcat

SEQ ID NO: 459 - RNA, 21 nt, Homo sapiens
  source 1..21: mol_type = transcribed RNA
cttccccccagtaatcttcat

SEQ ID NO: 460 - RNA, 21 nt, Homo sapiens
  source 1..21: mol_type = transcribed RNA
cggatccgagtcacggcacca

SEQ ID NO: 461 - RNA, 15 nt, Homo sapiens
  source 1..15: mol_type = transcribed RNA
ggatccgagtcacgg

SEQ ID NO: 462 - RNA, 21 nt, Homo sapiens
  source 1..21: mol_type = transcribed RNA
cccaggctggagcgagtgcag

SEQ ID NO: 463 - RNA, 15 nt, Homo sapiens
  source 1..15: mol_type = transcribed RNA
agctcactgcagcct

SEQ ID NO: 464 - RNA, 17 nt, Homo sapiens
  source 1..17: mol_type = transcribed RNA
tcctagtcacggcacca

SEQ ID NO: 465 - RNA, 17 nt, Homo sapiens
  source 1..17: mol_type = transcribed RNA
tcctagtcacggcacca

SEQ ID NO: 466 - RNA, 24 nt, Homo sapiens
  source 1..24: mol_type = transcribed RNA
gaggctgggaaggcaaagggacgt

SEQ ID NO: 467 - RNA, 15 nt, Homo sapiens
  source 1..15: mol_type = transcribed RNA
gaaggaggctgggaa

SEQ ID NO: 468 - RNA, 21 nt, Homo sapiens
  source 1..21: mol_type = transcribed RNA
gaggcgatgtggggatgtaga

SEQ ID NO: 469 - RNA, 20 nt, Homo sapiens
  source 1..20: mol_type = transcribed RNA
cccagtctcatttcctcatc

SEQ ID NO: 470 - RNA, 23 nt, Homo sapiens
  source 1..23: mol_type = transcribed RNA
ggcccggccgtgcctgaggtttc

SEQ ID NO: 471 - RNA, 15 nt, Homo sapiens
  source 1..15: mol_type = transcribed RNA
ggcggtgggatcccg

SEQ ID NO: 472 - RNA, 16 nt, Homo sapiens
  source 1..16: mol_type = transcribed RNA
ctgggggtcccccgac

SEQ ID NO: 473 - RNA, 15 nt, Homo sapiens
  source 1..15: mol_type = transcribed RNA
gtgtggagctggggc

SEQ ID NO: 474 - RNA, 25 nt, Homo sapiens
  source 1..25: mol_type = transcribed RNA
agcggggaggaagtgggcgctgctt

SEQ ID NO: 475 - RNA, 21 nt, Homo sapiens
  source 1..21: mol_type = transcribed RNA
agcggggaggaagtgggcgct

SEQ ID NO: 476 - RNA, 27 nt, Homo sapiens
  source 1..27: mol_type = transcribed RNA
aaaccgttaccattactgagtttagta

SEQ ID NO: 477 - RNA, 15 nt, Homo sapiens
  source 1..15: mol_type = transcribed RNA
gaaaccgttaccatt

SEQ ID NO: 478 - RNA, 22 nt, Homo sapiens
  source 1..22: mol_type = transcribed RNA
caggcacgggagctcaggtgag

SEQ ID NO: 479 - RNA, 17 nt, Homo sapiens
  source 1..17: mol_type = transcribed RNA
caggcacgggagctcag

SEQ ID NO: 480 - RNA, 23 nt, Homo sapiens
  source 1..23: mol_type = transcribed RNA
gggggtccccggtgctcggatct

SEQ ID NO: 481 - RNA, 16 nt, Homo sapiens
  source 1..16: mol_type = transcribed RNA
tcgggaggggcgggag

SEQ ID NO: 482 - RNA, 23 nt, Homo sapiens
  source 1..23: mol_type = transcribed RNA
cggggccgtagcactgtctgaga

SEQ ID NO: 483 - RNA, 20 nt, Homo sapiens
  source 1..20: mol_type = transcribed RNA
cggggccgtagcactgtctg

SEQ ID NO: 484 - RNA, 20 nt, Homo sapiens
  source 1..20: mol_type = transcribed RNA
atcccaccactgccaccatt

SEQ ID NO: 485 - RNA, 15 nt, Homo sapiens
  source 1..15: mol_type = transcribed RNA
atcccaccactgcca

SEQ ID NO: 486 - RNA, 23 nt, Homo sapiens
  source 1..23: mol_type = transcribed RNA
acaggagtgggggtgggacataa

SEQ ID NO: 487 - RNA, 20 nt, Homo sapiens
  source 1..20: mol_type = transcribed RNA
acaggagtgggggtgggaca

SEQ ID NO: 488 - RNA, 23 nt, Homo sapiens
  source 1..23: mol_type = transcribed RNA
ctgggggacgcgtgagcgcgagc

SEQ ID NO: 489 - RNA, 21 nt, Homo sapiens
  source 1..21: mol_type = transcribed RNA
ctgggggacgcgtgagcgcga

SEQ ID NO: 490 - RNA, 22 nt, Homo sapiens
  source 1..22: mol_type = transcribed RNA
cgcggcggggacggcgattggt

SEQ ID NO: 491 - RNA, 17 nt, Homo sapiens
  source 1..17: mol_type = transcribed RNA
cggcggggacggcgatt

SEQ ID NO: 492 - RNA, 23 nt, Homo sapiens
  source 1..23: mol_type = transcribed RNA
gagggccccccctcaatcctgtt

SEQ ID NO: 493 - RNA, 16 nt, Homo sapiens
  source 1..16: mol_type = transcribed RNA
agggccccccctcaat

SEQ ID NO: 494 - RNA, 30 nt, Homo sapiens
  source 1..30: mol_type = transcribed RNA
tgaggggcagagagcgagacttttctattt

SEQ ID NO: 495 - RNA, 15 nt, Homo sapiens
  source 1..15: mol_type = transcribed RNA
tgaggggcagagagc

SEQ ID NO: 496 - RNA, 24 nt, Homo sapiens
  source 1..24: mol_type = transcribed RNA
gtgagtgggagccggtggggctgg

SEQ ID NO: 497 - RNA, 15 nt, Homo sapiens
  source 1..15: mol_type = transcribed RNA
ggggctggagtaagg

SEQ ID NO: 498 - RNA, 23 nt, Homo sapiens
  source 1..23: mol_type = transcribed RNA
tgaggatatggcagggaagggga

SEQ ID NO: 499 - RNA, 19 nt, Homo sapiens
  source 1..19: mol_type = transcribed RNA
tgaggatatggcagggaag

SEQ ID NO: 500 - RNA, 23 nt, Homo sapiens
  source 1..23: mol_type = transcribed RNA
ctagtggaagaagatggcggaag

SEQ ID NO: 501 - RNA, 15 nt, Homo sapiens
  source 1..15: mol_type = transcribed RNA
tagtggaagaagatg

SEQ ID NO: 502 - RNA, 25 nt, Homo sapiens
  source 1..25: mol_type = transcribed RNA
tgggcaggggcttattgtaggagtc

SEQ ID NO: 503 - RNA, 19 nt, Homo sapiens
  source 1..19: mol_type = transcribed RNA
tgggcaggggcttattgta

SEQ ID NO: 504 - RNA, 23 nt, Homo sapiens
  source 1..23: mol_type = transcribed RNA
agaagaaggcggtcggtctgcgg

SEQ ID NO: 505 - RNA, 21 nt, Homo sapiens
  source 1..21: mol_type = transcribed RNA
aagaaggcggtcggtctgcgg

SEQ ID NO: 506 - RNA, 20 nt, Homo sapiens
  source 1..20: mol_type = transcribed RNA
atcccacctctgccaccaaa

SEQ ID NO: 507 - RNA, 15 nt, Homo sapiens
  source 1..15: mol_type = transcribed RNA
atcccacctctgcca

SEQ ID NO: 508 - RNA, 23 nt, Homo sapiens
  source 1..23: mol_type = transcribed RNA
ggaggcgcaggctcggaaaggcg

SEQ ID NO: 509 - RNA, 15 nt, Homo sapiens
  source 1..15: mol_type = transcribed RNA
gcaggctcggaaagg

SEQ ID NO: 510 - RNA, 17 nt, Homo sapiens
  source 1..17: mol_type = transcribed RNA
cctccgggacggctggg

SEQ ID NO: 511 - RNA, 15 nt, Homo sapiens
  source 1..15: mol_type = transcribed RNA
ctccgggacggctgg

SEQ ID NO: 512 - RNA, 29 nt, Homo sapiens
  source 1..29: mol_type = transcribed RNA
ggtcaggcggctcggactgagcaggtggg

SEQ ID NO: 513 - RNA, 15 nt, Homo sapiens
  source 1..15: mol_type = transcribed RNA
agagtgtggtcaggc

SEQ ID NO: 514 - RNA, 15 nt, Homo sapiens
  source 1..15: mol_type = transcribed RNA
ggcgcggggaggtgc

SEQ ID NO: 515 - RNA, 15 nt, Homo sapiens
  source 1..15: mol_type = transcribed RNA
ggcgcggggaggtgc

SEQ ID NO: 516 - RNA, 22 nt, Homo sapiens
  source 1..22: mol_type = transcribed RNA
accccactcctggtaccatagt

SEQ ID NO: 517 - RNA, 15 nt, Homo sapiens
  source 1..15: mol_type = transcribed RNA
accccactcctggta

SEQ ID NO: 518 - RNA, 15 nt, Homo sapiens
  source 1..15: mol_type = transcribed RNA
tgaagcgggggggcg

SEQ ID NO: 519 - RNA, 15 nt, Homo sapiens
  source 1..15: mol_type = transcribed RNA
tgaagcgggggggcg

SEQ ID NO: 520 - RNA, 21 nt, Homo sapiens
  source 1..21: mol_type = transcribed RNA
gaatggatttttggagcagga

SEQ ID NO: 521 - RNA, 15 nt, Homo sapiens
  source 1..15: mol_type = transcribed RNA
gaatggatttttgga

SEQ ID NO: 522 - RNA, 25 nt, Homo sapiens
  source 1..25: mol_type = transcribed RNA
tggcggcggtagttatgggcttctc

SEQ ID NO: 523 - RNA, 25 nt, Homo sapiens
  source 1..25: mol_type = transcribed RNA
tggcggcggtagttatgggcttctc

SEQ ID NO: 524 - RNA, 18 nt, Homo sapiens
  source 1..18: mol_type = transcribed RNA
gaggctgaaggaagatgg

SEQ ID NO: 525 - RNA, 15 nt, Homo sapiens
  source 1..15: mol_type = transcribed RNA
gaggctgaaggaaga

SEQ ID NO: 526 - RNA, 23 nt, Homo sapiens
  source 1..23: mol_type = transcribed RNA
aggaggggtcccgcactgggagg

SEQ ID NO: 527 - RNA, 15 nt, Homo sapiens
  source 1..15: mol_type = transcribed RNA
tgggaggggccctca

SEQ ID NO: 528 - RNA, 21 nt, Homo sapiens
  source 1..21: mol_type = transcribed RNA
gagggcagcgtgggtgtggcg

SEQ ID NO: 529 - RNA, 21 nt, Homo sapiens
  source 1..21: mol_type = transcribed RNA
gagggcagcgtgggtgtggcg

SEQ ID NO: 530 - RNA, 23 nt, Homo sapiens
  source 1..23: mol_type = transcribed RNA
caccttgccttgctgcccgggcc

SEQ ID NO: 531 - RNA, 22 nt, Homo sapiens
  source 1..22: mol_type = transcribed RNA
caccttgccttgctgcccgggc

SEQ ID NO: 532 - RNA, 19 nt, Homo sapiens
  source 1..19: mol_type = transcribed RNA
gtgggttggggcgggctct

SEQ ID NO: 533 - RNA, 19 nt, Homo sapiens
  source 1..19: mol_type = transcribed RNA
gtgggttggggcgggctct

SEQ ID NO: 534 - RNA, 24 nt, Homo sapiens
  source 1..24: mol_type = transcribed RNA
atatacagggggagactctcattt

SEQ ID NO: 535 - RNA, 15 nt, Homo sapiens
  source 1..15: mol_type = transcribed RNA
atatacagggggaga

SEQ ID NO: 536 - RNA, 26 nt, Homo sapiens
  source 1..26: mol_type = transcribed RNA
agggctggactcagcggcggagctgg

SEQ ID NO: 537 - RNA, 17 nt, Homo sapiens
  source 1..17: mol_type = transcribed RNA
gcggcggagctggctgc

SEQ ID NO: 538 - RNA, 22 nt, Homo sapiens
  source 1..22: mol_type = transcribed RNA
gtgggcgggggcaggtgtgtgg

SEQ ID NO: 539 - RNA, 15 nt, Homo sapiens
  source 1..15: mol_type = transcribed RNA
cgggggcaggtgtgt

SEQ ID NO: 540 - RNA, 16 nt, Homo sapiens
  source 1..16: mol_type = transcribed RNA
tgctggtgatgctttc

SEQ ID NO: 541 - RNA, 16 nt, Homo sapiens
  source 1..16: mol_type = transcribed RNA
tgctggtgatgctttc

SEQ ID NO: 542 - RNA, 22 nt, Homo sapiens
  source 1..22: mol_type = transcribed RNA
actcggctgcggtggacaagtc

SEQ ID NO: 543 - RNA, 20 nt, Homo sapiens
  source 1..20: mol_type = transcribed RNA
actcggctgcggtggacaag

SEQ ID NO: 544 - RNA, 18 nt, Homo sapiens
  source 1..18: mol_type = transcribed RNA
tctaggtggggagactga

SEQ ID NO: 545 - RNA, 16 nt, Homo sapiens
  source 1..16: mol_type = transcribed RNA
gtggggagactgacgg

SEQ ID NO: 546 - RNA, 26 nt, Homo sapiens
  source 1..26: mol_type = transcribed RNA
gtcccggggctgcgcgaggcacaggc

SEQ ID NO: 547 - RNA, 15 nt, Homo sapiens
  source 1..15: mol_type = transcribed RNA
ggcccggggggcggg

SEQ ID NO: 548 - RNA, 23 nt, Homo sapiens
  source 1..23: mol_type = transcribed RNA
gtgggggagaggctgtcttgtgt

SEQ ID NO: 549 - RNA, 15 nt, Homo sapiens
  source 1..15: mol_type = transcribed RNA
gtgtgggggagaggc

SEQ ID NO: 550 - RNA, 17 nt, Homo sapiens
  source 1..17: mol_type = transcribed RNA
ggtgagcgctcgctggc

SEQ ID NO: 551 - RNA, 15 nt, Homo sapiens
  source 1..15: mol_type = transcribed RNA
cggtgagcgctcgct

SEQ ID NO: 552 - RNA, 21 nt, Homo sapiens
  source 1..21: mol_type = transcribed RNA
gttggaggcgtgggttttaga

SEQ ID NO: 553 - RNA, 15 nt, Homo sapiens
  source 1..15: mol_type = transcribed RNA
gttggaggcgtgggt

SEQ ID NO: 554 - RNA, 25 nt, Homo sapiens
  source 1..25: mol_type = transcribed RNA
caggaaggatttagggacaggcttt

SEQ ID NO: 555 - RNA, 19 nt, Homo sapiens
  source 1..19: mol_type = transcribed RNA
caggaaggatttagggaca

SEQ ID NO: 556 - RNA, 15 nt, Homo sapiens
  source 1..15: mol_type = transcribed RNA
tctgggcgaggggtg

SEQ ID NO: 557 - RNA, 15 nt, Homo sapiens
  source 1..15: mol_type = transcribed RNA
tctgggcgaggggtg

SEQ ID NO: 558 - RNA, 23 nt, Homo sapiens
  source 1..23: mol_type = transcribed RNA
ggggggatgtgcatgctggttgg

SEQ ID NO: 559 - RNA, 15 nt, Homo sapiens
  source 1..15: mol_type = transcribed RNA
atcagcgtgcacttc

SEQ ID NO: 560 - RNA, 20 nt, Homo sapiens
  source 1..20: mol_type = transcribed RNA
ccccagggcgacgcggcggg

SEQ ID NO: 561 - RNA, 15 nt, Homo sapiens
  source 1..15: mol_type = transcribed RNA
cgcggcgggggcggc

SEQ ID NO: 562 - RNA, 23 nt, Homo sapiens
  source 1..23: mol_type = transcribed RNA
agggtcggggcagggagggcagg

SEQ ID NO: 563 - RNA, 15 nt, Homo sapiens
  source 1..15: mol_type = transcribed RNA
gggagaagggtcggg

SEQ ID NO: 564 - RNA, 18 nt, Homo sapiens
  source 1..18: mol_type = transcribed RNA
ggtgggcttcccggaggg

SEQ ID NO: 565 - RNA, 15 nt, Homo sapiens
  source 1..15: mol_type = transcribed RNA
ggtgggcttcccgga

SEQ ID NO: 566 - RNA, 20 nt, Homo sapiens
  source 1..20: mol_type = transcribed RNA
aagacacatttggagaggga

SEQ ID NO: 567 - RNA, 16 nt, Homo sapiens
  source 1..16: mol_type = transcribed RNA
agacacatttggagag

SEQ ID NO: 568 - RNA, 19 nt, Homo sapiens
  source 1..19: mol_type = transcribed RNA
gagggcgggtggaggagga

SEQ ID NO: 569 - RNA, 15 nt, Homo sapiens
  source 1..15: mol_type = transcribed RNA
gcgggtggaggagga

SEQ ID NO: 570 - RNA, 17 nt, Homo sapiens
  source 1..17: mol_type = transcribed RNA
gatcccagcggtgcctc

SEQ ID NO: 571 - RNA, 15 nt, Homo sapiens
  source 1..15: mol_type = transcribed RNA
gatcccagcggtgcc

SEQ ID NO: 572 - RNA, 23 nt, Homo sapiens
  source 1..23: mol_type = transcribed RNA
aagggaggaggagcggaggggcc

SEQ ID NO: 573 - RNA, 15 nt, Homo sapiens
  source 1..15: mol_type = transcribed RNA
gggaggaggagcgga

SEQ ID NO: 574 - RNA, 23 nt, Homo sapiens
  source 1..23: mol_type = transcribed RNA
ccaggaggcggaggaggtggagg

SEQ ID NO: 575 - RNA, 15 nt, Homo sapiens
  source 1..15: mol_type = transcribed RNA
acccaggaggcggag

SEQ ID NO: 576 - RNA, 15 nt, Homo sapiens
  source 1..15: mol_type = transcribed RNA
tggcagagcgctgtc

SEQ ID NO: 577 - RNA, 15 nt, Homo sapiens
  source 1..15: mol_type = transcribed RNA
tggcagagcgctgtc

SEQ ID NO: 578 - RNA, 22 nt, Homo sapiens
  source 1..22: mol_type = transcribed RNA
gagggttgggtggaggctctcc

SEQ ID NO: 579 - RNA, 15 nt, Homo sapiens
  source 1..15: mol_type = transcribed RNA
gagggttgggtggag

SEQ ID NO: 580 - RNA, 21 nt, Homo sapiens
  source 1..21: mol_type = transcribed RNA
cagcaggggagagagaggagt

SEQ ID NO: 581 - RNA, 20 nt, Homo sapiens
  source 1..20: mol_type = transcribed RNA
cagcaggggagagagaggag

SEQ ID NO: 582 - RNA, 22 nt, Homo sapiens
  source 1..22: mol_type = transcribed RNA
ccgggaacgtcgagactggagc

SEQ ID NO: 583 - RNA, 15 nt, Homo sapiens
  source 1..15: mol_type = transcribed RNA
cgggaacgtcgagac

SEQ ID NO: 584 - RNA, 17 nt, Homo sapiens
  source 1..17: mol_type = transcribed RNA
aggggctgggcgcgcgc

SEQ ID NO: 585 - RNA, 15 nt, Homo sapiens
  source 1..15: mol_type = transcribed RNA
caggggctgggcgcg

SEQ ID NO: 586 - RNA, 24 nt, Homo sapiens
  source 1..24: mol_type = transcribed RNA
cgggcgtggtggtgggggtgggtg

SEQ ID NO: 587 - RNA, 15 nt, Homo sapiens
  source 1..15: mol_type = transcribed RNA
cgggcgtggtggtgg

SEQ ID NO: 588 - RNA, 24 nt, Homo sapiens
  source 1..24: mol_type = transcribed RNA
tggggagctgaggctctgggggtg

SEQ ID NO: 589 - RNA, 15 nt, Homo sapiens
  source 1..15: mol_type = transcribed RNA
ggccctggggagctg

SEQ ID NO: 590 - RNA, 19 nt, Homo sapiens
  source 1..19: mol_type = transcribed RNA
tggcgggtgcgggggtggg

SEQ ID NO: 591 - RNA, 15 nt, Homo sapiens
  source 1..15: mol_type = transcribed RNA
tggcgggtgcggggg

SEQ ID NO: 592 - RNA, 25 nt, Homo sapiens
  source 1..25: mol_type = transcribed RNA
tgcggggctagggctaacagcagtc

SEQ ID NO: 593 - RNA, 15 nt, Homo sapiens
  source 1..15: mol_type = transcribed RNA
tgcggggctagggct

SEQ ID NO: 594 - RNA, 20 nt, Homo sapiens
  source 1..20: mol_type = transcribed RNA
tggctgttggagggggcagg

SEQ ID NO: 595 - RNA, 15 nt, Homo sapiens
  source 1..15: mol_type = transcribed RNA
ggagggggcaggctc

SEQ ID NO: 596 - RNA, 24 nt, Homo sapiens
  source 1..24: mol_type = transcribed RNA
aggcaggggctggtgctgggcggg

SEQ ID NO: 597 - RNA, 15 nt, Homo sapiens
  source 1..15: mol_type = transcribed RNA
gggcggggggcggcg

SEQ ID NO: 598 - RNA, 23 nt, Homo sapiens
  source 1..23: mol_type = transcribed RNA
gtgggctgggctgggctgggcca

SEQ ID NO: 599 - RNA, 15 nt, Homo sapiens
  source 1..15: mol_type = transcribed RNA
gggctgggctgggct

SEQ ID NO: 600 - RNA, 17 nt, Homo sapiens
  source 1..17: mol_type = transcribed RNA
ggggcgggggcgggggc

SEQ ID NO: 601 - RNA, 15 nt, Homo sapiens
  source 1..15: mol_type = transcribed RNA
cgcgccgggcccggg

SEQ ID NO: 602 - RNA, 23 nt, Homo sapiens
  source 1..23: mol_type = transcribed RNA
gccccggcgcgggcgggttctgg

SEQ ID NO: 603 - RNA, 16 nt, Homo sapiens
  source 1..16: mol_type = transcribed RNA
ggagccccggcgcggg

SEQ ID NO: 604 - RNA, 24 nt, Homo sapiens
  source 1..24: mol_type = transcribed RNA
agacacatttggagagggaacctc

SEQ ID NO: 605 - RNA, 16 nt, Homo sapiens
  source 1..16: mol_type = transcribed RNA
agacacatttggagag

SEQ ID NO: 606 - RNA, 22 nt, Homo sapiens
  source 1..22: mol_type = transcribed RNA
tgggaggggagaggcagcaagc

SEQ ID NO: 607 - RNA, 22 nt, Homo sapiens
  source 1..22: mol_type = transcribed RNA
tgggaggggagaggcagcaagc

SEQ ID NO: 608 - RNA, 21 nt, Homo sapiens
  source 1..21: mol_type = transcribed RNA
atccagttctctgagggggct

SEQ ID NO: 609 - RNA, 21 nt, Homo sapiens
  source 1..21: mol_type = transcribed RNA
atccagttctctgagggggct

SEQ ID NO: 610 - RNA, 23 nt, Homo sapiens
  source 1..23: mol_type = transcribed RNA
aatatacagggggagactcttat

SEQ ID NO: 611 - RNA, 15 nt, Homo sapiens
  source 1..15: mol_type = transcribed RNA
atatacagggggaga

SEQ ID NO: 612 - RNA, 24 nt, Homo sapiens
  source 1..24: mol_type = transcribed RNA
tcgaggactggtggaagggccttt

SEQ ID NO: 613 - RNA, 16 nt, Homo sapiens
  source 1..16: mol_type = transcribed RNA
tcgaggactggtggaa

SEQ ID NO: 614 - RNA, 18 nt, Homo sapiens
  source 1..18: mol_type = transcribed RNA
gagactggggtggggcct

SEQ ID NO: 615 - RNA, 16 nt, Homo sapiens
  source 1..16: mol_type = transcribed RNA
agactggggtggggcc

SEQ ID NO: 616 - RNA, 23 nt, Homo sapiens
  source 1..23: mol_type = transcribed RNA
gggggccgatacactgtacgaga

SEQ ID NO: 617 - RNA, 20 nt, Homo sapiens
  source 1..20: mol_type = transcribed RNA
gggggccgatacactgtacg

SEQ ID NO: 618 - RNA, 18 nt, Homo sapiens
  source 1..18: mol_type = transcribed RNA
cagcggggctgggcgcgc

SEQ ID NO: 619 - RNA, 15 nt, Homo sapiens
  source 1..15: mol_type = transcribed RNA
cagcggggctgggcg

SEQ ID NO: 620 - RNA, 24 nt, Homo sapiens
  source 1..24: mol_type = transcribed RNA
gctgcgggctgcggtcagggcgat

SEQ ID NO: 621 - RNA, 20 nt, Homo sapiens
  source 1..20: mol_type = transcribed RNA
gctgcgggctgcggtcaggg

SEQ ID NO: 622 - RNA, 23 nt, Homo sapiens
  source 1..23: mol_type = transcribed RNA
agggagtagaagggtggggagca

SEQ ID NO: 623 - RNA, 16 nt, Homo sapiens
  source 1..16: mol_type = transcribed RNA
tagggagtagaagggt

SEQ ID NO: 624 - RNA, 27 nt, Homo sapiens
  source 1..27: mol_type = transcribed RNA
gtgaacgggcgccatcccgaggctttg

SEQ ID NO: 625 - RNA, 16 nt, Homo sapiens
  source 1..16: mol_type = transcribed RNA
gtgaacgggcgccatc

SEQ ID NO: 626 - RNA, 25 nt, Homo sapiens
  source 1..25: mol_type = transcribed RNA
ttctgggcccgcggcgggcgtgggg

SEQ ID NO: 627 - RNA, 15 nt, Homo sapiens
  source 1..15: mol_type = transcribed RNA
cgcggcgggcgtggg

SEQ ID NO: 628 - RNA, 26 nt, Homo sapiens
  source 1..26: mol_type = transcribed RNA
aaaatcacattgccagggattaccac

SEQ ID NO: 629 - RNA, 15 nt, Homo sapiens
  source 1..15: mol_type = transcribed RNA
aatcacattgccagg

SEQ ID NO: 630 - RNA, 27 nt, Homo sapiens
  source 1..27: mol_type = transcribed RNA
atcacattgccagggatttccaaccga

SEQ ID NO: 631 - RNA, 15 nt, Homo sapiens
  source 1..15: mol_type = transcribed RNA
aatcacattgccagg

SEQ ID NO: 632 - RNA, 26 nt, Homo sapiens
  source 1..26: mol_type = transcribed RNA
gactatagaactttccccctcatccc

SEQ ID NO: 633 - RNA, 15 nt, Homo sapiens
  source 1..15: mol_type = transcribed RNA
aactttccccctcat

SEQ ID NO: 634 - RNA, 22 nt, Homo sapiens
  source 1..22: mol_type = transcribed RNA
cctcacacctgcctcgcccccc

SEQ ID NO: 635 - RNA, 15 nt, Homo sapiens
  source 1..15: mol_type = transcribed RNA
tcacacctgcctcgc

SEQ ID NO: 636 - RNA, 17 nt, Homo sapiens
  source 1..17: mol_type = transcribed RNA
tgcaggggcaggccagc

SEQ ID NO: 637 - RNA, 17 nt, Homo sapiens
  source 1..17: mol_type = transcribed RNA
tgcaggggcaggccagc

SEQ ID NO: 638 - RNA, 22 nt, Homo sapiens
  source 1..22: mol_type = transcribed RNA
ccggcagaggaggctgcagagg

SEQ ID NO: 639 - RNA, 19 nt, Homo sapiens
  source 1..19: mol_type = transcribed RNA
ccggcagaggaggctgcag

SEQ ID NO: 640 - RNA, 25 nt, Homo sapiens
  source 1..25: mol_type = transcribed RNA
gggtggggatttgttgcattacttg

SEQ ID NO: 641 - RNA, 20 nt, Homo sapiens
  source 1..20: mol_type = transcribed RNA
gggtggggatttgttgcatt

SEQ ID NO: 642 - RNA, 23 nt, Homo sapiens
  source 1..23: mol_type = transcribed RNA
ggctacaacacaggacccgggcg

SEQ ID NO: 643 - RNA, 21 nt, Homo sapiens
  source 1..21: mol_type = transcribed RNA
ggctacaacacaggacccggg

SEQ ID NO: 644 - RNA, 27 nt, Homo sapiens
  source 1..27: mol_type = transcribed RNA
tagcagcacgtaaatattggcgttaag

SEQ ID NO: 645 - RNA, 15 nt, Homo sapiens
  source 1..15: mol_type = transcribed RNA
tagcagcacgtaaat

SEQ ID NO: 646 - RNA, 24 nt, Homo sapiens
  source 1..24: mol_type = transcribed RNA
aatattgcactcgtcccggcctcc

SEQ ID NO: 647 - RNA, 15 nt, Homo sapiens
  source 1..15: mol_type = transcribed RNA
tattgcactcgtccc

SEQ ID NO: 648 - RNA, 24 nt, Homo sapiens
  source 1..24: mol_type = transcribed RNA
ctggtacaggcctgggggacaggg

SEQ ID NO: 649 - RNA, 18 nt, Homo sapiens
  source 1..18: mol_type = transcribed RNA
ctggtacaggcctggggg

SEQ ID NO: 650 - RNA, 18 nt, Homo sapiens
  source 1..18: mol_type = transcribed RNA
ctccgggcggcgccgtgt

SEQ ID NO: 651 - RNA, 18 nt, Homo sapiens
  source 1..18: mol_type = transcribed RNA
ctccgggcggcgccgtgt

SEQ ID NO: 652 - RNA, 23 nt, Homo sapiens
  source 1..23: mol_type = transcribed RNA
cacaggtgaggttcttgggagcc

SEQ ID NO: 653 - RNA, 15 nt, Homo sapiens
  source 1..15: mol_type = transcribed RNA
acaggtgaggttctt

SEQ ID NO: 654 - RNA, 26 nt, Homo sapiens
  source 1..26: mol_type = transcribed RNA
agggacgggacgcggtgcagtgttgt

SEQ ID NO: 655 - RNA, 15 nt, Homo sapiens
  source 1..15: mol_type = transcribed RNA
ggcgggcgggaggga

SEQ ID NO: 656 - RNA, 28 nt, Homo sapiens
  source 1..28: mol_type = transcribed RNA
gtatggtattgcacttgtcccggcctgt

SEQ ID NO: 657 - RNA, 15 nt, Homo sapiens
  source 1..15: mol_type = transcribed RNA
tattgcacttgtccc

SEQ ID NO: 658 - RNA, 26 nt, Homo sapiens
  source 1..26: mol_type = transcribed RNA
cggtgggatcccgcggccgtgttttc

SEQ ID NO: 659 - RNA, 15 nt, Homo sapiens
  source 1..15: mol_type = transcribed RNA
ggggcgccgcgggac

SEQ ID NO: 660 - RNA, 23 nt, Homo sapiens
  source 1..23: mol_type = transcribed RNA
taggggcagcagaggacctgggc

SEQ ID NO: 661 - RNA, 20 nt, Homo sapiens
  source 1..20: mol_type = transcribed RNA
taggggcagcagaggacctg

SEQ ID NO: 662 - RNA, 22 nt, Homo sapiens
  source 1..22: mol_type = transcribed RNA
tgtgggactgcaaatgggagct

SEQ ID NO: 663 - RNA, 22 nt, Homo sapiens
  source 1..22: mol_type = transcribed RNA
tgtgggactgcaaatgggagct

SEQ ID NO: 664 - RNA, 16 nt, Homo sapiens
  source 1..16: mol_type = transcribed RNA
gtgaaggcccggcgga

SEQ ID NO: 665 - RNA, 15 nt, Homo sapiens
  source 1..15: mol_type = transcribed RNA
gtgaaggcccggcgg

SEQ ID NO: 666 - RNA, 22 nt, Homo sapiens
  source 1..22: mol_type = transcribed RNA
ggggggcaggaggggctcaggg

SEQ ID NO: 667 - RNA, 16 nt, Homo sapiens
  source 1..16: mol_type = transcribed RNA
gtgggggggcaggagg

SEQ ID NO: 668 - RNA, 23 nt, Homo sapiens
  source 1..23: mol_type = transcribed RNA
tggggcggagcttccggaggccc

SEQ ID NO: 669 - RNA, 16 nt, Homo sapiens
  source 1..16: mol_type = transcribed RNA
atcgctggcctggtcg

SEQ ID NO: 670 - RNA, 26 nt, Homo sapiens
  source 1..26: mol_type = transcribed RNA
gaggggctctcgcttctggcgccaag

SEQ ID NO: 671 - RNA, 15 nt, Homo sapiens
  source 1..15: mol_type = transcribed RNA
ggtgaggcggggggg

SEQ ID NO: 672 - RNA, 21 nt, Homo sapiens
  source 1..21: mol_type = transcribed RNA
cagcctgagtgacagagcaag

SEQ ID NO: 673 - RNA, 15 nt, Homo sapiens
  source 1..15: mol_type = transcribed RNA
actgcactccagcct

SEQ ID NO: 674 - RNA, 17 nt, Homo sapiens
  source 1..17: mol_type = transcribed RNA
gcggggcggcaggggcc

SEQ ID NO: 675 - RNA, 15 nt, Homo sapiens
  source 1..15: mol_type = transcribed RNA
gggggcggggcggca

SEQ ID NO: 676 - RNA, 21 nt, Homo sapiens
  source 1..21: mol_type = transcribed RNA
aggaggcagtgggcgagcagg

SEQ ID NO: 677 - RNA, 21 nt, Homo sapiens
  source 1..21: mol_type = transcribed RNA
aggaggcagtgggcgagcagg

SEQ ID NO: 678 - RNA, 16 nt, Homo sapiens
  source 1..16: mol_type = transcribed RNA
ggcgcggagggcggac

SEQ ID NO: 679 - RNA, 15 nt, Homo sapiens
  source 1..15: mol_type = transcribed RNA
ggcgcggagggcgga

SEQ ID NO: 680 - RNA, 22 nt, Homo sapiens
  source 1..22: mol_type = transcribed RNA
accaggaggctgaggcccctca

SEQ ID NO: 681 - RNA, 15 nt, Homo sapiens
  source 1..15: mol_type = transcribed RNA
accaggaggctgagg

SEQ ID NO: 682 - RNA, 23 nt, Homo sapiens
  source 1..23: mol_type = transcribed RNA
cggggcagctcagtacaggatac

SEQ ID NO: 683 - RNA, 15 nt, Homo sapiens
  source 1..15: mol_type = transcribed RNA
agctcagtacaggat

SEQ ID NO: 684 - RNA, 19 nt, Homo sapiens
  source 1..19: mol_type = transcribed RNA
gggtgcgggccggcggggt

SEQ ID NO: 685 - RNA, 15 nt, Homo sapiens
  source 1..15: mol_type = transcribed RNA
tgcgggccggcgggg

SEQ ID NO: 686 - RNA, 27 nt, Homo sapiens
  source 1..27: mol_type = transcribed RNA
caaggtggctgggagagggttgtttac

SEQ ID NO: 687 - RNA, 15 nt, Homo sapiens
  source 1..15: mol_type = transcribed RNA
gtgagctcaaggtgg

SEQ ID NO: 688 - RNA, 15 nt, Homo sapiens
  source 1..15: mol_type = transcribed RNA
aggaggaggaggcag

SEQ ID NO: 689 - RNA, 15 nt, Homo sapiens
  source 1..15: mol_type = transcribed RNA
aggaggaggaggcag

SEQ ID NO: 690 - RNA, 26 nt, Homo sapiens
  source 1..26: mol_type = transcribed RNA
gcgggctgtccggaggggtcggcttt

SEQ ID NO: 691 - RNA, 16 nt, Homo sapiens
  source 1..16: mol_type = transcribed RNA
gctgtccggaggggtc

SEQ ID NO: 692 - RNA, 24 nt, Homo sapiens
  source 1..24: mol_type = transcribed RNA
tgggggagtgcagtgattgtggaa

SEQ ID NO: 693 - RNA, 19 nt, Homo sapiens
  source 1..19: mol_type = transcribed RNA
tgggggagtgcagtgattg

SEQ ID NO: 694 - RNA, 22 nt, Homo sapiens
  source 1..22: mol_type = transcribed RNA
actcaaactgtgggggcacttt

SEQ ID NO: 695 - RNA, 19 nt, Homo sapiens
  source 1..19: mol_type = transcribed RNA
actcaaactgtgggggcac

SEQ ID NO: 696 - RNA, 19 nt, Homo sapiens
  source 1..19: mol_type = transcribed RNA
agggggcgggctccggcgc

SEQ ID NO: 697 - RNA, 15 nt, Homo sapiens
  source 1..15: mol_type = transcribed RNA
gtagggggcgggctc

SEQ ID NO: 698 - RNA, 19 nt, Homo sapiens
  source 1..19: mol_type = transcribed RNA
agtgggaggccagggcacg

SEQ ID NO: 699 - RNA, 15 nt, Homo sapiens
  source 1..15: mol_type = transcribed RNA
agggggagctgcagg

SEQ ID NO: 700 - RNA, 25 nt, Homo sapiens
  source 1..25: mol_type = transcribed RNA
gggggagccatgagataagagcacc

SEQ ID NO: 701 - RNA, 20 nt, Homo sapiens
  source 1..20: mol_type = transcribed RNA
tgggggagccatgagataag

SEQ ID NO: 702 - RNA, 24 nt, Homo sapiens
  source 1..24: mol_type = transcribed RNA
actggctcagttcagcaggaacag

SEQ ID NO: 703 - RNA, 15 nt, Homo sapiens
  source 1..15: mol_type = transcribed RNA
tggctcagttcagca

SEQ ID NO: 704 - RNA, 20 nt, Homo sapiens
  source 1..20: mol_type = transcribed RNA
ctccccggtgtgcaaatgtg

SEQ ID NO: 705 - RNA, 15 nt, Homo sapiens
  source 1..15: mol_type = transcribed RNA
gtgtgcggtgttatg

SEQ ID NO: 706 - RNA, 22 nt, Homo sapiens
  source 1..22: mol_type = transcribed RNA
ggcaggaagcggaggaaccttg

SEQ ID NO: 707 - RNA, 17 nt, Homo sapiens
  source 1..17: mol_type = transcribed RNA
ggaggaaccttggagct

SEQ ID NO: 708 - RNA, 21 nt, Homo sapiens
  source 1..21: mol_type = transcribed RNA
tgggaatgggggtaagggcct

SEQ ID NO: 709 - RNA, 15 nt, Homo sapiens
  source 1..15: mol_type = transcribed RNA
cttctgagcccaggt

SEQ ID NO: 710 - RNA, 16 nt, Homo sapiens
  source 1..16: mol_type = transcribed RNA
cgggcccggcgttccc

SEQ ID NO: 711 - RNA, 15 nt, Homo sapiens
  source 1..15: mol_type = transcribed RNA
ccgggcccggcgttc

SEQ ID NO: 712 - RNA, 18 nt, Homo sapiens
  source 1..18: mol_type = transcribed RNA
aaaaggaagggggaggag

SEQ ID NO: 713 - RNA, 16 nt, Homo sapiens
  source 1..16: mol_type = transcribed RNA
aaggaagggggaggag

SEQ ID NO: 714 - RNA, 22 nt, Homo sapiens
  source 1..22: mol_type = transcribed RNA
tagggatgggaggccaggatga

SEQ ID NO: 715 - RNA, 20 nt, Homo sapiens
  source 1..20: mol_type = transcribed RNA
ggctccttggtctaggggta

SEQ ID NO: 716 - RNA, 23 nt, Homo sapiens
  source 1..23: mol_type = transcribed RNA
aggaagccctggaggggctggag

SEQ ID NO: 717 - RNA, 17 nt, Homo sapiens
  source 1..17: mol_type = transcribed RNA
ggggcgcggccggatcg

SEQ ID NO: 718 - RNA, 22 nt, Homo sapiens
  source 1..22: mol_type = transcribed RNA
tggggaaggcgtcagtgtcggg

SEQ ID NO: 719 - RNA, 21 nt, Homo sapiens
  source 1..21: mol_type = transcribed RNA
aaggcagggcccccgctcccc

SEQ ID NO: 720 - RNA, 24 nt, Homo sapiens
  source 1..24: mol_type = transcribed RNA
gtaggggcgtcccgggcgcgcggg

SEQ ID NO: 721 - RNA, 20 nt, Homo sapiens
  source 1..20: mol_type = transcribed RNA
aaaaggcgggagaagcccca

SEQ ID NO: 722 - RNA, 19 nt, Homo sapiens
  source 1..19: mol_type = transcribed RNA
cggcgcgaccggcccgggg

SEQ ID NO: 723 - RNA, 23 nt, Homo sapiens
  source 1..23: mol_type = transcribed RNA
tgagtggggctcccgggacggcg

SEQ ID NO: 724 - RNA, 23 nt, Homo sapiens
  source 1..23: mol_type = transcribed RNA
ctggcggagcccattccatgcca

SEQ ID NO: 725 - RNA, 22 nt, Homo sapiens
  source 1..22: mol_type = transcribed RNA
ctgggggtggggggctgggcgt

SEQ ID NO: 726 - RNA, 23 nt, Homo sapiens
  source 1..23: mol_type = transcribed RNA
ccagggggatgggcgagcttggg

SEQ ID NO: 727 - RNA, 21 nt, Homo sapiens
  source 1..21: mol_type = transcribed RNA
agccgcggggatcgccgaggg

SEQ ID NO: 728 - RNA, 23 nt, Homo sapiens
  source 1..23: mol_type = transcribed RNA
ccccggtgttggggcgcgtctgc

SEQ ID NO: 729 - RNA, 21 nt, Homo sapiens
  source 1..21: mol_type = transcribed RNA
atgcctcccccggccccgcag

SEQ ID NO: 730 - RNA, 69 nt, Homo sapiens
  source 1..69: mol_type = transcribed RNA
gggcttagggatgggaggccaggatgaagattaatccctaatccccaacactggccttgctatccccag

SEQ ID NO: 731 - RNA, 86 nt, Homo sapiens
  source 1..86: mol_type = transcribed RNA
aggagtgaccaaaagacaagagtgcgagccttctattatgcccagacagggccaccagagggctccttggtctaggggtaatgcca

SEQ ID NO: 732 - RNA, 118 nt, Homo sapiens
  source 1..118: mol_type = transcribed RNA
gcaggtgaactggcaggccaggaagaggaggaagccctggaggggctggaggtgatggatgttttcctccggttctcagggctccacctctttcgggccgtagagccagggctggtgc

SEQ ID NO: 733 - RNA, 84 nt, Homo sapiens
  source 1..84: mol_type = transcribed RNA
gaggctgggcggggcgcggccggatcggtcgagagcgtcctggctgatgacggtctcccgtgcccacgccccaaacgcagtctc

SEQ ID NO: 734 - RNA, 90 nt, Homo sapiens
  source 1..90: mol_type = transcribed RNA
gtgtctctctggagaccctgcagccttcccacccaccagggagctttccatgggctgtggggaaggcgtcagtgtcgggtgagggaacac

SEQ ID NO: 735 - RNA, 94 nt, Homo sapiens
  source 1..94: mol_type = transcribed RNA
gtgaggtgtgggcccggccccaggagcggggcctgggcagccccgtgtgttgaggaaggaaggcagggcccccgctccccgggcctgaccccac

SEQ ID NO: 736 - RNA, 98 nt, Homo sapiens
  source 1..98: mol_type = transcribed RNA
cgaggtaggggcgtcccgggcgcgcgggcgggtcccaggctgggcccctcggaggccgggtgctcactgccccgtcccggcgcccgtgtctcctccag

SEQ ID NO: 737 - RNA, 83 nt, Homo sapiens
  source 1..83: mol_type = transcribed RNA
gggtttcctctgcctttttttccaatgaaaataacgaaacctgttatttcccattgagggggaaaaaggcgggagaagcccca

SEQ ID NO: 738 - RNA, 54 nt, Homo sapiens
  source 1..54: mol_type = transcribed RNA
ggacaagggcggcgcgaccggcccggggctcttgggcggccgcgtttcccctcc

SEQ ID NO: 739 - RNA, 62 nt, Homo sapiens
  source 1..62: mol_type = transcribed RNA
gtgagtggggctcccgggacggcgcccgccctggccctggcccggcgacgtctcacggtccc

SEQ ID NO: 740 - RNA, 76 nt, Homo sapiens
  source 1..76: mol_type = transcribed RNA
cgcaggcctctggcggagcccattccatgccagatgctgagcgatggctggtgtgtgctgctccacaggcctggtg

SEQ ID NO: 741 - RNA, 65 nt, Homo sapiens
  source 1..65: mol_type = transcribed RNA
ctcctctgggggtggggggctgggcgtggtggacagcgatgcatccctcgccttctcaccctcag

SEQ ID NO: 742 - RNA, 67 nt, Homo sapiens
  source 1..67: mol_type = transcribed RNA
ggcagccagggggatgggcgagcttgggcccattcctttccttaccctaccccccatccccctgtag

SEQ ID NO: 743 - RNA, 180 nt, Homo sapiens
  source 1..180: mol_type = transcribed RNA
cgcgactgcggcggcggtggtggggggagccgcggggatcgccgagggccggtcggccgccccgggtgccgcgcggtgccgccggcggcggtgaggccccgcgcgtgtgtcccggctgcggtcggccgcgctcgaggggtccccgtggcgtccccttccccgccggccgcctttctcgcg

SEQ ID NO: 744 - RNA, 82 nt, Homo sapiens
  source 1..82: mol_type = transcribed RNA
gggaaagcggagggcgcgcccagctcccgggctgattgcgctaacagtggccccggtgttggggcgcgtctgccgctgcccc

SEQ ID NO: 745 - RNA, 63 nt, Homo sapiens
  source 1..63: mol_type = transcribed RNA
ggctccgcagggccctggcgcaggcatccagacagcgggcgaatgcctcccccggccccgcag

SEQ ID NO: 746 - RNA, 20 nt, Homo sapiens
  source 1..20: mol_type = transcribed RNA
ggctccttggtctaggggta

SEQ ID NO: 747 - RNA, 15 nt, Homo sapiens
  source 1..15: mol_type = transcribed RNA
cttggtctaggggta

SEQ ID NO: 748 - RNA, 25 nt, Homo sapiens
  source 1..25: mol_type = transcribed RNA
aggaagccctggaggggctggaggt

SEQ ID NO: 749 - RNA, 15 nt, Homo sapiens
  source 1..15: mol_type = transcribed RNA
aggaagaggaggaag

SEQ ID NO: 750 - RNA, 24 nt, Homo sapiens
  source 1..24: mol_type = transcribed RNA
gatcggtcgagagcgtcctggctg

SEQ ID NO: 751 - RNA, 15 nt, Homo sapiens
  source 1..15: mol_type = transcribed RNA
gctgggcggggcgcg

SEQ ID NO: 752 - RNA, 23 nt, Homo sapiens
  source 1..23: mol_type = transcribed RNA
tggggaaggcgtcagtgtcgggt

SEQ ID NO: 753 - RNA, 16 nt, Homo sapiens
  source 1..16: mol_type = transcribed RNA
tggggaaggcgtcagt

SEQ ID NO: 754 - RNA, 23 nt, Homo sapiens
  source 1..23: mol_type = transcribed RNA
aggaaggaaggcagggcccccgc

SEQ ID NO: 755 - RNA, 15 nt, Homo sapiens
  source 1..15: mol_type = transcribed RNA
gggcccccgctcccc

SEQ ID NO: 756 - RNA, 20 nt, Homo sapiens
  source 1..20: mol_type = transcribed RNA
ggaaaaaggcgggagaagcc

SEQ ID NO: 757 - RNA, 15 nt, Homo sapiens
  source 1..15: mol_type = transcribed RNA
ggcgggagaagcccc

SEQ ID NO: 758 - RNA, 20 nt, Homo sapiens
  source 1..20: mol_type = transcribed RNA
tgagtggggctcccgggacg

SEQ ID NO: 759 - RNA, 20 nt, Homo sapiens
  source 1..20: mol_type = transcribed RNA
tgagtggggctcccgggacg

SEQ ID NO: 760 - RNA, 22 nt, Homo sapiens
  source 1..22: mol_type = transcribed RNA
tggcggagcccattccatgcca

SEQ ID NO: 761 - RNA, 21 nt, Homo sapiens
  source 1..21: mol_type = transcribed RNA
ctggcggagcccattccatgc

SEQ ID NO: 762 - RNA, 29 nt, Homo sapiens
  source 1..29: mol_type = transcribed RNA
gggagccgcggggatcgccgagggccggt

SEQ ID NO: 763 - RNA, 15 nt, Homo sapiens
  source 1..15: mol_type = transcribed RNA
ggcggcggtggtggg

SEQ ID NO: 764 - RNA, 22 nt, Homo sapiens
  source 1..22: mol_type = transcribed RNA
ccccggtgttggggcgcgtctg

SEQ ID NO: 765 - RNA, 21 nt, Homo sapiens
  source 1..21: mol_type = transcribed RNA
cccggtgttggggcgcgtctg